(19) United States
(12) Patent Application Publication
(10) Pub. No.: US 2015/0328292 A1
(43) Pub. Date: Nov. 19, 2015

(54) CASPASE POLYPEPTIDES HAVING MODIFIED ACTIVITY AND USES THEREOF

(71) Applicant: BELLICUM PHARMACEUTICALS, INC., Houston, TX (US)

(72) Inventors: David SPENCER, Houston, TX (US); Wei-Chun CHANG, Houston, TX (US); Kevin SLAWIN, Houston, TX (US)

(21) Appl. No.: 14/640,553

(22) Filed: Mar. 6, 2015

Related U.S. Application Data
(60) Provisional application No. 61/949,787, filed on Mar. 7, 2014.
(60) Provisional application No. 62/007,902, filed on Jun. 4, 2014.
(60) Provisional application No. 62/047,870, filed on Sep. 9, 2014.

(51) Int. Cl. A61K 38/48; A61K 38/52; A61K 31/4545; C12N 9/64

(52) U.S. Cl. CPC A61K 38/4873; C12N 9/6472; A61K 38/52; A61K 31/4545; C12Y 304/22062; C12Y 502/01008; A61K 48/00

(57) ABSTRACT

The technology relates in part to compositions comprising modified caspase-9 polypeptides, compositions comprising nucleic acids coding for modified caspase-9 polypeptides, chimeric modified caspase-9 polypeptides, and methods of use thereof, including methods for cell therapy. Methods for cell therapy include modifying transfused cells to express an inducible modified caspase-9 protein, with reduced basal activity in the absence of the inducer.

### RELATED APPLICATIONS

Priority is claimed to U.S. Provisional Patent Application Ser. No. 61/949,787, filed Mar. 7, 2014, entitled “Caspase Polypeptides Having Modified Activity and Uses Thereof,” to U.S. Provisional Patent Application Ser. No. 62/007,902, filed Jun. 4, 2014, entitled “Caspase Polypeptides Having Modified Activity and Uses Thereof,” and to U.S. Provisional Patent Application Ser. No. 62/047,870, filed Sep. 9, 2014, entitled “Caspase Polypeptides Having Modified Activity and Uses Thereof,” which are all referred to and incorporated by reference thereof, in their entireties.

### FIELD

The technology relates generally to the field of immunology and relates in part to compositions comprising modified caspase-9 polypeptides, compositions comprising nucleic acids coding for modified caspase-9 polypeptides, chimeric modified caspase-9 polypeptides, and methods of use thereof, including methods for cell therapy. Methods for cell therapy include modifying transfused cells to express an inducible modified caspase-9 protein, with reduced basal activity in the absence of the inducer.

### SEQUENCE LISTING

The instant application contains a Sequence Listing which has been submitted electronically in ASCII format and is hereby incorporated by reference in its entirety. Said ASCII copy, created on Apr. 6, 2015, is named BEL-2015-UT_SL.txt and is 291,996 bytes in size.

### BACKGROUND

There is an increasing use of cellular therapy in which modified or unmodified cells are administered to a patient. An example of a cellular therapy is adoptive T cell transfer after CD34⁺ stem cell transplantation. Administering T cells after stem cell transfer helps to accelerate the reconstitution of an immune system in the patient recipient. When a matched related or unrelated donor is not available, or the disease is too aggressive for an extensive donor search, the use of an HLA haploidentical family donor may be effective. Such donors may be parents, siblings, or second-degree relatives. Such infusions may enhance immune recovery and thereby reduce virus infections and eliminate relapsing leukemia cells. However, the coexistence of alloreactive T cells in a donor stem cell graft may cause graft-versus-host disease (GvHD) in which the donor cells react against the recipient, which may progressively damage the skin, gut, liver, and other organs of the recipient, often with fatal consequences.

Other examples of cell therapies include using native cells or cells genetically engineered to express a heterologous gene. These treatments are used, for example, for blood disorders, but these therapies may have negative side effects. In other methods, immature progenitor cells that can differentiate into many types of mature cells, such as, for example, mesenchymal stromal cells, may be used to treat disorders by replacing the function of diseased cells.

Yet other examples of cell therapies comprise using therapeutic cells that express chimeric antigen receptors specific for a targeted cell. Chimeric antigen receptor-expressing T cells may be used in various therapies, including cancer therapies. Adverse events are possible, as the therapeutic cells may have toxic effects; in at least one reported case, the therapy had fatal consequences to the patient.

Methods for inducing selective apoptosis using a caspase-9 system, using ligand-mediated dimerization and activation of a chimeric caspase-9 polypeptide are discussed in U.S. patent application Ser. No. 13/112,739, filed May 20, 2011, and entitled METHODS FOR INDUCING SELECTIVE APOPTOSIS, naming Malcolm K. Brenner as inventor, in U.S. patent application Ser. No. 13/792,135, filed Mar. 10, 2013, entitled “Modified Caspase Polypeptides and Uses Thereof,” and to International Patent Application Serial Number PCT/US2014/022004, filed Mar. 7, 2014, published as WO2014/164348 on Oct. 9, 2014, and entitled “Modified Caspase Polypeptides and Uses Thereof,” which are hereby incorporated by reference herein in their entireties.

There is a need for a rapid and effective mechanism to remove possible negative effects of donor therapeutic cells used in cellular therapy, by providing an inducible Caspase system with lower basal activity.

### SUMMARY

Selective apoptosis may be induced in cells that express a chimeric caspase-9 polypeptide fused to a dimeric ligand-binding polypeptide, such as, for example, the AP1903-binding polypeptide FKBP12v36. Although the chimeric caspase-9 polypeptide is exquisitely sensitive to AP1903 (rimiducid) and triggers apoptosis in a variety of target cells with an EC₅₀˜10 to 100 pM, low-level ligand-independent activity is greatly amplified in producer cell lines, creating a manufacturing challenge, where genome levels can be orders of magnitude greater than typical functional levels. Moreover, even low-level basal activity reaches pro-apoptotic, super-threshold levels in a fraction of transduced or transfected target cells, reducing the cell product yield. Finally, it is likely that some cells will be more sensitive than others to this low, but measurable pro-apoptotic basal activity. To develop a chimeric caspase-9 polypeptide with reduced basal activity, site-directed mutagenesis was performed on targeted regions of caspase-9, including (i) the homo-dimerization interface, (ii) aspartic acid residues critical for Caspase-3-mediated or auto-cleavage, (iii) residues in direct contact with inhibitor of apoptosis, XIAP, and (iv) validated phosphorylation sites.

Chimeric polypeptides comprising modified caspase-9 polypeptides, including, for example, iCasp9 D330A, iCasp9 N405Q, and iCasp9 D330A N405Q, demonstrated low to undetectable basal activity, respectively, with a minimum deleterious effect on their AP1903 IC₅₀ in a SEAP reporter-based, surrogate killing assay.

In some embodiments, a cell is provided which comprises a polynucleotide that encodes a chimeric protein comprising a multimerization region and a modified caspase-9 polypeptide, wherein the modified caspase-9 polypeptide comprises an amino acid sequence having at least 90% sequence identity to SEQ ID NO: 9, and comprises at least one amino acid substitution selected from the group consisting of S144A, S144D, Y153A, Y153F, S183A, S195A, S196A, S196D, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, C403A, C403S, C403T, F404T, F404W, F404Y, N405A, N405F, N405Q, N405T, F406A, F406T, F406W, F406Y, G402A, G402I, G402Q, G402Y, C403P, F404A, F404S, and F406L. In some embodiments, the at least one amino acid substitution is selected from the group consisting of S144A, S144D, Y153A, Y153F, S183A, S195A, S196A, S196D, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, C403A, C403S, C403T, F404T, F404W, F404Y, N405A, N405F, N405Q, N405T, F406A, F406T, F406W, and F406Y. In some embodiments, the at least one amino acid substitution is selected from the group consisting of S144A, S144D, Y153A, Y153F, S183A, S195A, S196A, S196D, S307A, D315A, A316G, T317A, T317C, T317S, P318A, F319A, F319W, L329E, D330A, D330E, D330G, D330N, D330S, D330V, C403A, C403S, C403T, F404T, F404W, F404Y, N405A, N405F, N405Q, F406A, F406T, F406W, and F406Y. In some embodiments, the at least one amino acid substitution is S144A, S144D, Y153A, Y153F, S183A, S195A, S196A, S196D, S307A, D315A, A316G, T317A, T317S, F319W, L329E, D330A, D330E, D330G, D330N, D330S, D330V, F404T, F404W, F404Y, N405F, N405Q, F406A, F406T, F406W, and F406Y. In some embodiments, the at least one amino acid substitution is S144A, S144D, S183A, S195A, S196A, S196D, T317A, T317S, L329E, D330A, D330E, D330G, D330N, D330S, D330V, F404Y, N405Q, F406A, F406W, and F406Y. In some embodiments, the at least one amino acid substitution is selected from the group consisting of T317S, S144A, S133, and S196D. In some embodiments, the at least one amino acid substitution is selected from the group consisting of S183A, S195A, S196A, S196D, T317A, L329E, D330A, D330E, D330G, D330N, D330S, D330V, F404Y, N405Q, F406A, F406W, and F406Y. In some embodiments, the at least one amino acid substitution is D330A. In some embodiments, the at least one amino acid substitution is D330E. In some embodiments, the at least one amino acid substitution is N405Q. In some embodiments, the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, ⁴⁰²GCFNF⁴⁰⁶ISAQT (CASP-10) (SEQ ID NOS 224 and 225), ³¹⁶ATPF³¹⁹AVPI (SMAC/Diablo) (SEQ ID NOS 226 and 227), D330A-N405T, D315A-D330A, D330A-Y153A, D330A-Y153F, D330A-T317E, ⁴⁰²GCFNF⁴⁰⁶CIVSM (CASP-3) (SEQ ID NOS 224 and 228), ⁴⁰²GCFNF⁴⁰⁶AAAAA (SEQ ID NOS 224 and 229), ⁴⁰²GCFNF⁴⁰⁶YCSTL (CASP-2) (SEQ ID NOS 224 and 230), and ⁴⁰²GCFNF⁴⁰⁶QPTFT (CASP-8) (SEQ ID NOS 224 and 231). In some embodiments, the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, ⁴⁰²GCFNF⁴⁰⁶ISAQT (CASP-10) (SEQ ID NOS 224 and 225), ³¹⁶ATPF³¹⁹AVPI (SMAC/Diablo) (SEQ ID NOS 226 and 227), and D330A-N405T. In some embodiments, the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, ⁴⁰²GCFNF⁴⁰⁶ISAQT (CASP-10) (SEQ ID NOS 224 and 225), and ³¹⁶ATPF³¹⁹AVPI (SMAC/Diablo) (SEQ ID NOS 226 and 227). In some embodiments, the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, and ⁴⁰²GCFNF⁴⁰⁶ISAQT (CASP-10) (SEQ ID NOS 224 and 225). In some embodiments, the modified caspase-9 polypeptide comprises at least two amino acid substitutions selected from the group consisting of N405Q-S144Aco and N405Q-T317Sco. In some embodiments, the at least one amino acid substitution is selected from the group consisting of S144A, S144D, Y153A, Y153F, S183A, S195A, S196A, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, F404T, F404W, F404YN405F, N405Q, and F406T. In some embodiments, the at least one amino acid substitution is selected from the group consisting of S144A, S144D, Y153A, Y153F, S183A, S195A, S196A, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, F404T, F404W, F404Y, N405F, N405Q, and F406T. In some embodiments, the at least one amino acid substitution is selected from the group consisting of S144A, S144D, Y153A, Y153F, S183A, S195A, S196A, S307A, D315A, A316G, T317A, T317C, T317S, P318A, F319A, F319W, L329E, D330A, D330E, D330G, D330N, D330S, D330V, F404T, F404W, F404Y, N405F, N405Q, and F406T. In some embodiments, the at least one amino acid substitution is selected from the group consisting of S144A, S144D, Y153A, Y153F, S183A, S195A, S196A, S307A, D315A, A316G, T317A, T317S, F319W, D330A, F404T, F404W, F404Y, N405F, N405Q, and F406T. In some embodiments, the at least one amino acid substitution is selected from the group consisting of S144A, S144D, S183A, S195A, S196A, T317A, T317S, D330A, F404Y, and N405Q. In some embodiments, the at least one amino acid substitution is selected from the group consisting of S196D, T317C, T317E, P318A, F319A, F326K, D327G, D327K, D327R, Q328K, Q328R, L329E, L329G, L329K, D330E, D330G, D330N, D330S, D330V, A331K, C403A, C403S, C403T, N405A, N405T, F406A, F406W, F406Y, G402A, G402I, G402Q, G402Y, C403P, F404A, F404S, and F406L. In some embodiments, the at least one amino acid substitution is selected from the group consisting of S196D, T317C, T317E, P318A, F319A, F326K, D327G, D327K, D327R, Q328K, Q328R, L329E, L329G, L329K, D330E, D330G, D330N, D330S, D330V, A331K, C403A, C403S, C403T, N405A, N405T, F406A, F406W, and F406Y. In some embodiments, the at least one amino acid substitution is selected from the group consisting of S196D, T317C, P318A, F319A, L329E, D330E, D330G, D330N, D330S, D330V, C403A, C403S, C403T, N405A, F406A, F406T, F406W, and F406Y. In some embodiments, the at least one amino acid substitution is selected from the group consisting of S196D, L329E, D330E, D330G, D330N, D330S, D330V, F406A, F406T, F406W, and F406Y. In some embodiments, the at least one amino acid substitution is selected from the group consisting of S196D, L329E, D330E, D330G, D330N, D330S, D330V, F406A, F406W, and F406Y. 47. The cell of claim 1, wherein, the at least one amino acid substitution is selected from the group consisting of N405Q, F404Y, F406A, F406W, F406Y, F404T, F404W, N405F, F406T, C403A, C403S, C403T, N405A, and N405T. In some embodiments, the at least one amino acid substitution is selected from the group consisting of N405Q, F404Y, F406A, F406W, and F406Y. In some embodiments, the at least one amino acid substitution is selected from the group consisting of T317S, D330A, D330E, D330G, D330N, D330S, D330V, L329E, T317A, D315A, A316G, T317C, P318A, F319A, T317E, F326K, D327G, D327K, D327R, Q328K, Q328R, L329G, L329K, and A331K. In some embodiments, the at least one amino acid substitution is selected from the group consisting of T317S, D330A, D330E, D330G, D330N, D330S, D330V, L329E, and T317A. In some embodiments, the at least one amino acid substitution is selected from the group consisting of S144A, S144D, S196D, S183A, S195A, S196A, Y153A, Y153F, and S307A.

In some embodiments, the polynucleotide comprises optimized codons encoding the caspase-9 polypeptide, in some embodiments, the polynucleotide coding for the modified caspase-9 polypeptide comprises an amino acid substitution of N405Q, and comprises optimized codons. In some embodiments, the polynucleotide coding for the modified caspase-9 polypeptide comprises the nucleic acid sequence of SEQ ID NO: 39.

Also provided are modified caspase polypeptides comprising the amino acid sequences discussed herein as part of the cells which comprise polynucleotides coding for the chimeric modified caspase-9 polypeptides. Also provided are nucleic acids comprising polynucleotides coding for the modified caspase-9 polypeptides and the chimeric modified caspase-9 polypeptides. Also provided are vectors comprising the polynucleotides coding for the modified caspase polypeptides and the chimeric modified caspase polypeptides.

In some embodiments, the cell is a human cell. The cell of the present application may be any type of eukaryotic cell, for example a mammalian cell, for example a horse, dog, cat, cow, or human cell. In some embodiments, the cell is a progenitor cell. In some embodiments, the cell is a hematopoietic progenitor cell. In some embodiments, the cell is selected from the group consisting of mesenchymal stromal cells, embryonic stem cells, and inducible pluripotent stem cells. In some embodiments, the cell is a T cell. In some embodiments, the cell is obtained or prepared from bone marrow. In some embodiments, the cell is obtained or prepared from umbilical cord blood. In some embodiments, the cell is obtained or prepared from peripheral blood. In some embodiments, the cell is obtained or prepared from peripheral blood mononuclear cells.

In some aspects, the polynucleotide coding for the chimeric polypeptide or modified caspase-9 polypeptide is operably linked to a promoter. In some embodiments, the promoter is developmentally regulated and the caspase-9 polypeptide is expressed in developmentally differentiated cells. In some embodiments, the promoter is tissue-specific and the caspase-9 polypeptide is expressed in the specific tissue. In some embodiments, the promoter is activated in activated T cells. In some embodiments, the promoter comprises a 5′LTR sequence. In some embodiments, the chimeric protein further comprises a marker polypeptide, for example, but not limited to, a ΔCD19 polypeptide. In some embodiments, the caspase-9 polypeptide is a truncated caspase-9 polypeptide. In some embodiments, the caspase-9 polypeptide lacks the Caspase recruitment domain.

In some embodiments, the multimerization region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof. In some embodiments, the multimerization region is an FKBP12 region. In some embodiments, the FKB12 region is an FKB12v₃₆ region. In some embodiments, the multimerization region is Fv′Fvls. In some embodiments, the multimerization region binds a ligand selected from the group consisting of an FK506 dimer and a dimeric FK506 analog ligand. In some embodiments, the ligand is AP1903, in other embodiments, the ligand is AP20187. In some embodiments, wherein the multimerization region has an amino acid sequence of SEQ ID NO: 29 or a functional fragment thereof. In some embodiments, the multimerization region is encoded by a nucleotide sequence in SEQ ID NO: 30, or a functional fragment thereof. In some embodiments, the multimerization region further comprises a polypeptide having an amino acid sequence of SEQ ID NO: 32, or a functional fragment thereof. In some embodiments, the multimerization region further comprises a polypeptide encoded by a nucleotide sequence in SEQ ID NO: 31, or a functional fragment thereof. In some embodiments, the multimerization region further comprises a polypeptide having an amino acid sequence of SEQ ID NO: 32, or a functional fragment thereof. In some embodiments, the multimerization region further comprises a polypeptide encoded by a nucleotide sequence in SEQ ID NO: 31, or a functional fragment thereof. In some embodiments, the multimerization region further comprises a polypeptide having an amino acid sequence of SEQ ID NO: 29 or SEQ ID NO: 32, or a functional fragment thereof. In some embodiments, the multimerization region further comprises a polypeptide encoded by a nucleotide sequence in SEQ ID NO: 30 or SEQ ID NO: 31, or a functional fragment thereof.

In some aspects of the present application, the cells are transduced or transfected with a viral vector. The viral vector may be, for example, but not limited to, a retroviral vector, such as, for example, but not limited to, a murine leukemia virus vector; an SFG vector; and adenoviral vector, or a lentiviral vector.

In some aspects, the cells are further transfected or transduced with a gene expression vector. In some embodiments, the cells further comprise a polynucleotide that encodes the modified caspase-9 polypeptide and further comprise a second polynucleotide that encodes a heterologous polypeptide. By “heterologous polypeptide” is meant a polypeptide other than the chimeric caspase polypeptide of the present application that is expressed by a cell transfected or transduced by a polynucleotide. The heterologous polypeptide may be a polypeptide that is not naturally expressed in the cell, or may be a polypeptide that is not naturally expressed in a non-transfected or transduced, wild type, cell at the same level as in the transfected or transduced cell. In some embodiments, the heterologous polypeptide is a chimeric antigen receptor. In some embodiments, the cells further comprises a polynucleotide that encodes a chimeric protein comprising a multimerization region and a caspase-9 polypeptide or a second modified caspase-9 polypeptide, wherein the first and second caspase-9 polypeptides comprise different amino acid sequences and have different basal activities or different IC₅₀s. In some aspects is provided a cell, wherein the modified caspase-9 polypeptide and the caspase-9 polypeptide; or the modified caspase-9 polypeptide and the second modified caspase-9 polypeptide have different IC₅₀s or different elongated dose response curves, or different IC₅₀s and different elongated dose response curves to the multimeric ligand.

In some embodiments, the cell is isolated. In some embodiments, the cell is in a human subject. In some embodiments, the cell is transplanted in a human subject.

Also provided are methods of administering donor T cells to a human patient, comprising administering any of the cells of the present application to a human patient, wherein the cells are human donor T cells. In some embodiments, the cells are transduced or transfected in a donor cell culture. In some embodiments, the method further comprises detecting the presence of graft versus host disease in the patient after administering the cells to the patient; and administering a multimeric ligand that binds to the multimerization region to the patient for whom the presence of graft versus host disease is detected. In some embodiments, the effect of the graft versus host disease is reduced following administration of the multimeric ligand.

Also provided are methods of stem cell transplantation, comprising administering a stem cell transplant to a human patient; and administering cells of the present application to the patient, wherein the cells are donor T cells, to the patient. In some embodiments, the stem cell transplant is selected from the group consisting of a matched transplant, a partially-matched transplant, a haploidentical transplant, and a CD34⁺ haploidentical stem cell transplant. In some embodiments, the human donor T cells are matched, partially matched, or haploidentical to the patient's T cells.

Also provided in certain aspects are methods for controlling the survival of transplanted therapeutic cells in a patient, comprising administering cells of the present application to a human patient, and administering a multimeric ligand to the patient, wherein the multimeric ligand binds to the multimerization region, wherein the administered cells that express the caspase-9 polypeptide are killed following administration of the multimeric ligand. In some embodiments, the method comprises preparing cells of the present application for transplantation, and transplanting the therapeutic cells into the human patient.

In some embodiments, the patient has cancer. In some embodiments, the patient has a solid tumor. In some embodiments, the cancer is present in the blood or bone marrow of the patient. In some embodiments, the patient has a blood or bone marrow disease. In some embodiments, the patient has been diagnosed with any condition or disorder that can be alleviated by stem cell transplantation.

The methods of the present application may further comprise a selection step, wherein cells that express a marker are selected for administration to the patient. The marker may be, for example, but not limited to, ΔCD19. In some embodiments, the cells are transfected in a donor cell culture that is prepared from a bone marrow sample. In some embodiments, the cells are transfected in a donor cell culture that is prepared from peripheral blood. In some embodiments, the donor cell culture is prepared from donor peripheral blood mononuclear cells. In some embodiments, the donor T cells are allodepleted from the donor cell culture before transfection or transduction. In some embodiments, the donor T cells are not allodepleted from the donor cell culture before transfection or transduction. In some embodiments, the transduced or transfected T cells are cultured in the presence of IL-2 before administration to the patient.

In certain embodiments, the methods of the present application further comprise administering a multimeric ligand that binds to the multimerization region, such as, for example, AP1903 or AP20187. In some embodiments, the multimeric ligand is administered to treat graft versus host disease. In some embodiments, the patient exhibits graft versus host disease symptoms before the multimeric ligand is administered. In some embodiments, the patient exhibits one or more Stage 0, Stage 1, Stage 2, Stage 3, or Stage 4 graft versus host disease symptoms.

In certain embodiments of the methods, more than one dose of the multimeric ligand is administered. In some embodiments, after administration of the multimeric ligand, the number of alloreactive T cells is reduced. In some embodiments, the alloreactive T cells express the marker and CD3. In some embodiments, the number of alloreactive T cells is reduced by about 90% or more after administration of the multimeric ligand. In some embodiments, after administration of the multimeric ligand, donor T cells survive in the patient that are able to expand and are reactive to viruses and fungi. In some embodiments, after administration of the multimeric ligand, donor T cells survive in the patient that are able to expand and are reactive to tumor cells in the patient.

In some embodiments, the patient has received stem cell transplants before or at the same time as administration of the donor T cells. In some embodiments, the stem cell transplant is haploidentical. In some embodiments, the donor T cells are haploidentical and are not allodepleted before administration to the patient.

In some embodiments, at least 1×10⁵ transduced or transfected donor T cells are administered to the patient. In some embodiments, at least 1×10⁶ transduced or transfected donor T cells are administered to the patient. In some embodiments, at least 1×10⁷ transduced or transfected donor T cells are administered to the patient. In some embodiments, at least 1×10⁸ transduced or transfected donor T cells are administered to the patient. In some embodiments, at least 1×10⁹ transduced or transfected donor T cells are administered to the patient.

In some embodiments, at least 1×10⁵ transduced or transfected therapeutic cells are administered to the patient. In some embodiments, at least 1×10⁷ transduced or transfected donor T cells are administered to the patient. In some embodiments, at least 1×10⁸ transduced or transfected donor T cells are administered to the patient. In some embodiments, at least 1×10⁹ transduced or transfected donor T cells are administered to the patient. In some embodiments, at least 1×10⁶ transduced or transfected therapeutic cells are administered to the patient. In some embodiments, at least 1×10⁷ transduced or transfected therapeutic cells are administered to the patient. In some embodiments, at least 1×10⁸ transduced or transfected therapeutic cells are administered to the patient. In some embodiments, at least 1×10⁹ transduced or transfected therapeutic cells are administered to the patient.

In some embodiments, personalized treatment is provided wherein the stage or level of the disease or condition is determined before administration of the multimeric ligand, before the administration of an additional dose of the multimeric ligand, or in determining method and dosage involved in the administration of the multimeric ligand. These methods may be used in any of the methods of any of the diseases or conditions of the present application. Where these methods of assessing the patient before administering the ligand are discussed in the context of graft versus host disease, it is understood that these methods may be similarly applied to the treatment of other conditions and diseases. Thus, for example, in some embodiments of the present application, the method comprises administering therapeutic cells to a patient, and further comprises identifying a presence or absence of a condition in the patient that requires the removal of transfected or transduced therapeutic cells from the patient; and administering a multimeric ligand that binds to the multimerization region, maintaining a subsequent dosage of the multimeric ligand, or adjusting a subsequent dosage of the multimeric ligand to the patient based on the presence or absence of the condition identified in the patient. And, for example, in other embodiments of the present application, the method further comprises determining whether to administer an additional dose or additional doses of the multimeric ligand to the patient based upon the appearance of graft versus host disease symptoms in the patient. In some embodiments, the method further comprises identifying the presence, absence or stage of graft versus host disease in the patient, and administering a multimeric ligand that binds to the multimerization region, maintaining a subsequent dosage of the multimeric ligand, or adjusting a subsequent dosage of the multimeric ligand to the patient based on the presence, absence or stage of the graft versus host disease identified in the patient. In some embodiments, the method further comprises identifying the presence, absence or stage of graft versus host disease in the patient, and determining whether a multimeric ligand that binds to the multimerization region should be administered to the patient, or the dosage of the multimeric ligand subsequently administered to the patient is adjusted based on the presence, absence or stage of the graft versus host disease identified in the patient. In some embodiments, the method further comprises receiving information comprising the presence, absence or stage of graft versus host disease in the patient; and
administering a multimeric ligand that binds to the multimerization region, maintaining a subsequent dosage of the multimeric ligand, or adjusting a subsequent dosage of the multimeric ligand to the patient based on the presence, absence or stage of the graft versus host disease identified in the patient. In some embodiments, the method further comprises identifying the presence, absence or stage of graft versus host disease in the patient, and transmitting the presence, absence or stage of the graft versus host disease to a decision maker who administers a multimeric ligand that binds to the multimerization region, maintains a subsequent dosage of the multimeric ligand, or adjusts a subsequent dosage of the multimeric ligand administered to the patient based on the presence, absence or stage of the graft versus host disease identified in the subject. In some embodiments, the method further comprises identifying the presence, absence or stage of graft versus host disease in the patient, and transmitting an indication to administer a multimeric ligand that binds to the multimeric binding region, maintain a subsequent dosage of the multimeric ligand or adjust a subsequent dosage of the multimeric ligand administered to the patient based on the presence, absence or stage of the graft versus host disease identified in the subject.

In some aspects, methods are provided for treating graft versus host disease in a patient who has undergone cell therapy, wherein one or more of the cells introduced for the therapy is a cell of the present application, comprising administering a multimeric ligand that binds to the multimerization region to the patient. In some embodiments, after administration of the multimeric ligand that binds to the multimeric binding region, the number of alloreactive T cells is reduced. In some embodiments, alloreactive T cells that are not undergoing cell division are ablated. In some embodiments, within 2 hours of administration of the multimeric ligand, at least 90% of CD3+ΔCD19+ cells are ablated. In some embodiments, within 1 hour of administration of the multimeric ligand, at least 90% of CD3+ΔCD19+ cells are ablated. In some embodiments, within 30 minutes of administration of the multimeric ligand, at least 90% of CD3+ΔCD19+ cells are ablated. In some embodiments, within 24 hours of administration of the multimeric ligand, there is a further log reduction of CD3+ΔCD19+ cells compared to the amount of CD3+ΔCD19+ cells at 30 minutes after administration of the multimeric ligand. In some embodiments, the method further comprises a resolution of skin and liver GvHD within 24 hours after administration of the multimeric ligand.

In some embodiments the cells are therapeutic cells and are transduced or transfected with a second nucleic acid that encodes a second heterologous protein. In some embodiments, the therapeutic cells are transduced with a heterologous gene that expresses a chimeric antigen receptor. In some embodiments, the therapeutic cells are transduced with a heterologous gene that expresses a modified TGF-beta receptor. In some embodiments, the therapeutic cells are transduced with the heterologous gene before, at the same time as, or after being transduced with the nucleic acid encoding the chimeric protein comprising a multimerization region and a caspase-9 polypeptide.

Also provided is a method for administering donor T cells to a human patient, comprising administering a transduced or transfected T cell of the present application to a human patient, wherein the cells are non-allodepleted human donor T cells.

In some embodiments, the therapeutic cells are administered to a subject having a non-malignant disorder, or where the subject has been diagnosed with a non-malignant disorder, such as, for example, a primary immune deficiency disorder (for example, but not limited to, Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCK 8 Deficiency, IL-10 Deficiency/IL-10 Receptor Deficiency, GATA 2 deficiency, X-linked lymphoproliferative disease (XLP), Cartilage Hair Hypoplasia, and the like), Hemophagocytosis Lymphohistiocytosis (HLH) or other hemophagocytic disorders, Inherited Marrow Failure Disorders (such as, for example, but not limited to, Shwachman Diamond Syndrome, Diamond Blackfan Anemia, Dyskeratosis Congenita, Fanconi Anemia, Congenital Neutropenia, and the like), Hemoglobinopathies (such as, for example, but not limited to, Sickle Cell Disease, Thalassemia, and the like), Metabolic Disorders (such as, for example, but not limited to, Mucopolysaccharidosis, Sphingolipidoses, and the like), or an Osteoclast disorder (such as, for example, but not limited to Osteopetrosis).

The therapeutic cells may be, for example, any cell administered to a patient for a desired therapeutic result. The cells may be, for example, T cells, natural killer cells, B cells, macrophages, peripheral blood cells, hematopoietic progenitor cells, bone marrow cells, or tumor cells. The modified caspase-9 polypeptide can also be used to directly kill tumor cells. In one application, vectors comprising polynucleotides coding for the inducible modified caspase-9 polypeptide would be injected into a tumor and after 10-24 hours (to permit protein expression), the ligand inducer, such as, for example, AP1903, would be administered to trigger apoptosis, causing the release of tumor antigens to the microenvironment. To further improve the tumor microenvironment to be more immunogenic, the treatment may be combined with one or more adjuvants (e.g., IL-12, TLRs, IDO inhibitors, etc.). In some embodiments, the cells may be delivered to treat a solid tumor, such as, for example, delivery of the cells to a tumor bed. In some embodiments, a polynucleotide encoding the chimeric caspase-9 polypeptide may be administered as part of a vaccine, or by direct delivery to a tumor bed, resulting in expression of the chimeric caspase-9 polypeptide in the tumor cells, followed by apoptosis of tumor cells following administration of the ligand inducer. Thus, also provided in some embodiments are nucleic acid vaccines, such as DNA vaccines, wherein the vaccine comprises a nucleic acid comprising a polynucleotide that encodes an inducible, or modified inducible caspase-9 polypeptide of the present application. The vaccine may be administered to a subject, thereby transforming or transducing target cells in vivo. The ligand inducer is then administered following the methods of the present application.

In some embodiments, the modified caspase-9 polypeptide is a truncated modified caspase-9 polypeptide. In some embodiments, the modified caspase-9 polypeptide lacks the Caspase recruitment domain. In some embodiments, the caspase-9 polypeptide comprises the amino acid sequence of SEQ ID NO: 9, or a fragment thereof, or is encoded by the nucleotide sequence of SEQ ID NO: 8, or a fragment thereof.

In some embodiments, the methods further comprise administering a multimeric ligand that binds to the multimeric ligand binding region. In some embodiments, the multimeric ligand binding region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof. In some embodiments, the multimeric ligand binding region is an FKBP12 region. In some embodiments, the multimeric ligand is an FK506 dimer or a dimeric FK506-like analog ligand. In some embodiments, the multimeric ligand is AP1903. In some embodiments, the multimeric ligand is administered to treat graft versus host disease. In some embodiments, the patient exhibits graft versus host disease symptoms before the multimeric ligand is administered. In some embodiments, the patient exhibits one or more Stage 0 graft versus host disease symptoms. In some embodiments, the patient exhibits one or more Stage 1 graft versus host disease symptoms. In some embodiments, the patient exhibits one or more Stage 2 graft versus host disease symptoms. In some embodiments, the patient exhibits one or more Stage 3 graft versus host disease symptoms. In some embodiments, the patient exhibits one or more Stage 4 graft versus host disease symptoms. In some embodiments, more than one dose of the multimeric ligand is administered. In some embodiments, after administration of the multimeric ligand, the number of alloreactive T cells is reduced. In some embodiments, the number of alloreactive T cells is reduced by from about 60% to 99%, about 70% to 95%, from 80% to 90% or about 90% or more after administration of the multimeric ligand. In some embodiments, after administration of the multimeric ligand, donor T cells survive in the patient that are able to expand and are reactive to viruses and fungi. In some embodiments, after administration of the multimeric ligand, donor T cells survive in the patient that are able to expand and are reactive to tumor cells in the patient.

In some embodiments, the subject is a mammal. In some embodiments, the subject is a human.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1A illustrates iCasp9 expression vectors as discussed herein in Example 1. FIG. 1B is a representative western blot of full length and truncated caspase-9 protein produced by the expression vectors shown in FIG. 1A. FIG. 1A discloses “GCCACC” as SEQ ID NO: 259 and “Ser-Gly-Gly-Gly-Ser” as SEQ ID NO: 236.

FIGS. 2A-2D graphically present results of experiments performed to evaluate the effect of expression of iCasp9 expression constructs on the phenotype of cells transduced with various iCasp9 expression vectors. FIG. 2A illustrates levels of cell surface markers in transduced and nontransduced cells. FIG. 2B illustrates levels of secretion of Th1 and Th2 type cytokines upon antigen stimulation in transduced and nontransduced cells. FIG. 2C illustrates levels of cytolytic activity against autologous EVB-transformed lymphoblastoid B-cell line (LCL), HLA-mismatched LCL, and HSB-2 in transduced and nontransduced cells. FIG. 2D illustrates the persistence of antigen dependence on iCasp9 transduced cell lines. Note the steady decline of T cells after antigen stimulation is discontinued. Further discussion of experimental conditions and results are presented in the Examples.

FIGS. 3A-3D provide the results of various experiments performed to determine the efficacy of a chemical inducer of dimerization (CID), in cells expressing iCasp9 expression constructs. FIG. 3A provides FACS plots of cells after treatment with CID or carrier. FACS plots are presented for unselected cells (top row of FIG. 3A) and cells selected for high GFP expression (bottom row of FIG. 3A). FIG. 3B provides the results of overnight treatment of iCasp9 transduced cells with CID. The treated panel shows cells exhibiting characteristics of apoptosis. FIG. 3C provides the results of CID treated and untreated cells stained for annexin-V and 7-AAD. FIG. 3D provides a graph of a dose response curve for the CID AP20187. Further discussion of experimental conditions and results are presented in the Examples.

FIGS. 4A-4C provide the results of various experiments performed to measure the correlation between transgene expression level and function of iCasp9. FIG. 4A shows the results of cell population selection based on GFP expression. FIG. 4B provides the results of cells treated overnight with CID treated and stained for annexin-V and 7-AAD. FIG. 4C shows the results of selected T cells that were mixed 1:1 with non-transduced T-cells and incubated with 10 nM CID following antigenic stimulation. Indicated is the percentage of residual GFP-positive T-cells on day 7. Further discussion of experimental conditions and results are presented in the Examples.

FIGS. 5A-5C provide the results of various experiments comparing the functionality of iFas and iCasp9 in T cells. FIG. 5A provides the results of cells transduced with an iFas or iCasp9 expression construct and sorted according to GFP expression. FIG. 5B provides a graph of the results of GFP expression measurements after treatment with CID. FIG. 5C shows the results of expression studies performed in the human derived cell lines Jurkat and MT-2. The cell lines were stained with annexin-V and 7-AAD. Further discussion of experimental conditions and results are presented in the Examples.

FIG. 6 graphically illustrates the function of iCasp9 when co-expressed with II-2.

FIG. 7 graphically illustrates the function of iCasp9 in vivo. Further discussion of experimental conditions and results are presented in the Examples.

FIG. 8A illustrates the structure of the iCasp9 expression construct SFG.iCasp9.2A.ΔCD19. FIG. 8B illustrates the protocol used to produce the cell product expression iCasp9 in allodepleted cells. Further discussion of experimental conditions and results are presented in the Examples. FIG. 8A discloses “Ser-Gly-Gly-Gly-Ser” as SEQ ID NO: 236.

FIG. 9 provides a graph showing that allodepleted cells could be successfully expanded following transduction.

FIG. 10 shows that cells transduced with the suicide gene construct could be enriched to high purity by CD19 immunomagnetic selection. Further discussion of experimental conditions and results are presented in the Examples.

FIGS. 11A-11C provide the results of various experiments performed to show that gene modified allodepleted cells retain their anti-viral repertoire and functionality. FIG. 11A shows the interferon-γ secretion in response to viral antigens as assessed by ELISpot. FIG. 11B shows the results of a cytotoxicity assay after allodepleted cells were stimulated with EBV-LCLs. FIG. 11C shows the frequency of T cells specific for HLA-B8-RAKFKQLL (SEQ ID NO: 232), an epitope from an EBV lytic antigen (BZLF1).

FIGS. 12A and 12B illustrate the results of various experiments performed to show that regulatory T cells could be isolated from gene modified end product cells despite initial allodepletion using CD25 immunotoxin. FIG. 12A shows the levels of Foxp3 expression. FIG. 12B illustrates the results of the functional assay performed to show that addition of CD4⁺/CD25⁺ gene modified depleted cells significantly reduced cell proliferation. Further discussion of experimental conditions and results are presented in the Examples.

FIGS. 13A-13CB provide the results of various experiments performed to show that gene modified allodepleted cells are rapidly and efficiently eliminated by AP20187, and that transgene expression and killing efficiency diminished with extended culture, and could be restored upon T cell reactivation. FIG. 13A shows representative FACS analysis of cells stained with annexin-V and 7-AAD. FIG. 13B graphically presents the results of reactivation of T cells on killing when AP20187 is administered. FIGS. 13CA and 13CB show representative FACS plots showing the effect of extended culture and T cell activation on suicide gene function. Further discussion of experimental conditions and results are presented in the Examples.

FIGS. 14A and 14B provide the results of various experiments performed to show that viral-specific T cells are partially retained after treatment of allostimulated cells with dimerizer. FIG. 14A shows the results for EBV-specific T cells. FIG. 14B shows the results for CMV-specific T cells. Cells were quantified by pentamer analysis before allostimulation, after allosimulation and after treatment of allostimulated cells with dimerizer. Further discussion of experimental conditions and results are presented in the Examples.

FIGS. 15A and 15B provide the results of an analysis of mesenchymal stromal cells (MSCs) from healthy individuals. FIG. 15A shows the mononuclear adherent fraction isolated from bone marrow was homogenously positive for CD73, CD90 and CD105 and was negative for hematopoietic markers. FIG. 15B presents analysis showing the cells were able to differentiate into other cell lineages. Further discussion of experimental conditions and results are presented in the Examples.

FIGS. 16A and 16B provide the results of experiments performed to show that human MSCs are readily transformed with iCasp9-ΔCD19 and maintain their phenotype. FIG. 16A provides the percentage of CD19 positive cells (e.g., an indicator of successful transduction of iCasp9) remains substantially constant for more than 2 weeks. FIG. 16B shows that successfully transduced and non-transduced cells retain the characteristic MSC surface phenotype. Further discussion of experimental conditions and results are presented in the Examples.

FIGS. 17A and 17B provide the results of experiments performed to show that human MSCs expressing iCasp9 are selectively driven to apoptosis in vitro after exposure to the CID. FIG. 17A shows the results of FACS analysis of cells treated with CID for 24 hours. FIG. 17B shows the results of magnetic purification of iCasp9⁺/CD19⁺ cells. Further discussion of experimental conditions and results are presented in the Examples.

FIG. 18 provides the results of experiments performed to determine the efficacy of apoptosis and identify apoptosis resistant populations.

FIG. 19 panels A-Q provide human MSCs expressing iCasp9 stained to highlight specific cell lineages, showing that the transduced cells retain the differentiation potential of unmodified MSCs. Further discussion of experimental conditions and results are presented in the Examples.

FIG. 20 is a graph showing that the differentiated progeny of human MSCs expressing iCasp9 are killed by exposure to CID in vitro.

FIGS. 21A-21C provide the results of experiments performed to show that human MSCs expressing iCasp9 are selectively killed in vivo after exposure to CID. FIG. 21A shows the results of whole animal imaging. FIG. 21B graphically shows a time course of the killing of iCasp9+ cells after exposure to CID. FIG. 21C shows the results of serial examination of animals after subcutaneous inoculation of MSC. Further discussion of experimental conditions and results are presented in the Examples.

FIG. 22 is a schematic that shows how the suicide gene product and the CID interact to cause apoptosis.

FIG. 23 illustrates an overview of the protocol used for production of suicide gene modified allodepleted cells.

FIG. 24 illustrates the use of immunomagnetic enrichment of iCasp9 expressing allodepleted T cells.

FIG. 25 illustrates the iCasp9-ΔCD19 expression construct and the method of transducing cells to harbor the expression construct. Further discussion of experimental conditions and results are presented in the Examples. FIG. 25 discloses “Ser-Gly-Gly-Gly-Ser” as SEQ ID NO: 236.

FIG. 26 illustrates and provides data showing the effect of CID treatment on gene modified T cells (e.g., iCasp9 expressing cells).

FIGS. 27A and 27B provide graphs showing the detection of iCasp9-transduced T cells in the peripheral blood of patients. FIG. 27A provides cell counts and FIG. 27B provides copies/ug of DNA based on FACS analysis for iCasp9-transduced T cells (CD3⁺ CD19⁺, CD4⁺ CD19⁺, or CD8⁺ CD19⁺) from four patients receiving cellular therapy following HLA-haploidentical stem cell transplantation for relapsed leukemia. Patients 1, 2, and 4 developed skin/liver GvHD and received a single dose of the dimerizing drug AP1903.

FIG. 28 provides a graph showing cell lineage expansion of transduced iCasp9 T cells, as indicated by cell surface markers.

FIG. 29 provides a graph showing cell lineage expansion of transduced iCasp9 T cells, as indicated by cell surface markers.

FIGS. 30A and 30B provide a graph and photographs of the rapid reversal of GvHD after treatment with the dimerizing drug AP1903. FIG. 30A is a graph depicting the normalization of bilirubin concentration in patient 1 within 24 hours post-treatment. FIG. 30B provides photographs showing the disappearance of skin rash from patient 2 within 24 hours post treatment.

FIG. 31 provides a graph of the onset of acute liver GvHD (grade 2) after iCasp9 T cell expansion.

FIG. 32 provides a graph and photograph of the onset of acute liver GvHD (grade 2) after iCasp9 T cell expansion. FIG. 32 also pictorially illustrates a patient exhibiting symptoms of GvHD.

FIG. 33 is a graph of the rapid and efficient elimination of iCasp9 T cells after AP1903 (e.g., the CID) is administered to patients.

FIG. 34 is a graph of the rapid and efficient elimination of iCasp9 T cells after AP1903 (e.g., the CID) is administered to patients.

FIGS. 35A-35C provide graphs and FACs analysis showing the persistence of drug sensitivity and antiviral function of CD3⁺/CD19⁺ precursors after treatment with AP1903 in vivo. FIG. 35A CD3⁺ CD19⁺ T cells remain within the CD3⁺ population in the peripheral blood 5 months after treatment with AP1903 (patient 2). These CD3⁺ CD19⁺ cells retain sensitivity to AP1903 in vitro as assessed both by reduction of CD3⁺ CD19⁺ cell number on FACS analysis and FIG. 35B by quantitative PCR analysis of the icasp9 gene before and after exposure to the dimerizing drug. FIG. 35C CD3⁺ CD19⁺ gene-modified T cells collected from patient 2 were responsive to CMV peptide mixtures at 6 days prior to AP1903, but not to negative control surviving peptide mixtures, as shown by the presence of IFN-gamma-positive CD3⁺ CD19⁺ T cells in the CMV-stimulated cultures. Assessment of the recovering CD3⁺ CD19⁺ population at 6 and 14 days after AP1903 infusion to treat GvHD showed the persistence of virus-specific cells in the absence of recurrent GvHD.

FIG. 36 is a graph showing that iCasp9 allodepleted cells are able to expand after AP1903 treatment without signs of GvHD.

FIG. 37 is a graph showing reconstitution of naïve, central memory and effector memory T cell after AP1903 treatment.

FIG. 39 is a graph showing iCasp9 allodepleted T cell expansion and restoration of donor chimerism. Further discussion of experimental conditions and results are presented in the Examples.

FIG. 40 is a graph showing virus specific T cells pre and post T cell infusion.

FIG. 41 is a graph showing the levels of intracellular IFN-γ production by patient PBMCs in response to aspergillus antigen.

FIG. 42 is a graph showing iCasp9 T cells expansion. Further discussion of experimental conditions and results are presented in the Examples.

FIG. 43 is a schematic of the portion of the expression construct coding for chimeric iCaspase9 and CD19 polypeptides.

FIG. 44 is a graphic of a protein structure of a caspase-9 polypeptide. To modify the basal signaling, site-directed mutagenesis was done on residues previously reported to be crucial in homo-dimerization (G402-C-F-N-F406 (SEQ ID NO: 224)), proteolysis and interaction with XIAP-BIR3 domain (pink), the inhibitor of caspase-9 (gray) (D315-A-P-F319 (SEQ ID NO: 233), D330-A-I-S-S334 (SEQ ID NO: 234)), and phosphorylation sites on caspase-9. The crystallography is based on 1 nw9 (RCSB Protein Data Bank). Sixty-five iCasp9 mutants were tested, and promising candidates with lower basal activity included S183A, S196D, D330A, and N405Q.

FIG. 45 provides a schematic of an analysis of SEAP assays. To examine both basal signaling and AP1903 induced activity, 106 early-passage HEK293T/16 cells were co-transfected with various amount of wild type Caspase and 500 ng of an expression plasmid that uses an SRα promoter to drive SEAP, a marker for cell viability. Following manufacturer's suggestions, 1 mL of IMDM+10% FBS without antibiotics was added to each mixture. 1000-ul of the mixture was seeded onto each well of a 96-well plate. 100-ul of AP1903 was added at least three hours post-transfection. After addition of AP1903 for at least 24 hours, 100-ul of supernatant was transferred to a 96-well plate and heat denatured at 68 C for 30 minutes to inactivate endogenous alkaline phosphatases. For the assay, 4-methylumbelliferyl phosphate substrate was hydrolyzed by SEAP to 4-methylumbelliferon, a metabolite that can be excited with 364 nm and detected with an emission filter of 448 nm. Since SEAP is used as a marker for cell viability, reduced SEAP reading corresponds with increased icaspase-9 activities. Thus, a higher SEAP reading in the absence of AP1903 would indicate lower basal activity. Desired caspase mutants would have diminished basal signaling with increased sensitivity (i.e., lower IC₅₀) to AP1903. The goal of the study is to reduce basal signaling without significantly impairing IC₅₀.

FIGS. 46A-46C provide graphs and a chart of data related to basal and AP1903-induced signaling of various chimeric modified caspase-9 polypeptides. (FIG. 46A) SEAP assay of HEK293/16 cells transiently transfected with 1 μg of DNA coding for chimeric modified caspase-9 polypeptides and 0.5 μg of pSH1-kSEAP per million HEK293 cells, 72 hours post-transfection. iCasp9 D330A, N405Q, and D330A-N405Q double mutant all showed lower basal signaling. (FIG. 46B) HEK293/16 cells transfected with 2 μg of DNA coding for chimeric modified caspase-9 polypeptides along with 0.5 μg pSH1-kSEAP per million HEK293 cells. (FIG. 46C) Summary of estimated AP1903 IC₅₀s of chimeric modified caspase-9 polypeptides. All mutations adversely increased IC₅₀ to AP1903. Data points are the average of two wells, and the data shown is representative of two independent experiments.

FIGS. 47A and 47B include photographs of Western blots analyzing protein expression and proteolysis of chimeric wild type (unmodified) caspase-9 polypeptides and chimeric modified caspase-9 polypeptides. (FIG. 47A) Western blot of HEK293T/16 cells transiently transfected with 1 or 2 μg of pSH1-iCasp9 WT, D330A, N405Q, or D330A-N405Q double mutant 72 hours post-transfection. 33 μg of protein lysates were loaded per lane in both blots. The blots were labeled with 1:1000 diluted rabbit anti-human caspase-9 polyclonal antibody targeting residues 299-318 of human caspase-9 to detect both unprocessed and p30 cleavage products. iCasp9 D330A, N405Q, and D330A-N405Q were expressed at similar or higher levels than wild-type iCasp9. (FIG. 47B) Labeling of stripped blots with anti-actin polyclonal antibody showed equivalent amount of protein loaded in 4A.

FIGS. 48A and 48B provide a graph and a chart of basal and AP1903-induced signaling of top modified caspase mutants. FIG. 48A) SEAP assay of HEK293/16 transiently transfected with 2 ug of mutant caspase polypeptide and 0.5 ug of pSH1-kSEAP per million HEK293 72 hours post-transfection. iCASP-9 F404Y, F404W, N405Q, and F406T all show lower basal signaling than WT iCaspase9 (FIG. 48B) Summary of the basal activity and estimated IC₅₀ of caspase mutants to AP1903. All mutations shift the IC₅₀ to AP1903. The data points reflect the average of two wells, and the data shown is representative of two experiments.

FIGS. 49A and 49B provide graphs and charts of basal and AP1903 induced signaling of top modified caspase mutants. (49A) SEAP assay of HEK293/16 transiently transfected with 1 or 2 ug of mutant caspase polypeptide and 0.5 ug of pSH1-kSEAP per million HEK293 72 hours post-transfection. iCASP-9 D330A, N405Q, and D330A-N405Q double mutant all showed lower basal signaling compared to the wild-type caspase-9 (dash line). (49B) Summary of estimated IC₅₀ of caspase mutant polypeptides to AP1903. N405Q adversely increased IC₅₀ to AP1903. Combining D330A to N405Q failed to improve IC₅₀. The data points were averages of triplicates, and the data shown is representative of seven experiments. (49C) Western blot of HEK293T/16 transiently transfected with 1 or 2 ug of pSH1-iCASP9 WT, D330A, N405Q, or D330A-N405Q double mutant 72 hours post-transfection. 33 ug of proteins were loaded per lane in both blots. The blots were labeled with 1:1000 diluted rabbit anti-human caspase-9 polyclonal antibody targeting residues 299-318 of human caspase-9 to detect both unprocessed and p30 cleavage products. iCASP9 D330A, N405Q, and D330A-N405Q were expressed at similar or higher level than the wild-type iCASP9. iCASP9 N405Q and D330A-N405Q mutants showed impaired cleavage in response to AP1903.

FIG. 50 provides a graph of the SEAP assay showing the decrease in basal activity observed with the T317A and T317S mutants. T317A and T317S mutations are likely to reduce XIAP binding, which would be expected to increase basal signaling, the opposite of what was observed.

FIG. 51 provides a bar chart of a SEAP assay of the T317A and T317S mutants. Although T317A and T317S have lower basal activity, they are almost equally sensitive to AP1903 as wild type caspase-9, making these good candidates for new mutants.

FIG. 52 provides a graph of -basal and AP1903-induced activation of D330A variants. SEAP assay of transiently transfected HEK293/16 cells with 1 or 2 ug of mutant caspase polypeptides and 0.5 ug of pSH1-kSEAP per million HEK293 cells, 72 hours post-transfection. Normalized data based on 2 ug of each expression plasmid (including WT) are mixed with normalized data from 1 ug-based transfections. iCasp9-D330A, -D330E, and -D330S showed statistically lower basal signaling than wildtype caspase-9.

FIG. 53 is a -western blot of HEK293T/16 cells transiently transfected with 2 ug of pSH1-iCasp9 WT, D330A, D330E, D330N, D330V, D330G, and D330S, 72 hours post-transfection. The blots were labeled with 1:1000 diluted rabbit polyclonal anti-“2A” peptide that recognizes the 2A sequence, derived from several picornaviruses. iCasp9-D330A, -D330E, and -D330G were expressed at similar or higher levels than the wild-type iCasp9. Impaired cleavage in response to AP1903 was observed. * annotates SuperSignal MW Protein ladders (Thermo-Fisher Scientific) and [symbol] annotates Precision Plus Protein Dual Color Standards (Bio-Rad).

FIG. 54 is a graph showing the -effects of various caspase mutations on viral titers derived from PG13 packaging cells cross-transduced with VSV-G envelope-based retroviral supernatants. To examine the effect of CaspaClDe-derived basal signaling on retrovirus master cell line production, retrovirus packaging cell line, PG13, was cross-transduced five times with VSV-G-based retroviral supernatants in the presence of 4 μg/ml transfection-enhancer, polybrene. CaspaClDe-transduced PG13 cells were subsequently stained with PE-conjugated anti-human CD19 antibody, as an indication of transduction. CaspaClDe-D330A, -D330E, and -N405Q-transduced PG13 cells showed enhanced CD19 mean fluorescence intensity (MFI), indicating higher retroviral copy numbers, implying lower basal activity. To more directly examine the viral titer of the PG13 transductants, HT1080 cells were treated with viral supernatant and 8 ug/ml polybrene. The enhanced CD19 MFIs of iCasp9-D330A, -N405Q, and -D330E transductants vs WT iCasp9 in PG13 cells are positively correlated with higher viral titers, as observed in HT1080 cells. Due to the initially low viral titers (approximately 1E5 transduction units (TU)/ml), no differences in viral titers were observed in the absence of HAT treatment to increase virus yields. Upon HAT media treatment, PG13 cells transduced with CaspaClDe-D330A, -N405Q, or -D330E demonstrated higher viral titers. Viral titer (transducing units) is calculated with the formula: Viral titer=(# cells on the day of transduction)*(% CD19⁺)/Volume of supernatant (ml). In order to further investigate the effect of CaspaClDe mutants with lower basal activity, individual clones (colonies) of CaspaClDe-transduced PG13 cells were selected and expanded. CaspaClDe-N405Q clones with higher CD19 MFIs than the other cohorts were observed.

FIG. 55 is a graph showing AP1903 dose-dependent elimination of T cells transduced with iCasp9 mutants. Primary T cells from healthy donors (n=6) were transduced with retrovirus encoding mutant or wild-type iCasp9 and the ΔCD19 cell surface marker. Following transduction, iCasp9-transduced T cells were purified using CD19-microbeads and a magnetic column. T cells were then exposed to AP1903 (0-10 nM) and measured for CD3+CD19+ T cells by flow cytometry after 24 hours.

FIG. 56 is a graph showing iCasp9-D330A mutant demonstrates improved AP1903-dependent cytotoxicity in transduced T cells. Primary T cells from healthy donors (n=6) were transduced with retrovirus encoding mutant or wild-type iCasp9 or iCasp9-D330A, and the ΔCD19 cell surface marker. Following transduction, iCasp9-transduced T cells were purified using CD19-microbeads and a magnetic column. T cells were then exposed to AP1903 (0-100 nM) and measured for CD3+CD19+ T cells by flow cytometry after 24 hours.

FIG. 57 is a graph showing iCasp9-D330A mutant demonstrates improved AP1903-dependent cytotoxicity in transduced T cells. Primary T cells from healthy donors (n=6) were transduced with retrovirus encoding mutant or wild-type iCasp9 or iCasp9-D330A, and the ΔCD19 cell surface marker. Following transduction, iCasp9-transduced T cells were purified using CD19-microbeads and a magnetic column. T cells were then exposed to AP1903 (0-100 nM) and measured for CD3+CD19+ T cells by flow cytometry after 24 hours. The IC₅₀ of iCasp9-D330A was significantly lower (p=0.002) than wild-type iCasp9.

FIG. 58 is a graph showing D330 family members demonstrate similar AP1903-dependent cytotoxicity as D330A in transduced T cells. Primary T cells from healthy donors (n=2) were transduced with retrovirus encoding D330 mutants or wild-type iCasp9 and the ΔCD19 cell surface marker. Following transduction, iCasp9-transduced T cells were purified using CD19-microbeads and a magnetic column. T cells were then exposed to AP1903 (0-100 nM) and measured for CD3+CD19+ T cells by flow cytometry after 24 hours.

FIG. 59 is a graph showing iCasp9 mutants with lower sensitivity to AP1903 still control T cell proliferation in vitro. Activated T cells were transduced with retrovirus containing iCasp9 mutants and treated with AP1903 (arrow) on days 0 and 4 and subsequently enumerated for 10 days. Both wild-type, D330A and other iCasp9 mutants halted T cell proliferation and decreased T cell survival after 10 days.

FIG. 60 is a graph showing (improved transgene expression of N405Q mutants improves AP1903-dependent cytotoxicity in transduced T cells. T cells were transduced with RD114 pseudotyped retrovirus encoding iCasp9 wild-type, N405Q, codon-optimized N405Q and Fv.Fv′.N405Q and then treated with a varying dose of AP1903 (0-100 nM). After 24 hours, CD3+CD19+ T cells were measured by flow cytometry. Percent remaining was normalized to the frequency of CD3+CD19+ T cells without AP1903.

FIGS. 61A and 61B are graphs showing AP1903 dose-dependent elimination in vivo of T cells transduced with wild-type iCasp9. T cells were transduced with SFG-iCasp9-2A-ΔCD19 retrovirus and injected i.v. into immune deficient mice (NSG). After 24 hours, mice were injected i.p. with AP1903 (0-5 mg/kg). After an additional 24 hours, mice were sacrificed and lymphocytes from the spleen (FIG. 61A) and peripheral blood (FIG. 61B) were isolated and analyzed by flow cytometry for the frequency of human CD3+CD19+ T cells.

FIG. 62 is a graph showing AP1903 dose-dependent elimination in vivo of T cells transduced with D330E iCasp9. T cells were transduced with SFG-iCasp9-D330E-2A-ΔCD19 retrovirus and injected i.v. into immune deficient mice (NSG). After 24 hours, mice were injected i.p. with AP1903 (0-5 mg/kg). After an additional 24 hours, mice were sacrificed and lymphocytes from the spleen (A) were isolated and analyzed by flow cytometry for the frequency of human CD3+CD19+ T cells. This shows that iCasp9-D330E demonstrates a similar in vivo cytotoxicity profile in response to AP1903 as wild-type iCasp9.

FIG. 63 is a schematic of a plasmid map for a plasmid that encodes an inducible caspase-9 polypeptide separated by a 2A polypeptide from a CD19-specific chimeric antigen receptor; the two polypeptides are separated during translation. FIG. 63 discloses SEQ ID NO: 259.

FIG. 64 is a schematic of a plasmid map for a plasmid that encodes an inducible caspase-9 polypeptide separated by a 2A polypeptide from a Her2-specific chimeric antigen receptor, the two polypeptides are separated during translation. The chimeric antigen receptor includes MyD88 and CD40 polypeptides.

### DETAILED DESCRIPTION

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms.

The term “allogeneic” as used herein, refers to HLA or MHC loci that are antigenically distinct between the host and donor cells.

The term “autologous” as used herein, refers to HLA or MHC loci that are not antigenically distinct between the host and donor cells, for example, where the donor cells are obtained from the host.

Thus, cells or tissue transferred from the same species can be antigenically distinct. Syngeneic mice can differ at one or more loci (congenics) and allogeneic mice can have the same background.

The term “antigen” as used herein is defined as a molecule that provokes an immune response. This immune response may involve either antibody production, or the activation of specific immunologically-competent cells, or both.

An “antigen recognition moiety” may be any polypeptide or fragment thereof, such as, for example, an antibody fragment variable domain, either naturally-derived, or synthetic, which binds to an antigen. Examples of antigen recognition moieties include, but are not limited to, polypeptides derived from antibodies, such as, for example, single chain variable fragments (scFv), Fab, Fab′, F(ab′)2, and Fv fragments; polypeptides derived from T Cell receptors, such as, for example, TCR variable domains; and any ligand or receptor fragment that binds to the extracellular cognate protein.

The term “cancer” as used herein is defined as a hyperproliferation of cells whose unique trait—loss of normal controls—results in unregulated growth, lack of differentiation, local tissue invasion, and metastasis. Examples include but are not limited to, melanoma, non-small cell lung, small-cell lung, lung, hepatocarcinoma, leukemia, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder.

Donor: The term “donor” refers to a mammal, for example, a human, that is not the patient recipient. The donor may, for example, have HLA identity with the recipient, or may have partial or greater HLA disparity with the recipient.

Haploidentical: The term “haploidentical” as used with reference to cells, cell types and/or cell lineages, herein refers to cells sharing a haplotype or cells having substantially the same alleles at a set of closely linked genes on one chromosome. A haploidentical donor does not have complete HLA identity with the recipient, there is a partial HLA disparity.

Blood disease: The terms “blood disease”, “blood disease” and/or “diseases of the blood” as used herein, refers to conditions that affect the production of blood and its components, including but not limited to, blood cells, hemoglobin, blood proteins, the mechanism of coagulation, production of blood, production of blood proteins, the like and combinations thereof. Non-limiting examples of blood diseases include anemias, leukemias, lymphomas, hematological neoplasms, albuminemias, haemophilias and the like.

Bone marrow disease: The term “bone marrow disease” as used herein, refers to conditions leading to a decrease in the production of blood cells and blood platelets. In some bone marrow diseases, normal bone marrow architecture can be displaced by infections (e.g., tuberculosis) or malignancies, which in turn can lead to the decrease in production of blood cells and blood platelets. Non-limiting examples of bone marrow diseases include leukemias, bacterial infections (e.g., tuberculosis), radiation sickness or poisoning, apnocytopenia, anemia, multiple myeloma and the like.

T cells and Activated T cells (include that this means CD3+ cells): T cells (also referred to as T lymphocytes) belong to a group of white blood cells referred to as lymphocytes. Lymphocytes generally are involved in cell-mediated immunity. The “T” in “T cells” refers to cells derived from or whose maturation is influenced by the thymus. T cells can be distinguished from other lymphocytes types such as B cells and Natural Killer (NK) cells by the presence of cell surface proteins known as T cell receptors. The term “activated T cells” as used herein, refers to T cells that have been stimulated to produce an immune response (e.g., clonal expansion of activated T cells) by recognition of an antigenic determinant presented in the context of a Class II major histo-compatibility (MHC) marker. T-cells are activated by the presence of an antigenic determinant, cytokines and/or lymphokines and cluster of differentiation cell surface proteins (e.g., CD3, CD4, CD8, the like and combinations thereof). Cells that express a cluster of differential protein often are said to be “positive” for expression of that protein on the surface of T-cells (e.g., cells positive for CD3 or CD 4 expression are referred to as or CD4⁺). CD3 and CD4 proteins are cell surface receptors or co-receptors that may be directly and/or indirectly involved in signal transduction in T cells.

Peripheral blood: The term “peripheral blood” as used herein, refers to cellular components of blood (e.g., red blood cells, white blood cells and platelets), which are obtained or prepared from the circulating pool of blood and not sequestered within the lymphatic system, spleen, liver or bone marrow.

Umbilical cord blood: Umbilical cord blood is distinct from peripheral blood and blood sequestered within the lymphatic system, spleen, liver or bone marrow. The terms “umbilical cord blood”, “umbilical blood” or “cord blood”, which can be used interchangeably, refers to blood that remains in the placenta and in the attached umbilical cord after child birth. Cord blood often contains stem cells including hematopoietic cells.

By “obtained or prepared” as, for example, in the case of cells, is meant that the cells or cell culture are isolated, purified, or partially purified from the source, where the source may be, for example, umbilical cord blood, bone marrow, or peripheral blood. The terms may also apply to the case where the original source, or a cell culture, has been cultured and the cells have replicated, and where the progeny cells are now derived from the original source.

By “kill” or “killing” as in a percent of cells killed, is meant the death of a cell through apoptosis, as measured using any method known for measuring apoptosis, and, for example, using the assays discussed herein, such as, for example the SEAP assays or T cell assays discussed herein. The term may also refer to cell ablation.

Allodepletion: The term “allodepletion” as used herein, refers to the selective depletion of alloreactive T cells. The term “alloreactive T cells” as used herein, refers to T cells activated to produce an immune response in reaction to exposure to foreign cells, such as, for example, in a transplanted allograft. The selective depletion generally involves targeting various cell surface expressed markers or proteins, (e.g., sometimes cluster of differentiation proteins (CD proteins), CD19, or the like), for removal using immunomagnets, immunotoxins, flow sorting, induction of apoptosis, photodepletion techniques, the like or combinations thereof. In the present methods, the cells may be transduced or transfected with the chimeric protein-encoding vector before or after allodepletion. Also, the cells may be transduced or transfected with the chimeric protein-encoding vector without an allodepletion step, and the non-allodepleted cells may be administered to the patient. Because of the added “safety switch” it is, for example, possible to administer the non allo-depleted T cells because an adverse event such as, for example, graft versus host disease, may be alleviated upon the administration of the multimeric ligand.

Graft versus host disease: The terms “graft versus host disease” or “GvHD”, refer to a complication often associated with allogeneic bone marrow transplantation and sometimes associated with transfusions of un-irradiated blood to immunocompromised patients. Graft versus host disease sometimes can occur when functional immune cells in the transplanted marrow recognize the recipient as “foreign” and mount an immunologic response. GvHD can be divided into an acute form and a chronic form. Acute GVHD (aGVHD) often is observed within the first 100 days following transplant or transfusion and can affect the liver, skin, mucosa, immune system (e.g., the hematopoietic system, bone marrow, thymus, and the like), lungs and gastrointestinal tract. Chronic GVHD (cGVHD) often begins 100 days or later post transplant or transfusion and can attack the same organs as acute GvHD, but also can affect connective tissue and exocrine glands. Acute GvHD of the skin can result in a diffuse maculopapular rash, sometimes in a lacy pattern.

Donor T cell: The term “donor T cell” as used here refers to T cells that often are administered to a recipient to confer anti-viral and/or anti-tumor immunity following allogeneic stem cell transplantation. Donor T cells often are utilized to inhibit marrow graft rejection and increase the success of alloengraftment, however the same donor T cells can cause an alloaggressive response against host antigens, which in turn can result in graft versus host disease (GVHD). Certain activated donor T cells can cause a higher or lower GvHD response than other activated T cells. Donor T cells may also be reactive against recipient tumor cells, causing a beneficial graft vs. tumor effect.

Mesenchymal stromal cell: The terms “mesenchymal stromal cell” or “bone marrow derived mesenchymal stromal cell” as used herein, refer to multipotent stem cells that can differentiate ex vivo, in vitro and in vivo into adipocytes, osteoblasts and chondroblasts, and may be further defined as a fraction of mononuclear bone marrow cells that adhere to plastic culture dishes in standard culture conditions, are negative for hematopoietic lineage markers and are positive for CD73, CD90 and CD105.

Embryonic stem cell: The term “embryonic stem cell” as used herein, refers to pluripotent stem cells derived from the inner cell mass of the blastocyst, an early-stage embryo of between 50 to 150 cells. Embryonic stem cells are characterized by their ability to renew themselves indefinitely and by their ability to differentiate into derivatives of all three primary germ layers, ectoderm, endoderm and mesoderm. Pluripotent is distinguished from mutipotent in that pluripotent cells can generate all cell types, while multipotent cells (e.g., adult stem cells) can only produce a limited number of cell types.

Inducible pluripotent stem cell: The terms “inducible pluripotent stem cell” or “induced pluripotent stem cell” as used herein refers to adult, or differentiated cells, that are “reprogrammed” or induced by genetic (e.g., expression of genes that in turn activates pluripotency), biological (e.g., treatment viruses or retroviruses) and/or chemical (e.g., small molecules, peptides and the like) manipulation to generate cells that are capable of differentiating into many if not all cell types, like embryonic stem cells. Inducible pluripotent stem cells are distinguished from embryonic stem cells in that they achieve an intermediate or terminally differentiated state (e.g., skin cells, bone cells, fibroblasts, and the like) and then are induced to dedifferentiate, thereby regaining some or all of the ability to generate multipotent or pluripotent cells.

CD34⁺ cell: The term “CD34⁺ cell” as used herein refers to a cell expressing the CD34 protein on its cell surface. “CD34” as used herein refers to a cell surface glycoprotein (e.g., sialomucin protein) that often acts as a cell-cell adhesion factor and is involved in T cell entrance into lymph nodes, and is a member of the “cluster of differentiation” gene family. CD34 also may mediate the attachment of stem cells to bone marrow, extracellular matrix or directly to stromal cells. CD34⁺ cells often are found in the umbilical cord and bone marrow as hematopoietic cells, a subset of mesenchymal stem cells, endothelial progenitor cells, endothelial cells of blood vessels but not lymphatics (except pleural lymphatics), mast cells, a sub-population of dendritic cells (which are factor XIIIa negative) in the interstitium and around the adnexa of dermis of skin, as well as cells in certain soft tissue tumors (e.g., alveolar soft part sarcoma, pre-B acute lymphoblastic leukemia (Pre-B-ALL), acute myelogenous leukemia (AML), AML-M7, dermatofibrosarcoma protuberans, gastrointestinal stromal tumors, giant cell fibroblastoma, granulocytic sarcoma, Kaposi's sarcoma, liposarcoma, malignant fibrous histiocytoma, malignant peripheral nerve sheath tumors, mengingeal hemangiopericytomas, meningiomas, neurofibromas, schwannomas, and papillary thyroid carcinoma).

Gene expression vector: The terms “gene expression vector”, “nucleic acid expression vector”, or “expression vector” as used herein, which can be used interchangeably throughout the document, generally refers to a nucleic acid molecule (e.g., a plasmid, phage, autonomously replicating sequence (ARS), artificial chromosome, yeast artificial chromosome (e.g., YAC)) that can be replicated in a host cell and be utilized to introduce a gene or genes into a host cell. The genes introduced on the expression vector can be endogenous genes (e.g., a gene normally found in the host cell or organism) or heterologous genes (e.g., genes not normally found in the genome or on extra-chromosomal nucleic acids of the host cell or organism). The genes introduced into a cell by an expression vector can be native genes or genes that have been modified or engineered. The gene expression vector also can be engineered to contain 5′ and 3′ untranslated regulatory sequences that sometimes can function as enhancer sequences, promoter regions and/or terminator sequences that can facilitate or enhance efficient transcription of the gene or genes carried on the expression vector. A gene expression vector sometimes also is engineered for replication and/or expression functionality (e.g., transcription and translation) in a particular cell type, cell location, or tissue type. Expression vectors sometimes include a selectable marker for maintenance of the vector in the host or recipient cell.

Developmentally regulated promoter: The term “developmentally regulated promoter” as used herein refers to a promoter that acts as the initial binding site for RNA polymerase to transcribe a gene which is expressed under certain conditions that are controlled, initiated by or influenced by a developmental program or pathway. Developmentally regulated promoters often have additional control regions at or near the promoter region for binding activators or repressors of transcription that can influence transcription of a gene that is part of a development program or pathway. Developmentally regulated promoters sometimes are involved in transcribing genes whose gene products influence the developmental differentiation of cells.

Developmentally differentiated cells: The term “developmentally differentiated cells”, as used herein refers to cells that have undergone a process, often involving expression of specific developmentally regulated genes, by which the cell evolves from a less specialized form to a more specialized form in order to perform a specific function. Non-limiting examples of developmentally differentiated cells are liver cells, lung cells, skin cells, nerve cells, blood cells, and the like. Changes in developmental differentiation generally involve changes in gene expression (e.g., changes in patterns of gene expression), genetic re-organization (e.g., remodeling or chromatin to hide or expose genes that will be silenced or expressed, respectively), and occasionally involve changes in DNA sequences (e.g., immune diversity differentiation). Cellular differentiation during development can be understood as the result of a gene regulatory network. A regulatory gene and its cis-regulatory modules are nodes in a gene regulatory network that receive input (e.g., protein expressed upstream in a development pathway or program) and create output elsewhere in the network (e.g., the expressed gene product acts on other genes downstream in the developmental pathway or program).

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

As used herein, the term “icaspase-9” molecule, polypeptide, or protein is defined as an inducible caspase-9. The term “icaspase-9” embraces icaspase-9 nucleic acids, icaspase-9 polypeptides and/or icaspase-9 expression vectors. The term also encompasses either the natural icaspase-9 nucleotide or amino acid sequence, or a truncated sequence that is lacking the CARD domain.

As used herein, the term “iCaspase 1 molecule”, “iCaspase 3 molecule”, or “iCaspase 8 molecule” is defined as an inducible Caspase 1, 3, or 8, respectively. The term iCaspase 1, iCaspase 3, or iCaspase 8, embraces iCaspase 1, 3, or 8 nucleic acids, iCaspase 1, 3, or 8 polypeptides and/or iCaspase 1, 3, or 8 expression vectors, respectively. The term also encompasses either the natural CaspaseiCaspase-1, -3, or -8 nucleotide or amino acid sequence, respectively, or a truncated sequence that is lacking the CARD domain. By “wild type” caspase-9 in the context of the experimental details provided herein, is meant the caspase-9 molecule lacking the CARD domain.

Modified caspase-9 polypeptides comprise at least one amino acid substitution that affects basal activity or IC₅₀, in a chimeric polypeptide comprising the modified caspase-9 polypeptide. Methods for testing basal activity and IC₅₀ are discussed herein. Non-modified caspase-9 polypeptides do not comprise this type of amino acid substitution. Both modified and non-modified caspase-9 polypeptides may be truncated, for example, to remove the CARD domain.

“Function-conservative variants” are proteins or enzymes in which a given amino acid residue has been changed without altering overall conformation and function of the protein or enzyme, including, but not limited to, replacement of an amino acid with one having similar properties, including polar or non-polar character, size, shape and charge. Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

Amino acids other than those indicated as conserved may differ in a protein or enzyme so that the percent protein or amino acid sequence similarity between any two proteins of similar function may vary and can be, for example, at least 70%, preferably at least 80%, more preferably at least 90%, and most preferably at least 95%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA. When using any of these programs, the preferred settings are those that results in the highest sequence similarity.

The amino acid residue numbers referred to herein reflect the amino acid position in the non-truncated and non-modified caspase-9 polypeptide, for example, that of SEQ ID NO: 9. SEQ ID NO: 9 provides an amino acid sequence for the truncated caspase-9 polypeptide, which does not include the CARD domain. Thus SEQ ID NO: 9 commences at amino acid residue number 135, and ends at amino acid residue number 416, with reference to the full length caspase-9 amino acid sequence. Those of ordinary skill in the art may align the sequence with other sequences of caspase-9 polypeptides to, if desired, correlate the amino acid residue number, for example, using the sequence alignment methods discussed herein.

As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There are times when the full or partial genomic sequence is used, such as where the non-coding regions are required for optimal expression or where non-coding regions such as introns are to be targeted in an antisense strategy.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. The term “therapeutic construct” may also be used to refer to the expression construct or transgene. The expression construct or transgene may be used, for example, as a therapy to treat hyperproliferative diseases or disorders, such as cancer, thus the expression construct or transgene is a therapeutic construct or a prophylactic construct.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra.

As used herein, the term “ex vivo” refers to “outside” the body. The terms “ex vivo” and “in vitro” can be used interchangeably herein.

As used herein, the term “functionally equivalent,” as it relates to caspase-9, or truncated caspase-9, for example, refers to a caspase-9 nucleic acid fragment, variant, or analog, refers to a nucleic acid that codes for a caspase-9 polypeptide, or a caspase-9 polypeptide, that stimulates an apoptotic response. “Functionally equivalent” refers, for example, to a caspase-9 polypeptide that is lacking the CARD domain, but is capable of inducing an apoptotic cell response. When the term “functionally equivalent” is applied to other nucleic acids or polypeptides, such as, for example, CD19, the 5′LTR, the multimeric ligand binding region, or CD3, it refers to fragments, variants, and the like that have the same or similar activity as the reference polypeptides of the methods herein.

As used herein, the term “gene” is defined as a functional protein, polypeptide, or peptide-encoding unit. As will be understood, this functional term includes genomic sequences, cDNA sequences, and smaller engineered gene segments that express, or are adapted to express, proteins, polypeptides, domains, peptides, fusion proteins, and mutants.

The term “hyperproliferative disease” is defined as a disease that results from a hyperproliferation of cells. Exemplary hyperproliferative diseases include, but are not limited to cancer or autoimmune diseases. Other hyperproliferative diseases may include vascular occlusion, restenosis, atherosclerosis, or inflammatory bowel disease.

The term “immunogenic composition” or “immunogen” refers to a substance that is capable of provoking an immune response. Examples of immunogens include, e.g., antigens, autoantigens that play a role in induction of autoimmune diseases, and tumor-associated antigens expressed on cancer cells.

The term “immunocompromised” as used herein is defined as a subject that has reduced or weakened immune system. The immunocompromised condition may be due to a defect or dysfunction of the immune system or to other factors that heighten susceptibility to infection and/or disease. Although such a categorization allows a conceptual basis for evaluation, immunocompromised individuals often do not fit completely into one group or the other. More than one defect in the body's defense mechanisms may be affected. For example, individuals with a specific T-lymphocyte defect caused by HIV may also have neutropenia caused by drugs used for antiviral therapy or be immunocompromised because of a breach of the integrity of the skin and mucous membranes. An immunocompromised state can result from indwelling central lines or other types of impairment due to intravenous drug abuse; or be caused by secondary malignancy, malnutrition, or having been infected with other infectious agents such as tuberculosis or sexually transmitted diseases, e.g., syphilis or hepatitis.

As used herein, the term “pharmaceutically or pharmacologically acceptable” refers to molecular entities and compositions that do not produce adverse, allergic, or other untoward reactions when administered to an animal or a human.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells presented herein, its use in therapeutic compositions is contemplated. Supplementary active ingredients also can be incorporated into the compositions.

As used herein, the term “polynucleotide” is defined as a chain of nucleotides. Furthermore, nucleic acids are polymers of nucleotides. Thus, nucleic acids and polynucleotides as used herein are interchangeable. Nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, polynucleotides include mutations of the polynucleotides, include but are not limited to, mutation of the nucleotides, or nucleosides by methods well known in the art. A nucleic acid may comprise one or more polynucleotides.

As used herein, the term “polypeptide” is defined as a chain of amino acid residues, usually having a defined sequence. As used herein the term polypeptide is interchangeable with the terms “peptides” and “proteins”.

As used herein, the term “promoter” is defined as a DNA sequence recognized by the synthetic machinery of the cell, or introduced synthetic machinery, required to initiate the specific transcription of a gene.

The term “transfection” and “transduction” are interchangeable and refer to the process by which an exogenous DNA sequence is introduced into a eukaryotic host cell. Transfection (or transduction) can be achieved by any one of a number of means including electroporation, microinjection, gene gun delivery, retroviral infection, lipofection, superfection and the like.

As used herein, the term “syngeneic” refers to cells, tissues or animals that have genotypes that are identical or closely related enough to allow tissue transplant, or are immunologically compatible. For example, identical twins or animals of the same inbred strain. Syngeneic and isogeneic can be used interchangeably.

The terms “patient” or “subject” are interchangeable, and, as used herein include, but are not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

By “T cell activation molecule” is meant a polypeptide that, when incorporated into a T cell expressing a chimeric antigen receptor, enhances activation of the T cell. Examples include, but are not limited to, ITAM-containing, Signal 1 conferring molecules such as, for example, CD3 ζ polypeptide, and Fc receptor gamma, such as, for example, Fc epsilon receptor gamma (FcεR1γ) subunit (Haynes, N. M., et al. J. Immunol. 166:182-7 (2001)).

As used herein, the term “under transcriptional control” or “operatively linked” is defined as the promoter is in the correct location and orientation in relation to the nucleic acid to control RNA polymerase initiation and expression of the gene.

As used herein, the terms “treatment”, “treat”, “treated”, or “treating” refer to prophylaxis and/or therapy. When used with respect to a solid tumor, such as a cancerous solid tumor, for example, the term refers to prevention by prophylactic treatment, which increases the subject's resistance to solid tumors or cancer. In some examples, the subject may be treated to prevent cancer, where the cancer is familial, or is genetically associated. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse.

As used herein, the term “vaccine” refers to a formulation which contains a composition presented herein which is in a form that is capable of being administered to an animal. Typically, the vaccine comprises a conventional saline or buffered aqueous solution medium in which the composition is suspended or dissolved. In this form, the composition can be used conveniently to prevent, ameliorate, or otherwise treat a condition. Upon introduction into a subject, the vaccine is able to provoke an immune response including, but not limited to, the production of antibodies, cytokines and/or other cellular responses.

In some embodiments, the nucleic acid is contained within a viral vector. In certain embodiments, the viral vector is a retroviral vector. In certain embodiments, the viral vector is an adenoviral vector, a retroviral vector, or a lentiviral vector. It is understood that in some embodiments, the cell is contacted with the viral vector ex vivo, and in some embodiments, the cell is contacted with the viral vector in vivo.

### Hematopoietic Stem Cells and Cell Therapy

Hematopoietic stem cells include hematopoietic progenitor cells, immature, multipotent cells that can differentiate into mature blood cell types. These stem cells and progenitor cells may be isolated from bone marrow and umbilical cord blood, and, in some cases, from peripheral blood. Other stem and progenitor cells include, for example, mesenchymal stromal cells, embryonic stem cells, and inducible pluripotent stem cells.

Bone marrow derived mesenchymal stromal cells (MSCs) have been defined as a fraction of mononuclear bone marrow cells that adhere to plastic culture dishes in standard culture conditions, are negative for hematopoietic lineage markers and positive for CD73, CD90 and CD105, and able to differentiate in vitro into adipocytes, osteoblasts, and chondroblasts. While one physiologic role is presumed to be the support of hematopoiesis, several reports have also established that MSCs are able to incorporate and possibly proliferate in areas of active growth, such as cicatricial and neoplastic tissues, and to home to their native microenvironment and replace the function of diseased cells. Their differentiation potential and homing ability make MSCs attractive vehicles for cellular therapy, either in their native form for regenerative applications, or through their genetic modification for delivery of active biological agents to specific microenvironments such as diseased bone marrow or metastatic deposits. In addition, MSCs possess potent intrinsic immunosuppressive activity, and to date have found their most frequent application in the experimental treatment of graft-versus-host disease and autoimmune disorders (Pittenger, M. F., et al. (1999). Science 284: 143-147; Dominici, M., et al. (2006). Cytotherapy 8: 315-317; Prockop, D. J. (1997). Science 276: 71-74; Lee, R. H., et al. (2006). Proc Natl Acad Sci USA 103: 17438-17443; Studeny, M., et al., (2002). Cancer Res 62: 3603-3608; Studeny, M., et al. (2004). J Natl Cancer Inst 96: 1593-1603; Horwitz, E. M., et al. (1999). Nat Med 5: 309-313; Chamberlain, G., et al., (2007). Stem Cells 25: 2739-2749; Phinney, D. G., and Prockop, D. J. (2007). Stem Cells 25: 2896-2902; Horwitz, E. M., et al. (2002). Proc Natl Acad Sci USA 99: 8932-8937; Hall, B., et al., (2007). Int J Hematol 86: 8-16; Nauta, A. J., and Fibbe, W. E. (2007). Blood 110: 3499-3506; Le Blanc, K., et al. (2008). Lancet 371: 1579-1586; Tyndall, A., and Uccelli, A. (2009). Bone Marrow Transplant).

MSCs have been infused in hundreds of patients with minimal reported side effects. However, follow-up is limited, long term side effects are unknown, and little is known of the consequences that will be associated with future efforts to induce their in vivo differentiation, for example to cartilage or bone, or to genetically modify them to enhance their functionality. Several animal models have raised safety concerns. For instance, spontaneous osteosarcoma formation in culture has been observed in murine derived MSCs. Furthermore, ectopic ossification and calcification foci have been described in mouse and rat models of myocardial infarction after local injection of MSC, and their proarrhythmic potential has also been apparent in co-culture experiments with neonatal rat ventricular myocytes. Moreover, bilateral diffuse pulmonary ossification has been observed after bone marrow transplant in a dog, presumably due to the transplanted stromal components (Horwitz, E. M., et al., (2007). Biol Blood Marrow Transplant 13: 53-57; Tolar, J., et al. (2007). Stem Cells 25: 371-379; Yoon, Y.-S., et al., (2004). Circulation 109: 3154-3157; Breitbach, M., et al. (2007). Blood 110: 1362-1369; Chang, M. G., et al. (2006). Circulation 113: 1832-1841; Sale, G. E., and Storb, R. (1983). Exp Hematol 11: 961-966).

In another example of cell therapy, T cells transduced with a nucleic acid encoding a chimeric antigen receptor have been administered to patients to treat cancer (Zhong, X.-S., (2010) Molecular Therapy 18:413-420). Chimeric antigen receptors (CARs) are artificial receptors designed to convey antigen specificity to T cells without the requirement for MHC antigen presentation. They include an antigen-specific component, a transmembrane component, and an intracellular component selected to activate the T cell and provide specific immunity. Chimeric antigen receptor-expressing T cells may be used in various therapies, including cancer therapies. Costimulating polypeptides may be used to enhance the activation of CAR-expressing T cells against target antigens, and therefore increase the potency of adoptive immunotherapy.

For example, therapeutic cells may be T cells expressing a chimeric antigen receptor based on the humanized monoclonal antibody Trastuzumab (Herceptin) has been used to treat cancer patients. Adverse events are possible, however, and in at least one reported case, the therapy had fatal consequences to the patient (Morgan, R. A., et al., (2010) Molecular Therapy 18:843-851). Transducing the cells with a chimeric caspase-9-based safety switch as presented herein, would provide a safety switch that could stop the adverse event from progressing. Therefore, in some embodiments are provided nucleic acids, cells, and methods wherein the modified T cell also expresses an inducible caspase-9 polypeptide. If there is a need, for example, to reduce the number of chimeric antigen receptor modified T cells, an inducible ligand may be administered to the patient, thereby inducing apoptosis of the modified T cells.

The antitumor efficacy from immunotherapy with T cells engineered to express chimeric antigen receptors (CARs) has steadily improved as CAR molecules have incorporated additional signaling domains to increase their potency. T cells transduced with first generation CARs, containing only the CD3ζ intracellular signaling molecule, have demonstrated poor persistence and expansion in vivo following adoptive transfer (Till B G, Jensen M C, Wang J, et al: CD20-specific adoptive immunotherapy for lymphoma using a chimeric antigen receptor with both CD28 and 4-1 BB domains: pilot clinical trial results. Blood 119:3940-50, 2012; Pule M A, Savoldo B, Myers G D, et al: Virus-specific T cells engineered to coexpress tumor-specific receptors: persistence and antitumor activity in individuals with neuroblastoma. Nat Med 14:1264-70, 2008; Kershaw M H, Westwood J A, Parker L L, et al: A phase I study on adoptive immunotherapy using gene-modified T cells for ovarian cancer. Clin Cancer Res 12:6106-15, 2006), as tumor cells often lack the requisite costimulating molecules necessary for complete T cell activation. Second generation CAR T cells were designed to improve proliferation and survival of the cells. Second generation CAR T cells that incorporate the intracellular costimulating domains from either CD28 or 4-1 BB (Carpenito C, Milone M C, Hassan R, et al: Control of large, established tumor xenografts with genetically retargeted human T cells containing CD28 and CD137 domains. Proc Natl Acad Sci USA 106:3360-5, 2009; Song D G, Ye Q, Poussin M, et al: CD27 costimulation augments the survival and antitumor activity of redirected human T cells in vivo. Blood 119:696-706, 2012), show improved survival and in vivo expansion following adoptive transfer, and more recent clinical trials using anti-CD19 CAR-modified T cells containing these costimulating molecules have shown remarkable efficacy for the treatment of CD19+ leukemia. (Kalos M, Levine B L, Porter D L, et al: T cells with chimeric antigen receptors have potent antitumor effects and can establish memory in patients with advanced leukemia. Sci Transl Med 3:95ra73, 2011; Porter D L, Levine B L, Kalos M, et al: Chimeric antigen receptor-modified T cells in chronic lymphoid leukemia. N Engl J Med 365:725-33, 2011; Brentjens R J, Davila M L, Riviere I, et al: CD19-targeted T cells rapidly induce molecular remissions in adults with chemotherapy-refractory acute lymphoblastic leukemia. Sci Transl Med 5:177ra38, 2013).

While others have explored additional signaling molecules from tumor necrosis factor (TNF)-family proteins, such as OX40 and 4-1 BB, called “third generation” CAR T cells, (Finney H M, Akbar A N, Lawson A D: Activation of resting human primary T cells with chimeric receptors: costimulation from CD28, inducible costimulator, CD134, and CD137 in series with signals from the TCR zeta chain. J Immunol 172:104-13, 2004; Guedan S, Chen X, Madar A, et al: ICOS-based chimeric antigen receptors program bipolar TH17/TH1 cells. Blood, 2014), other molecules which induce T cell signaling distinct from the CD3ζ nuclear factor of activated T cells (NFAT) pathway may provide necessary costimulation for T cell survival and proliferation, and possibly endow CAR T cells with additional, valuable functions, not supplied by more conventional costimulating molecules. Some second and third-generation CAR T cells have been implicated in patient deaths, due to cytokine storm and tumor lysis syndrome caused by highly activated T cells.

By “chimeric antigen receptor” or “CAR” is meant, for example, a chimeric polypeptide which comprises a polypeptide sequence that recognizes a target antigen (an antigen-recognition domain) linked to a transmembrane polypeptide and intracellular domain polypeptide selected to activate the T cell and provide specific immunity. The antigen-recognition domain may be a single-chain variable fragment (ScFv), or may, for example, be derived from other molecules such as, for example, a T cell receptor or Pattern Recognition Receptor. The intracellular domain comprises at least one polypeptide which causes activation of the T cell, such as, for example, but not limited to, CD3 zeta, and, for example, co-stimulatory molecules, for example, but not limited to, CD28, OX40 and 4-1 BB. The term “chimeric antigen receptor” may also refer to chimeric receptors that are not derived from antibodies, but are chimeric T cell receptors. These chimeric T cell receptors may comprise a polypeptide sequence that recognizes a target antigen, where the recognition sequence may be, for example, but not limited to, the recognition sequence derived from a T cell receptor or an scFv. The intracellular domain polypeptides are those that act to activate the T cell. Chimeric T cell receptors are discussed in, for example, Gross, G., and Eshar, Z., FASEB Journal 6:3370-3378 (1992), and Zhang, Y., et al., PLOS Pathogens 6:1-13 (2010).

In one type of chimeric antigen receptor (CAR), the variable heavy (VH) and light (VL) chains for a tumor-specific monoclonal antibody are fused in-frame with the CD3 zeta chain (ζ) from the T cell receptor complex. The VH and VL are generally connected together using a flexible glycine-serine linker, and then attached to the transmembrane domain by a spacer (CH2CH3) to extend the scFv away from the cell surface so that it can interact with tumor antigens. Following transduction, T cells now express the CAR on their surface, and upon contact and ligation with a tumor antigen, signal through the CD3 zeta chain inducing cytotoxicity and cellular activation.

Investigators have noted that activation of T cells through CD3 zeta is sufficient to induce a tumor-specific killing, but is insufficient to induce T cell proliferation and survival. Early clinical trials using T cells modified with first generation CARs expressing only the zeta chain showed that gene-modified T cells exhibited poor survival and proliferation in vivo.

As co-stimulation through the B7 axis is necessary for complete T cell activation, investigators added the co-stimulating polypeptide CD28 signaling domain to the CAR construct. This region generally contains the transmembrane region (in place of the CD3 zeta version) and the YMNM motif for binding PI3K and Lck. In vivo comparisons between T cells expressing CARs with only zeta or CARs with both zeta and CD28 demonstrated that CD28 enhanced expansion in vivo, in part due to increased IL-2 production following activation. The inclusion of CD28 is called a 2nd generation CAR. The most commonly used costimulating molecules include CD28 and 4-1 BB, which, following tumor recognition, can initiate a signaling cascade resulting in NF-κB activation, which promotes both T cell proliferation and cell survival.

The use of co-stimulating polypeptides 4-1 BB or OX40 in CAR design has further improved T cell survival and efficacy. 4-1 BB in particular appears to greatly enhance T cell proliferation and survival. This 3rd generation design (with 3 signaling domains) has been used in PSMA CARs (Zhong X S, et al., Mol Ther. 2010 February; 18(2):413-20) and in CD19 CARs, most notably for the treatment of CLL (Milone, M. C., et al., (2009) Mol. Ther. 17:1453-1464; Kalos, M., et al., Sci. Transl. Med. (2011) 3:95ra73; Porter, D., et al., (2011) N. Engl. J. Med. 365: 725-533). These cells showed impressive function in 3 patients, expanding more than a 1000-fold in vivo, and resulted in sustained remission in all three patients.

It is understood that by “derived” is meant that the nucleotide sequence or amino acid sequence may be derived from the sequence of the molecule. The intracellular domain comprises at least one polypeptide which causes activation of the T cell, such as, for example, but not limited to, CD3 zeta, and, for example, co-stimulatory molecules, for example, but not limited to, CD28, OX40 and 4-1 BB.

In another example of cell therapy, T cells are modified so that express a non-functional TGF-beta receptor, rendering them resistant to TGF-beta. This allows the modified T cells to avoid the cytotoxicity caused by TGF-beta, and allows the cells to be used in cellular therapy (Bollard, C. J., et al., (2002) Blood 99:3179-3187; Bollard, C. M., et al., (2004) J. Exptl. Med. 200:1623-1633). However, it also could result in a T cell lymphoma, or other adverse effect, as the modified T cells now lack part of the normal cellular control; these therapeutic T cells could themselves become malignant. Transducing these modified T cells with a chimeric caspase-9-based safety switch as presented herein, would provide a safety switch that could avoid this result.

Cells used in cellular therapy, that express a heterologous gene, such as a modified receptor, or a chimeric receptor, may be transduced with nucleic acid that encodes a chimeric caspase-9-based safety switch before, after, or at the same time, as the cells are transduced with the heterologous gene.

### Haploidentical Stem Cell Transplantation

While stem cell transplantation has proven an effective means of treating a wide variety of diseases involving hematopoietic stem cells and their progeny, a shortage of histocompatible donors has proved a major impediment to the widest application of the approach. The introduction of large panels of unrelated stem cell donors and or cord blood banks has helped to alleviate the problem, but many patients remain unsuited to either source. Even when a matched donor can be found, the elapsed time between commencing the search and collecting the stem cells usually exceeds three months, a delay that may doom many of the neediest patients. Hence there has been considerable interest in making use of HLA haploidentical family donors. Such donors may be parents, siblings or second-degree relatives. The problem of graft rejection may be overcome by a combination of appropriate conditioning and large doses of stem cells, while graft versus host disease (GvHD) may be prevented by extensive T cell-depletion of the donor graft. The immediate outcomes of such procedures have been gratifying, with engraftment rate>90% and a severe GvHD rate of <10% for both adults and children even in the absence of post transplant immunosuppression. Unfortunately the profound immunosuppression of the grafting procedure, coupled with the extensive T cell-depletion and HLA mismatching between donor and recipient lead to an extremely high rate of post-transplant infectious complications, and contributed to high incidence of disease relapse.

Donor T cell infusion is an effective strategy for conferring anti-viral and anti-tumor immunity following allogeneic stem cell transplantation. Simple addback of T cells to the patients after haploidentical transplantation, however, cannot work; the frequency of alloreactive T cells is several orders of magnitude higher than the frequency of, for example, virus specific T lymphocytes. Methods are being developed to accelerate immune reconstitution by administrating donor T cells that have first been depleted of alloreactive cells. One method of achieving this is stimulating donor T cells with recipient EBV-transformed B lymphoblastoid cell lines (LCLs). Alloreactive T cells upregulate CD25 expression, and are eliminated by a CD25 Mab immunotoxin conjugate, RFT5-SMPT-dgA. This compound consists of a murine IgG1 anti-CD25 (IL-2 receptor alpha chain) conjugated via a hetero-bifunctional crosslinker [N-succinimidyloxycarbonyl-alpha-methyl-d-(2-pyridylthio) toluene] to chemically deglycosylated ricin A chain (dgA).

Treatment with CD25 immunotoxin after LCL stimulation depletes>90% of alloreactive cells. In a phase I clinical study, using CD25 immunotoxin to deplete alloreactive lymphocytes immune reconstitution after allodepleted donor T cells were infused at 2 dose levels into recipients of T-cell-depleted haploidentical SCT. Eight patients were treated at 10⁴ cells/kg/dose, and 8 patients received 10⁵ cells/kg/dose. Patients receiving 10⁵ cells/kg/dose showed significantly improved T-cell recovery at 3, 4, and 5 months after SCT compared with those receiving 10⁴ cells/kg/dose (P<0.05). Accelerated T-cell recovery occurred as a result of expansion of the effector memory (CD45RA(−)CCR-7(−)) population (P<0.05), suggesting that protective T-cell responses are likely to be long lived. T-cell-receptor signal joint excision circles (TRECs) were not detected in reconstituting T cells in dose-level 2 patients, indicating they are likely to be derived from the infused allodepleted cells. Spectratyping of the T cells at 4 months demonstrated a polyclonal Vbeta repertoire. Using tetramer and enzyme-linked immunospot (ELISpot) assays, cytomegalovirus (CMV)- and Epstein-Barr virus (EBV)-specific responses in 4 of 6 evaluable patients at dose level 2 as early as 2 to 4 months after transplantation, whereas such responses were not observed until 6 to 12 months in dose-level 1 patients. The incidence of significant acute (2 of 16) and chronic graft-versus-host disease (GvHD; 2 of 15) was low. These data demonstrate that allodepleted donor T cells can be safely used to improve T-cell recovery after haploidentical SCT. The amount of cells infused was subsequently escalated to 10⁶ cells/kg without evidence of GvHD.

Although this approach reconstituted antiviral immunity, relapse remained a major problem and 6 patients transplanted for high risk leukemia relapsed and died of disease. Higher T cell doses are therefore useful to reconstitute anti-tumor immunity and to provide the hoped-for anti-tumor effect, since the estimated frequency of tumor-reactive precursors is 1 to 2 logs less than frequency of viral-reactive precursors. However, in some patients, these doses of cells will be sufficient to trigger GvHD even after allodepletion (Hurley C K, et al., Biol Blood Marrow Transplant 2003; 9:610-615; Dey B R, et al., Br. J Haematol. 2006; 135:423-437; Aversa F, et al., N Engl J Med 1998; 339:1186-1193; Aversa F, et al., J Clin. Oncol. 2005; 23:3447-3454; Lang P, Mol. Dis. 2004; 33:281-287; Kolb H J, et al., Blood 2004; 103:767-776; Gottschalk S, et al., Annu. Rev. Med 2005; 56:29-44; Bleakley M, et al., Nat. Rev. Cancer 2004; 4:371-380; Andre-Schmutz I, et al., Lancet 2002; 360:130-137; Solomon S R, et al., Blood 2005; 106:1123-1129; Amrolia P J, et al., Blood 2006; 108:1797-1808; Amrolia P J, et al., Blood 2003; Ghetie V, et al., J Immunol Methods 1991; 142:223-230; Molldrem J J, et al., Cancer Res 1999; 59:2675-2681; Rezvani K, et al., Clin. Cancer Res. 2005; 1 1:8799-8807; Rezvani K, et al., Blood 2003; 102:2892-2900).

### Graft Versus Host Disease (GvHD)

Graft versus Host Disease is a condition that sometimes occurs after the transplantation of donor immunocompetent cells, for example, T cells, into a recipient. The transplanted cells recognize the recipient's cells as foreign, and attack and destroy them. This condition can be a dangerous effect of T cell transplantation, especially when associated with haploidentical stem cell transplantation. Sufficient T cells should be infused to provide the beneficial effects, such as, for example, the reconstitution of an immune system and the graft anti-tumor effect. But, the number of T cells that can be transplanted can be limited by the concern that the transplant will result in severe graft versus host disease.

Graft versus Host Disease may be staged as indicated in the following tables:

Staging

| | Stage 0 | Stage 1 | Stage 2 | Stage 3 | Stage 4 |
|---|---|---|---|---|---|
| Skin | No rash | Rash <25% | 25-50% | >50% | Plus bullae |
| | | BSA | | Generalized | and |
| | | | | erythroderma | desquamation |
| Gut | <500 mL | 501-1000 mL/day | 1001-1500 mL/day | >1500 mL/day | Severe |
| (for | diarrhea/day | 5 cc/kg-10 cc/kg/day | 10 cc/kg-15 cc/kg/day | >15 cc/kg/day | abdominal |
| pediatric | | | | | pain and ileus |
| patients) | | | | | |
| UGI | | Severe | | | |
| | | nausea/vomiting | | | |
| Liver | Bilirubins | 2.1-3 mg/di | 3.1-6 mg/di | 6.1-15 mg/di | >15 mg/di |
| | 2 mg/di | | | | |

Acute GvHD grading may be performed by the consensus conference criteria (Przepiorka D et al., 1994 Consensus Conference on Acute GVHD Grading. Bone Marrow Transplant 1995; 15:825-828).

Grading Index of Acute GvHD

| | Skin | Liver | Gut | Upper GI |
|---|---|---|---|---|
| 0 | None and | None and | None and | None |
| I | Stage 1-2 and | None and | None | None |
| II | Stage 3 and/or | Stage 1 and/or | Stage 1 and/or | Stage 1 |
| III | None-Stage 3 | Stage 2-3 or | Stage 2-4 | N/A |
| | with | | | |
| IV | Stage 4 or | Stage 4 | N/A | N/A |

### Inducible Caspase-9 as a “Safety Switch” for Cell Therapy and for Genetically Engineered Cell Transplantation

By reducing the effect of graft versus host disease is meant, for example, a decrease in the GvHD symptoms so that the patient may be assigned a lower level stage, or, for example, a reduction of a symptom of graft versus host disease by about or at least 20%, 30%, 40%, 50%, 60%, 70%, 80%, 90%, 95%, or 99%. A reduction in the effect of graft versus host disease may also be measured by detection of a reduction in activated T cells involved in the GvHD reaction, such as, for example, a reduction of cells that express the marker protein, for example CD19, and express CD3 (CD3⁺ CD19⁺ cells, for example) by at least 30%, 40%, 50%, 60%, 70%, 75%, 80%, 85%, 90%, 95%, or 99%.

Provided herein is an alternative suicide gene strategy that is based on human proapoptotic molecules fused with an FKBP variant that is optimized to bind a chemical inducer of dimerization (CID) (Clackson T, et al., Proc Natl Acad Sci USA. 1998, 95:10437-10442), AP1903, or rimiducid, a synthetic drug that has proven safe in healthy volunteers (Iuliucci J D, et al., J Clin Pharmacol. 2001, 41:870-879). Administration of this small molecule results in cross-linking and activation of the proapoptotic target molecules. The application of this inducible system in human T lymphocytes has been explored using Fas or the death effector domain (DED) of the Fas-associated death domain-containing protein (FADD) as proapoptotic molecules. Up to 90% of T cells transduced with these inducible death molecules underwent apoptosis after administration of CID (Thomis D C, et al., Blood. 2001, 97:1249-1257; Spencer D M, et al., Curr Biol. 1996, 6: 839-847; Fan L, et al., Hum Gene Ther. 1999, 10: 2273-2285; Berger C, et al., Blood. 2004, 103:1261-1269; Junker K, et al., Gene Ther. 2003, 10:1189-197). This suicide gene strategy may be used in any appropriate cell used for cell therapy including, for example, hematopoietic stem cells, and other progenitor cells, including, for example, mesenchymal stromal cells, embryonic stem cells, and inducible pluripotent stem cells.

Therefore, this safety switch, catalyzed by caspase-9, may be used where there is a condition in the cell therapy patient that requires the removal of the transfected or transduced therapeutic cells. Conditions where the cells may need to be removed include, for example, GvHD, inappropriate differentiation of the cells into more mature cells of the wrong tissue or cell type, and other toxicities. To activate the caspase-9 switch in the case of inappropriate differentiation, it is possible to use tissue specific promoters. For example, where a progenitor cell differentiates into bone and fat cells, and the fat cells are not desired, the vector used to transfect or transduce the progenitor cell may have a fat cell specific promoter that is operably linked to the caspase-9 nucleotide sequence. In this way, should the cells differentiate into fat cells, upon administration of the multimer ligand, apoptosis of the inappropriately differentiated fat cells should result.

The methods may be used, for example, for any disorder that can be alleviated by cell therapy, including cancer, cancer in the blood or bone marrow, other blood or bone marrow borne diseases such as sickle cell anemia and metachromic leukodystrophy, and any disorder that can be alleviated by a stem cell transplantation, for example blood or bone marrow disorders such as sickle cell anemia or metachromal leukodystrophy.

The efficacy of adoptive immunotherapy may be enhanced by rendering the therapeutic T cells resistant to immune evasion strategies employed by tumor cells. In vitro studies have shown that this can be achieved by transduction with a dominant-negative receptor or an immunomodulatory cytokine (Bollard C M, et al., Blood. 2002, 99:3179-3187: Wagner H J, et al., Cancer Gene Ther. 2004, 11:81-91). Moreover, transfer of antigen-specific T-cell receptors allows for the application of T-cell therapy to a broader range of tumors (Pule M, et al., Cytotherapy. 2003, 5:211-226; Schumacher T N, Nat Rev Immunol. 2002, 2:512-519). A suicide system for engineered human T cells was developed and tested to allow their subsequent use in clinical studies. caspase-9 has been modified and shown to be stably expressed in human T lymphocytes without compromising their functional and phenotypic characteristics while demonstrating sensitivity to CID, even in T cells that have upregulated antiapoptotic molecules. (Straathof, K. C., et al., 2005, Blood 105:4248-54).

In genetically modified cells used for gene therapy, the gene may be a heterologous polynucleotide sequence derived from a source other than the cell that ising used to express the gene. The gene is derived from a prokaryotic or eukaryotic source such as a bacterium, a virus, yeast, a parasite, a plant, or even an animal. The heterologous DNA also is derived from more than one source, i.e., a multigene construct or a fusion protein. The heterologous DNA also may include a regulatory sequence, which is derived from one source and the gene from a different source. Or, the heterologous DNA may include regulatory sequences that are used to change the normal expression of a cellular endogenous gene.

### Other Caspase Molecules

Caspase polypeptides other than caspase-9 that may be encoded by the chimeric polypeptides of the current technology include, for example, Caspase-1, Caspase-3, and Caspase-8. Discussions of these Caspase polypeptides may be found in, for example, MacCorkle, R. A., et al., Proc. Natl. Acad. Sci. U.S.A. (1998) 95:3655-3660; and Fan, L., et al. (1999) Human Gene Therapy 10:2273-2285).

### Engineering Expression Constructs

Expression constructs encode a multimeric ligand binding region and a caspase-9 polypeptide, or, in certain embodiments a multimeric ligand binding region and a caspase-9 polypeptide linked to a marker polypeptide, all operatively linked. In general, the term “operably linked” is meant to indicate that the promoter sequence is functionally linked to a second sequence, wherein, for example, the promoter sequence initiates and mediates transcription of the DNA corresponding to the second sequence. The caspase-9 polypeptide may be full length or truncated. In certain embodiments, the marker polypeptide is linked to the caspase-9 polypeptide. For example, the marker polypeptide may be linked to the caspase-9 polypeptide via a polypeptide sequence, such as, for example, a cleavable 2A-like sequence. The marker polypeptide may be, for example, CD19, or may be, for example, a heterologous protein, selected to not affect the activity of the chimeric caspase polypeptide.

In some embodiments, the polynucleotide may encode the caspase-9 polypeptide and a heterologous protein, which may be, for example a marker polypeptide and may be, for example, a chimeric antigen receptor. The heterologous polypeptide, for example, the chimeric antigen receptor, may be linked to the caspase-9 polypeptide via a polypeptide sequence, such as, for example, a cleavable 2A-like sequence.

In certain examples, a nucleic acid comprising a polynucleotide coding for a chimeric antigen receptor is included in the same vector, such as, for example, a viral or plasmid vector, as a polynucleotide coding for a second polypeptide. This second polypeptide may be, for example, a caspase polypeptide, as discussed herein, or a marker polypeptide. In these examples, the construct may be designed with one promoter operably linked to a nucleic acid comprising a polynucleotide coding for the two polypeptides, linked by a cleavable 2A polypeptide. In this example, the first and second polypeptides are separated during translation, resulting in a chimeric antigen receptor polypeptide, and the second polypeptide. In other examples, the two polypeptides may be expressed separately from the same vector, where each nucleic acid comprising a polynucleotide coding for one of the polypeptides is operably linked to a separate promoter. In yet other examples, one promoter may be operably linked to the two nucleic acids, directing the production of two separate RNA transcripts, and thus two polypeptides. Therefore, the expression constructs discussed herein may comprise at least one, or at least two promoters.

2A-like sequences, or “cleavable” 2A sequences, are derived from, for example, many different viruses, including, for example, from Thosea asigna. These sequences are sometimes also known as “peptide skipping sequences.” When this type of sequence is placed within a cistron, between two peptides that are intended to be separated, the ribosome appears to skip a peptide bond, in the case of Thosea asigna sequence, the bond between the Gly and Pro amino acids is omitted. This leaves two polypeptides, in this case the caspase-9 polypeptide and the marker polypeptide. When this sequence is used, the peptide that is encoded 5′ of the 2A sequence may end up with additional amino acids at the carboxy terminus, including the Gly residue and any upstream in the 2A sequence. The peptide that is encoded 3′ of the 2A sequence may end up with additional amino acids at the amino terminus, including the Pro residue and any downstream in the 2A sequence. “2A” or “2A-like” sequences are part of a large family of peptides that can cause peptide bond-skipping. Various 2A sequences have been characterized (e.g., F2A, P2A, T2A), and are examples of 2A-like sequences that may be used in the polypeptides of the present application.

The expression construct may be inserted into a vector, for example a viral vector or plasmid. The steps of the methods provided may be performed using any suitable method, these methods include, without limitation, methods of transducing, transforming, or otherwise providing nucleic acid to the antigen-presenting cell, presented herein. In some embodiments, the truncated caspase-9 polypeptide is encoded by the nucleotide sequence of SEQ ID NO 8, SEQ ID NO: 23, SEQ ID NO: 25, SEQ ID NO: 27, or a functionally equivalent fragment thereof, with or without DNA linkers, or has the amino acid sequence of SEQ ID NO: 9, SEQ ID NO: 24, SEQ ID NO: 26, or SEQ ID NO: 28or a functionally equivalent fragment thereof. In some embodiments, the CD19 polypeptide is encoded by the nucleotide sequence of SEQ ID NO 14, or a functionally equivalent fragment thereof, with or without DNA linkers, or has the amino acid sequence of SEQ ID NO: 15, or a functionally equivalent fragment thereof. A functionally equivalent fragment of the caspase-9 polypeptide has substantially the same ability to induce apoptosis as the polypeptide of SEQ ID NO: 9, with at least 50%, 60%, 70%, 80%, 90%, or 95% of the activity of the polypeptide of SEQ ID NO: 9. A functionally equivalent fragment of the CD19 polypeptide has substantially the same ability as the polypeptide of SEQ ID No: 15, to act as a marker to be used to identify and select transduced or transfected cells, with at least 50%, 60%, 70%, 80%, 90%, or 95% of the marker polypeptide being detected when compared to the polypeptide of SEQ ID NO: 15, using standard detection techniques.

More particularly, more than one ligand-binding domain or multimerization region may be used in the expression construct. Yet further, the expression construct contains a membrane-targeting sequence. Appropriate expression constructs may include a co-stimulatory polypeptide element on either side of the above FKBP ligand-binding elements.

In certain examples, the polynucleotide coding for the inducible caspase polypeptide is included in the same vector, such as, for example, a viral or plasmid vector, as a polynucleotide coding for a chimeric antigen receptor. In these examples, the construct may be designed with one promoter operably linked to a nucleic acid comprising a nucleotide sequence coding for the two polypeptides, linked by a cleavable 2A polypeptide. In this example, the first and second polypeptides are cleaved after expression, resulting in a chimeric antigen receptor polypeptide and an inducible caspase-9 polypeptide. In other examples, the two polypeptides may be expressed separately from the same vector, where each nucleic acid comprising a nucleotide sequence coding for one of the polypeptides is operably linked to a separate promoter. In yet other examples, one promoter may be operably linked to the two nucleic acids, directing the production of two separate RNA transcripts, and thus two polypeptides. Therefore, the expression constructs discussed herein may comprise at least one or at least two promoters.

In yet other examples, two polypeptides may be expressed in a cell using two separate vectors. The cells may be co-transfected or co-transformed with the vectors, or the vectors may be introduced to the cells at different times.

Ligand-Binding Regions

The ligand-binding (“dimerization”) domain, or multimerization region, of the expression construct can be any convenient domain that will allow for induction using a natural or unnatural ligand, for example, an unnatural synthetic ligand. The multimerization region can be internal or external to the cellular membrane, depending upon the nature of the construct and the choice of ligand. A wide variety of ligand-binding proteins, including receptors, are known, including ligand-binding proteins associated with the cytoplasmic regions indicated above. As used herein the term “ligand-binding domain” can be interchangeable with the term “receptor”. Of particular interest are ligand-binding proteins for which ligands (for example, small organic ligands) are known or may be readily produced. These ligand-binding domains or receptors include the FKBPs and cyclophilin receptors, the steroid receptors, the tetracycline receptor, the other receptors indicated above, and the like, as well as “unnatural” receptors, which can be obtained from antibodies, particularly the heavy or light chain subunit, mutated sequences thereof, random amino acid sequences obtained by stochastic procedures, combinatorial syntheses, and the like. In certain embodiments, the ligand-binding region is selected from the group consisting of FKBP ligand-binding region, cyclophilin receptor ligand-binding region, steroid receptor ligand-binding region, cyclophilin receptors ligand-binding region, and tetracycline receptor ligand-binding region. Often, the ligand-binding region comprises a Fᵥ_{^{′}}Fᵥₗₛ sequence. Sometimes, the Fᵥ_{^{′}}Fᵥₗₛ sequence further comprises an additional Fᵥ_{^{′}} sequence. The FKBP12 region may have, for example, an amino acid substitution at position 36 selected from the group consisting of valine, leucine, isoleucine and alanine. Examples include, for example, those discussed in Kopytek, S. J., et al., Chemistry & Biology 7:313-321 (2000) and in Gestwicki, J. E., et al., Combinatorial Chem. & High Throughput Screening 10:667-675 (2007); Clackson T (2006) Chem Biol Drug Des 67:440-2; Clackson, T., in Chemical Biology: From Small Molecules to Systems Biology and Drug Design (Schreiber, s., et al., eds., Wiley, 2007)).

For the most part, the ligand-binding domains or receptor domains will be at least about 50 amino acids, and fewer than about 350 amino acids, usually fewer than 200 amino acids, either as the natural domain or truncated active portion thereof. The binding domain may, for example, be small (<25 kDa, to allow efficient transfection in viral vectors), monomeric, nonimmunogenic, have synthetically accessible, cell permeable, nontoxic ligands that can be configured for dimerization.

The receptor domain can be intracellular or extracellular depending upon the design of the expression construct and the availability of an appropriate ligand. For hydrophobic ligands, the binding domain can be on either side of the membrane, but for hydrophilic ligands, particularly protein ligands, the binding domain will usually be external to the cell membrane, unless there is a transport system for internalizing the ligand in a form in which it is available for binding. For an intracellular receptor, the construct can encode a signal peptide and transmembrane domain 5′ or 3′ of the receptor domain sequence or may have a lipid attachment signal sequence 5′ of the receptor domain sequence. Where the receptor domain is between the signal peptide and the transmembrane domain, the receptor domain will be extracellular.

The portion of the expression construct encoding the receptor can be subjected to mutagenesis for a variety of reasons. The mutagenized protein can provide for higher binding affinity, allow for discrimination by the ligand of the naturally occurring receptor and the mutagenized receptor, provide opportunities to design a receptor-ligand pair, or the like. The change in the receptor can involve changes in amino acids known to be at the binding site, random mutagenesis using combinatorial techniques, where the codons for the amino acids associated with the binding site or other amino acids associated with conformational changes can be subject to mutagenesis by changing the codon(s) for the particular amino acid, either with known changes or randomly, expressing the resulting proteins in an appropriate prokaryotic host and then screening the resulting proteins for binding.

Antibodies and antibody subunits, e.g., heavy or light chain, particularly fragments, more particularly all or part of the variable region, or fusions of heavy and light chain to create high-affinity binding, can be used as the binding domain. Antibodies that are contemplated include ones that are an ectopically expressed human product, such as an extracellular domain that would not trigger an immune response and generally not expressed in the periphery (i.e., outside the CNS/brain area). Such examples, include, but are not limited to low affinity nerve growth factor receptor (LNGFR), and embryonic surface proteins (i.e., carcinoembryonic antigen).

Yet further, antibodies can be prepared against haptenic molecules, which are physiologically acceptable, and the individual antibody subunits screened for binding affinity. The cDNA encoding the subunits can be isolated and modified by deletion of the constant region, portions of the variable region, mutagenesis of the variable region, or the like, to obtain a binding protein domain that has the appropriate affinity for the ligand. In this way, almost any physiologically acceptable haptenic compound can be employed as the ligand or to provide an epitope for the ligand. Instead of antibody units, natural receptors can be employed, where the binding domain is known and there is a useful ligand for binding.

Oligomerization

The transduced signal will normally result from ligand-mediated oligomerization of the chimeric protein molecules, i.e., as a result of oligomerization following ligand-binding, although other binding events, for example allosteric activation, can be employed to initiate a signal. The construct of the chimeric protein will vary as to the order of the various domains and the number of repeats of an individual domain.

For multimerizing the receptor, the ligand for the ligand-binding domains/receptor domains of the chimeric surface membrane proteins will usually be multimeric in the sense that it will have at least two binding sites, with each of the binding sites capable of binding to the ligand receptor domain. By “multimeric ligand binding region” is meant a ligand binding region that binds to a multimeric ligand. The term “multimeric ligands” include dimeric ligands. A dimeric ligand will have two binding sites capable of binding to the ligand receptor domain. Desirably, the subject ligands will be a dimer or higher order oligomer, usually not greater than about tetrameric, of small synthetic organic molecules, the individual molecules typically being at least about 150 Da and less than about 5 kDa, usually less than about 3 kDa. A variety of pairs of synthetic ligands and receptors can be employed. For example, in embodiments involving natural receptors, dimeric FK506 can be used with an FKBP12 receptor, dimerized cyclosporin A can be used with the cyclophilin receptor, dimerized estrogen with an estrogen receptor, dimerized glucocorticoids with a glucocorticoid receptor, dimerized tetracycline with the tetracycline receptor, dimerized vitamin D with the vitamin D receptor, and the like. Alternatively higher orders of the ligands, e.g., trimeric can be used. For embodiments involving unnatural receptors, e.g., antibody subunits, modified antibody subunits, single chain antibodies comprised of heavy and light chain variable regions in tandem, separated by a flexible linker domain, or modified receptors, and mutated sequences thereof, and the like, any of a large variety of compounds can be used. A significant characteristic of these ligand units is that each binding site is able to bind the receptor with high affinity and they are able to be dimerized chemically. Also, methods are available to balance the hydrophobicity/hydrophilicity of the ligands so that they are able to dissolve in serum at functional levels, yet diffuse across plasma membranes for most applications.

In certain embodiments, the present methods utilize the technique of chemically induced dimerization (CID) to produce a conditionally controlled protein or polypeptide. In addition to this technique being inducible, it also is reversible, due to the degradation of the labile dimerizing agent or administration of a monomeric competitive inhibitor.

The CID system uses synthetic bivalent ligands to rapidly crosslink signaling molecules that are fused to ligand-binding domains. This system has been used to trigger the oligomerization and activation of cell surface (Spencer, D. M., et al., Science, 1993. 262: p. 1019-1024; Spencer D. M. et al., Curr Biol 1996, 6:839-847; Blau, C. A. et al., Proc Natl Acad. Sci. USA 1997, 94:3076-3081), or cytosolic proteins (Luo, Z. et al., Nature 1996, 383:181-185; MacCorkle, R. A. et al., Proc Natl Acad Sci USA 1998, 95:3655-3660), the recruitment of transcription factors to DNA elements to modulate transcription (Ho, S. N. et al., Nature 1996, 382:822-826; Rivera, V. M. et al., Nat. Med. 1996, 2:1028-1032) or the recruitment of signaling molecules to the plasma membrane to stimulate signaling (Spencer D. M. et al., Proc. Natl. Acad. Sci. USA 1995, 92:9805-9809; Holsinger, L. J. et al., Proc. Natl. Acad. Sci. USA 1995, 95:9810-9814).

The CID system is based upon the notion that surface receptor aggregation effectively activates downstream signaling cascades. In the simplest embodiment, the CID system uses a dimeric analog of the lipid permeable immunosuppressant drug, FK506, which loses its normal bioactivity while gaining the ability to crosslink molecules genetically fused to the FK506-binding protein, FKBP12. By fusing one or more FKBPs to caspase-9, one can stimulate caspase-9 activity in a dimerizer drug-dependent, but ligand and ectodomain-independent manner. This provides the system with temporal control, reversibility using monomeric drug analogs, and enhanced specificity. The high affinity of third-generation AP20187/AP1903 CIDs for their binding domain, FKBP12, permits specific activation of the recombinant receptor in vivo without the induction of non-specific side effects through endogenous FKBP12. FKBP12 variants having amino acid substitutions and deletions, such as FKBP12v36, that bind to a dimerizer drug, may also be used. In addition, the synthetic ligands are resistant to protease degradation, making them more efficient at activating receptors in vivo than most delivered protein agents.

The ligands used are capable of binding to two or more of the ligand-binding domains. The chimeric proteins may be able to bind to more than one ligand when they contain more than one ligand-binding domain. The ligand is typically a non-protein or a chemical. Exemplary ligands include, but are not limited to dimeric FK506 (e.g., FK1012).

Other ligand binding regions may be, for example, dimeric regions, or modified ligand binding regions with a wobble substitution, such as, for example, FKBP12(V36): The human 12 kDa FK506-binding protein with an F36 to V substitution, the complete mature coding sequence (amino acids 1-107), provides a binding site for synthetic dimerizer drug AP1903 (Jemal, A. et al., CA Cancer J. Clinic. 58, 71-96 (2008); Scher, H. I. and Kelly, W. K., Journal of Clinical Oncology 11, 1566-72 (1993)). Two tandem copies of the protein may also be used in the construct so that higher-order oligomers are induced upon cross-linking by AP1903.

F36V′-FKBP: F36V′-FKBP is a codon-wobbled version of F36V-FKBP. It encodes the identical polypeptide sequence as F36V-FKPB but has only 62% homology at the nucleotide level.

F36V′-FKBP was designed to reduce recombination in retroviral vectors (Schellhammer, P. F. et al., J. Urol. 157, 1731-5 (1997)). F36V′-FKBP was constructed by a PCR assembly procedure. The transgene contains one copy of F36V′-FKBP linked directly to one copy of F36V-FKBP.

In some embodiments, the ligand is a small molecule. The appropriate ligand for the selected ligand-binding region may be selected. Often, the ligand is dimeric, sometimes, the ligand is a dimeric FK506 or a dimeric FK506-like analog. In certain embodiments, the ligand is AP1903 (CAS Index Name: 2-Piperidinecarboxylic acid, 1-[(2S)-1-oxo-2-(3,4,5-trimethoxyphenyl)butyl]-, 1,2-ethanediylbis[imino(2-oxo-2,1-ethanediyl)oxy-3,1-phenylene[(1R)-3-(3,4-dimethoxyphenyl)propylidene]]ester, [2S-[1(R*),2R*[S*[S*[1(R*),2R*]]]]]-(9Cl) CAS Registry Number: 195514-63-7; Molecular Formula: C78H98N4O20 Molecular Weight: 1411.65). In certain embodiments, the ligand is AP20187. In certain embodiments, the ligand is an AP20187 analog, such as, for example, AP1510. In some embodiments, certain analogs will be appropriate for the FKBP12, and certain analogs appropriate for the wobbled version of FKBP12. In certain embodiments, one ligand binding region is included in the chimeric protein. In other embodiments, two or more ligand binding regions are included. Where, for example, the ligand binding region is FKBP12, where two of these regions are included, one may, for example, be the wobbled version.

Other dimerization systems contemplated include the coumermycin/DNA gyrase B system. Coumermycin-induced dimerization activates a modified Raf protein and stimulates the MAP kinase cascade. See Farrar et al., 1996.

AP1903 for Injection

AP1903 API is manufactured by Alphora Research Inc. and AP1903 Drug Product for Injection is made by Formatech Inc. It is formulated as a 5 mg/mL solution of AP1903 in a 25% solution of the non-ionic solubilizer Solutol HS 15 (250 mg/mL, BASF). At room temperature, this formulation is a clear, slightly yellow solution. Upon refrigeration, this formulation undergoes a reversible phase transition, resulting in a milky solution. This phase transition is reversed upon re-warming to room temperature. The fill is 2.33 mL in a 3 mL glass vial (˜10 mg AP1903 for Injection total per vial).

AP1903 is removed from the refrigerator the night before the patient is dosed and stored at a temperature of approximately 21° C. overnight, so that the solution is clear prior to dilution. The solution is prepared within 30 minutes of the start of the infusion in glass or polyethylene bottles or non-DEHP bags and stored at approximately 21° C. prior to dosing.

All study medication is maintained at a temperature between 2 degrees C. and 8 degrees C., protected from excessive light and heat, and stored in a locked area with restricted access.

Upon determining a need to administer AP1903 and induce the inducible caspase-9 polypeptide, patients may be, for example, administered a single fixed dose of AP1903 for Injection (0.4 mg/kg) via IV infusion over 2 hours, using a non-DEHP, non-ethylene oxide sterilized infusion set. The dose of AP1903 is calculated individually for all patients, and is not to be recalculated unless body weight fluctuates by ≧10%. The calculated dose is diluted in 100 mL in 0.9% normal saline before infusion.

In a previous Phase I study of AP1903, 24 healthy volunteers were treated with single doses of AP1903 for Injection at dose levels of 0.01, 0.05, 0.1, 0.5 and 1.0 mg/kg infused IV over 2 hours. AP1903 plasma levels were directly proportional to dose, with mean Cₘₐₓ values ranging from approximately 10-1275 ng/mL over the 0.01-1.0 mg/kg dose range. Following the initial infusion period, blood concentrations demonstrated a rapid distribution phase, with plasma levels reduced to approximately 18, 7, and 1% of maximal concentration at 0.5, 2 and 10 hours post-dose, respectively. AP1903 for Injection was shown to be safe and well tolerated at all dose levels and demonstrated a favorable pharmacokinetic profile. Iuliucci J D, et al., J Clin Pharmacol. 41: 870-9, 2001.

The fixed dose of AP1903 for injection used, for example, may be 0.4 mg/kg intravenously infused over 2 hours. The amount of AP1903 needed in vitro for effective signaling of cells is 0.1-10 nM (1412 Da MW). This equates to 0.14-14 μg/L or ˜0.14-14 μg/kg. Doses up to 1 mg/kg were well-tolerated in the Phase I study of AP1903 described above. Therefore, 0.4 mg/kg may be a safe and effective dose of AP1903 for this Phase I study in combination with the therapeutic cells.

The multimeric ligand, such as, for example, AP1903, may be delivered, for example at doses of about 0.01 to 1 mg/kg subject weight, of about 0.05 to 0.5 mg/kg subject weight, 0.1 to 2 mg/kg subject weight, of about 0.05 to 1.0 mg/kg subject weight, of about 0.1 to 5 mg/kg subject weight, of about 0.2 to 4 mg/kg subject weight, of about 0.3 to 3 mg/kg subject weight, of about 0.3 to 2 mg/kg subject weight, or about 0.3 to 1 mg/kg subject weight, for example, about 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, or 10 mg/kg subject weight. In some embodiments, the ligand is provided at 0.4 mg/kg per dose, for example at a concentration of 5 mg/mL. Vials or other containers may be provided containing the ligand at, for example, a volume per vial of about 0.25 ml to about 10 ml, for example, about 0.25, 0.5, 1, 1.5, 2, 2.5, 3, 3.5, 4, 4.5, 5, 5.5, 6, 6.5, 7, 7.5, 8, 8.5, 9, 9.5, or 10 ml, for example, about 2 ml.

Selectable Markers

In certain embodiments, the expression constructs contain nucleic acid constructs whose expression is identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as Herpes Simplex Virus-I thymidine kinase (tk) are employed. Immunologic surface markers containing the extracellular, non-signaling domains or various proteins (e.g. CD34, CD19, LNGFR) also can be employed, permitting a straightforward method for magnetic or fluorescence antibody-mediated sorting. The selectable marker employed is not believed to be important, so long as it is capable of being expressed simultaneously with the nucleic acid encoding a gene product. Further examples of selectable markers include, for example, reporters such as GFP, EGFP, beta-gal or chloramphenicol acetyltransferase (CAT). In certain embodiments, the marker protein, such as, for example, CD19 is used for selection of the cells for transfusion, such as, for example, in immunomagnetic selection. As discussed herein, a CD19 marker is distinguished from an anti-CD19 antibody, or, for example, an scFv, TCR, or other antigen recognition moiety that binds to CD19.

In some embodiments, a polypeptide may be included in the expression vector to aid in sorting cells. For example, the CD34 minimal epitope may be incorporated into the vector. In some embodiments, the expression vectors used to express the chimeric antigen receptors or chimeric stimulating molecules provided herein further comprise a polynucleotide that encodes the 16 amino acid CD34 minimal epitope. In some embodiments, such as certain embodiments provided in the examples herein, the CD34 minimal epitope is incorporated at the amino terminal position of the CD8 stalk.

Transmembrane Regions

A chimeric antigen receptor herein may include a single-pass or multiple pass transmembrane sequence (e.g., at the N-terminus or C-terminus of the chimeric protein). Single pass transmembrane regions are found in certain CD molecules, tyrosine kinase receptors, serine/threonine kinase receptors, TGFβ, BMP, activin and phosphatases. Single pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane. Multiple pass proteins include ion pumps, ion channels, and transporters, and include two or more helices that span the membrane multiple times. All or substantially all of a multiple pass protein sometimes is incorporated in a chimeric protein. Sequences for single pass and multiple pass transmembrane regions are known and can be selected for incorporation into a chimeric protein molecule.

In some embodiments, the transmembrane domain is fused to the extracellular domain of the CAR. In one embodiment, the transmembrane domain that naturally is associated with one of the domains in the CAR is used. In other embodiments, a transmembrane domain that is not naturally associated with one of the domains in the CAR is used. In some instances, the transmembrane domain can be selected or modified by amino acid substitution (e.g., typically charged to a hydrophobic residue) to avoid binding of such domains to the transmembrane domains of the same or different surface membrane proteins to minimize interactions with other members of the receptor complex.

Transmembrane domains may, for example, be derived from the alpha, beta, or zeta chain of the T cell receptor, CD3-ε, CD3ζ, CD4, CD5, CD8, CD8α, CD9, CD16, CD22, CD28, CD33, CD38, CD64, CD80, CD86, CD134, CD137, or CD154. Or, in some examples, the transmembrane domain may be synthesized de novo, comprising mostly hydrophobic residues, such as, for example, leucine and valine. In certain embodiments a short polypeptide linker may form the linkage between the transmembrane domain and the intracellular domain of the chimeric antigen receptor. The chimeric antigen receptors may further comprise a stalk, that is, an extracellular region of amino acids between the extracellular domain and the transmembrane domain. For example, the stalk may be a sequence of amino acids naturally associated with the selected transmembrane domain. In some embodiments, the chimeric antigen receptor comprises a CD8 transmembrane domain, in certain embodiments, the chimeric antigen receptor comprises a CD8 transmembrane domain, and additional amino acids on the extracellular portion of the transmembrane domain, in certain embodiments, the chimeric antigen receptor comprises a CD8 transmembrane domain and a CD8 stalk. The chimeric antigen receptor may further comprise a region of amino acids between the transmembrane domain and the cytoplasmic domain, which are naturally associated with the polypeptide from which the transmembrane domain is derived.

Control Regions

1. Promoters

The particular promoter employed to control the expression of a polynucleotide sequence of interest is not believed to be important, so long as it is capable of directing the expression of the polynucleotide in the targeted cell. Thus, where a human cell is targeted the polynucleotide sequence-coding region may, for example, be placed adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it is desirable to prohibit or reduce expression of one or more of the transgenes. Examples of transgenes that are toxic to the producer cell line are pro-apoptotic and cytokine genes. Several inducible promoter systems are available for production of viral vectors where the transgene products are toxic (add in more inducible promoters).

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter, which drives expression of the gene of interest, is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that may be useful is the Tet-Off™ or Tet-On™ system (Clontech, Palo Alto, Calif.) originally developed by Gossen and Bujard (Gossen and Bujard, Proc. Natl. Acad. Sci. USA, 89:5547-5551, 1992; Gossen et al., Science, 268:1766-1769, 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli, he tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tetracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system may be used so that the producer cells could be grown in the presence of tetracycline or doxycycline and prevent expression of a potentially toxic transgene, but when the vector is introduced to the patient, the gene expression would be constitutively on.

In some circumstances, it is desirable to regulate expression of a transgene in a gene therapy vector. For example, different viral promoters with varying strengths of activity are utilized depending on the level of expression desired. In mammalian cells, the CMV immediate early promoter is often used to provide strong transcriptional activation. The CMV promoter is reviewed in Donnelly, J. J., et al., 1997. Annu. Rev. Immunol. 15:617-48. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that are used depending on the desired effect include SV40, RSV LTR, HIV-1 and HIV-2 LTR, adenovirus promoters such as from the E1A, E2A, or MLP region, AAV LTR, HSV-TK, and avian sarcoma virus.

In other examples, promoters may be selected that are developmentally regulated and are active in particular differentiated cells. Thus, for example, a promoter may not be active in a pluripotent stem cell, but, for example, where the pluripotent stem cell differentiates into a more mature cell, the promoter may then be activated.

Similarly tissue specific promoters are used to effect transcription in specific tissues or cells so as to reduce potential toxicity or undesirable effects to non-targeted tissues. These promoters may result in reduced expression compared to a stronger promoter such as the CMV promoter, but may also result in more limited expression, and immunogenicity (Bojak, A., et al., 2002. Vaccine. 20:1975-79; Cazeaux., N., et al., 2002. Vaccine 20:3322-31). For example, tissue specific promoters such as the PSA associated promoter or prostate-specific glandular kallikrein, or the muscle creatine kinase gene may be used where appropriate.

Examples of tissue specific or differentiation specific promoters include, but are not limited to, the following: B29 (B cells); CD14 (monocytic cells); CD43 (leukocytes and platelets); CD45 (hematopoietic cells); CD68 (macrophages); desmin (muscle); elastase-1 (pancreatic acinar cells); endoglin (endothelial cells); fibronectin (differentiating cells, healing tissues); and Flt-1 (endothelial cells); GFAP (astrocytes).

In certain indications, it is desirable to activate transcription at specific times after administration of the gene therapy vector. This is done with such promoters as those that are hormone or cytokine regulatable. Cytokine and inflammatory protein responsive promoters that can be used include K and T kininogen (Kageyama et al., (1987) J. Biol. Chem., 262, 2345-2351), c-fos, TNF-alpha, C-reactive protein (Arcone, et al., (1988) Nucl. Acids Res., 16(8), 3195-3207), haptoglobin (Oliviero et al., (1987) EMBO J., 6, 1905-1912), serum amyloid A2, C/EBP alpha, IL-1, IL-6 (Poli and Cortese, (1989) Proc. Nat'l Acad. Sci. USA, 86, 8202-8206), Complement C3 (Wilson et al., (1990) Mol. Cell. Biol., 6181-6191), IL-8, alpha-1 acid glycoprotein (Prowse and Baumann, (1988) Mol Cell Biol, 8, 42-51), alpha-1 antitrypsin, lipoprotein lipase (Zechner et al., Mol. Cell. Biol., 2394-2401, 1988), angiotensinogen (Ron, et al., (1991) Mol. Cell. Biol., 2887-2895), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 anti-chymotrypsin. Other promoters include, for example, SV40, MMTV, Human Immunodeficiency Virus (MV), Moloney virus, ALV, Epstein Barr virus, Rous Sarcoma virus, human actin, myosin, hemoglobin, and creatine.

It is envisioned that any of the above promoters alone or in combination with another can be useful depending on the action desired. Promoters, and other regulatory elements, are selected such that they are functional in the desired cells or tissue. In addition, this list of promoters should not be construed to be exhaustive or limiting; other promoters that are used in conjunction with the promoters and methods disclosed herein.

2. Enhancers

Enhancers are genetic elements that increase transcription from a promoter located at a distant position on the same molecule of DNA. Early examples include the enhancers associated with immunoglobulin and T cell receptors that both flank the coding sequence and occur within several introns. Many viral promoters, such as CMV, SV40, and retroviral LTRs are closely associated with enhancer activity and are often treated like single elements. Enhancers are organized much like promoters. That is, they are composed of many individual elements, each of which binds to one or more transcriptional proteins. The basic distinction between enhancers and promoters is operational. An enhancer region as a whole stimulates transcription at a distance and often independent of orientation; this need not be true of a promoter region or its component elements. On the other hand, a promoter has one or more elements that direct initiation of RNA synthesis at a particular site and in a particular orientation, whereas enhancers lack these specificities. Promoters and enhancers are often overlapping and contiguous, often seeming to have a very similar modular organization. A subset of enhancers includes locus-control regions (LCRs) that can not only increase transcriptional activity, but (along with insulator elements) can also help to insulate the transcriptional element from adjacent sequences when integrated into the genome. Any promoter/enhancer combination (as per the Eukaryotic Promoter Data Base EPDB) can be used to drive expression of the gene, although many will restrict expression to a particular tissue type or subset of tissues (reviewed in, for example, Kutzler, M. A., and Weiner, D. B., 2008. Nature Reviews Genetics 9:776-88). Examples include, but are not limited to, enhancers from the human actin, myosin, hemoglobin, muscle creatine kinase, sequences, and from viruses CMV, RSV, and EBV. Appropriate enhancers may be selected for particular applications. Eukaryotic cells can support cytoplasmic transcription from certain bacterial promoters if the appropriate bacterial polymerase is provided, either as part of the delivery complex or as an additional genetic expression construct.

3. Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the present methods, and any such sequence is employed such as human or bovine growth hormone and SV40 polyadenylation signals and LTR polyadenylation signals. One non-limiting example is the SV40 polyadenylation signal present in the pCEP3 plasmid (Invitrogen, Carlsbad, Calif.). Also, contemplated as an element of the expression cassette is a terminator. These elements can serve to enhance message levels and to minimize read through from the cassette into other sequences. Termination or poly(A) signal sequences may be, for example, positioned about 11-30 nucleotides downstream from a conserved sequence (AAUAAA) at the 3′ end of the mRNA (Montgomery, D. L., et al., 1993. DNA Cell Biol. 12:777-83; Kutzler, M. A., and Weiner, D. B., 2008. Nature Rev. Gen. 9:776-88).

4. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. The initiation codon is placed in-frame with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements is used to create multigene, or polycistronic messages. IRES elements are able to bypass the ribosome-scanning model of 5′ methylated cap-dependent translation and begin translation at internal sites (Pelletier and Sonenberg, Nature, 334:320-325, 1988). IRES elements from two members of the picornavirus family (polio and encephalomyocarditis) have been discussed (Pelletier and Sonenberg, 1988), as well an IRES from a mammalian message (Macejak and Sarnow, Nature, 353:90-94, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

Sequence Optimization

Protein production may also be increased by optimizing the codons in the transgene. Species specific codon changes may be used to increase protein production. Also, codons may be optimized to produce an optimized RNA, which may result in more efficient translation. By optimizing the codons to be incorporated in the RNA, elements such as those that result in a secondary structure that causes instability, secondary mRNA structures that can, for example, inhibit ribosomal binding, or cryptic sequences that can inhibit nuclear export of mRNA can be removed (Kutzler, M. A., and Weiner, D. B., 2008. Nature Rev. Gen. 9:776-88; Yan, J. et al., 2007. Mol. Ther. 15:411-21; Cheung, Y. K., et al., 2004. Vaccine 23:629-38; Narum., D. L., et al., 2001. 69:7250-55; Yadava, A., and Ockenhouse, C. F., 2003. Infect. Immun. 71:4962-69; Smith., J. M., et al., 2004. AIDS Res. Hum. Retroviruses 20:1335-47; Zhou, W., et al., 2002. Vet. Microbiol. 88:127-51; Wu, X., et al., 2004. Biochem. Biophys. Res. Commun. 313:89-96; Zhang, W., et al., 2006. Biochem. Biophys. Res. Commun. 349:69-78; Deml, L. A., et al., 2001. J. Virol. 75:1099-11001; Schneider, R. M., et al., 1997. J. Virol. 71:4892-4903; Wang, S. D., et al., 2006. Vaccine 24:4531-40; zur Megede, J., et al., 2000. J. Virol. 74:2628-2635). For example, the FKBP12 polypeptide, the Caspase polypeptide, and the CD19 sequences may be optimized by changes in the codons.

Leader Sequences

Leader sequences may be added to enhance the stability of mRNA and result in more efficient translation. The leader sequence is usually involved in targeting the mRNA to the endoplasmic reticulum. Examples include the signal sequence for the HIV-1 envelope glycoprotein (Env), which delays its own cleavage, and the IgE gene leader sequence (Kutzler, M. A., and Weiner, D. B., 2008. Nature Rev. Gen. 9:776-88; Li, V., et al., 2000. Virology 272:417-28; Xu, Z. L., et al. 2001. Gene 272:149-56; Malin, A. S., et al., 2000. Microbes Infect. 2:1677-85; Kutzler, M. A., et al., 2005. J. Immunol. 175:112-125; Yang, J. S., et al., 2002. Emerg. Infect. Dis. 8:1379-84; Kumar., S., et al., 2006. DNA Cell Biol. 25:383-92; Wang, S., et al., 2006. Vaccine 24:4531-40). The IgE leader may be used to enhance insertion into the endoplasmic reticulum (Tepler, I, et al. (1989) J. Biol. Chem. 264:5912).

Expression of the transgenes may be optimized and/or controlled by the selection of appropriate methods for optimizing expression. These methods include, for example, optimizing promoters, delivery methods, and gene sequences, (for example, as presented in Laddy, D. J., et al., 2008. PLoS. ONE 3 e2517; Kutzler, M. A., and Weiner, D. B., 2008. Nature Rev. Gen. 9:776-88).

### Nucleic Acids

A “nucleic acid” as used herein generally refers to a molecule (one, two or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.” Nucleic acids may be, be at least, be at most, or be about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length.

Nucleic acids herein provided may have regions of identity or complementarity to another nucleic acid. It is contemplated that the region of complementarity or identity can be at least 5 contiguous residues, though it is specifically contemplated that the region is, is at least, is at most, or is about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides.

As used herein, “hybridization”, “hybridizes” or “capable of hybridizing” is understood to mean forming a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are known, and are often used for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42 degrees C. to about 70 degrees C. It is understood that the temperature and ionic strength of a desired stringency are determined in part by the length of the particular nucleic acid(s), the length and nucleobase content of the target sequence(s), the charge composition of the nucleic acid(s), and the presence or concentration of formamide, tetramethylammonium chloride or other solvent(s) in a hybridization mixture.

It is understood that these ranges, compositions and conditions for hybridization are mentioned by way of non-limiting examples only, and that the desired stringency for a particular hybridization reaction is often determined empirically by comparison to one or more positive or negative controls. Depending on the application envisioned varying conditions of hybridization may be employed to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20 degrees C. to about 50 degrees C. The low or high stringency conditions may be further modified to suit a particular application.

### Nucleic Acid Modification

Any of the modifications discussed below may be applied to a nucleic acid. Examples of modifications include alterations to the RNA or DNA backbone, sugar or base, and various combinations thereof. Any suitable number of backbone linkages, sugars and/or bases in a nucleic acid can be modified (e.g., independently about 5%, 10%, 15%, 20%, 25%, 30%, 35%, 40%, 45%, 50%, 55%, 60%, 65%, 70%, 75%, 80%, 85%, 90%, 95%, up to 100%). An unmodified nucleoside is any one of the bases adenine, cytosine, guanine, thymine, or uracil joined to the 1′ carbon of beta-D-ribo-furanose.

A modified base is a nucleotide base other than adenine, guanine, cytosine and uracil at a 1′ position. Non-limiting examples of modified bases include inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e. g., 5-methylcytidine), 5-alkyluridines (e. g., ribothymidine), 5-halouridine (e. g., 5-bromouridine) or 6-azapyrimidines or 6-alkylpyrimidines (e. g. 6-methyluridine), propyne, and the like. Other non-limiting examples of modified bases include nitropyrrolyl (e.g., 3-nitropyrrolyl), nitroindolyl (e.g., 4-, 5-, 6-nitroindolyl), hypoxanthinyl, isoinosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, difluorotolyl, 4-fluoro-6-methylbenzimidazole, 4-methylbenzimidazole, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenyl, tetracenyl, pentacenyl and the like.

In some embodiments, for example, a nucleic acid may comprise modified nucleic acid molecules, with phosphate backbone modifications. Non-limiting examples of backbone modifications include phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl modifications. In certain instances, a ribose sugar moiety that naturally occurs in a nucleoside is replaced with a hexose sugar, polycyclic heteroalkyl ring, or cyclohexenyl group. In certain instances, the hexose sugar is an allose, altrose, glucose, mannose, gulose, idose, galactose, talose, or a derivative thereof. The hexose may be a D-hexose, glucose, or mannose. In certain instances, the polycyclic heteroalkyl group may be a bicyclic ring containing one oxygen atom in the ring. In certain instances, the polycyclic heteroalkyl group is a bicyclo[2.2.1]heptane, a bicyclo[3.2.1]octane, or a bicyclo[3.3.1]nonane.

Nitropyrrolyl and nitroindolyl nucleobases are members of a class of compounds known as universal bases. Universal bases are those compounds that can replace any of the four naturally occurring bases without substantially affecting the melting behavior or activity of the oligonucleotide duplex. In contrast to the stabilizing, hydrogen-bonding interactions associated with naturally occurring nucleobases, oligonucleotide duplexes containing 3-nitropyrrolyl nucleobases may be stabilized solely by stacking interactions. The absence of significant hydrogen-bonding interactions with nitropyrrolyl nucleobases obviates the specificity for a specific complementary base. In addition, 4-, 5- and 6-nitroindolyl display very little specificity for the four natural bases. Procedures for the preparation of 1-(2′-O-methyl-.beta.-D-ribofuranosyl)-5-nitroindole are discussed in Gaubert, G.; Wengel, J. Tetrahedron Letters 2004, 45, 5629. Other universal bases include hypoxanthinyl, isoinosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, and structural derivatives thereof.

Difluorotolyl is a non-natural nucleobase that functions as a universal base. Difluorotolyl is an isostere of the natural nucleobase thymine. But unlike thymine, difluorotolyl shows no appreciable selectivity for any of the natural bases. Other aromatic compounds that function as universal bases are 4-fluoro-6-methylbenzimidazole and 4-methylbenzimidazole. In addition, the relatively hydrophobic isocarbostyrilyl derivatives 3-methyl isocarbostyrilyl, 5-methyl isocarbostyrilyl, and 3-methyl-7-propynyl isocarbostyrilyl are universal bases which cause only slight destabilization of oligonucleotide duplexes compared to the oligonucleotide sequence containing only natural bases. Other non-natural nucleobases include 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenyl, tetracenyl, pentacenyl, and structural derivates thereof. For a more detailed discussion, including synthetic procedures, of difluorotolyl, 4-fluoro-6-methylbenzimidazole, 4-methylbenzimidazole, and other non-natural bases mentioned above, see: Schweitzer et al., J. Org. Chem., 59:7238-7242 (1994);

In addition, chemical substituents, for example cross-linking agents, may be used to add further stability or irreversibility to the reaction. Non-limiting examples of cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl) dithio]propioimidate.

A nucleotide analog may also include a “locked” nucleic acid. Certain compositions can be used to essentially “anchor” or “lock” an endogenous nucleic acid into a particular structure. Anchoring sequences serve to prevent disassociation of a nucleic acid complex, and thus not only can prevent copying but may also enable labeling, modification, and/or cloning of the endogenous sequence. The locked structure may regulate gene expression (i.e. inhibit or enhance transcription or replication), or can be used as a stable structure that can be used to label or otherwise modify the endogenous nucleic acid sequence, or can be used to isolate the endogenous sequence, i.e. for cloning.

Nucleic acid molecules need not be limited to those molecules containing only RNA or DNA, but further encompass chemically-modified nucleotides and non-nucleotides. The percent of non-nucleotides or modified nucleotides may be from 1% to 100% (e.g., about 5, 10, 15, 20, 25, 30, 35, 40, 45, 50, 55, 60, 65, 70, 75, 80, 85, 90 or 95%).

### Nucleic Acid Preparation

In some embodiments, a nucleic acid is provided for use as a control or standard in an assay, or therapeutic, for example. A nucleic acid may be made by any technique known in the art, such as for example, chemical synthesis, enzymatic production or biological production. Nucleic acids may be recovered or isolated from a biological sample. The nucleic acid may be recombinant or it may be natural or endogenous to the cell (produced from the cell's genome). It is contemplated that a biological sample may be treated in a way so as to enhance the recovery of small nucleic acid molecules. Generally, methods may involve lysing cells with a solution having guanidinium and a detergent.

Nucleic acid synthesis may also be performed according to standard methods. Non-limiting examples of a synthetic nucleic acid (e.g., a synthetic oligonucleotide), include a nucleic acid made by in vitro chemical synthesis using phosphotriester, phosphite, or phosphoramidite chemistry and solid phase techniques or via deoxynucleoside H-phosphonate intermediates. Various different mechanisms of oligonucleotide synthesis have been disclosed elsewhere.

Nucleic acids may be isolated using known techniques. In particular embodiments, methods for isolating small nucleic acid molecules, and/or isolating RNA molecules can be employed. Chromatography is a process used to separate or isolate nucleic acids from protein or from other nucleic acids. Such methods can involve electrophoresis with a gel matrix, filter columns, alcohol precipitation, and/or other chromatography. If a nucleic acid from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

Methods may involve the use of organic solvents and/or alcohol to isolate nucleic acids. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column is effective for such isolation procedures.

A nucleic acid isolation processes may sometimes include: a) lysing cells in the sample with a lysing solution comprising guanidinium, where a lysate with a concentration of at least about 1 M guanidinium is produced; b) extracting nucleic acid molecules from the lysate with an extraction solution comprising phenol; c) adding to the lysate an alcohol solution to form a lysate/alcohol mixture, wherein the concentration of alcohol in the mixture is between about 35% to about 70%; d) applying the lysate/alcohol mixture to a solid support; e) eluting the nucleic acid molecules from the solid support with an ionic solution; and, f) capturing the nucleic acid molecules. The sample may be dried down and resuspended in a liquid and volume appropriate for subsequent manipulation.

### Methods of Gene Transfer

In order to mediate the effect of the transgene expression in a cell, it will be necessary to transfer the expression constructs into a cell. Such transfer may employ viral or non-viral methods of gene transfer. This section provides a discussion of methods and compositions of gene transfer.

A transformed cell comprising an expression vector is generated by introducing into the cell the expression vector. Suitable methods for polynucleotide delivery for transformation of an organelle, a cell, a tissue or an organism for use with the current methods include virtually any method by which a polynucleotide (e.g., DNA) can be introduced into an organelle, a cell, a tissue or an organism.

A host cell can, and has been, used as a recipient for vectors. Host cells may be derived from prokaryotes or eukaryotes, depending upon whether the desired result is replication of the vector or expression of part or all of the vector-encoded polynucleotide sequences. Numerous cell lines and cultures are available for use as a host cell, and they can be obtained through the American Type Culture Collection (ATCC), which is an organization that serves as an archive for living cultures and genetic materials. In specific embodiments, the host cell is a T cell, a tumor-infiltrating lymphocyte, a natural killer cell, or a natural killer T cell.

An appropriate host may be determined. Generally this is based on the vector backbone and the desired result. A plasmid or cosmid, for example, can be introduced into a prokaryote host cell for replication of many vectors. Bacterial cells used as host cells for vector replication and/or expression include DH5alpha, JM109, and KC8, as well as a number of commercially available bacterial hosts such as SURE® Competent Cells and SOLOPACK Gold Cells (STRATAGENE®, La Jolla, Calif.). Alternatively, bacterial cells such as E. coli LE392 could be used as host cells for phage viruses. Eukaryotic cells that can be used as host cells include, but are not limited to yeast, insects and mammals. Examples of mammalian eukaryotic host cells for replication and/or expression of a vector include, but are not limited to, HeLa, NIH3T3, Jurkat, 293, COS, CHO, Saos, and PC12. Examples of yeast strains include, but are not limited to, YPH499, YPH500 and YPH501.

Nucleic acid vaccines may include, for example, non-viral DNA vectors, “naked” DNA and RNA, and viral vectors. Methods of transforming cells with these vaccines, and for optimizing the expression of genes included in these vaccines are known and are also discussed herein.

Examples of Methods of Nucleic Acid or Viral Vector Transfer

Any appropriate method may be used to transfect or transduce the cells, or to administer the nucleotide sequences or compositions of the present methods. Certain examples are presented herein, and further include methods such as delivery using cationic polymers, lipid like molecules, and certain commercial products such as, for example, IN-VIVO-JET PEI.

1. Ex Vivo Transformation

Various methods are available for transfecting vascular cells and tissues removed from an organism in an ex vivo setting. For example, canine endothelial cells have been genetically altered by retroviral gene transfer in vitro and transplanted into a canine (Wilson et al., Science, 244:1344-1346, 1989). In another example, Yucatan minipig endothelial cells were transfected by retrovirus in vitro and transplanted into an artery using a double-balloon catheter (Nabel et al., Science, 244(4910):1342-1344, 1989). Thus, it is contemplated that cells or tissues may be removed and transfected ex vivo using the polynucleotides presented herein. In particular aspects, the transplanted cells or tissues may be placed into an organism.

2. Injection

In certain embodiments, an antigen presenting cell or a nucleic acid or viral vector may be delivered to an organelle, a cell, a tissue or an organism via one or more injections (i.e., a needle injection), such as, for example, subcutaneous, intradermal, intramuscular, intravenous, intraprotatic, intratumor, intraperitoneal, etc. Methods of injection include, for example, injection of a composition comprising a saline solution. Further embodiments include the introduction of a polynucleotide by direct microinjection. The amount of the expression vector used may vary upon the nature of the antigen as well as the organelle, cell, tissue or organism used.

Intradermal, intranodal, or intralymphatic injections are some of the more commonly used methods of DC administration. Intradermal injection is characterized by a low rate of absorption into the bloodstream but rapid uptake into the lymphatic system. The presence of large numbers of Langerhans dendritic cells in the dermis will transport intact as well as processed antigen to draining lymph nodes. Proper site preparation is necessary to perform this correctly (i.e., hair is clipped in order to observe proper needle placement). Intranodal injection allows for direct delivery of antigen to lymphoid tissues. Intralymphatic injection allows direct administration of DCs.

3. Electroporation

In certain embodiments, a polynucleotide is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference).

Transfection of eukaryotic cells using electroporation has been quite successful. Mouse pre-B lymphocytes have been transfected with human kappa-immunoglobulin genes (Potter et al., (1984) Proc. Nat'l Acad. Sci. USA, 81, 7161-7165), and rat hepatocytes have been transfected with the chloramphenicol acetyltransferase gene (Tur-Kaspa et al., (1986) Mol. Cell Biol., 6, 716-718) in this manner.

In vivo electroporation for vaccines, or eVac, is clinically implemented through a simple injection technique. A DNA vector encoding a polypeptide is injected intradermally in a patient. Then electrodes apply electrical pulses to the intradermal space causing the cells localized there, especially resident dermal dendritic cells, to take up the DNA vector and express the encoded polypeptide. These polypeptide-expressing cells activated by local inflammation can then migrate to lymph-nodes, presenting antigens, for example. A nucleic acid is electroporetically administered when it is administered using electroporation, following, for example, but not limited to, injection of the nucleic acid or any other means of administration where the nucleic acid may be delivered to the cells by electroporation

Methods of electroporation are discussed in, for example, Sardesai, N. Y., and Weiner, D. B., Current Opinion in Immunotherapy 23:421-9 (2011) and Ferraro, B. et al., Human Vaccines 7:120-127 (2011), which are hereby incorporated by reference herein in their entirety.

4. Calcium Phosphate

In other embodiments, a polynucleotide is introduced to the cells using calcium phosphate precipitation. Human KB cells have been transfected with adenovirus 5 DNA (Graham and van der Eb, (1973) Virology, 52, 456-467) using this technique. Also in this manner, mouse L(A9), mouse C127, CHO, CV-1, BHK, NIH3T3 and HeLa cells were transfected with a neomycin marker gene (Chen and Okayama, Mol. Cell Biol., 7(8):2745-2752, 1987), and rat hepatocytes were transfected with a variety of marker genes (Rippe et al., Mol. Cell Biol., 10:689-695, 1990).

5. DEAE-Dextran

In another embodiment, a polynucleotide is delivered into a cell using DEAE-dextran followed by polyethylene glycol. In this manner, reporter plasmids were introduced into mouse myeloma and erythroleukemia cells (Gopal, T. V., Mol Cell Biol. 1985 May; 5(5):1188-90).

6. Sonication Loading

Additional embodiments include the introduction of a polynucleotide by direct sonic loading. LTK-fibroblasts have been transfected with the thymidine kinase gene by sonication loading (Fechheimer et al., (1987) Proc. Nat'l Acad. Sci. USA, 84, 8463-8467).

7. Liposome-Mediated Transfection

In a further embodiment, a polynucleotide may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine (Gibco BRL) or Superfect (Qiagen).

8. Receptor Mediated Transfection

Still further, a polynucleotide may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity.

Certain receptor-mediated gene targeting vehicles comprise a cell receptor-specific ligand and a polynucleotide-binding agent. Others comprise a cell receptor-specific ligand to which the polynucleotide to be delivered has been operatively attached. Several ligands have been used for receptor-mediated gene transfer (Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085), which establishes the operability of the technique. Specific delivery in the context of another mammalian cell type has been discussed (Wu and Wu, Adv. Drug Delivery Rev., 12:159-167, 1993; incorporated herein by reference). In certain aspects, a ligand is chosen to correspond to a receptor specifically expressed on the target cell population.

In other embodiments, a polynucleotide delivery vehicle component of a cell-specific polynucleotide-targeting vehicle may comprise a specific binding ligand in combination with a liposome. The polynucleotide(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a polynucleotide to cells that exhibit upregulation of the EGF receptor.

In still further embodiments, the polynucleotide delivery vehicle component of a targeted delivery vehicle may be a liposome itself, which may, for example, comprise one or more lipids or glycoproteins that direct cell-specific binding. For example, lactosyl-ceramide, a galactose-terminal asialoganglioside, have been incorporated into liposomes and observed an increase in the uptake of the insulin gene by hepatocytes (Nicolau et al., (1987) Methods Enzymol., 149, 157-176). It is contemplated that the tissue-specific transforming constructs may be specifically delivered into a target cell in a similar manner.

9. Microprojectile Bombardment

Microprojectile bombardment techniques can be used to introduce a polynucleotide into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., (1987) Nature, 327, 70-73). There are a wide variety of microprojectile bombardment techniques known in the art, many of which are applicable to the present methods.

In this microprojectile bombardment, one or more particles may be coated with at least one polynucleotide and delivered into cells by a propelling force. Several devices for accelerating small particles have been developed. One such device relies on a high voltage discharge to generate an electrical current, which in turn provides the motive force (Yang et al., (1990) Proc. Nat'l Acad. Sci. USA, 87, 9568-9572). The microprojectiles used have consisted of biologically inert substances such as tungsten or gold particles or beads. Exemplary particles include those comprised of tungsten, platinum, and, in certain examples, gold, including, for example, nanoparticles. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

Examples of Methods of Viral Vector-Mediated Transfer

Any viral vector suitable for administering nucleotide sequences, or compositions comprising nucleotide sequences, to a cell or to a subject, such that the cell or cells in the subject may express the genes encoded by the nucleotide sequences may be employed in the present methods. In certain embodiments, a transgene is incorporated into a viral particle to mediate gene transfer to a cell. Typically, the virus simply will be exposed to the appropriate host cell under physiologic conditions, permitting uptake of the virus. The present methods are advantageously employed using a variety of viral vectors, as discussed below.

1. Adenovirus

Adenovirus is particularly suitable for use as a gene transfer vector because of its mid-sized DNA genome, ease of manipulation, high titer, wide target-cell range, and high infectivity. The roughly 36 kb viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, M. J. (1990) Radiother Oncol., 19, 197-218). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence, which makes them useful for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present methods, it is possible to achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay, R. T., et al., J Mol Biol. 1984 Jun. 5; 175(4):493-510). Therefore, inclusion of these elements in an adenoviral vector may permits replication.

In addition, the packaging signal for viral encapsulation is localized between 194-385 bp (0.5-1.1 map units) at the left end of the viral genome (Hearing et al., J. (1987) Virol., 67, 2555-2558). This signal mimics the protein recognition site in bacteriophage lambda DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., Gene, 101:195-202, 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element derives from the packaging function of adenovirus.

It has been shown that a packaging signal for adenovirus exists in the left end of the conventional adenovirus map (Tibbetts et. al. (1977) Cell, 12, 243-249). Later studies showed that a mutant with a deletion in the E1A (194-358 bp) region of the genome grew poorly even in a cell line that complemented the early (E1A) function (Hearing and Shenk, (1983) J. Mol. Biol. 167, 809-822). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved toward the interior of the Ad5 DNA molecule (Hearing et al., J. (1987) Virol., 67, 2555-2558).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals is packaged selectively when compared to the helpers. If the preference is great enough, stocks approaching homogeneity may be achieved.

To improve the tropism of ADV constructs for particular tissues or species, the receptor-binding fiber sequences can often be substituted between adenoviral isolates. For example the Coxsackie-adenovirus receptor (CAR) ligand found in adenovirus 5 can be substituted for the CD46-binding fiber sequence from adenovirus 35, making a virus with greatly improved binding affinity for human hematopoietic cells. The resulting “pseudotyped” virus, Ad5f35, has been the basis for several clinically developed viral isolates. Moreover, various biochemical methods exist to modify the fiber to allow re-targeting of the virus to target cells. Methods include use of bifunctional antibodies (with one end binding the CAR ligand and one end binding the target sequence), and metabolic biotinylation of the fiber to permit association with customized avidin-based chimeric ligands. Alternatively, one could attach ligands (e.g. anti-CD205 by heterobifunctional linkers (e.g. PEG-containing), to the adenovirus particle.

2. Retrovirus

The retroviruses are a group of single-stranded RNA viruses characterized by an ability to convert their RNA to double-stranded DNA in infected cells by a process of reverse-transcription (Coffin, (1990) In: Virology, ed., New York: Raven Press, pp. 1437-1500). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990). Thus, for example, the present technology includes, for example, cells whereby the polynucleotide used to transduce the cell is integrated into the genome of the cell.

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and psi components is constructed (Mann et al., (1983) Cell, 33, 153-159). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas, J. F., and Rubenstein, J. L. R., (1988) In: Vectors: a Survey of Molecular Cloning Vectors and Their Uses, Rodriquez and Denhardt, Eds.). Nicolas and Rubenstein; Temin et al., (1986) In: Gene Transfer, Kucherlapati (ed.), New York: Plenum Press, pp. 149-188; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., (1975) Virology, 67, 242-248).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, may be desired.

A different approach to targeting of recombinant retroviruses was designed, which used biotinylated antibodies against a retroviral envelope protein and against a specific cell receptor. The antibodies were coupled via the biotin components by using streptavidin (Roux et al., (1989) Proc. Nat'l Acad. Sci. USA, 86, 9079-9083). Using antibodies against major histocompatibility complex class I and class II antigens, the infection of a variety of human cells that bore those surface antigens was demonstrated with an ecotropic virus in vitro (Roux et al., 1989).

3. Adeno-Associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low-level expression of AAV rep proteins believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., J. Virol., 61:3096-3101 (1987)), or by other methods, including but not limited to chemical or enzymatic synthesis of the terminal repeats based upon the published sequence of AAV. It can be determined, for example, by deletion analysis, the minimum sequence or part of the AAV ITRs which is required to allow function, i.e., stable and site-specific integration. It can also be determined which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, (1995) Ann. N.Y. Acad. Sci., 770; 79-90; Chatteijee, et al., (1995) Ann. N.Y. Acad. Sci., 770, 79-90; Ferrari et al., (1996) J. Virol., 70, 3227-3234; Fisher et al., (1996) J. Virol., 70, 520-532; Flotte et al., Proc. Nat'l Acad. Sci. USA, 90, 10613-10617, (1993); Goodman et al. (1994), Blood, 84, 1492-1500; Kaplitt et al., (1994) Nat'l Genet., 8, 148-153; Kaplitt, M. G., et al., Ann Thorac Surg. 1996 December; 62(6):1669-76; Kessler et al., (1996) Proc. Nat'l Acad. Sci. USA, 93, 14082-14087; Koeberl et al., (1997) Proc. Nat'l Acad. Sci. USA, 94, 1426-1431; Mizukami et al., (1996) Virology, 217, 124-130).

AAV-mediated efficient gene transfer and expression in the lung has led to clinical trials for the treatment of cystic fibrosis (Carter and Flotte, 1995; Flotte et al., Proc. Nat'l Acad. Sci. USA, 90, 10613-10617, (1993)). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., (1996) J. Virol., 70, 520-532; Flotte et al., 1993; Kaplitt et al., 1994; 1996; Koeberl et al., 1997; McCown et al., (1996) Brain Res., 713, 99-107; Ping et al., (1996) Microcirculation, 3, 225-228; Xiao et al., (1996) J. Virol., 70, 8098-8108).

4. Other Viral Vectors

Other viral vectors are employed as expression constructs in the present methods and compositions. Vectors derived from viruses such as vaccinia virus (Ridgeway, (1988) In: Vectors: A survey of molecular cloning vectors and their uses, pp. 467-492; Baichwal and Sugden, (1986) In, Gene Transfer, pp. 117-148; Coupar et al., Gene, 68:1-10, 1988) canary poxvirus, and herpes viruses are employed. These viruses offer several features for use in gene transfer into various mammalian cells.

Once the construct has been delivered into the cell, the nucleic acid encoding the transgene are positioned and expressed at different sites. In certain embodiments, the nucleic acid encoding the transgene is stably integrated into the genome of the cell. This integration is in the cognate location and orientation via homologous recombination (gene replacement) or it is integrated in a random, non-specific location (gene augmentation). In yet further embodiments, the nucleic acid is stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

### Methods for Treating a Disease

The present methods also encompass methods of treatment or prevention of a disease where administration of cells by, for example, infusion, may be beneficial.

Cells, such as, for example, T cells, tumor infiltrating lymphocytes, natural killer cells, natural killer T cells, TCR-expressing cells, or progenitor cells, such as, for example, hematopoietic stem cells, mesenchymal stromal cells, stem cells, pluripotent stem cells, and embryonic stem cells may be used for cell therapy. The cells may be from a donor, or may be cells obtained from the patient. The cells may, for example, be used in regeneration, for example, to replace the function of diseased cells. The cells may also be modified to express a heterologous gene so that biological agents may be delivered to specific microenvironments such as, for example, diseased bone marrow or metastatic deposits. Mesenchymal stromal cells have also, for example, been used to provide immunosuppressive activity, and may be used in the treatment of graft versus host disease and autoimmune disorders. The cells provided in the present application contain a safety switch that may be valuable in a situation where following cell therapy, the activity of the therapeutic cells needs to be decreased. For example, where T cells that express a chimeric antigen receptor are provided to the patient, in some situations there may be an adverse event, such as off-target toxicity. Administering the ligand would reduce the number of activated T cells that cause the toxicity/Or, for example, the therapeutic cell may work to decrease the tumor cell, or tumor size, and may no longer be needed.

Tumor infiltrating lymphocytes (TILs) refer to T cells having various receptors which infiltrate tumors and kill tumor cells in a targeted manor. Regulating the activity of the TILs using the methods of the present application would allow for more direct control of the elimination of tumor cells.

By “therapeutic cell” is meant a cell used for cell therapy, that is, a cell administered to a subject to treat or prevent a condition or disease. In such cases, where the cells have a negative effect, the present methods may be used to remove the therapeutic cells through selective apoptosis.

By “reduce the number” of T cells, therapeutic cells, or other cells of the present application is meant to reduce the number or percentage of the cells present in a sample, such as a blood sample, obtained from the subject after administration of the multimeric ligand as compared to before the administration of the multimeric ligand. Reducing the number may include having a number cells present in the sample obtained after multimeric ligand administration compared to a sample obtained before multimeric ligand administration that is reduced by at least 10%, 20%, 30%, 40%, 50%, 60%, 70%, 80%, 90%, 95%, 97%, or 99%. The cells may be quantified using any method, including, for example, measuring the level of a marker polypeptide, or counting cells expressing a marker, in the two samples.

In other examples, T cells are used to treat various diseases and conditions, and as a part of stem cell transplantation. An adverse event that may occur after haploidentical T cell transplantation is graft versus host disease (GvHD). The likelihood of GvHD occurring increases with the increased number of T cells that are transplanted. This limits the number of T cells that may be infused. By having the ability to selectively remove the infused T cells in the event of GvHD in the patient, a greater number of T cells may be infused, increasing the number to greater than 10⁶, greater than 10⁷, greater than 10⁸, or greater than 10⁹ cells. The number of T cells/kg body weight that may be administered may be, for example, from about 1×10⁴ T cells/kg body weight to about 9×10⁷ T cells/kg body weight, for example about 1, 2, 3, 4, 5, 6, 7, 8, or 9×10⁴; about 1, 2, 3, 4, 5, 6, 7, 8, or 9×10⁵; about 1, 2, 3, 4, 5, 6, 7, 8, or 9×10⁶; or about 1, 2, 3, 4, 5, 6, 7, 8, or 9×10⁷ T cells/kg body weight. In other examples, therapeutic cells other than T cells may be used. The number of therapeutic cells/kg body weight that may be administered may be, for example, from about 1×10⁴ T cells/kg body weight to about 9×10⁷ T cells/kg body weight, for example about 1, 2, 3, 4, 5, 6, 7, 8, or 9×10⁴; about 1, 2, 3, 4, 5, 6, 7, 8, or 9×10⁵; about 1, 2, 3, 4, 5, 6, 7, 8, or 9×10⁶; or about 1, 2, 3, 4, 5, 6, 7, 8, or 9×10⁷ therapeutic cells/kg body weight.

The term “unit dose” as it pertains to the inoculum refers to physically discrete units suitable as unitary dosages for mammals, each unit containing a predetermined quantity of pharmaceutical composition calculated to produce the desired immunogenic effect in association with the required diluent. The specifications for the unit dose of an inoculum are dictated by and are dependent upon the unique characteristics of the pharmaceutical composition and the particular immunologic effect to be achieved.

An effective amount of the pharmaceutical composition, such as the multimeric ligand presented herein, would be the amount that achieves this selected result of selectively removing the cells that include the caspase-9 vector, such that over 20%, 30%, 40%, 50%, 60%, 70%, 80%, 85%, 90%, 95%, 97%, or 99% of the caspase-9 expressing cells are killed. The term is also synonymous with “sufficient amount.” The effective amount may also be the amount that achieves the desired therapeutic response, such as, the reduction of the amount of therapeutic cells, compared to the time before the ligand inducer is administered.

The effective amount for any particular application can vary depending on such factors as the disease or condition being treated, the particular composition being administered, the size of the subject, and/or the severity of the disease or condition. One can empirically determine the effective amount of a particular composition presented herein without necessitating undue experimentation.

The terms “contacted” and “exposed,” when applied to a cell, tissue or organism, are used herein to describe the process by which the pharmaceutical composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the pharmaceutical composition and/or additional agent(s) are delivered to one or more cells in a combined amount effective to kill the cell(s) or prevent them from dividing.

The administration of the pharmaceutical composition may precede, be co-current with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered within of from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition presented herein and one or more agents may be employed.

### Optimized and Personalized Therapeutic Treatment

The induction of apoptosis after administration of the dimer may be optimized by determining the stage of graft versus host disease, or the number of undesired therapeutic cells that remain in the patient.

For example, determining that a patient has GvHD, and the stage of the GvHD, provides an indication to a clinician that it may be necessary to induce caspase-9 associated apoptosis by administering the multimeric ligand. In another example, determining that a patient has a reduced level of GvHD after treatment with the multimeric ligand may indicate to the clinician that no additional dose of the multimeric ligand is needed.

Similarly, after treatment with the multimeric ligand, determining that the patient continues to exhibit GvHD symptoms, or suffers a relapse of GvHD may indicate to the clinician that it may be necessary to administer at least one additional dose of multimeric ligand. The term “dosage” is meant to include both the amount of the dose and the frequency of administration, such as, for example, the timing of the next dose

In other embodiments, following administration of therapeutic cells, for example, therapeutic cells which express a chimeric antigen receptor in addition to the inducible caspase-9 polypeptide, in the event of a need to reduce the number of modified cells or in vivo modified cells, the multimeric ligand may be administered to the patient. In these embodiments, the methods may comprise determining the presence or absence of a negative symptom or condition, such as Graft vs Host Disease, or off target toxicity, and administering a dose of the multimeric ligand. In other examples, there may be a need to reduce the number of administered therapeutic cells before symptoms of Graft vs Host Disease, or other negative symptoms appear. For example, modified cells that express the chimeric caspase polypeptide may be administered to a subject as part of a stem cell or other transplant. Following the transplant, if there is no longer a need for the modified cells, the multimeric ligand may be administered to the subject in order to reduce the number of modified cells. In yet another embodiment, the chimeric caspase polypeptide may be under the control of a tissue-specific, or stem cell-specific promoter, wherein modified cells, or progeny cells may be eliminated by administration of the multimeric ligand.

The methods may further comprise monitoring the symptom or condition and administering an additional dose of the multimeric ligand in the event the symptom or condition persists. This monitoring and treatment schedule may continue while the therapeutic cells that express chimeric antigen receptors or chimeric signaling molecules remain in the patient.

An indication of adjusting or maintaining a subsequent drug dose, such as, for example, a subsequence dose of the multimeric ligand, and/or the subsequent drug dosage, can be provided in any convenient manner. An indication may be provided in tabular form (e.g., in a physical or electronic medium) in some embodiments. For example, the graft versus host disease observed symptoms may be provided in a table, and a clinician may compare the symptoms with a list or table of stages of the disease. The clinician then can identify from the table an indication for subsequent drug dose. In certain embodiments, an indication can be presented (e.g., displayed) by a computer, after the symptoms or the GvHD stage is provided to the computer (e.g., entered into memory on the computer). For example, this information can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

Methods as presented herein include without limitation the delivery of an effective amount of an activated cell, a nucleic acid or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease. In some embodiments there may be a step of monitoring the biomarkers to evaluate the effectiveness of treatment and to control toxicity.

### Formulations and Routes for Administration to Patients

Where clinical applications are contemplated, it will be necessary to prepare pharmaceutical compositions-expression constructs, expression vectors, fused proteins, transfected or transduced cells, in a form appropriate for the intended application. Generally, this will entail preparing compositions that are essentially free of pyrogens, as well as other impurities that could be harmful to humans or animals.

The multimeric ligand, such as, for example, AP1903, may be delivered, for example at doses of about 0.1 to 10 mg/kg subject weight, of about 0.1 to 5 mg/kg subject weight, of about 0.2 to 4 mg/kg subject weight, of about 0.3 to 3 mg/kg subject weight, of about 0.3 to 2 mg/kg subject weight, or about 0.3 to 1 mg/kg subject weight, for example, about 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, or 10 mg/kg subject weight. In some embodiments, the ligand is provided at 0.4 mg/kg per dose, for example at a concentration of 5 mg/mL. Vials or other containers may be provided containing the ligand at, for example, a volume per vial of about 0.25 ml to about 10 ml, for example, about 0.25, 0.5, 1, 1.5, 2, 2.5, 3, 3.5, 4, 4.5, 5, 5.5, 6, 6.5, 7, 7.5, 8, 8.5, 9, 9.5, or 10 ml, for example, about 2 ml.

One may generally desire to employ appropriate salts and buffers to render delivery vectors stable and allow for uptake by target cells. Buffers also may be employed when recombinant cells are introduced into a patient. Aqueous compositions comprise an effective amount of the vector to cells, dissolved or dispersed in a pharmaceutically acceptable carrier or aqueous medium. Such compositions also are referred to as inocula. A pharmaceutically acceptable carrier includes any and all solvents, dispersion media, coatings, antibacterial and antifungal agents, isotonic and absorption delaying agents and the like. The use of such media and agents for pharmaceutically active substances is known. Except insofar as any conventional media or agent is incompatible with the vectors or cells, its use in therapeutic compositions is contemplated. Supplementary active ingredients also can be incorporated into the compositions.

The active compositions may include classic pharmaceutical preparations. Administration of these compositions will be via any common route so long as the target tissue is available via that route. This includes, for example, oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, discussed herein.

The pharmaceutical forms suitable for injectable use include sterile aqueous solutions or dispersions and sterile powders for the extemporaneous preparation of sterile injectable solutions or dispersions. In all cases the form is sterile and is fluid to the extent that easy syringability exists. It is stable under the conditions of manufacture and storage and is preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In certain examples, isotonic agents, for example, sugars or sodium chloride may be included. Prolonged absorption of the injectable compositions can be brought about by the use in the compositions of agents delaying absorption, for example, aluminum monostearate and gelatin.

For oral administration, the compositions may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including, for example: gels, pastes, powders and slurries. The active ingredient may be added in a therapeutically effective amount to a paste dentifrice that may include, for example, water, binders, abrasives, flavoring agents, foaming agents, and humectants.

The compositions may be formulated in a neutral or salt form. Pharmaceutically-acceptable salts include, for example, the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media can be employed. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations may meet sterility, pyrogenicity, and general safety and purity standards as required by FDA Office of Biologics standards.

### EXAMPLES

The examples set forth below illustrate certain embodiments and do not limit the technology.

Mechanisms for selectively ablating the donor cells have been studied as safety switches for cellular therapies, but there have been complications. Some experience with safety-switch genes to date has been in T lymphocytes since immunotherapy with these cells has proved efficacious as treatment for viral infections and malignancies (Walter, E. A., et al., N. Engl. J. Med. 1995, 333:1038-44; Rooney, C. M., et al., Blood. 1998, 92:1549-55; Dudley, M. E., et al., Science 2002, 298:850-54; Marjit, W. A., et al., Proc. Natl. Acad. Sci. USA 2003, 100:2742-47). The herpes simplex virus I-derived thymidine kinase (HSVTK) gene has been used as an in vivo suicide switch in donor T-cell infusions to treat recurrent malignancy and Epstein Barr virus (EBV) lymphoproliferation after hematopoietic stem cell transplantation (Bonini C, et al., Science. 1997, 276:1719-1724; Tiberghien P, et al., Blood. 2001, 97:63-72). However, destruction of T cells causing graft-versus-host disease was incomplete, and the use of gancyclovir (or analogs) as a pro-drug to activate HSV-TK precludes administration of gancyclovir as an antiviral drug for cytomegalovirus infections. This mechanism of action also requires interference with DNA synthesis, relying on cell division, so that cell killing may be protracted over several days and incomplete, producing a lengthy delay in clinical benefit (Ciceri, F., et al., Lancet Oncol. 2009, 262:1019-24). Moreover, HSV-TK-directed immune responses have resulted in elimination of HSV-TK-transduced cells, even in immunosuppressed human immunodeficiency virus and bone marrow transplant patients, compromising the persistence and hence efficacy of the infused T cells. HSV-TK is also virus-derived, and therefore potentially immunogenic (Bonini C, et al., Science. 1997, 276:1719-1724; Riddell S R, et al., Nat Med. 1996, 2:216-23). The E coli-derived cytosine deaminase gene has also been used clinically (Freytag S O, et al., Cancer Res. 2002, 62:4968-4976), but as a xenoantigen it may be immunogenic and thus incompatible with T-cell-based therapies that require long-term persistence. Transgenic human CD20, which can be activated by a monoclonal chimeric anti-CD20 antibody, has been proposed as a nonimmunogenic safety system (Intona M, et al., Hum Gene Ther. 2000, 11: 611-620).

The following section provides examples of method of providing a safety switch in cells used for cellular therapy, using a caspase-9 chimeric protein.

### Example 1

### Construction and Evaluation of Caspase-9 Suicide Switch Expression Vectors

Vector Construction and Confirmation of Expression

A safety switch that can be stably and efficiently expressed in human T cells is presented herein. The system includes human gene products with low potential immunogenicity that have been modified to interact with a small molecule dimerizer drug that is capable of causing the selective elimination of transduced T cells expressing the modified gene. Additionally the inducible caspase-9 maintains function in T cells overexpressing antiapoptotic molecules.

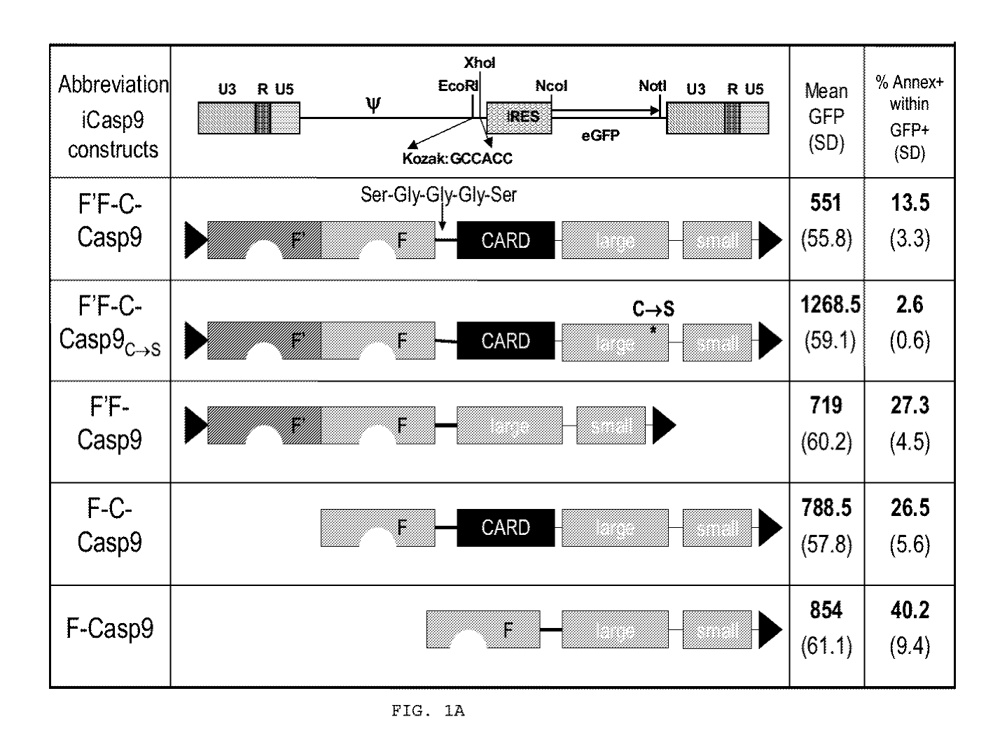

Expression vectors suitable for use as a therapeutic agent were constructed that included a modified human caspase-9 activity fused to a human FK506 binding protein (FKBP), such as, for example, FKBP12v36. The caspase-9/FK506 hybrid activity can be dimerized using a small molecule pharmaceutical. Full length, truncated, and modified versions of the caspase-9 activity were fused to the ligand binding domain, or multimerization region, and inserted into the retroviral vector MSCV.IRES.GRP, which also allows expression of the fluorescent marker, GFP. FIG. 1A illustrates the full length, truncated and modified caspase-9 expression vectors constructed and evaluated as a suicide switch for induction of apoptosis.

The full-length inducible caspase-9 molecule (F′-F-C-Casp9) includes 2, 3, or more FK506 binding proteins (FKBPs—for example, FKBP12v36 variants) linked with a Gly-Ser-Gly-Gly-Gly-Ser linker (SEQ ID NO: 235) to the small and large subunit of the Caspase molecule (see FIG. 1A). Full-length inducible caspase-9 (F′F-C-Casp9.I.GFP) has a full-length caspase-9, also includes a Caspase recruitment domain (CARD; GenBank NM001 229) linked to 2 12-kDa human FK506 binding proteins (FKBP12; GenBank AH002 818) that contain an F36V mutation (FIG. 1A). The amino acid sequence of one or more of the FKBPs (F′) was codon-wobbled (e.g., the 3^{rd} nucleotide of each amino acid codon was altered by a silent mutation that maintained the originally encoded amino acid) to prevent homologous recombination when expressed in a retrovirus. F′F-C-Casp9C3S includes a cysteine to serine mutation at position 287 that disrupts its activation site. In constructs F′F-Casp9, F-C-Casp9, and F′-Casp9, either the Caspase activation domain (CARD), one FKBP, or both, were deleted, respectively. All constructs were cloned into MSCV.IRES.GFP as EcoRI-Xhol fragments.

293T cells were transfected with each of these constructs and 48 hours after transduction expression of the marker gene GFP was analyzed by flow cytometry. In addition, 24 hours after transfection, 293T cells were incubated overnight with 100 nM CID and subsequently stained with the apoptosis marker annexin V. The mean and standard deviation of transgene expression level (mean GFP) and number of apoptotic cells before and after exposure to the chemical inducer of dimerization (CID) (% annexin V within GFP˜ cells) from 4 separate experiments are shown in the second through fifth columns of the table in FIG. 1A. In addition to the level of GFP expression and staining for annexin V, the expressed gene products of the full length, truncated and modified caspase-9 were also analyzed by western blot to confirm the caspase-9 genes were being expressed and the expressed product was the expected size. The results of the western blot are presented in FIG. 1B.

Coexpression of the inducible caspase-9 constructs of the expected size with the marker gene GFP in transfected 293T cells was demonstrated by Western blot using a caspase-9 antibody specific for amino acid residues 299-318, present both in the full-length and truncated Caspase molecules as well as a GFP-specific antibody. Western blots were performed as presented herein.

Transfected 293T cells were resuspended in lysis buffer (50% Tris/Gly, 10% sodium dodecyl sulfate [SDS], 4% beta-mercaptoethanol, 10% glycerol, 12% water, 4% bromophenol blue at 0.5%) containing aprotinin, leupeptin, and phenylmethylsulfonyl fluoride (Boehringer, Ingelheim, Germany) and incubated for 30 minutes on ice. After a 30-minute centrifugation, supernatant was harvested; mixed 1:2 with Laemmli buffer (Bio-Rad, Hercules, Calif.), boiled and loaded on a 10% SDS-polyacrylamide gel. The membrane was probed with rabbit anti-caspase-9 (amino acid residues 299-3 18) immunoglobulin G (IgG; Affinity BioReagents, Golden, Colo.; 1:500 dilution) and with mouse anti-GFP IgG (Covance, Berkeley, Calif.; 1:25,000 dilution). Blots were then exposed to appropriate peroxidase-coupled secondary antibodies and protein expression was detected with enhanced chemiluminescence (ECL; Amersham, Arlington Heights, Ill.). The membrane was then stripped and reprobed with goat polyclonal antiactin (Santa Cruz Biotechnology; 1:500 dilution) to check equality of loading.

Additional smaller size bands, seem in FIG. 1B, likely represent degradation products. Degradation products for the F′F-C-Casp9 and F′F-Casp9 constructs may not be detected due to a lower expression level of these constructs as a result of their basal activity. Equal loading of each sample was confirmed by the substantially equal amounts of actin shown at the bottom of each lane of the western blot, indicating substantially similar amounts of protein were loaded in each lane.

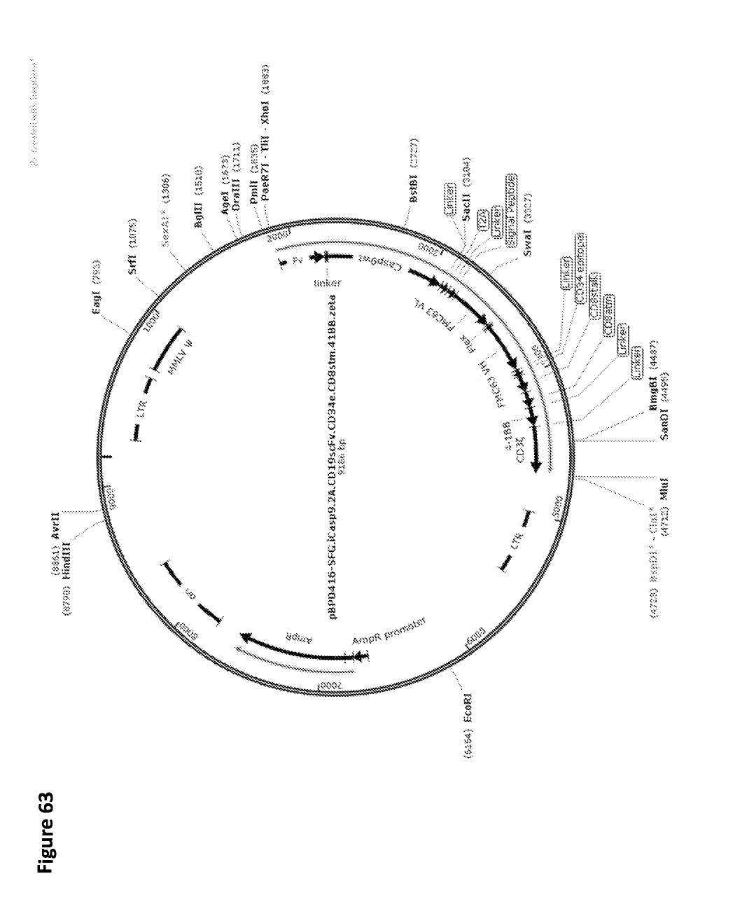

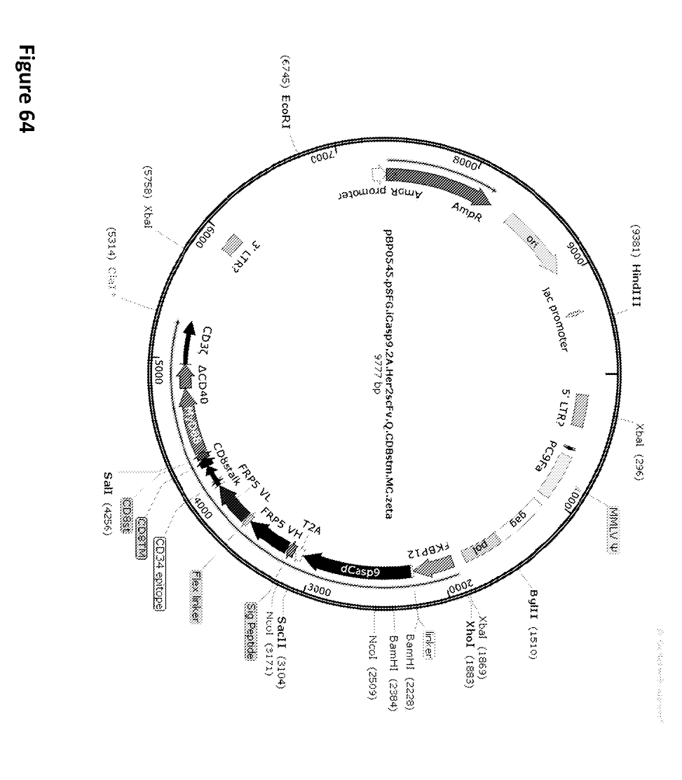

An example of a chimeric polypeptide that may be expressed in the modified cells is provided in FIG. 63. In this example, a single polypeptide is encoded by the nucleic acid vector. The inducible caspase-9 polypeptide is separated from the CAR polypeptide during translation, due to skipping of a peptide bond. (Donnelly, M L 2001, J. Gen. Virol. 82:1013-25). FIG. 63 includes an example of one CAR polypeptide that targets CD19. Other CAR polypeptides targeting any other antigen may be used in the constructs of the present application including, but not limited to, CAR polypeptides targeting PSMA, PSCA, MUC1, CD19, ROR1, Mesothelin, GD2, CD123, MUC16 or Her2/Neu.

Evaluation of Caspase-9 Suicide Switch Expression Constructs.

Cell Lines

B 95-8 EBV transformed B-cell lines (LCLs), Jurkat, and MT-2 cells (kindly provided by Dr S. Marriott, Baylor College of Medicine, Houston, Tex.) were cultured in RPMI 1640 (Hyclone, Logan, Utah) containing 10% fetal bovine serum (FBS; Hyclone). Polyclonal EBV-specific T-cell lines were cultured in 45% RPMI/45% Clicks (Irvine Scientific, Santa Ana, Calif.)/10% FBS and generated as previously reported. Briefly, peripheral blood mononuclear cells (2×10⁶ per well of a 24-well plate) were stimulated with autologous LCLs irradiated at 4000 rads at a responder-to-stimulator (R/S) ratio of 40:1. After 9 to 12 days, viable cells were restimulated with irradiated LCLs at an R/S ratio of 4:1. Subsequently, cytotoxic T cells (CTLs) were expanded by weekly restimulation with LCLs in the presence of 40 U/mL to 100 U/mL recombinant human interleukin-2 (rhIL-2; Proleukin; Chiron, Emeryville, Calif.).

Retrovirus Transduction

For the transient production of retrovirus, 293T cells were transfected with iCasp9/iFas constructs, along with plasmids encoding gag-pol and RD 114 envelope using GeneJuice transfection reagent (Novagen, Madison, Wis.). Virus was harvested 48 to 72 hours after transfection, snap frozen, and stored at ˜80° C. until use. A stable FLYRD 18-derived retroviral producer line was generated by multiple transductions with VSV-G pseudotyped transient retroviral supernatant. FLYRD18 cells with highest transgene expression were single-cell sorted, and the clone that produced the highest virus titer was expanded and used to produce virus for lymphocyte transduction. The transgene expression, function, and retroviral titer of this clone was maintained during continuous culture for more than 8 weeks. For transduction of human lymphocytes, a non-tissue-culture-treated 24-well plate (Becton Dickinson, San Jose, Calif.) was coated with recombinant fibronectin fragment (FN CH-296; Retronectin; Takara Shuzo, Otsu, Japan; 4 μg/mL in PBS, overnight at 4° C.) and incubated twice with 0.5 mL retrovirus per well for 30 minutes at 37° C. Subsequently, 3×10⁵ to 5×10⁵ T cells per well were transduced for 48 to 72 hours using 1 mL virus per well in the presence of 100 U/mL IL-2. Transduction efficiency was determined by analysis of expression of the coexpressed marker gene green fluorescent protein (GFP) on a FACScan flow cytometer (Becton Dickinson). For functional studies, transduced CTLs were either non-selected or segregated into populations with low, intermediate, or high GFP expression using a MoFlo cytometer (Dako Cytomation, Ft Collins, Colo.) as indicated.

Induction and Analysis of Apoptosis

CID (AP20187; ARIAD Pharmaceuticals) at indicated concentrations was added to transfected 293T cells or transduced CTLs. Adherent and nonadherent cells were harvested and washed with annexin binding buffer (BD Pharmingen, San Jose, Calif.). Cells were stained with annexin-V and 7-amino-actinomycin D (7-AAD) for 15 minutes according to the manufacturer's instructions (BD Pharmingen). Within 1 hour after staining, cells were analyzed by flow cytometry using CellQuest software (Becton Dickinson).

Cytotoxicity Assay

The cytotoxic activity of each CTL line was evaluated in a standard 4-hour ⁵¹Cr release assay, as previously presented. Target cells included autologous LCLs, human leukocyte antigen (HLA) class I-mismatched LCLs and the lymphokine-activated killer cell-sensitive T-cell lymphoma line HSB-2. Target cells incubated in complete medium or 1% Triton X-100 (Sigma, St Louis, Mo.) were used to determine spontaneous and maximum ⁵¹Cr release, respectively. The mean percentage of specific lysis of triplicate wells was calculated as 100× (experimental release−spontaneous release)/(maximal release−spontaneous release).

Phenotyping

Cell-surface phenotype was investigated using the following monoclonal antibodies: CD3, CD4, CD8 (Becton Dickinson) and CD56 and TCR-α/β (Immunotech, Miami, Fla.). ΔNGFR-iFas was detected using anti-NGFR antibody (Chromaprobe, Aptos, Calif.). Appropriate matched isotype controls (Becton Dickinson) were used in each experiment. Cells were analyzed with a FACSscan flow cytometer (Becton Dickinson).

Analysis of Cytokine Production

The concentration of interferon-γ (IFN-γ), IL-2, IL-4, IL-5, IL-10, and tumor necrosis factor-α (TNFα) in CTL culture supernatants was measured using the Human Th1/Th2 cytokine cytometric Bead Array (BD Pharmingen) and the concentration of IL-12 in the culture supernatants was measured by enzyme-linked immunosorbent assay (ELISA; R&D Systems, Minneapolis, Minn.) according to the instructions of the manufacturer.

In Vivo Experiments

Non-obese diabetic severe combined immunodeficient (NOD/SCID) mice, 6 to 8 weeks of age, were irradiated (250 rad) and injected subcutaneously in the right flank with 10×10⁶ to 15×10⁶ LCLs resuspended in Matrigel (BD Bioscience). Two weeks later mice bearing tumors that were approximately 0.5 cm in diameter were injected into the tail vein with a 1:1 mixture of nontransduced and iCasp9.I.GFPhigh-transduced EBV CTLs (total 15×10⁶). At 4 to 6 hours prior and 3 days after CTL infusion, mice were injected intraperitoneally with recombinant hIL-2 (2000 U; Proleukin; Chiron). On day 4, the mice were randomly segregated in 2 groups: 1 group received CID (50 μg AP20187, intraperitoneally) and 1 group received carrier only (16.7% propanediol, 22.5% PEG400, and 1.25% Tween 80, intraperitoneally). On day 7, all mice were killed. Tumors were homogenized and stained with antihuman CD3 (BD Pharmingen). By FACS analysis, the number of GFP+ cells within the gated CD3⁺ population was evaluated. Tumors from a control group of mice that received only nontransduced CTLs (total 15×10⁶) were used as a negative control in the analysis of CD3⁺/GFP⁺ cells.

Optimization of Expression and Function of Inducible Caspase-9

Caspases 3, 7, and 9 were screened for their suitability as inducible safety-switch molecules both in transfected 293T cells and in transduced human T cells. Only inducible caspase-9 (iCasp9) was expressed at levels sufficient to confer sensitivity to the chosen CID (e.g., chemical inducer of dimerization). An initial screen indicated that the full length iCasp9 could not be maintained stably at high levels in T cells, possibly due to transduced cells being eliminated by the basal activity of the transgene. The CARD domain is involved in physiologic dimerization of caspase-9 molecules, by a cytochrome C and adenosine triphosphate (ATP)-driven interaction with apoptotic protease-activating factor 1 (Apaf-1). Because of the use of a CID to induce dimerization and activation of the suicide switch, the function of the CARD domain is superfluous in this context and removal of the CARD domain was investigated as a method of reducing basal activity. Given that only dimerization rather than multimerization is required for activation of caspase-9, a single FKBP12v36 domain also was investigated as a method to effect activation.

The activity of the resultant truncated and/or modified forms of caspase-9 (e.g., the CARD domain, or one of the 2 FKBP domains, or both, are removed) were compared. A construct with a disrupted activation site, F′F-C-Casp9_{C->S}, provided a nonfunctional control (see FIG. 1A). All constructs were cloned into the retroviral vector MSCV²⁶ in which retroviral long terminal repeats (LTRs) direct transgene expression and enhanced GFP is coexpressed from the same mRNA by use of an internal ribosomal entry site (IRES). In transfected 293T cells, expression of all inducible caspase-9 constructs at the expected size as well as coexpression of GFP was demonstrated by Western blot (see FIG. 1B). Protein expression (estimated by mean fluorescence of GFP and visualized on Western blot) was highest in the nonfunctional construct F′F-C-Casp9_{C->S} and greatly diminished in the full-length construct F′F-C-Casp9. Removal of the CARD (F′F-Casp9), one FKBP (F-C-Casp9), or both (F-Casp9) resulted in progressively higher expression of both inducible caspase-9 and GFP, and correspondingly enhanced sensitivity to CID FIG. 1A). Based on these results, the F-Casp9 construct (henceforth referred to as iCasp9_{M}) was used for further study in human T lymphocytes.

Stable Expression of iCasp9_{M} in Human T Lymphocytes

The long-term stability of suicide gene expression is of utmost importance, since suicide genes must be expressed for as long as the genetically engineered cells persist. For T-cell transduction, a FLYRD18-derived retroviral producer clone that produces high-titer RD114-pseudotyped virus was generated to facilitate the transduction of T cells. iCasp9_{M} expression in EBV-specific CTL lines (EBV-CTL) was evaluated since EBV-specific CTL lines have well-characterized function and specificity and are already being used as in vivo therapy for prevention and treatment of EBV-associated malignancies. Consistent transduction efficiencies of EBV-CTLs of more than 70% (mean, 75.3%; range, 71.4%-83.0% in 5 different donors) were obtained after a single transduction with retrovirus. The expression of iCasp9_{M} in EBV-CTLs was stable for at least 4 weeks after transduction without selection or loss of transgene function.

iCasp9_{M} does not Alter Transduced T-Cell Characteristics

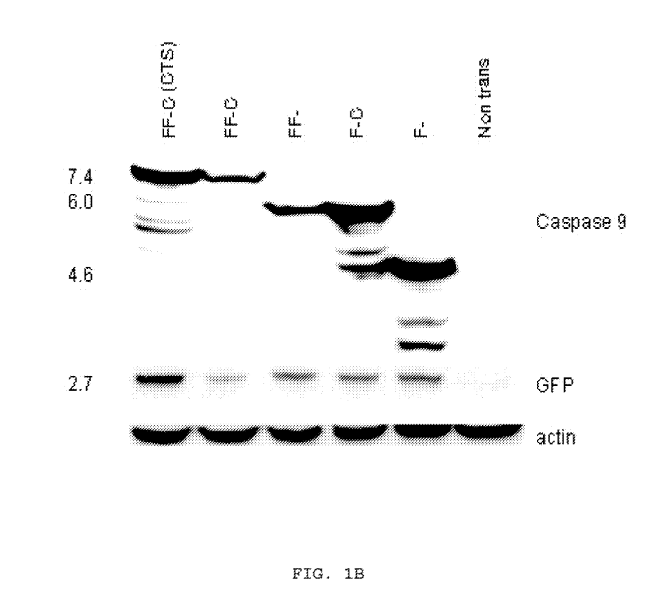

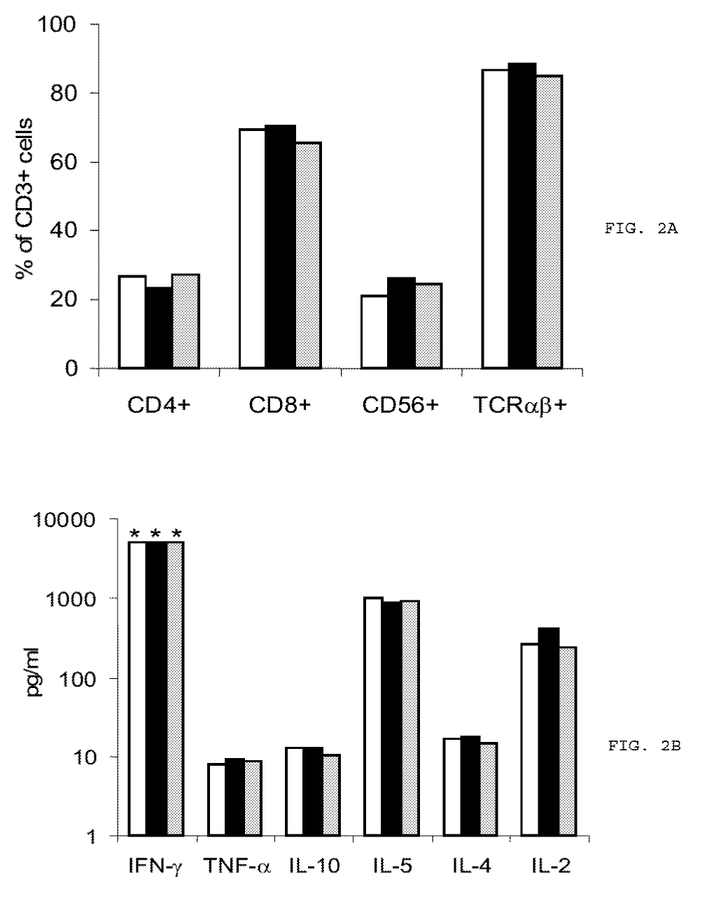

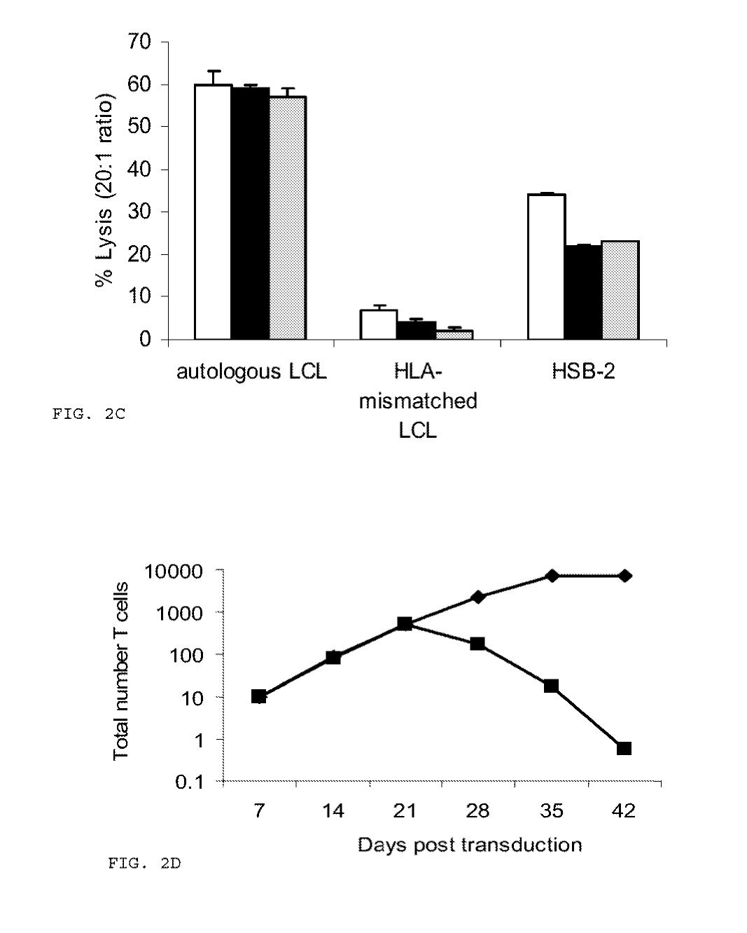

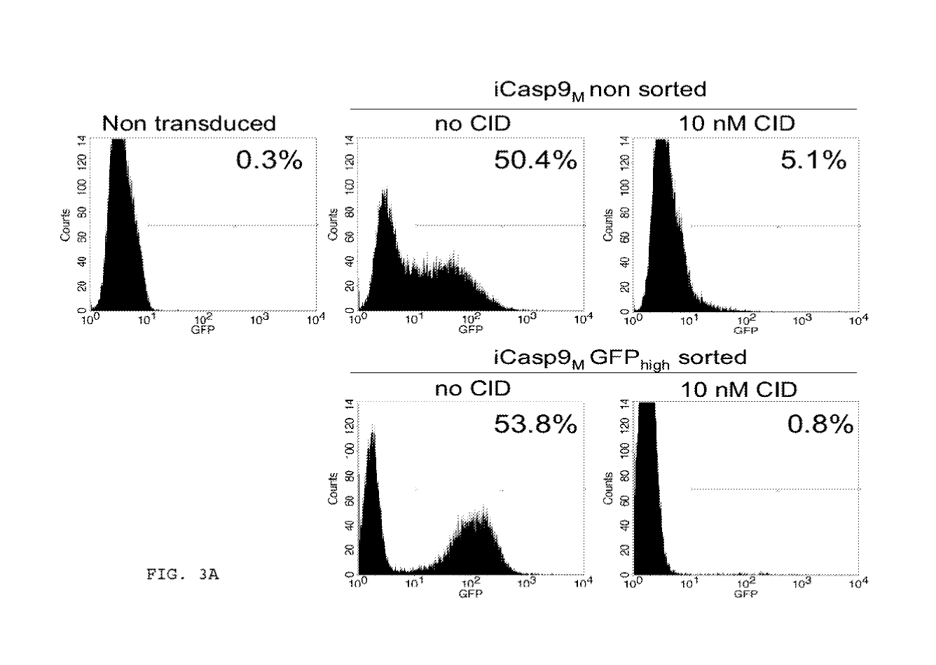

To ensure that expression of iCasp9_{M} did not alter T-cell characteristics, the phenotype, antigen-specificity, proliferative potential, and function of nontransduced or nonfunctional iCasp9_{C->s}-transduced EBV-CTLs was compared with that of iCasp9_{M}-transduced EBV-CTLs. In 4 separate donors, transduced and nontransduced CTLs consisted of equal numbers of CD4⁺, CD8⁺, CD56⁺, and TCR α/β+ cells (see FIG. 2A). Similarly, production of cytokines including IFN-γ, TNFα, IL-10, IL-4, IL-5, and IL-2 was unaltered by iCasp9_{M} expression (see FIG. 2B). iCasp9_{M}-transduced EBV-CTLs specifically lysed autologous LCLs comparable to nontransduced and control-transduced CTLs (see FIG. 2C). Expression of iCasp9M did not affect the growth characteristics of exponentially growing CTLs, and importantly, dependence on antigen and IL-2 for proliferation was preserved (see FIG. 2D). FIGS. 2A and 2B graphically phenotypic and secretion data of type TH1 and TH2 cytokines upon antigen stimulation. FIG. 2C graphically illustrates the level of cytotoxic activity against autologous EBV-transformed lymphoblastoid B-cell line (LCL), HLA-mismatched LCL, and HSB-2 (a LAK cell target) were compared in nontransduced (white bars), F-Casp9_{M}-transduced (black bars), and F′F-C-Casp9_{C->S}-transduced (stipled bars) EBV-specific CTLs (EBV-CTLs) on day 15 to day 18 after transduction (2 antigenic stimulations after transduction). The mean and standard deviation of triplicate wells are shown. Examples of experiments using EBV-CTLs from 4 different donors are shown. FIG. 2D graphically illustrates the antigen dependence of iCasp9_{M}-transduced CTLs. On day 21 after transduction the normal weekly antigenic stimulation with autologous LCLs and IL-2 was continued (black diamonds) or discontinued (black squares). Discontinuation of antigen stimulation resulted in a steady decline of T cells.

Elimination of More than 99% of T Lymphocytes Selected for High Transgene Expression In Vitro

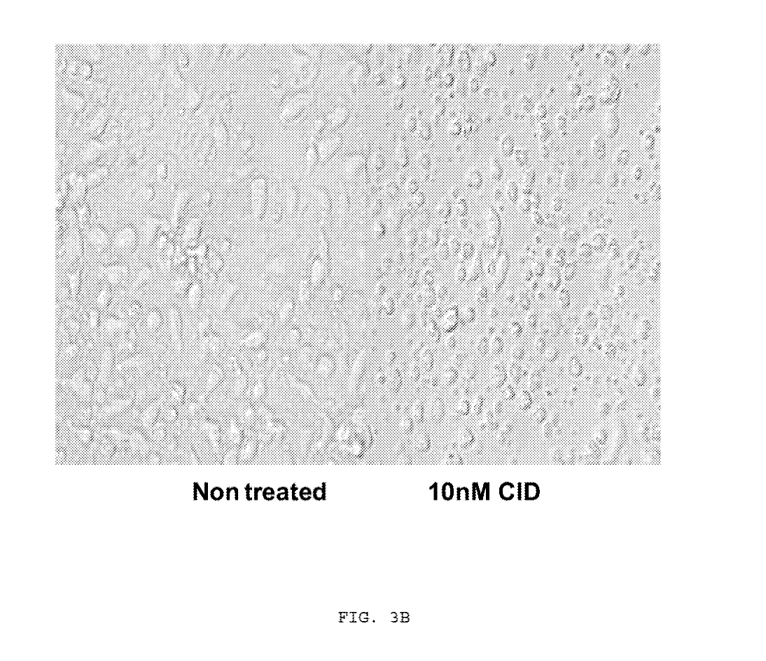

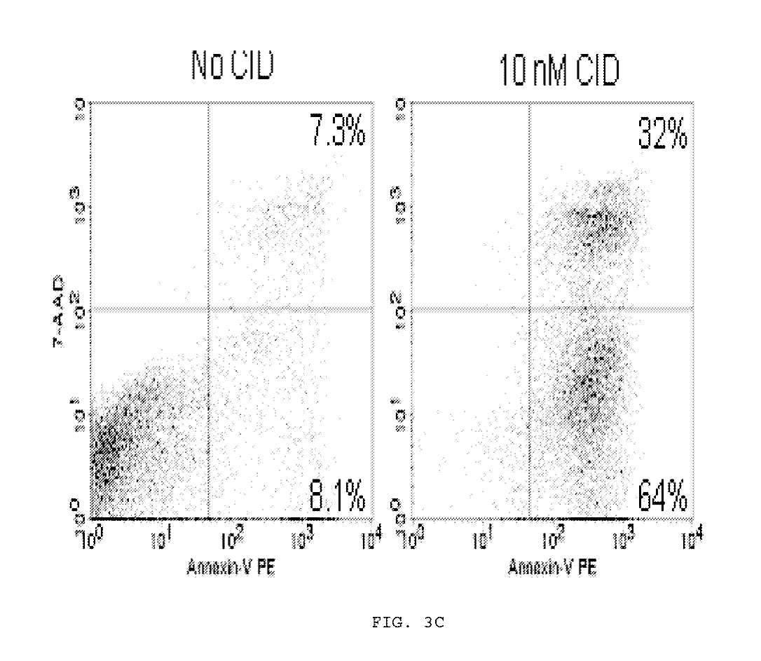

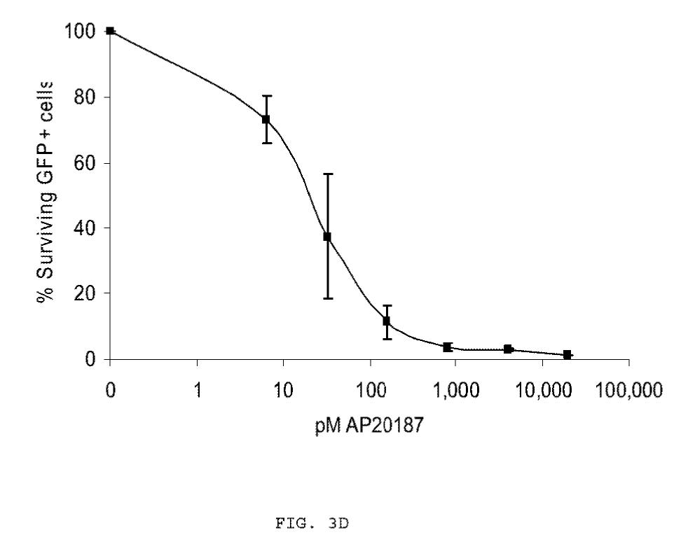

Inducible iCasp9_{M} proficiency in CTLs was tested by monitoring loss of GFP-expressing cells after administration of CID; 91.3% (range, 89.5%-92.6% in 5 different donors) of GFP⁺ cells were eliminated after a single 10-nM dose of CID (see FIG. 3A). Similar results were obtained regardless of exposure time to CID (range, 1 hour-continuous). In all experiments, CTLs that survived CID treatment had low transgene expression with a 70% (range, 55%-82%) reduction in mean fluorescence intensity of GFP after CID. No further elimination of the surviving GFP⁺ T cells could be obtained by an antigenic stimulation followed by a second 10-nM dose of CID. Therefore, the non-responding CTLs most likely expressed insufficient iCasp9_{M} for functional activation by CID. To investigate the correlation between low levels of expression and CTL non-response to CID, CTLs were sorted for low, intermediate, and high expression of the linked marker gene GFP and mixed 1:1 with nontransduced CTLs from the same donor to allow for an accurate quantitation of the number of transduced T cells responding to CID-induced apoptosis.

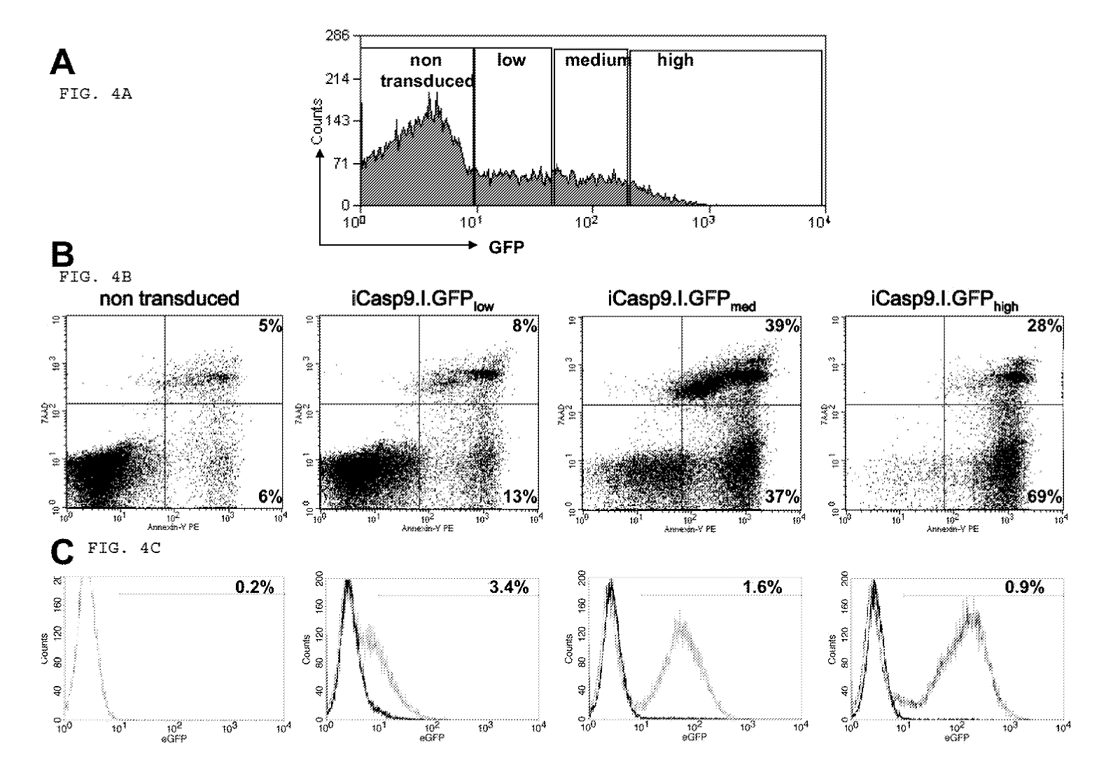

The number of transduced T cells eliminated increased with the level of GFP transgene expression (see FIGS. 4A, 4B and 4C). To determine the correlation between transgene expression and function of iCasp9_{M}, iCasp9_{M} IRES.GFP-transduced EBV-CTL were selected for low (mean 21), intermediate (mean 80) and high (mean 189) GFP expression (see FIG. 4A). Selected T-cells were incubated overnight with 10 nM CID and subsequently stained with annexin V and 7-AAD. Indicated are the percentages of annexin V+/7-AAD- and annexin V+/7-AAD+ T-cells (see FIG. 4B). Selected T-cells were mixed 1:1 with non-transduced T-cells and incubated with 10 nM CID following antigenic stimulation. Indicated is the percentage of residual GFP-positive T-cells on day 7 (see FIG. 4C).

For GFP_{high}-selected cells, 10 nM CID led to deletion of 99.1% (range, 98.7%-99.4%) of transduced cells (see FIG. 3A). On the day of antigen stimulation, F-Casp9_{M}.I.GFP-transduced CTLs were either untreated or treated with 10 nM CID. Seven days later, the response to CID was measured by flow cytometry for GFP. The percentage of transduced T cells was adjusted to 50% to allow for an accurate measurement of residual GFP⁺ cells after CID treatment. The responses to CID in unselected (top row of FIG. 3A) and GFP_{high}-selected CTLs (bottom row of FIG. 3A) was compared. The percentage of residual GFP+ cells is indicated (see FIG. 3A).

Rapid induction of apoptosis in the GFP_{high}-selected cells is demonstrated by apoptotic characteristics such as cell shrinkage and fragmentation within 14 hours of CID administration (see FIG. 3B). After overnight incubation with 10 nM CID, F-Casp9_{M}.I.GFP_{high}-transduced T cells had apoptotic characteristics such as cell shrinkage and fragmentation by microscopic evaluation. Of the T cells selected for high expression, 64% (range, 59%-69%) had an apoptotic (annexin-V⁺+/7-AAD⁻) and 30% (range, 26%-32%) had a necrotic (annexinV⁺/7-AAD⁺) phenotype (see FIG. 3C). Staining with markers of apoptosis showed that 64% of T cells had an apoptotic phenotype (annexin V⁺, 7-AAD⁻, lower right quadrant) and 32% a necrotic phenotype (annexin 7-AAD⁺, upper right quadrant). A representative example of 3 separate experiments is shown.

In contrast, the induction of apoptosis was significantly lower in T cells selected for intermediate or low GFP expression (see FIGS. 4A, 4B and 4C). For clinical applications therefore, versions of the expression constructs with selectable markers that allow selection for high copy number, high levels of expression, or both high copy number and high levels of expression may be desirable. CID-induced apoptosis was inhibited by the panCaspase inhibitor zVAD-fmk (100 μM for 1 hour prior to adding CID. Titration of CID showed that 1 nM CID was sufficient to obtain the maximal deletion effect (FIG. 3D). A dose-response curve using the indicated amounts of CID (AP20187) shows the sensitivity of F-Casp9_{M}.I.GFP_{high} to CID. Survival of GFP+ cells is measured on day 7 after administration of the indicated amount of CID. The mean and standard deviation for each point are given. Similar results were obtained using another chemical inducer of dimerization (CID), AP1903, which was clinically shown to have substantially no adverse effects when administered to healthy volunteers. The dose response remained unchanged for at least 4 weeks after transduction.

iCasp9_{M} is Functional in Malignant Cells that Express Antiapoptotic Molecules

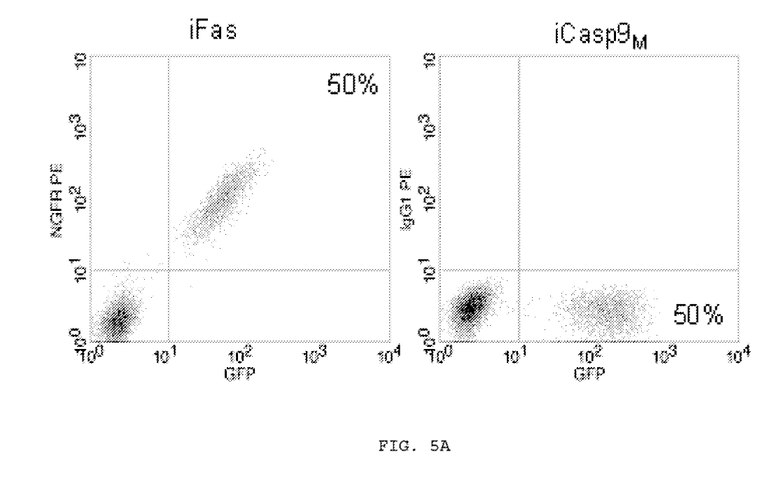

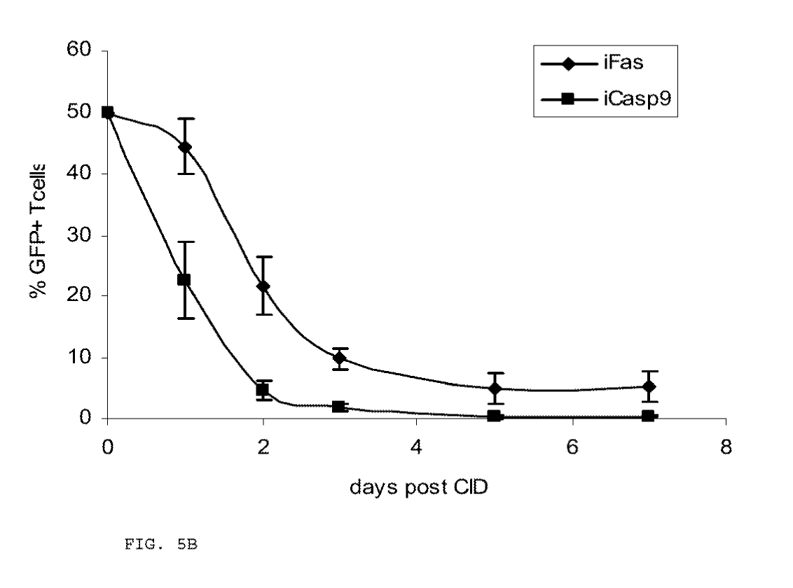

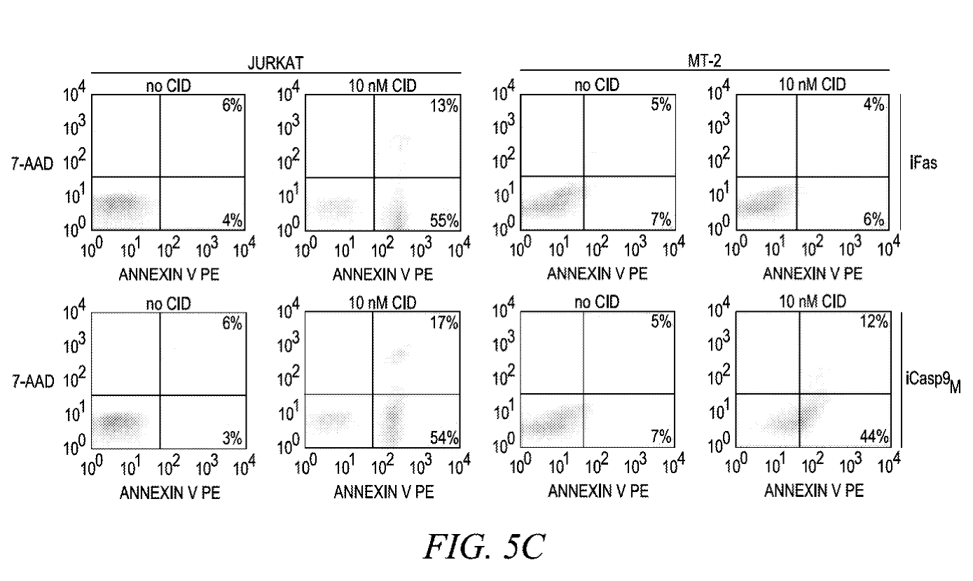

Caspase-9 was selected as an inducible proapoptotic molecule for clinical use rather than previously presented iFas and iFADD, because caspase-9 acts relatively late in apoptosis signaling and therefore is expected to be less susceptible to inhibition by apoptosis inhibitors. Thus, suicide function should be preserved not only in malignant, transformed T-cell lines that express antiapoptotic molecules, but also in subpopulations of normal T cells that express elevated antiapoptotic molecules as part of the process to ensure long-term preservation of memory cells. To further investigate the hypothesis, the function of iCasp9_{M} and iFas was first compared in EBV-CTLs. To eliminate any potential vector based difference, inducible Fas also was expressed in the MSCV.IRES.GFP vector, like iCasp9. For these experiments both ΔNGFR.iFas.I.GFP and iCasp9_{M}.I.GFP-transduced CTLs were sorted for GFP_{high} expression and mixed with nontransduced CTLs at a 1:1 ratio to obtain cell populations that expressed either iFas or iCasp9_{M} at equal proportions and at similar levels (see FIG. 5A). EBV-CTLs transduced with ΔNGFR-iFas.I.GFP are shown in the left panel of FIG. 5A. EBV-CTLs transduced with iCasp9_{M}.I.GFP are shown in the right panel of FIG. 5A. The EBV-CTLs were sorted for high GFP expression and mixed 1:1 with nontransduced CTLs as presented. The percentages of ΔNGFR⁺/GFP⁺ and GFP⁺ T cells are indicated.

Elimination of GFP⁺ cells after administration of 10 nM CID was more rapid and more efficient in iCasp9_{M} than in iFas-transduced CTLs (99.2%+/−0.14% of iCasp9_{M}-transduced cells compared with 89.3%+/−4.9% of iFas-transduced cells at day 7 after CID; P<0.05; see FIG. 5B). On the day of LCL stimulation, 10 nM CID was administered, and GFP was measured at the time points indicated to determine the response to CID. Black diamonds represent data for ΔNGFR-iFas.I.GFP; black squares represent data for iCasp9_{M}.I.GFP. Mean and standard deviation of 3 experiments are shown.

The function of iCasp9M and iFas was also compared in 2 malignant T-cell lines: Jurkat, an apoptosis-sensitive T-cell leukemia line, and MT-2, an apoptosis-resistant T-cell line, due to c-FLIP and bcl-xL expression. Jurkat cells and MT-2 cells were transduced with iFas and iCasp9_{M} with similar efficiencies (92% vs 84% in Jurkat, 76% vs 70% in MT-2) and were cultured in the presence of 10 nM CID for 8 hours. annexin-V staining showed that although iFas and iCasp9_{M} induced apoptosis in an equivalent number of Jurkat cells (56.4%+/−15.6% and 57.2%+/−18.9%, respectively), only activation of iCasp9_{M} resulted in apoptosis of MT-2 cells (19.3%+/−8.4% and 57.9%+/−11.9% for iFas and iCasp9_{M}, respectively; see FIG. 5C).

The human T-cell lines Jurkat (left) and MT-2 (right) were transduced with ΔNGFR-iFas.I.GFP (top row of FIG. 5C) or iCasp9_{M}.I.GFP (bottom row of FIG. 5C). An equal percentage of T cells were transduced with each of the suicide genes: 92% for ΔNGFR-iFas.I.GFP versus 84% for iCasp9_{M}.I.GFP in Jurkat, and 76% for ΔNGFR-iFas.I.GFP versus 70% for iCasp9_{M}.I.GFP in MT-2. T cells were either nontreated or incubated with 10 nM CID. Eight hours after exposure to CID, apoptosis was measured by staining for annexin V and 7-AAD. A representative example of 3 experiments is shown. PE indicates phycoerythrin. These results demonstrate that in T cells overexpressing apoptosis-inhibiting molecules, the function of iFas can be blocked, while iCasp9_{M} can still effectively induce apoptosis.

iCasp9M-Mediated Elimination of T Cells Expressing an Immunomodulatory Transgene

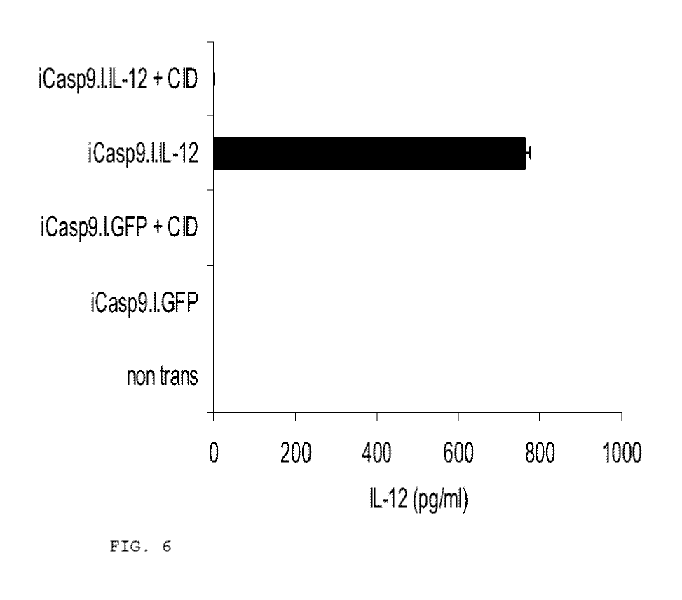

To determine whether iCasp9M could effectively destroy cells genetically modified to express an active transgene product, the ability of iCasp9_{M} to eliminate EBV-CTLs stably expressing IL-12 was measured. While IL-12 was undetectable in the supernatant of nontransduced and iCasp9_{M}.IRES.GFP-transduced CTLs, the supernatant of iCasp9_{M}.IRES.IL-12-transduced cells contained 324 pg/mL to 762 pg/mL IL-12. After administration of 10 nM CID, however, the IL-12 in the supernatant fell to undetectable levels (<7.8 μg/mL). Thus, even without prior sorting for high transgene expressing cells, activation of iCasp9_{M} is sufficient to completely eliminate all T cells producing biologically relevant levels of IL-12 (FIG. 6). The function of iCasp9M when coexpressed with IL-12 is graphically represented by bar graphs in FIG. 6. The marker gene GFP in the iCasp9_{M}.I.GFP constructs was replaced by flexi IL-12, encoding the p40 and p35 subunits of human IL-12. iCasp9_{M}.I.GFP- and iCasp9_{M}.I.IL-12-transduced EBV-CTLs were stimulated with LCLs, and then left untreated or exposed to 10 nM CID. Three days after a second antigenic stimulation, the levels of IL-12 in the culture supernatant were measured by IL-12 ELISA (detection limit of this assay is 7.8 pg/mL). The mean and standard deviation of triplicate wells are indicated. Results of 1 of 2 experiments with CTLs from 2 different donors are shown.

Elimination of More than 99% of T Cells Selected for High Transgene Expression In Vivo

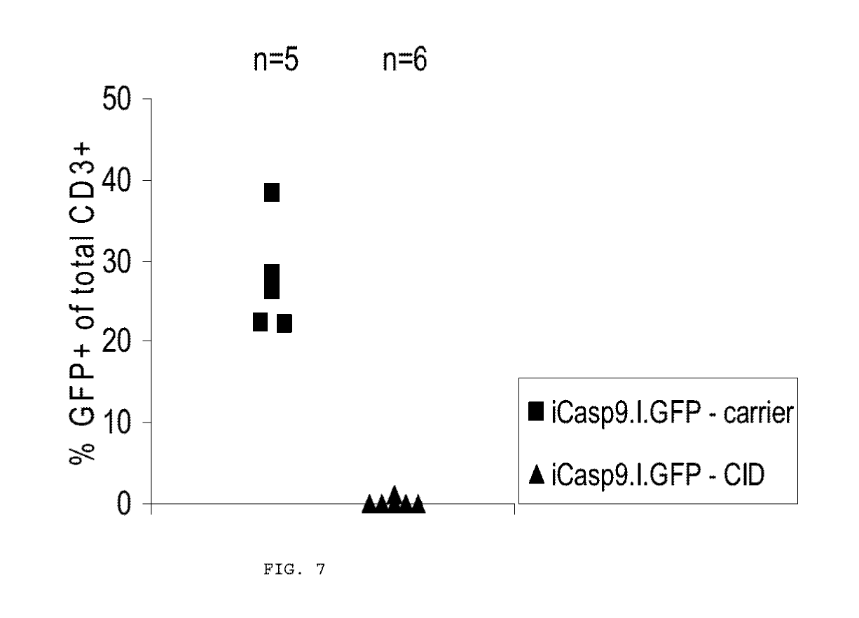

The function of iCasp9_{M} also was evaluated in transduced EBV-CTLs in vivo. A SCID mouse-human xenograft model was used for adoptive immunotherapy. After intravenous infusion of a 1:1 mixture of nontransduced and iCasp9_{M}.IRES.GFP_{high}-transduced CTLs into SCID mice bearing an autologous LCL xenograft, mice were treated either with a single dose of CID or carrier only. Three days after CID/carrier administration, tumors were analyzed for human CD3⁺/GFP⁺ cells. Detection of the nontransduced component of the infusion product, using human anti-CD3 antibodies, confirmed the success of the tail-vein infusion in mice that received CID. In mice treated with CID, there was more than a 99% reduction in the number of human CD3+/GFP+ T cells, compared with infused mice treated with carrier alone, demonstrating equally high sensitivity of iCasp9_{M}-transduced T cells in vivo and in vitro (see FIG. 7).

The function of iCasp9_{M} in vivo is graphically illustrated in FIG. 7. NOD/SCID mice were irradiated and injected subcutaneously with 10×10⁶ to 15×10⁶ LCLs. After 14 days, mice bearing tumors of 0.5 cm in diameter received a total of 15×10⁶ EBV-CTLs (50% of these cells were nontransduced and 50% were transduced with iCasp9_{M}.I.GFP and sorted for high GFP expression). On day 3 after CTL administration, mice received either CID (50 μg AP20187; (black diamonds, n=6) or carrier only (black squares, n=5) and on day 6 the presence of human CD3+/GFP+ T cells in the tumors was analyzed. Human CD3+ T cells isolated from the tumors of a control group of mice that received only nontransduced CTLs (15×10⁶ CTLs; n=4) were used as a negative control for the analysis of CD3+/GFP+ T cells within the tumors.

### DISCUSSION

Presented herein are expression vectors expressing suicide genes suitable for eliminating gene-modified T cells in vivo, in some embodiments. Suicide gene expression vectors presented herein have certain non-limiting advantageous features including stable coexpression in all cells carrying the modifying gene, expression at levels high enough to elicit cell death, low basal activity, high specific activity, and minimal susceptibility to endogenous antiapoptotic molecules. Presented herein, in certain embodiments, is an inducible caspase-9, iCasp9_{M}, which has low basal activity allowing stable expression for more than 4 weeks in human T cells. A single 10-nM dose of a small molecule chemical inducer of dimerization (CID) is sufficient to kill more than 99% of iCasp9_{M}-transduced cells selected for high transgene expression both in vitro and in vivo. Moreover, when coexpressed with Th1 cytokine IL-12, activation of iCasp9_{M} eliminated all detectable IL-12-producing cells, even without selection for high transgene expression. Caspase-9 acts downstream of most antiapoptotic molecules, therefore a high sensitivity to CID is preserved regardless of the presence of increased levels of antiapoptotic molecules of the bcl-2 family. Thus, iCasp9_{M} also may prove useful for inducing destruction even of transformed T cells and memory T cells that are relatively resistant to apoptosis.

Unlike other Caspase molecules, proteolysis does not appear sufficient for activation of caspase-9. Crystallographic and functional data indicate that dimerization of inactive caspase-9 monomers leads to conformational change-induced activation. The concentration of pro-caspase-9, in a physiologic setting, is in the range of about 20 nM, well below the threshold needed for dimerization.

Without being limited by theory, it is believed the energetic barrier to dimerization can be overcome by homophilic interactions between the CARD domains of Apaf-1 and caspase-9, driven by cytochrome C and ATP. Overexpression of caspase-9 joined to 2 FKBPs may allow spontaneous dimerization to occur and can account for the observed toxicity of the initial full length caspase-9 construct. A decrease in toxicity and an increase in gene expression was observed following removal of one FKBP, most likely due to a reduction in toxicity associated with spontaneous dimerization. While multimerization often is involved in activation of surface death receptors, dimerization of caspase-9 should be sufficient to mediate activation. Data presented herein indicates that iCasp9 constructs with a single FKBP function as effectively as those with 2 FKBPs. Increased sensitivity to CID by removal of the CARD domain may represent a reduction in the energetic threshold of dimerization upon CID binding.

The persistence and function of virus- or bacteria-derived lethal genes, such as HSV-TK and cytosine deaminase, can be impaired by unwanted immune responses against cells expressing the virus or bacteria derived lethal genes. The FKBPs and proapoptotic molecules that form the components of iCasp9_{M} are human-derived molecules and are therefore less likely to induce an immune response. Although the linker between FKBP and caspase-9 and the single point mutation in the FKBP domain introduce novel amino acid sequences, the sequences were not immunologically recognized by macaque recipients of iFas-transduced T cells. Additionally, because the components of iCasp9_{M} are human-derived molecules, no memory T cells specific for the junction sequences should be present in a recipient, unlike virus-derived proteins such as HSV-TK, thereby reducing the risk of immune response-mediated elimination of iCasp9_{M}-transduced T cells.

Previous studies using inducible Fas or the death effector domains (DED) of Fas associated death domain proteins (FADD) showed that approximately 10% of transduced cells were unresponsive to activation of the destructive gene. As observed in experiments presented here, a possible explanation for unresponsiveness to CID is low expression of the transgene. The iCasp9_{M}-transduced T cells in our study and iFas-transduced T cells in studies by others that survived after CID administration had low levels of transgene expression. In an attempt to overcome a perceived retroviral “positional effect”, increased levels of homogeneous expression of the transgene were achieved by flanking retroviral integrants with the chicken beta-globin chromatin insulator. Addition of the chromatin insulator dramatically increased the homogeneity of expression in transduced 293T cells, but had no significant effect in transduced primary T cell. Selection of T cells with high expression levels minimized variability of response to the dimerizer. Over 99% of transduced T cells sorted for high GFP expression were eliminated after a single 10-nM CID dose. This demonstration supports the hypothesis that cells expressing high levels of suicide gene can be isolated using a selectable marker.

A very small number of resistant residual cells may cause a resurgence of toxicity; a deletion efficiency of up to 2 logs will significantly decrease this possibility. For clinical use, coexpression with a nonimmunogenic selectable marker such as truncated human NGFR, CD20, or CD34 (e.g., instead of GFP) will allow for selection of high transgene-expressing T cells. Coexpression of the suicide switch (e.g., iCASP9_{M}) and a suitable selectable marker (e.g., truncated human NGFR, CD20, CD34, the like and combinations thereof) can be obtained using either an internal ribosome entry site (IRES) or posttranslational modification of a fusion protein containing a self-cleaving sequence (e.g., 2A). In contrast, in situations where the sole safety concern is the transgene-mediated toxicity (e.g., artificial T-cell receptors, cytokines, the like or combinations thereof), this selection step may be unnecessary, as tight linkage between iCasp9_{M} and transgene expression enables elimination of substantially all cells expressing biologically relevant levels of the therapeutic transgene. This was demonstrated by coexpressing iCasp9_{M} with IL-12. Activation of iCasp9_{M} substantially eliminated any measurable IL-12 production. The success of transgene expression and subsequent activation of the “suicide switch” may depend on the function and the activity of the transgene.

Another possible explanation for unresponsiveness to CID is that high levels of apoptosis inhibitors may attenuate CID-mediated apoptosis. Examples of apoptosis inhibitors include c-FLIP, bcl-2 family members and inhibitors of apoptosis proteins (IAPs), which normally regulate the balance between apoptosis and survival. For instance, upregulation of c-FLIP and bcl-2 render a subpopulation of T cells, destined to establish the memory pool, resistant to activation-induced cell death in response to cognate target or antigen-presenting cells. In several T-lymphoid tumors, the physiologic balance between apoptosis and survival is disrupted in favor of cell survival. A suicide gene should delete substantially all transduced T cells including memory and malignantly transformed cells. Therefore, the chosen inducible suicide gene should retain a significant portion if not substantially all of its activity in the presence of increased levels of antiapoptotic molecules.

The apical location of iFas (or iFADD) in the apoptosis signaling pathway may leave it especially vulnerable to inhibitors of apoptosis, thus making these molecules less well suited to being the key component of an apoptotic safety switch. Caspase 3 or 7 would seem well suited as terminal effector molecules; however neither could be expressed at functional levels in primary human T cells. Therefore caspase-9, was chosen as the suicide gene, because capsase 9 functions late enough in the apoptosis pathway that it bypasses the inhibitory effects of c-FLIP and antiapoptotic bcl-2 family members, and caspase-9 also could be expressed stably at functional levels. Although X-linked inhibitor of apoptosis (XIAP) could in theory reduce spontaneous caspase-9 activation, the high affinity of AP20187 (or AP1903) for FKBP_{V36} may displace this noncovalently associated XIAP. In contrast to iFas, iCasp9_{M} remained functional in a transformed T-cell line that overexpresses antiapoptotic molecules, including bcl-xL.

Presented herein is an inducible safety switch, designed specifically for expression from an oncoretroviral vector by human T cells. iCasp9_{M} can be activated by AP1903 (or analogs), a small chemical inducer of dimerization that has proven safe at the required dose for optimum deletional effect, and unlike ganciclovir or rituximab has no other biologic effects in vivo. Therefore, expression of this suicide gene in T cells for adoptive transfer can increase safety and also may broaden the scope of clinical applications.

### Example 2

### Using the iCasp9 Suicide Gene to Improve the Safety of Allodepleted T Cells after Haploidentical Stem Cell Transplantation

Presented in this example are expression constructs and methods of using the expression constructs to improve the safety of allodepleted T cells after haploidentical stem cell transplantation. A retroviral vector encoding iCasp9 and a selectable marker (truncated CD19) was generated as a safety switch for donor T cells. Even after allodepletion (using anti-CD25 immunotoxin), donor T cells could be efficiently transduced, expanded, and subsequently enriched by CD19 immunomagnetic selection to >90% purity. The engineered cells retained anti-viral specificity and functionality, and contained a subset with regulatory phenotype and function. Activating iCasp9 with a small-molecule dimerizer rapidly produced>90% apoptosis. Although transgene expression was downregulated in quiescent T cells, iCasp9 remained an efficient suicide gene, as expression was rapidly upregulated in activated (alloreactive) T cells.

Materials and Methods

Generation of Allodepleted T Cells

Allodepleted cells were generated from healthy volunteers as previously presented. Briefly, peripheral blood mononuclear cells (PBMCs) from healthy donors were co-cultured with irradiated recipient Epstein Barr virus (EBV)-transformed lymphoblastoid cell lines (LCL) at responder-to-stimulator ratio of 40:1 in serum-free medium (AIM V; Invitrogen, Carlsbad, Calif.). After 72 hours, activated T cells that expressed CD25 were depleted from the co-culture by overnight incubation in RFT5-SMPT-dgA immunotoxin. Allodepletion was considered adequate if the residual CD3⁺CD25⁺ population was <1% and residual proliferation by 3H-thymidine incorporation was <10%.

### Plasmid and Retrovirus

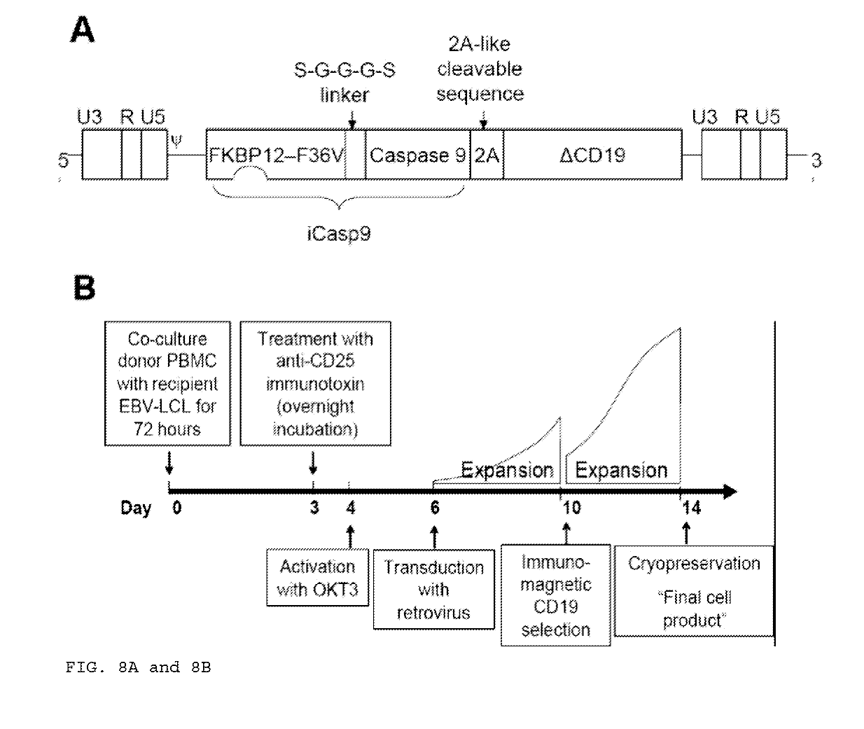

SFG.iCasp9.2A.CD19 consists of inducible caspase-9 (iCasp9) linked, via a cleavable 2A-like sequence, to truncated human CD19 (C D19; see FIG. 8A). iCasp9 consists of a human FK5 06-binding protein (FKBP12; GenBank AH002 818) with an F36V mutation, connected via a Ser-Gly-Gly-Gly-Ser linker (SEQ ID NO: 236) to human caspase-9 (CASP9; GenBank NM 001229). The F36V mutation increases the binding affinity of FKBP12 to the synthetic homodimerizer, AP20187 or AP1903. The Caspase recruitment domain (CARD) has been deleted from the human caspase-9 sequence because its physiological function has been replaced by FKBP12, and its removal increases transgene expression and function. The 2A-like sequence encodes an 20 amino acid peptide from Thosea asigna insect virus, which mediates>99% cleavage between a glycine and terminal proline residue, resulting in 19 extra amino acids in the C terminus of iCasp9, and one extra proline residue in the N terminus of CD19. CD19 consists of full-length CD19 (GenBank NM 001770) truncated at amino acid 333 (TDPTRRF (SEQ ID NO: 237)), which shortens the intracytoplasmic domain from 242 to 19 amino acids, and removes all conserved tyrosine residues that are potential sites for phosphorylation.

A stable PG13 clone producing Gibbon ape leukemia virus (Gal-V) pseudotyped retrovirus was made by transiently transfecting Phoenix Eco cell line (ATCC product #SD3444; ATCC, Manassas, Va.) with SFG.iCasp9.2A.CD19. This produced Eco-pseudotyped retrovirus. The PG13 packaging cell line (ATCC) was transduced three times with Eco-pseudotyped retrovirus to generate a producer line that contained multiple SFG.iCasp9.2A.CD19 proviral integrants per cell. Single cell cloning was performed, and the PG13 clone that produced the highest titer was expanded and used for vector production.

Retroviral Transduction

Culture medium for T cell activation and expansion consisted of 45% RPMI 1640 (Hyclone, Logan, Utah), 45% Clicks (Irvine Scientific, Santa Ana, Calif.) and 10% fetal bovine serum (FBS; Hyclone). Allodepleted cells were activated by immobilized anti-CD3 (OKT3; Ortho Biotech, Bridgewater, N.J.) for 48 hours before transduction with retroviral vector (see FIG. 8B). FIG. 8B presents an overview of the process for production of the “final cell product” that expresses the transduced transgene. Selective allodepletion was performed by co-culturing donor PBMC with recipient EBV-LCL to activate alloreactive cells: activated cells expressed CD25 and were subsequently eliminated by anti-CD25 immunotoxin. The allodepleted cells were activated by OKT3 and transduced with the retroviral vector 48 hours later. Immunomagnetic selection was performed on day 4 of transduction; the positive fraction was expanded for a further 4 days and cryopreserved.

In small-scale experiments, non-tissue culture-treated 24-well plates (Becton Dickinson, San Jose, Calif.) were coated with OKT3 1 g/ml for 2 to 4 hours at 37° C. Allodepleted cells were added at 1×10⁶ cells per well. At 24 hours, 100 U/ml of recombinant human interleukin-2 (IL-2) (Proleukin; Chiron, Emeryville, Calif.) was added. Retroviral transduction was performed 48 hours after activation. Non-tissue culture-treated 24-well plates were coated with 3.5 μg/cm² recombinant fibronectin fragment (CH-296; Retronectin; Takara Mirus Bio, Madison, Wis.) and the wells loaded twice with retroviral vector-containing supernatant at 0.5 ml per well for 30 minutes at 37° C., following which OKT3-activated cells were plated at 5×10⁵ cells per well in fresh retroviral vector-containing supernatant and T cell culture medium at a ratio of 3:1, supplemented with 100 U/ml IL-2. Cells were harvested after 2 to 3 days and expanded in the presence of 50 U/ml IL-2.

Scaling-Up Production of Gene-Modified Allodepleted Cells

Scale-up of the transduction process for clinical application used non-tissue culture-treated T75 flasks (Nunc, Rochester, N.Y.), which were coated with 10 ml of OKT3 1 μg/ml or 10 ml of fibronectin 7 μg/ml at 4° C. overnight. Fluorinated ethylene propylene bags corona-treated for increased cell adherence (2PF-0072AC, American Fluoroseal Corporation, Gaithersburg, Md.) were also used. Allodepleted cells were seeded in OKT3-coated flasks at 1×10⁶ cells/ml. 100 U/ml IL-2 was added the next day. For retroviral transduction, retronectin-coated flasks or bags were loaded once with 10 ml of retrovirus-containing supernatant for 2 to 3 hours. OKT3-activated T cells were seeded at 1×10⁶ cells/ml in fresh retroviral vector-containing medium and T cell culture medium at a ratio of 3:1, supplemented with 100 U/ml IL-2. Cells were harvested the following morning and expanded in tissue-culture treated T75 or T175 flasks in culture medium supplemented with between about 50 to 100 U/ml IL-2 at a seeding density of between about 5×10⁵ cells/ml to 8×10⁵ cells/ml.

CD19 Immunomagnetic Selection

Immunomagnetic selection for CD19 was performed 4 days after transduction. Cells were labeled with paramagnetic microbeads conjugated to monoclonal mouse anti-human CD19 antibodies (Miltenyi Biotech, Auburn, Calif.) and selected on MS or LS columns in small scale experiments and on a CliniMacs Plus automated selection device in large scale experiments. CD19-selected cells were expanded for a further 4 days and cryopreserved on day 8 post transduction. These cells were referred to as “gene-modified allodepleted cells”.

Immunophenotyping and Pentamer Analysis

Flow cytometric analysis (FACSCalibur and CellQuest software; Becton Dickinson) was performed using the following antibodies: CD3, CD4, CD8, CD19, CD25, CD27, CD28, CD45RA, CD45RO, CD56 and CD62L. CD19-PE (Clone 4G7; Becton Dickinson) was found to give optimum staining and was used in all subsequent analysis. A Non-transduced control was used to set the negative gate for CD19. An HLA-pentamer, HLA-B8-RAKFKQLL (SEQ ID NO: 232) (Proimmune, Springfield, Va.) was used to detect T cells recognizing an epitope from EBV lytic antigen (BZLF1). HLA-A2-NLVPMVATV (SEQ ID NO: 238) pentamer was used to detect T cells recognizing an epitope from CMV-pp65 antigen.

Interferon-ELISpot Assay for Anti-Viral Response

Interferon-ELISpot for assessment of responses to EBV, CMV and adenovirus antigens was performed using known methods. Gene-modified allodepleted cells cryopreserved at 8 days post transduction were thawed and rested overnight in complete medium without IL-2 prior to use as responder cells. Cryopreserved PBMCs from the same donor were used as comparators. Responder cells were plated in duplicate or triplicate in serial dilutions of 2×10⁵, 1×10⁵, 5×10⁴ and 2.5×10⁴ cells per well. Stimulator cells were plated at 1×10⁵ per well. For response to EBV, donor-derived EBV-LCLs irradiated at 40Gy were used as stimulators. For response to adenovirus, donor-derived activated monocytes infected with Ad5f35 adenovirus were used.

Briefly, donor PBMCs were plated in X-Vivo 15 (Cambrex, Walkersville, Md.) in 24-well plates overnight, harvested the next morning, infected with Ad5f35 at a multiplicity of infection (MOI) of 200 for 2 hours, washed, irradiated at 30Gy, and used as stimulators. For anti-CMV response, a similar process using Ad5f35 adenovirus encoding the CMV pp65 transgene (Ad5f35-pp65) at an MOI of 5000 was used. Specific spot-forming units (SFU) were calculated by subtracting SFU from responder-alone and stimulator-alone wells from test wells. Response to CMV was the difference in SFU between Ad5f35-pp65 and Ad5f35 wells.

EBV-Specific Cytotoxicity

Gene-modified allodepleted cells were stimulated with 40Gy-irradiated donor-derived EBVLCL at a responder: stimulator ratio of 40:1. After 9 days, the cultures were restimulated at a responder: stimulator ratio of 4:1. Restimulation was performed weekly as indicated. After two or three rounds of stimulation, cytotoxicity was measured in a 4-hour 51 Cr-release assay, using donor EBV-LCL as target cells and donor OKT3 blasts as autologous controls. NK activity was inhibited by adding 30-fold excess of cold K562 cells.

Induction of Apoptosis with Chemical Inducer of Dimerization, AP20187

Suicide gene functionality was assessed by adding a small molecule synthetic homodimerizer, AP20187 (Ariad Pharmaceuticals; Cambridge, Mass.), at 10 nM final concentration the day following CD19 immunomagnetic selection. Cells were stained with annexin V and 7-aminoactinomycin (7-AAD)(BD Pharmingen) at 24 hours and analyzed by flow cytometry. Cells negative for both annexin V and 7-AAD were considered viable, cells that were annexin V positive were apoptotic, and cells that were both annexin V and 7-AAD positive were necrotic. The percentage killing induced by dimerization was corrected for baseline viability as follows: Percentage killing=100%−(% Viability in AP20187-treated cells÷ % Viability in non-treated cells).

Assessment of Transgene Expression Following Extended Culture and Reactivation

Cells were maintained in T cell medium containing 50 U/ml IL-2 until 22 days after transduction. A portion of cells was reactivated on 24-well plates coated with 1 g/ml OKT3 and 1 μg/ml anti-CD28 (Clone CD28.2, BD Pharmingen, San Jose, Calif.) for 48 to 72 hours. CD19 expression and suicide gene function in both reactivated and non-reactivated cells were measured on day 24 or 25 post transduction.

In some experiments, cells also were cultured for 3 weeks post transduction and stimulated with 30G-irradiated allogeneic PBMC at a responder:stimulator ratio of 1:1. After 4 days of co-culture, a portion of cells was treated with 10 nM AP20187. Killing was measured by annexin V/7-AAD staining at 24 hours, and the effect of dimerizer on bystander virus-specific T cells was assessed by pentamer analysis on AP20187-treated and untreated cells.

Regulatory T Cells

CD4, CD25 and Foxp3 expression was analyzed in gene-modified allodepleted cells using flow cytometry. For human Foxp3 staining, the eBioscience (San Diego, Calif.) staining set was used with an appropriate rat IgG2a isotype control. These cells were co-stained with surface CD25-FITC and CD4-PE. Functional analysis was performed by co-culturing CD4⁺25⁺ cells selected after allodepletion and gene modification with carboxyfluorescein diacetate N-succinimidyl ester (CFSE)-labeled autologous PBMC. CD4⁺25⁺ selection was performed by first depleting CD8⁺ cells using anti-CD 8 microbeads (Miltenyi Biotec, Auburn, Calif.), followed by positive selection using anti-CD25 microbeads (Miltenyi Biotec, Auburn, Calif.). CFSE-labeling was performed by incubating autologous PBMC at 2×10⁷/ml in phosphate buffered saline containing 1.5 μM CFSE for 10 minutes. The reaction was stopped by adding an equivalent volume of FBS and incubating for 10 minutes at 37° C. Cells were washed twice before use. CFSE-labeled PBMCs were stimulated with OKT3 500 ng/ml and 40 G-irradiated allogeneic PBMC feeders at a PBMC:allogeneic feeder ratio of 5:1. The cells were then cultured with or without an equal number of autologous CD4⁺25⁺ gene-modified allodepleted cells. After 5 days of culture, cell division was analyzed by flow cytometry; CD19 was used to gate out non-CFSE-labeled CD4⁺ CD25⁺ gene-modified T cells.

Statistical Analysis

Paired, 2-tailed Student's t test was used to determine the statistical significance of differences between samples. All data are represented as mean±1 standard deviation.

Results

Selectively Allodepleted T Cells can be Efficiently Transduced with iCasp9 and Expanded

Selective allodepletion was performed in accordance with clinical protocol procedures. Briefly, 3/6 to 5/6 HLA-mismatched PBMC and lymphoblastoid cell lines (LCL) were co-cultured. RFT5-SMPT-dgA immunotoxin was applied after 72 hours of co-culture and reliably produced allodepleted cells with <10% residual proliferation (mean 4.5±2.8%; range 0.74 to 9.1%; 10 experiments) and containing<1% residual CD3⁺ CD25⁺ cells (mean 0.23±0.20%; range 0.06 to 0.73%; 10 experiments), thereby fulfilling the release criteria for selective allodepletion, and serving as starting materials for subsequent manipulation.

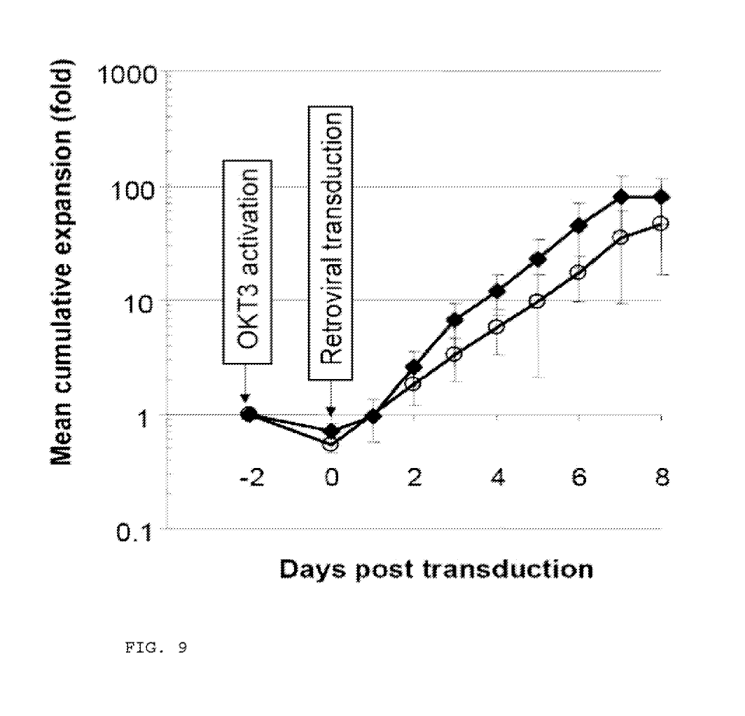

Allodepleted cells activated on immobilized OKT3 for 48 hours could be efficiently transduced with Gal-V pseudotyped retrovirus vector encoding SFG.iCasp9.2A.CD19. Transduction efficiency assessed by FACS analysis for CD19 expression 2 to 4 days after transduction was about 53%±8%, with comparable results for small-scale (24-well plates) and large-scale (T75 flasks) transduction (about 55±8% versus about 50%±10% in 6 and 4 experiments, respectively). Cell numbers contracted in the first 2 days following OKT3 activation such that only about 61%±12% (range of about 45% to 80%) of allodepleted cells were recovered on the day of transduction (see FIG. 9). Illustrated in FIG. 9 are graphical results of experiments performed to determine if allodepleted cells could be successfully expanded following transduction. Black diamonds denote large scale experiments performed in flasks and bags. Open circles denote small-scale experiments performed in 24 well plates. Thereafter, the cells showed significant expansion, with a mean expansion in the range of about 94±46-fold (range of about 40 to about 153) over the subsequent 8 days, resulting in a net 58±33-fold expansion. Cell expansion in both small- and large-scale experiments was similar, with net expansion of about 45±29 fold (range of about 25 to about 90) in 5 small-scale experiments and about 79±34 fold (range of about 50 to about 116) in 3 large-scale experiments.

ΔCD19 Enables Efficient and Selective Enrichment of Transduced Cells on Immunomagnetic Columns

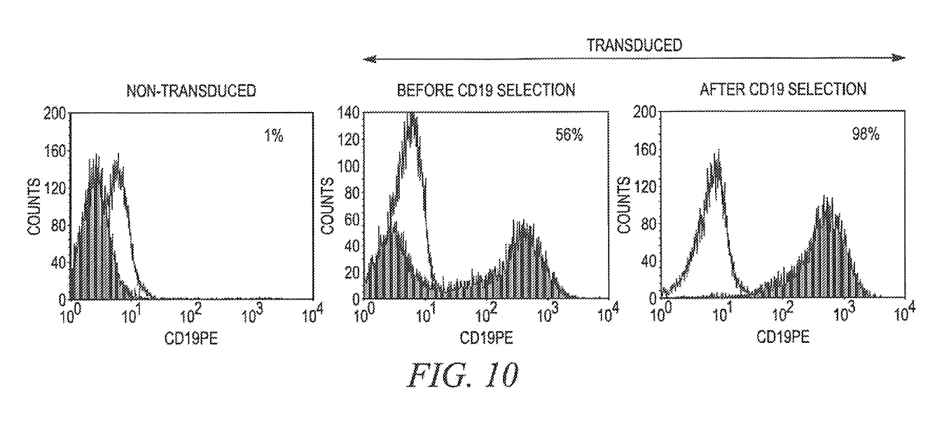

The efficiency of suicide gene activation sometimes depends on the functionality of the suicide gene itself, and sometimes on the selection system used to enrich for gene-modified cells. The use of CD19 as a selectable marker was investigated to determine if CD19 selection enabled the selection of gene-modified cells with sufficient purity and yield, and whether selection had any deleterious effects on subsequent cell growth. Small-scale selection was performed according to manufacturer's instruction; however, it was determined that large-scale selection was optimum when 10I of CD19 microbeads was used per 1.3×10⁷ cells. FACS analysis was performed at 24 hours after immunomagnetic selection to minimize interference from anti-CD19 microbeads. The purity of the cells after immunomagnetic selection was consistently greater than 90%: mean percentage of CD19+ cells was in the range of about 98.3%±0.5% (n=5) in small-scale selections and in the range of about 97.4%±0.9% (n=3) in large-scale CliniMacs selections (see FIG. 10). Shown in FIG. 10 are representative FACS analysis traces of the immunomagnetic selection performed 2 days post-transduction.

The absolute yield of small- and large-scale selections were about 31%±11% and about 28%±6%, respectively; after correction for transduction efficiency. The mean recovery of transduced cells was about 54%±14% in small-scale and about 72%±18% in large-scale selections. The selection process did not have any discernable deleterious effect on subsequent cell expansion. In 4 experiments, the mean cell expansion over 3 days following CD19 immunomagnetic selection was about 3.5 fold for the CD19 positive fraction versus about 4.1 fold for non-selected transduced cells (p=0.34) and about 3.7 fold for non-transduced cells (p=0.75).

Immunophenotype of Gene-Modified Allodepleted Cells

The final cell product (gene-modified allodepleted cells that had been cryopreserved 8 days after transduction) was immunophenotyped and was found to contain both CD4 and CD8 cells, with CD8 cells predominant, at 62%±11% CD8⁺ versus 23%±8% CD4⁺, as shown in the table below. NS=not significant, SD=standard deviation.

TABLE 1

| | Unmanipulated | Gene-modified | |
|---|---|---|---|
| | PBMC | allodepleted cells | |
| | (mean % ± SD) | (mean % ± SD) | |
| T cells: Total CD3⁺ | 82 ± 6 | 95 ± 6 | NS |
| CD3+ 4+ | 54 ± 5 | 23 ± 8 | p < 0.01 |
| CD3+ 8+ | 26 ± 9 | 62 ± 11 | p < 0.001 |
| NK cells: CD3⁻ 56+ | 6 ± 3 | 2 ± 1 | NS |
| Memory phenotype | | | |
| CD45RA⁺ | 66 ± 3 | 10 ± 5 | p < 0.001 |
| CD45RO⁺ | 26 ± 2 | 78 ± 7 | p < 0.001 |
| CD45RA⁻ CD62L⁺ | 19 ± 1 | 24 ± 7 | NS |
| CD45RA⁻ CD62L⁻ | 9 ± 1 | 64 ± 7 | p < 0.001 |
| CD27⁺ CD28⁺ | 67 ± 7 | 19 ± 9 | p < 0.001 |
| CD27⁺ CD28⁻ | 7 ± 3 | 9 ± 4 | NS |
| CD27⁻ CD28⁺ | 4 ± 1 | 19 ± 8 | p < 0.05 |
| CD27⁻ CD28⁻ | 22 ± 8 | 53 ± 18 | p < 0.05 |

The majority of cells were CD45RO⁺ and had the surface immunophenotype of effector memory T cells. Expression of memory markers, including CD62L, CD27 and CD28, was heterogeneous. Approximately 24% of cells expressed CD62L, a lymph node-homing molecule predominantly expressed on central memory cells.

Gene-Modified Allodepleted Cells Retained Antiviral Repertoire and Functionality

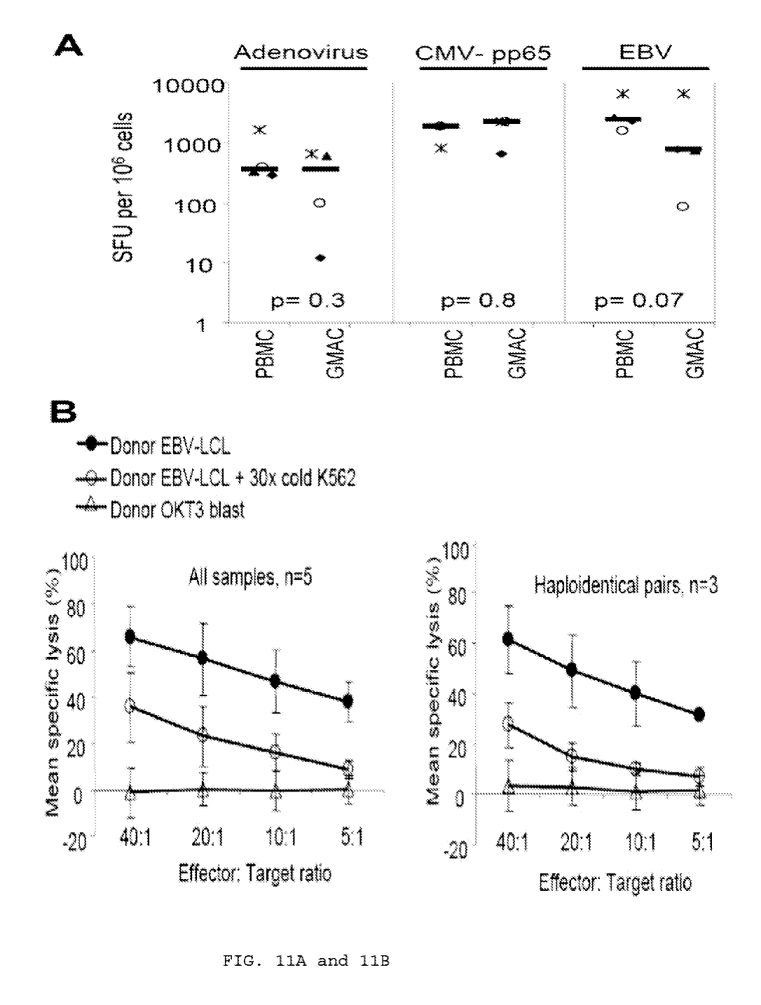

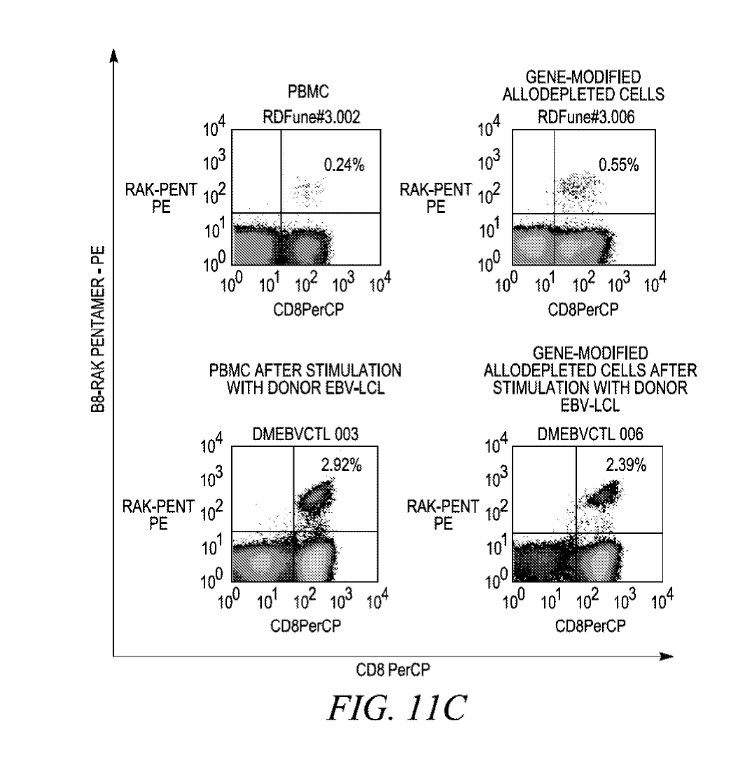

The ability of end-product cells to mediate antiviral immunity was assessed by interferon-ELISpot, cytotoxicity assay, and pentamer analysis. The cryopreserved gene-modified allodepleted cells were used in all analyses, since they were representative of the product currently being evaluated for use in a clinical study. Interferon-γ secretion in response to adenovirus, CMV or EBV antigens presented by donor cells was preserved although there was a trend towards reduced anti-EBV response in gene-modified allodepleted cells versus unmanipulated PBMC (see FIG. 11A). Illustrated in FIG. 11A are the results of the interferon secretion studies. The response to viral antigens was assessed by ELISpot in 4 pairs of unmanipulated PBMC and gene-modified allodepleted cells (GMAC). Adenovirus and CMV antigens were presented by donor-derived activated monocytes through infection with Ad5f35 null vector and Ad5f35-pp65 vector, respectively. EBV antigens were presented by donor EBV-LCL. The number of spot-forming units (SFU) was corrected for stimulator- and responder-alone wells. Only three of four donors were evaluable for CMV response, one seronegative donor was excluded. In FIG. 11A the horizontal bars represent the median.

Cytotoxicity was assessed using donor-derived EBV-LCL as targets. Gene-modified allodepleted cells that had undergone 2 or 3 rounds of stimulation with donor-derived EBV-LCL could efficiently lyse virus-infected autologous target cells (see FIG. 11B). Presented in FIG. 11B are the results of the cytotoxicity assay. Gene-modified allodepleted cells were stimulated with donor EBV-LCL for 2 or 3 cycles. ⁵¹Cr release assay was performed using donor-derived EBV-LCL and donor OKT3 blasts as targets. NK activity was blocked with 30-fold excess cold K562. The left panel shows results from 5 independent experiments using totally or partially mismatched donor-recipient pairs. The right panel shows results from 3 experiments using unrelated HLA haploidentical donor-recipient pairs. Error bars indicate standard deviation.

EBV-LCLs were used as antigen-presenting cells during selective allodepletion, therefore it was possible that EBV-specific T cells could be significantly depleted when the donor and recipient were haploidentical. To investigate this hypothesis, three experiments using unrelated HLA-haploidentical donor-recipient pairs were included, and the results showed that cytotoxicity against donor-derived EBV-LCL was retained. The results were corroborated by pentamer analysis for T cells recognizing HLA-B8-RAKFKQLL (SEQ ID NO: 232), an EBV lytic antigen (BZLF1) epitope, in two informative donors following allodepletion against HLA-B8 negative haploidentical recipients (see FIG. 11C). FIG. 11C illustrates the frequency of T cells specific for the BZLF1 epitope. Unmanipulated PBMC were used as comparators. The RAK-pentamer positive population was retained in gene-modified allodepleted cells and could be expanded following several rounds of in vitro stimulation with donor-derived EBV-LCL. The percentages shown in graph presented in FIG. 11C indicate percentage of pentamer positive cells within the CD8 population. Together, these results indicate that gene-modified allodepleted cells retained significant anti-viral functionality.

Regulatory T Cells in the Gene-Modified Allodepleted Cell Population

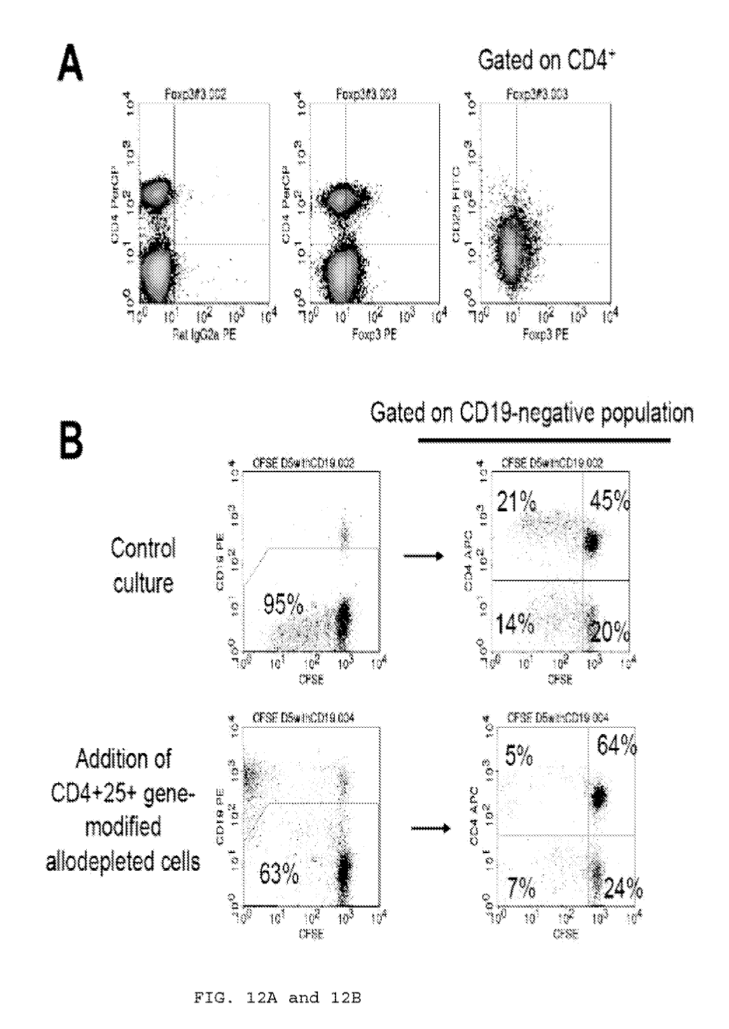

Flow cytometry and functional analysis were used to determine whether regulatory T cells were retained in our allodepleted, gene modified, T cell product. A Foxp3⁺ CD4⁺25⁺ population was found, as shown in FIG. 12A. Following immunomagnetic separation, the CD4⁺ CD25⁺ enriched fraction demonstrated suppressor function when co-cultured with CFSE-labeled autologous PBMC in the presence of OKT3 and allogeneic feeders (see FIG. 12B). FIG. 12B illustrates the results of a CD4⁺ CD25⁺ functional assay. Donor-derived PBMC was labeled with CFSE and stimulated with OKT3 and allogeneic feeders. CD4⁺ CD25⁺ cells were immunomagnetically selected from the gene-modified cell population and added at 1:1 ratio to test wells. Flow cytometry was performed after 5 days. Gene-modified T cells were gated out by CD19 expression. The addition of CD4⁺ CD25⁺ gene-modified cells (bottom panel) significantly reduced cell proliferation. Thus, allodepleted T cells may reacquire regulatory phenotype even after exposure to a CD25 depleting immunotoxin.

Gene-Modified Allodepleted Cells were Efficiently and Rapidly Eliminated by Addition of Chemical Inducer of Dimerization

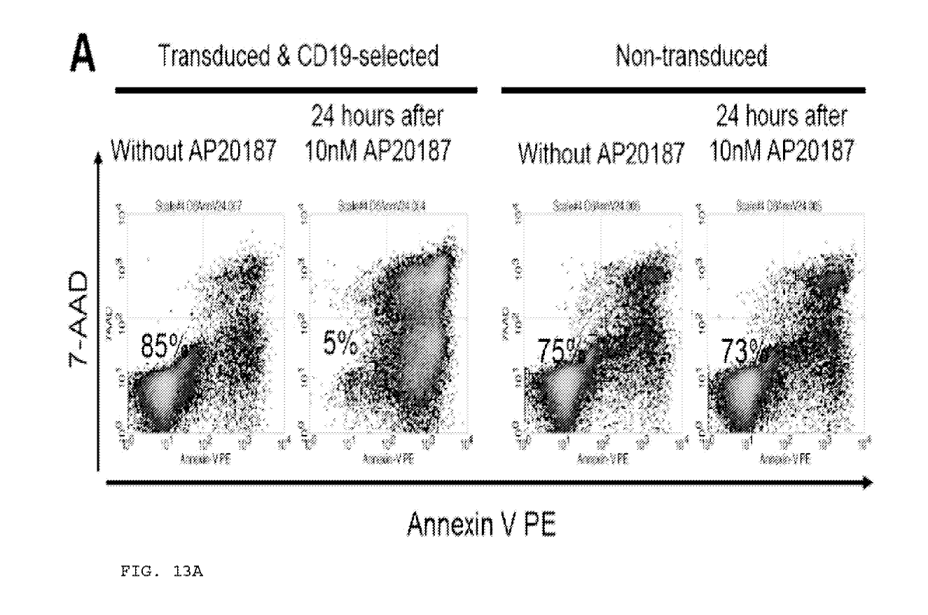

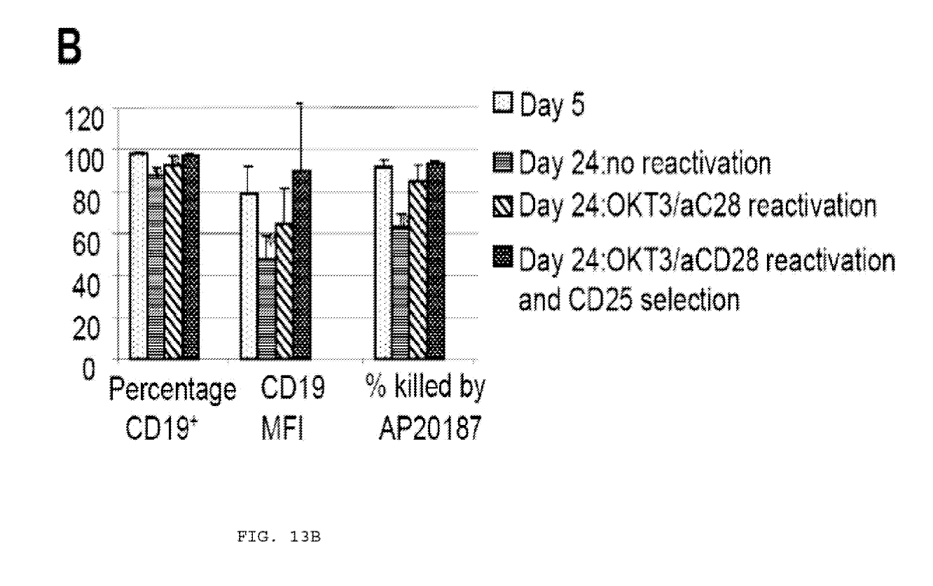

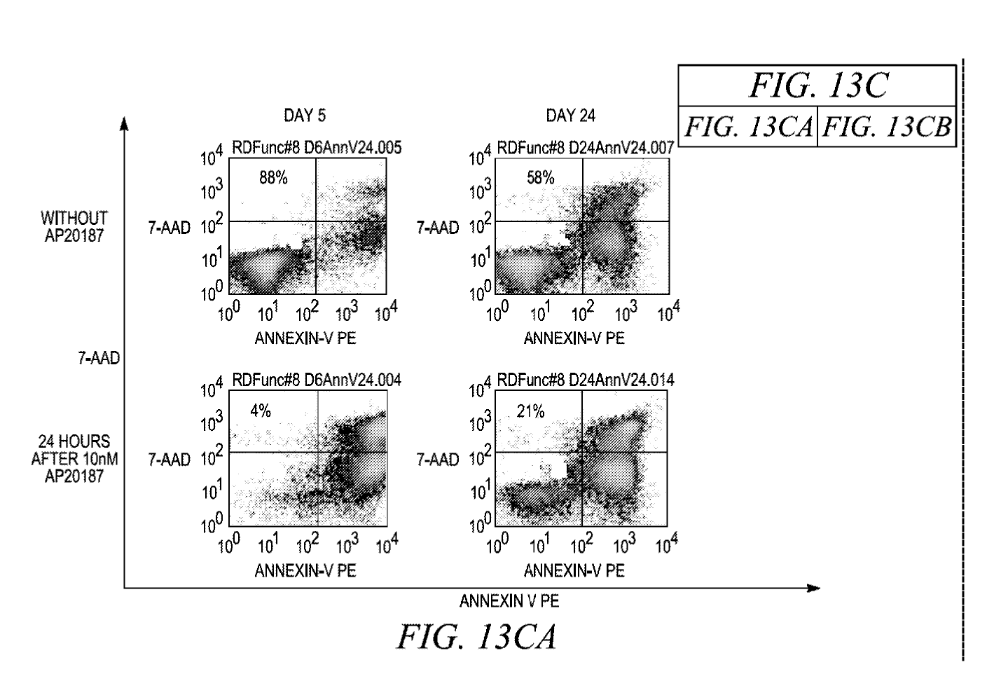

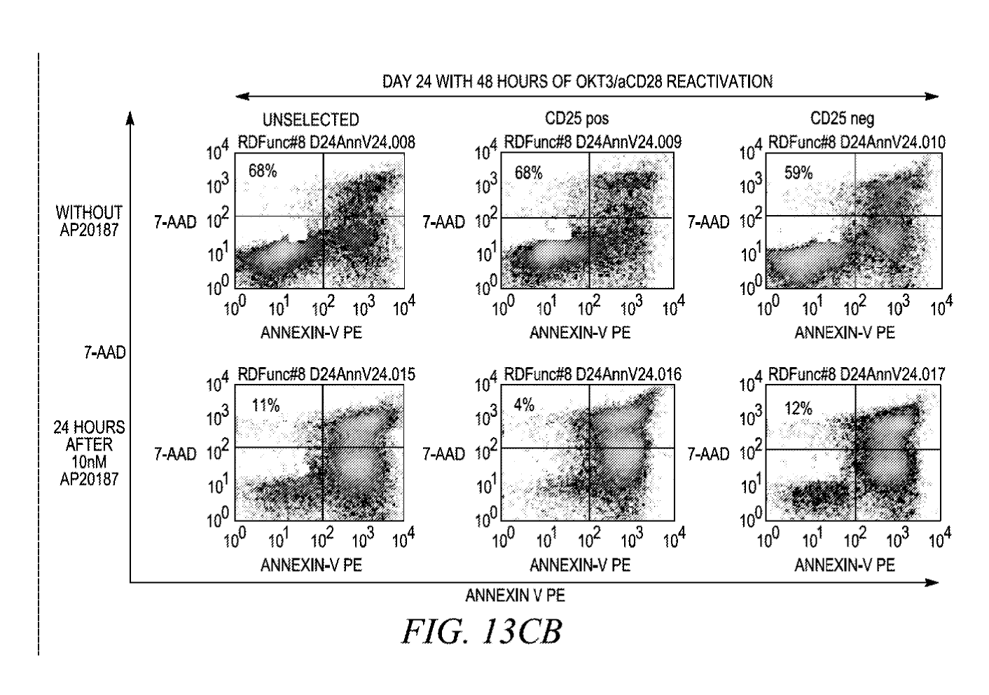

The day following immunomagnetic selection, 10 nM of the chemical inducer of dimerization, AP20187, was added to induce apoptosis, which appeared within 24 hours. FACS analysis with annexin V and 7-AAD staining at 24 hours showed that only about 5.5%±2.5% of AP20187-treated cells remained viable, whereas about 81.0%±9.0% of untreated cells were viable (see FIG. 13A). Killing efficiency after correction for baseline viability was about 92.9%±3.8%. Large-scale CD19 selection produced cells that were killed with similar efficiency as small-scale selection: mean viability with and without AP20187, and percentage killing, in large and small scale were about 3.9%, about 84.0%, about 95.4% (n=3) and about 6.6%, about 79.3%, about 91.4% (n=5) respectively. AP20187 was non-toxic to non-transduced cells: viability with and without AP20187 was about 86%±9% and 87%±8% respectively (n=6).

Transgene Expression and Function Decreased with Extended Culture but were Restored Upon Cell Reactivation

To assess the stability of transgene expression and function, cells were maintained in T cell culture medium and low dose IL-2 (50 U/ml) until 24 days after transduction. A portion of cells was then reactivated with OKT3/anti-CD28. CD19 expression was analyzed by flow cytometry 48 to 72 hours later, and suicide gene function was assessed by treatment with 10 nM AP20187. The results shown in FIG. 13B are for cells from day 5 post transduction (i.e., 1 day after CD 19 selection) and day 24 post transduction, with or without 48-72 hours of reactivation (5 experiments). In 2 experiments, CD25 selection was performed after OKT3/aCD28 activation to further enrich activated cells. Error bars represent standard deviation. * indicates p<0.05 when compared to cells from day 5 post transduction. By day 24, surface CD19 expression fell from about 98%±1% to about 88%±4% (p<0.05) with a parallel decrease in mean fluorescence intensity (MFI) from 793±128 to 478±107 (p<0.05) (see FIG. 13B). Similarly, there was a significant reduction in suicide gene function: residual viability was 19.6±5.6% following treatment with AP20187; after correction for baseline viability of 54.8±20.9%, this equated to killing efficiency of only 63.1±6.2%.

To determine whether the decrease in transgene expression with time was due to reduced transcription following T cell quiescence or to elimination of transduced cells, a portion of cells were reactivated on day 22 post transduction with OKT3 and anti-CD28 antibody. At 48 to 72 hours (day 24 or 25 post transduction), OKT3/aCD28-reactivated cells had significantly higher transgene expression than non-reactivated cells. CD19 expression increased from about 88%±4% to about 93%±4% (p<0.01) and CD19 MFI increased from 478±107 to 643±174 (p<0.01). Additionally, suicide gene function also increased significantly from about a 63.1%±6.2% killing efficiency to about a 84.6%±8.0% (p<0.01) killing efficiency. Furthermore, killing efficiency was completely restored if the cells were immunomagnetically sorted for the activation marker CD25: killing efficiency of CD25 positive cells was about 93%.2±1.2%, which was the same as killing efficiency on day 5 post transduction (93.1±3.5%) (see FIG. 13C). Killing of the CD25 negative fraction was 78.6±9.1%. Illustrated in FIG. 13C are representative FACS plots showing the effect of extended culture and T cell activation on suicide gene function.

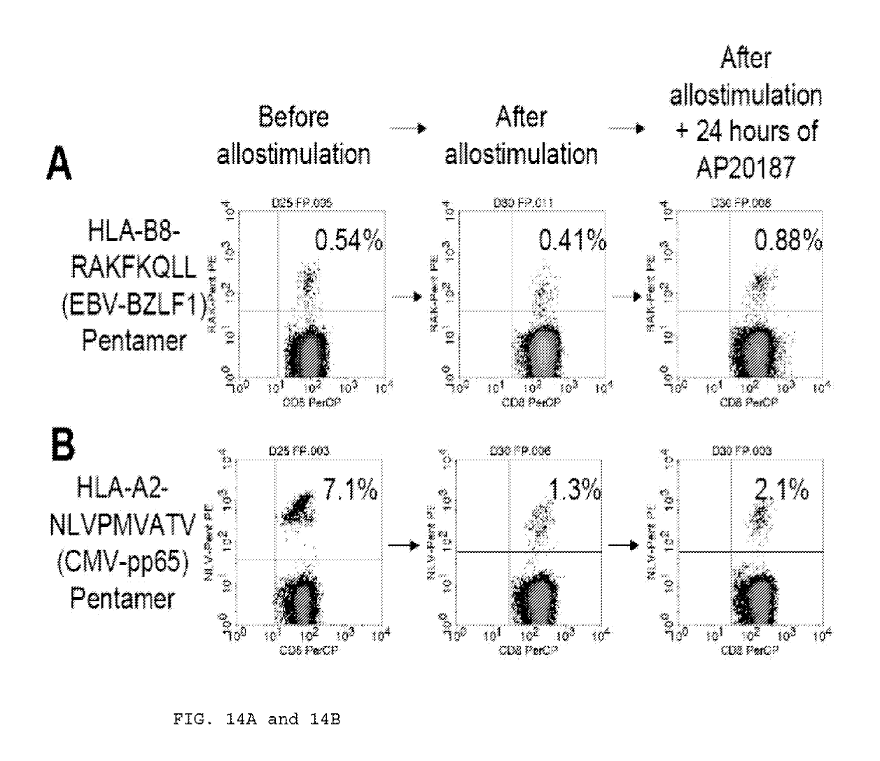

An observation of note was that many virus-specific T cells were spared when dimerizer was used to deplete gene-modified cells that have been re-activated with allogeneic PBMC, rather than by non-specific mitogenic stimuli. After 4 days reactivation with allogeneic cells, as shown in FIGS. 14A and 14B, treatment with AP20187 spares (and thereby enriches) viral reactive subpopulations, as measured by the proportion of T cells reactive with HLA pentamers specific for peptides derived from EBV and CMV. Gene-modified allodepleted cells were maintained in culture for 3 weeks post-transduction to allow transgene down-modulation. Cells were stimulated with allogeneic PBMC for 4 days, following which a portion was treated with 10 nM AP20187. The frequency of EBV-specific T cells (see FIG. 14A) and CMV-specific T cells (see FIG. 14B) were quantified by pentamer analysis before allostimulation, after allostimulation, and after treatment of allostimulated cells with dimerizer. The percentage of virus-specific T cells decreased after allostimulation. Following treatment with dimerizer, virus-specific T cells were partially and preferentially retained.

### DISCUSSION

The feasibility of engineering allogeneic T cells with two distinct safety mechanisms, selective allodepletion and suicide gene-modification has been demonstrated herein. In combination, these modifications can enhance and/or enable addback of substantial numbers of T cells with anti-viral and anti-tumor activity, even after haploidentical transplantation. The data presented herein show that the suicide gene, iCasp9, functions efficiently (>90% apoptosis after treatment with dimerizer) and that down-modulation of transgene expression that occurred with time was rapidly reversed upon T cell activation, as would occur when alloreactive T cells encountered their targets. Data presented herein also show that CD19 is a suitable selectable marker that enabled efficient and selective enrichment of transduced cells to >90% purity. Furthermore the data presented herein indicate that these manipulations had no discernable effects on the immunological competence of the engineered T cells with retention of antiviral activity, and regeneration of a CD4⁺ CD25⁺ Foxp3⁺ population with Treg activity.

Given that the overall functionality of suicide genes depends on both the suicide gene itself and the marker used to select the transduced cells, translation into clinical use requires optimization of both components, and of the method used to couple expression of the two genes. The two most widely used selectable markers, currently in clinical practice, each have drawbacks. Neomycin phosphotransferase (neo) encodes a potentially immunogenic foreign protein and requires a 7-day culture in selection medium, which not only increases the complexity of the system, but is also potentially damaging to virus-specific T cells. A widely used surface selection marker, LNGFR, has recently had concerns raised, regarding its ocogenic potential and potential correlation with leukemia, in a mouse model, despite its apparent clinical safety. Furthermore, LNGFR selection is not widely available, because it is used almost exclusively in gene therapy. A number of alternative selectable markers have been suggested. CD34 has been well-studied in vitro, but the steps required to optimize a system configured primarily for selection of rare hematopoietic progenitors, and more critically, the potential for altered in vivo T cell homing, make CD34 sub-optimal for use as a selectable marker for a suicide switch expression construct. CD19 was chosen as an alternative selectable marker, since clinical grade CD19 selection is readily available as a method for B-cell depletion of stem cell autografts. The results presented herein demonstrated that CD19 enrichment could be performed with high purity and yield and, furthermore, the selection process had no discernable effect on subsequent cell growth and functionality.

The effectiveness of suicide gene activation in CD19-selected iCasp9 cells compared very favorably to that of neo- or LNGFR-selected cells transduced to express the HSVtk gene. The earlier generations of HSVtk constructs provided 80-90% suppression of ³H-thymidine uptake and showed similar reduction in killing efficiency upon extended in vitro culture, but were nonetheless clinically efficacious. Complete resolution of both acute and chronic GVHD has been reported with as little as 80% in vivo reduction in circulating gene-modified cells. These data support the hypothesis that transgene down-modulation seen in vitro is unlikely to be an issue because activated T cells responsible for GVHD will upregulate suicide gene expression and will therefore be selectively eliminated in vivo. Whether this effect is sufficient to allow retention of virus- and leukemia-specific T cells in vivo will be tested in a clinical setting. By combining in vitro selective allodepletion prior to suicide gene modification, the need to activate the suicide gene mechanism may be significantly reduced, thereby maximizing the benefits of addback T cell based therapies.

The high efficiency of iCasp9-mediated suicide seen in vitro has been replicated in vivo. In a SCID mouse-human xenograft model, more than 99% of iCasp9-modified T cells were eliminated after a single dose of dimerizer. AP1903, which has extremely close functional and chemical equivalence to AP20187, and currently is proposed for use in a clinical application, has been safety tested on healthy human volunteers and shown to be safe. Maximal plasma level of between about 10 ng/ml to about 1275 ng/ml AP1903 (equivalent to between about 7 nM to about 892 nM) was attained over a 0.01 mg/kg to 1.0 mg/kg dose range administered as a 2-hour intravenous infusion. There were substantially no significant adverse effects. After allowing for rapid plasma redistribution, the concentration of dimerizer used in vitro remains readily achievable in vivo.

Optimal culture conditions for maintaining the immunological competence of suicide gene-modified T cells must be determined and defined for each combination of safety switch, selectable marker and cell type, since phenotype, repertoire and functionality can all be affected by the stimulation used for polyclonal T cell activation, the method for selection of transduced cells, and duration of culture. The addition of CD28 co-stimulation and the use of cell-sized paramagnetic beads to generate gene modified-cells that more closely resemble unmanipulated PBMC in terms of CD4:CD8 ratio, and expression of memory subset markers including lymph node homing molecules CD62L and CCR7, may improve the in vivo functionality of gene-modified T cells. CD28 co-stimulation also may increase the efficiency of retroviral transduction and expansion. Interestingly however, the addition of CD28 co-stimulation was found to have no impact on transduction of allodepleted cells, and the degree of cell expansion demonstrated was higher when compared to the anti-CD3 alone arm in other studies. Furthermore, iCasp9-modified allodepleted cells retained significant anti-viral functionality, and approximately one fourth retained CD62L expression. Regeneration of CD4⁺ CD25⁺ Foxp3⁺ regulatory T cells was also seen. The allodepleted cells used as the starting material for T cell activation and transduction may have been less sensitive to the addition of anti-CD28 antibody as co-stimulation. CD25-depleted PBMC/EBV-LCL co-cultures contained T cells and B cells that already express CD86 at significantly higher level than unmanipulated PBMCs and may themselves provide co-stimulation. Depletion of CD25⁺ regulatory T cells prior to polyclonal T cell activation with anti-CD3 has been reported to enhance the immunological competence of the final T cell product. In order to minimize the effect of in vitro culture and expansion on functional competence, a relatively brief culture period was used in some experiments presented herein, whereby cells were expanded for a total of 8 days post-transduction with CD19-selection being performed on day 4.

Finally, scaled up production was demonstrated such that sufficient cell product can be produced to treat adult patients at doses of up to 10⁷ cells/kg: allodepleted cells can be activated and transduced at 4×10⁷ cells per flask, and a minimum of 8-fold return of CD19-selected final cell product can be obtained on day 8 post-transduction, to produce at least 3×10⁸ allodepleted gene-modified cells per original flask. The increased culture volume is readily accommodated in additional flasks or bags.

The allodepletion and iCasp9-modification presented herein may significantly improve the safety of adding back T cells, particularly after haploidentical stem cell allografts. This should in turn enable greater dose-escalation, with a higher chance of producing an anti-leukemia effect.

### Example 3

### CASPALLO—Phase I Clinical Trial of Allodepleted T Cells Transduced with Inducible Caspase-9 Suicide Gene after Haploidentical Stem Cell Transplantation

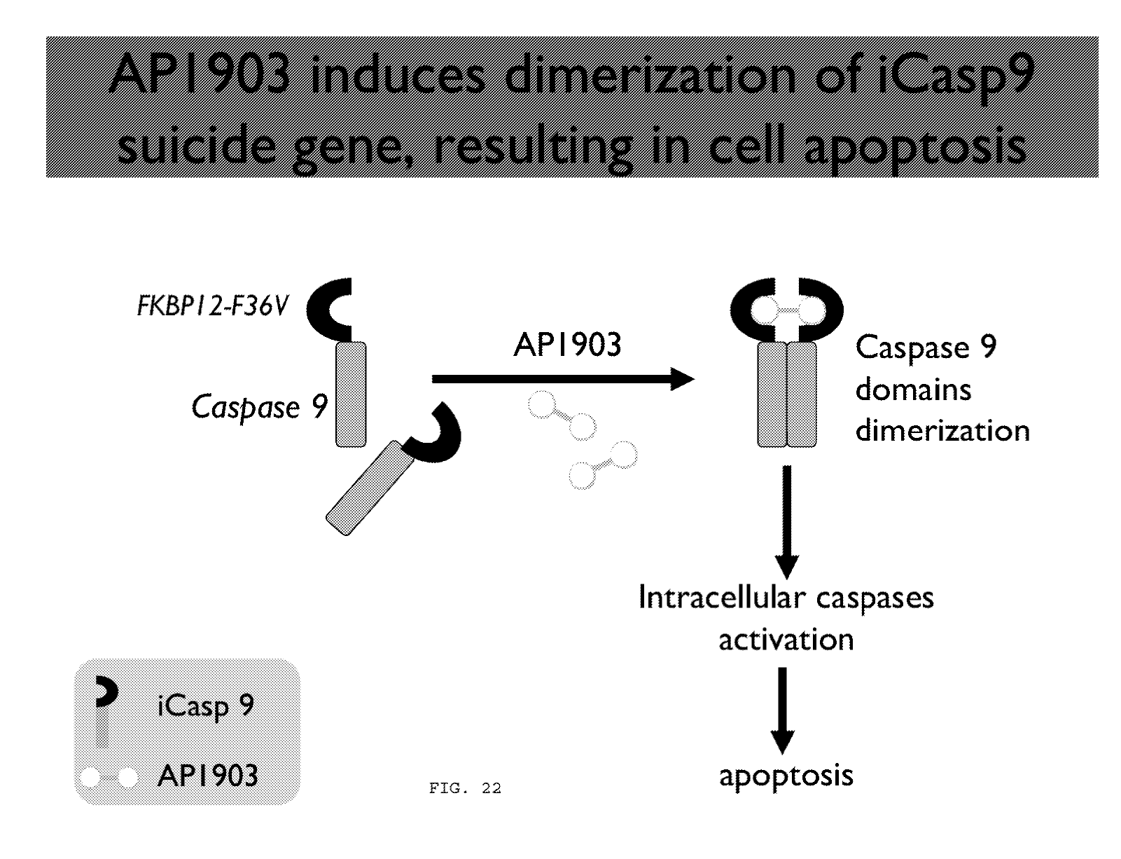

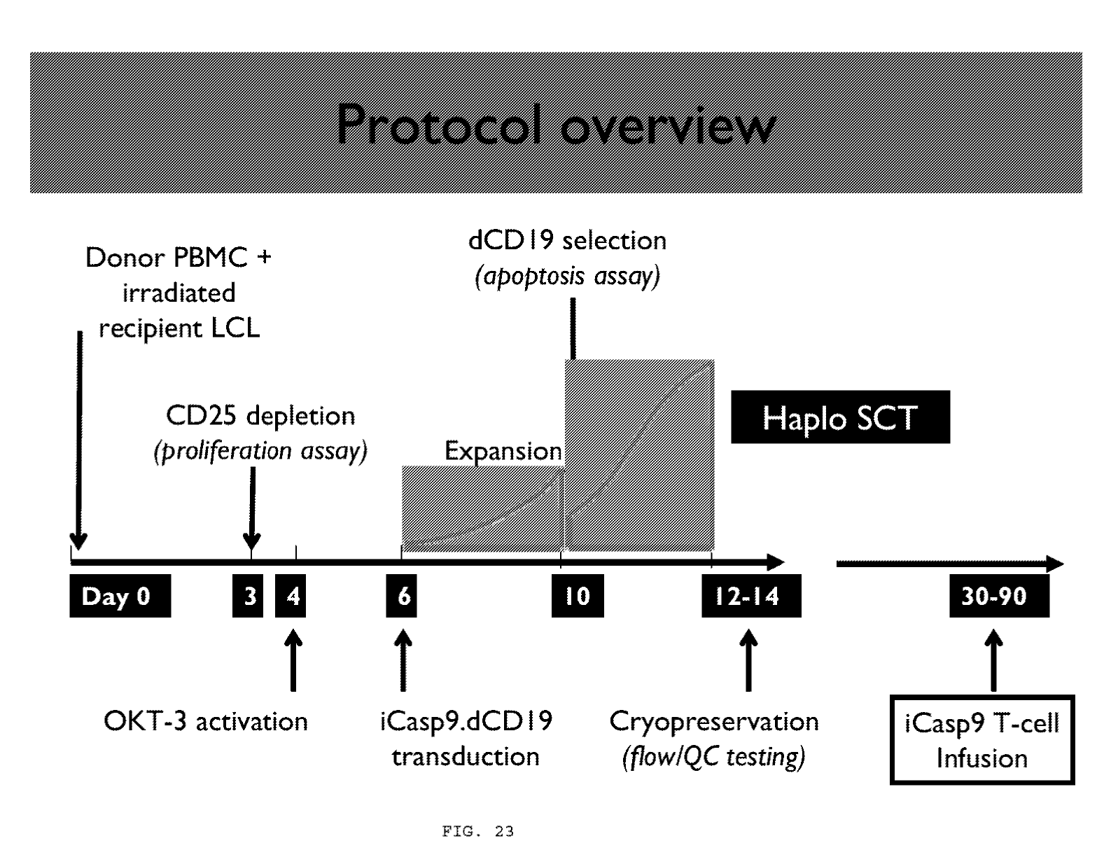

This example presents results of a phase 1 clinical trial using the alternative suicide gene strategy illustrated in FIG. 22. Briefly, donor peripheral blood mononuclear cells were co-cultured with recipient irradiated EBV-transformed lymphoblastoid cells (40:1) for 72 hrs, allodepleted with a CD25 immunotoxin and then transduced with a retroviral supernatant carrying the iCasp9 suicide gene and a selection marker (ΔCD19); ΔCD19 allowed enrichment to >90% purity via immunomagnetic selection., as illustrated in FIG. 23.

An example of a protocol for generation of a cell therapy product is provided herein.

Source Material

Up to 240 ml (in 2 collections) of peripheral blood was obtained from the transplant donor according to established protocols. In some cases, dependent on the size of donor and recipient, a leukopheresis was performed to isolate sufficient T cells. 10 cc-30 cc of blood also was drawn from the recipient and was used to generate the Epstein Barr virus (EBV)-transformed lymphoblastoid cell line used as stimulator cells. In some cases, dependent on the medical history and/or indication of a low B cell count, the LCLs were generated using appropriate 1st degree relative (e.g., parent, sibling, or offspring) peripheral blood mononuclear cells.

Generation of Allodepleted Cells

Allodepleted cells were generated from the transplant donors as presented herein. Peripheral blood mononuclear cells (PBMCs) from healthy donors were co-cultured with irradiated recipient Epstein Barr virus (EBV)-transformed lymphoblastoid cell lines (LCL) at responder-to-stimulator ratio of 40:1 in serum-free medium (AIM V; Invitrogen, Carlsbad, Calif.). After 72 hours, activated T cells that express CD25 were depleted from the co-culture by overnight incubation in RFT5-SMPT-dgA immunotoxin. Allodepletion is considered adequate if the residual CD3⁺ CD25⁺ population was <1% and residual proliferation by ³H-thymidine incorporation was <10%.

Retroviral Production

A retroviral producer line clone was generated for the iCasp9-CD19 construct. A master cell-bank of the producer also was generated. Testing of the master-cell bank was performed to exclude generation of replication competent retrovirus and infection by Mycoplasma, HIV, HBV, HCV and the like. The producer line was grown to confluency, supernatant harvested, filtered, aliquoted and rapidly frozen and stored at −80° C. Additional testing was performed on all batches of retroviral supernatant to exclude Replication Competent Retrovirus (RCR) and issued with a certificate of analysis, as per protocol.

Transduction of Allodepleted Cells

Allodepleted T-lymphocytes were transduced using Fibronectin. Plates or bags were coated with recombinant Fibronectin fragment CH-296 (Retronectin™, Takara Shuzo, Otsu, Japan). Virus was attached to retronectin by incubating producer supernatant in coated plates or bags. Cells were then transferred to virus coated plates or bags. After transduction allodepleted T cells were expanded, feeding them with IL-2 twice a week to reach the sufficient number of cells as per protocol.

CD19 Immunomagnetic Selection

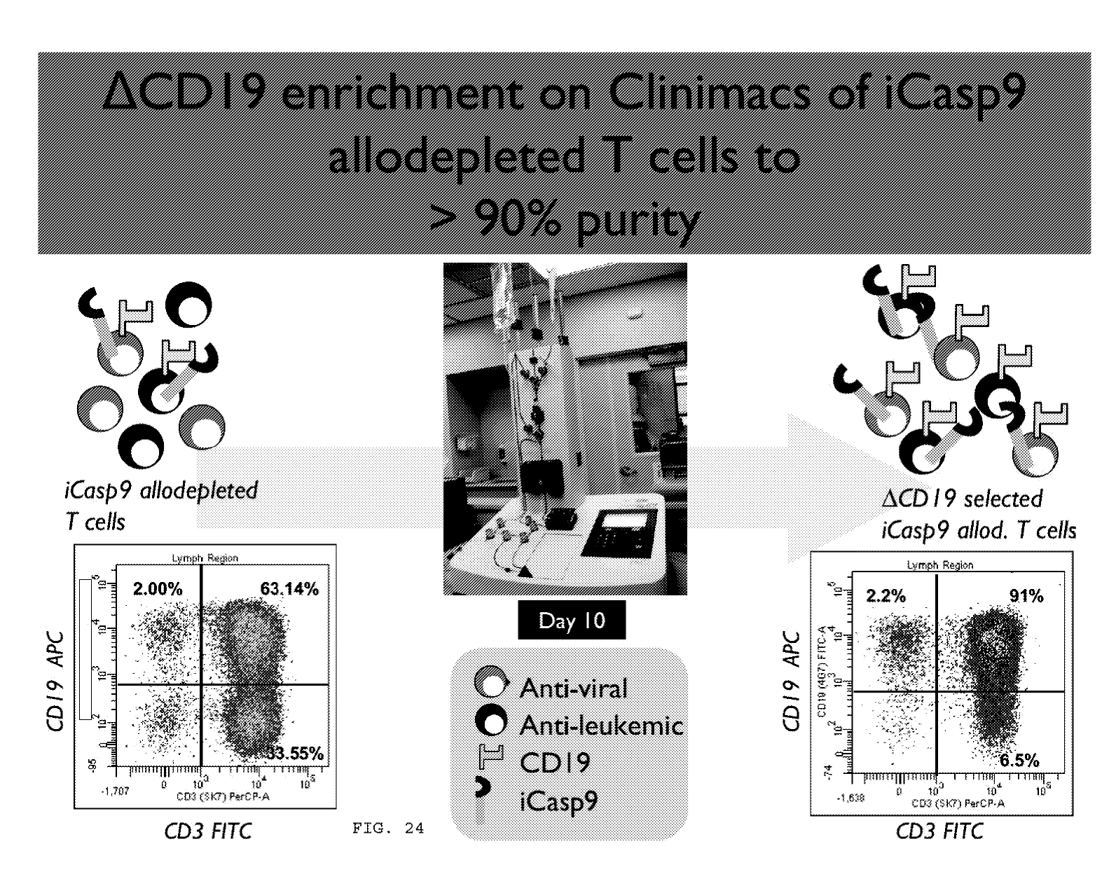

Immunomagnetic selection for CD19 was performed 4 days after transduction. Cells are labeled with paramagnetic microbeads conjugated to monoclonal mouse anti-human CD19 antibodies (Miltenyi Biotech, Auburn, Calif.) and selected on a CliniMacs Plus automated selection device (see FIG. 24). Depending upon the number of cells required for clinical infusion cells were either cryopreserved after the CliniMacs selection or further expanded with IL-2 and cryopreserved on day 6 or day 8 post transduction.

Freezing

Aliquots of cells were removed for testing of transduction efficiency, identity, phenotype and microbiological culture as required for final release testing by the FDA. The cells were cryopreserved prior to administration according to protocol.

Study Drugs

RFT5-SMPT-dgA

RFT5-SMPT-dgA is a murine IgG1 anti-CD25 (IL-2 receptor alpha chain) conjugated via a hetero-bifunctional crosslinker [N-succinimidyloxycarbonyl-alpha-methyl-d-(2-pyridylthio) toluene] (SMPT) to chemically deglycosylated ricin A chain (dgA). RFT5-SMPT-dgA is formulated as a sterile solution at 0.5 mg/ml.

Synthetic Homodimerizer, AP1903

Mechanism of Action: AP1903-inducible cell death is achieved by expressing a chimeric protein comprising the intracellular portion of the human (caspase-9 protein) receptor, which signals apoptotic cell death, fused to a drug-binding domain derived from human FK506-binding protein (FKBP). This chimeric protein remains quiescent inside cells until administration of AP1903, which cross-links the FKBP domains, initiating Caspase signaling and apoptosis.

Toxicology: AP1903 has been evaluated as an Investigational New Drug (IND) by the FDA and has successfully completed a phase I clinical safety study. No significant adverse effects were noted when API 903 was administered over a 0.01 mg/kg to 1.0 mg/kg dose range.

Pharmacology/Pharmacokinetics: Patients received 0.4 mg/kg of AP1903 as a 2 h infusion—based on published Pk data which show plasma concentrations of 10 ng/mL-I275 ng/mL over the 0.01 mg/kg to 1.0 mg/kg dose range with plasma levels falling to 18% and 7% of maximum at 0.5 and 2 hrs post dose.

Side Effect Profile in Humans: No serious adverse events occurred during the Phase 1 study in volunteers. The incidence of adverse events was very low following each treatment, with all adverse events being mild in severity. Only one adverse event was considered possibly related to API903. This was an episode of vasodilatation, presented as “facial flushing” for 1 volunteer at the 1.0 mg/kg API903 dosage. This event occurred at 3 minutes after the start of infusion and resolved after 32 minutes duration. All other adverse events reported during the study were considered by the investigator to be unrelated or to have improbable relationship to the study drug. These events included chest pain, flu syndrome, halitosis, headache, injection site pain, vasodilatation, increased cough, rhinitis, rash, gum hemorrhage, and ecchymosis.

Patients developing grade 1 GVHD were treated with 0.4 mg/kg API903 as a 2-hour infusion. Protocols for administration of AP1903 to patients grade 1 GVHD were established as follows. Patients developing GvHD after infusion of allodepleted T cells are biopsied to confirm the diagnosis and receive 0.4 mg/kg of AP1903 as a 2 h infusion. Patients with Grade I GVHD received no other therapy initially, however if they showed progression of GvHD conventional GvHD therapy was administered as per institutional guidelines. Patients developing grades 2-4 GVHD were administered standard systemic immunosuppressive therapy per institutional guidelines, in addition to the AP1903 dimerizer drug.

Instructions for preparation and infusion: AP1903 for injection is obtained as a concentrated solution of 2.33 ml in a 3 ml vial, at a concentration of 5 mg/mi, (i.e., 10.66 mg per vial). Prior to administration, the calculated dose was diluted to 100 mL in 0.9% normal saline for infusion. AP1903 for injection (0.4 mg/kg) in a volume of 100 ml was administered via IV infusion over 2 hours, using a non-DEHP, non-ethylene oxide sterilized infusion set and infusion pump.

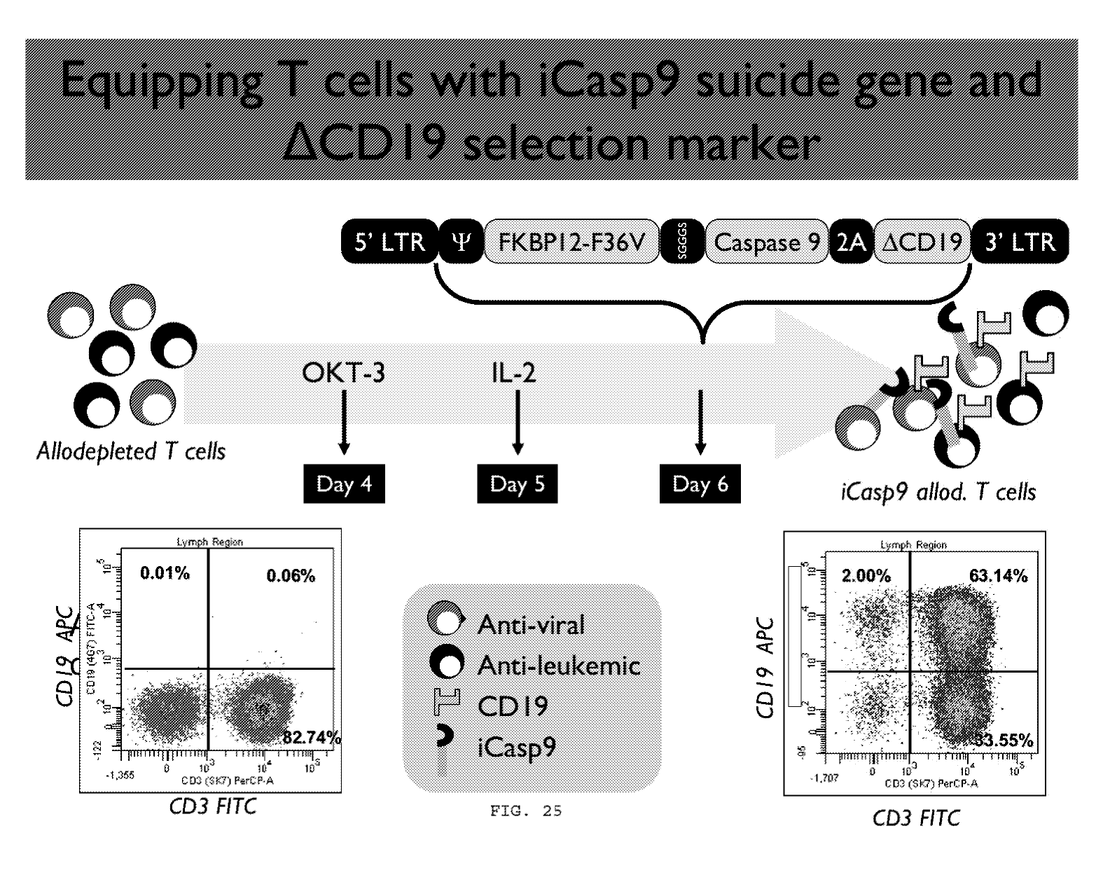

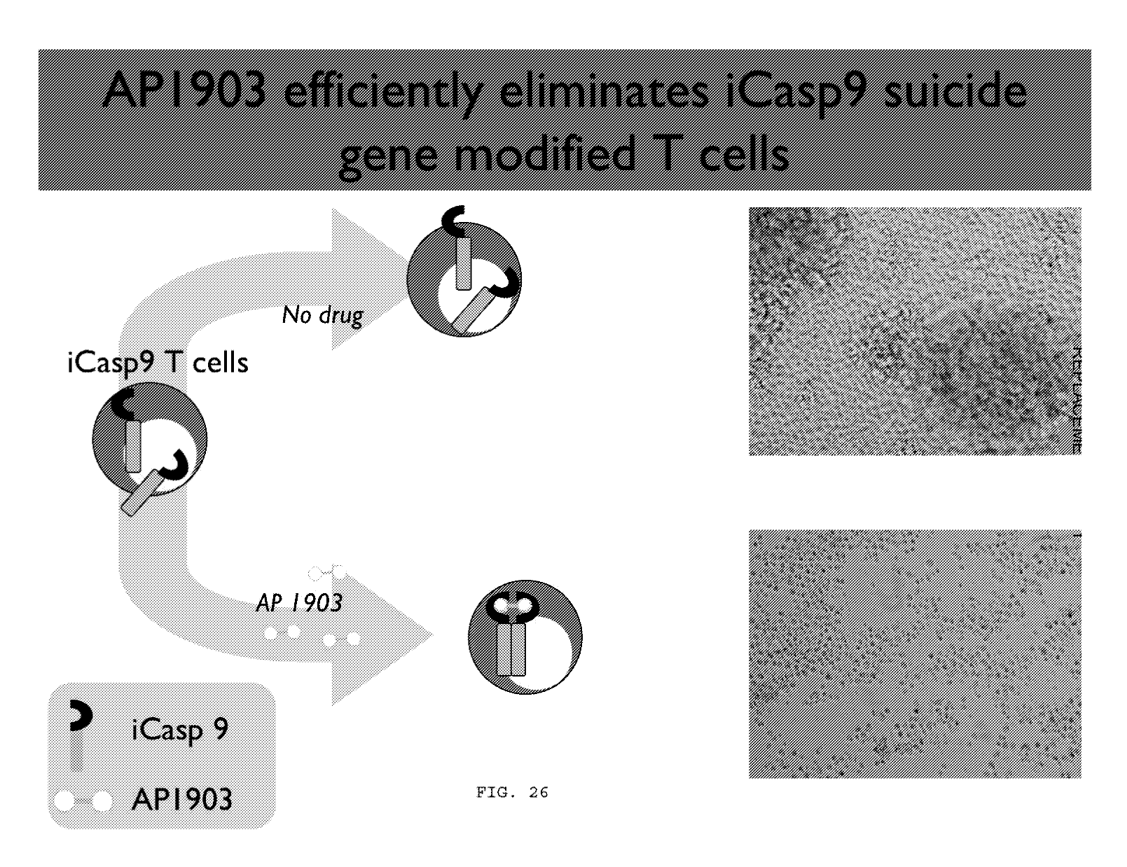

The iCasp9 suicide gene expression construct (e.g., SFG.iCasp9.2A.ACD19), shown in FIG. 25, consists of inducible caspase-9 (iCasp9) linked, via a cleavable 2A-like sequence, to truncated human CD19 (ACD19). iCasp9 includes a human FK506-binding protein (FKBP12; GenBank AH002 818) with an F36V mutation, connected via a Ser-Gly-Gly-Gly-Ser-Gly linker (SEQ ID NO: 239) to human caspase-9 (CASP9; GenBank NM 001229). The F36V mutation may increase the binding affinity of FKBP12 to the synthetic homodimerizer, AP20187 or API903. The Caspase recruitment domain (CARD) has been deleted from the human caspase-9 sequence and its physiological function has been replaced by FKBP12. The replacement of CARD with FKBP12 increases transgene expression and function. The 2A-like sequence encodes an 18 amino acid peptide from Thosea Asigna insect virus, which mediates>99% cleavage between a glycine and terminal proline residue, resulting in 17 extra amino acids in the C terminus of iCasp9, and one extra proline residue in the N terminus of CD19. ΔCD19 consists of full length CD19 (GenBank NM 001770) truncated at amino acid 333 (TDPTRRF (SEQ ID NO: 237)), which shortens the intracytoplasmic domain from 242 to 19 amino acids, and removes all conserved tyrosine residues that are potential sites for phosphorylation. Illustrated in FIG. 26 is the result of iCasp9 and AP1903 in eliminating gene modified T cells carrying the iCasp9 suicide switch.

In Vivo Studies

Three patients received iCasp9+ T cells after haplo-CD34⁺ stem cell transplantation (SCT), at dose levels between about 1×10⁶ to about 3×10⁶ cells/kg.

TABLE 2
Characteristics of the patients and clinical outcome.

| | | | | Days | | | |
|---|---|---|---|---|---|---|---|
| | | | | from | Number | | Clini- |
| Pa- | Sex | | Disease | SCT to | of cells | | cal |
| tient | (age | Diagno- | status | T-cell | infused | Acute | out- |
| # | (yr)) | sis | at SCT | infusion | per kg | GvHD | come |
| P1 | M(3) | MDS/ | CR2 | 63 | 1 × 10⁶ | Grade | Alive in |
| | | AML | | | | 1/2 | CR > 12 |
| | | | | | | (skin, | months |
| | | | | | | liver) | No |
| | | | | | | | GvHD |
| P2 | F(17) | B-ALL | CR2 | 80 and | (1 × | Grade 1 | Alive in |
| | | | | 112 | 10⁶)2 | (skin) | CR > 12 |
| | | | | | | | months |
| | | | | | | | No |
| | | | | | | | GvHD |
| P3 | M(8) | T-ALL | PIF/CR1 | 93 | 3 × 10⁶ | None | Alive in |
| | | | | | | | CR > 12 |
| | | | | | | | No |
| | | | | | | | GvHD |
| P4 | F(4) | T-ALL | Active | 30 | 3 × 10⁶ | Grade 1 | Alive in |
| | | | disease | | | (skin) | CR > 12 |
| | | | | | | | No |
| | | | | | | | GvHD |

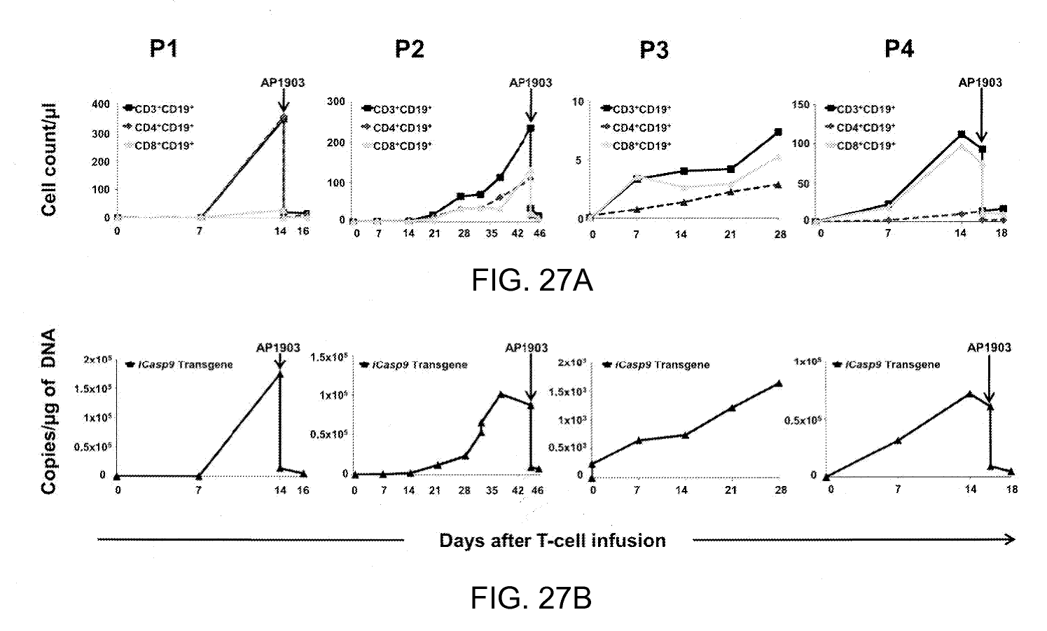

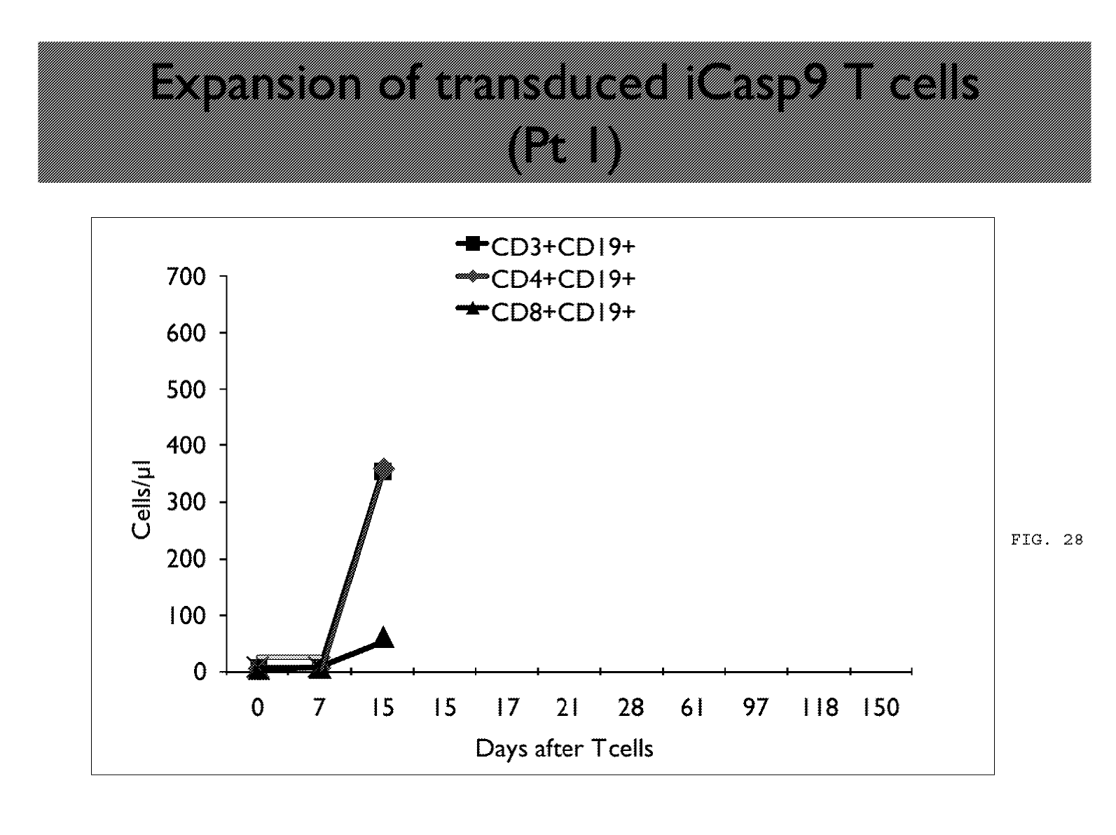

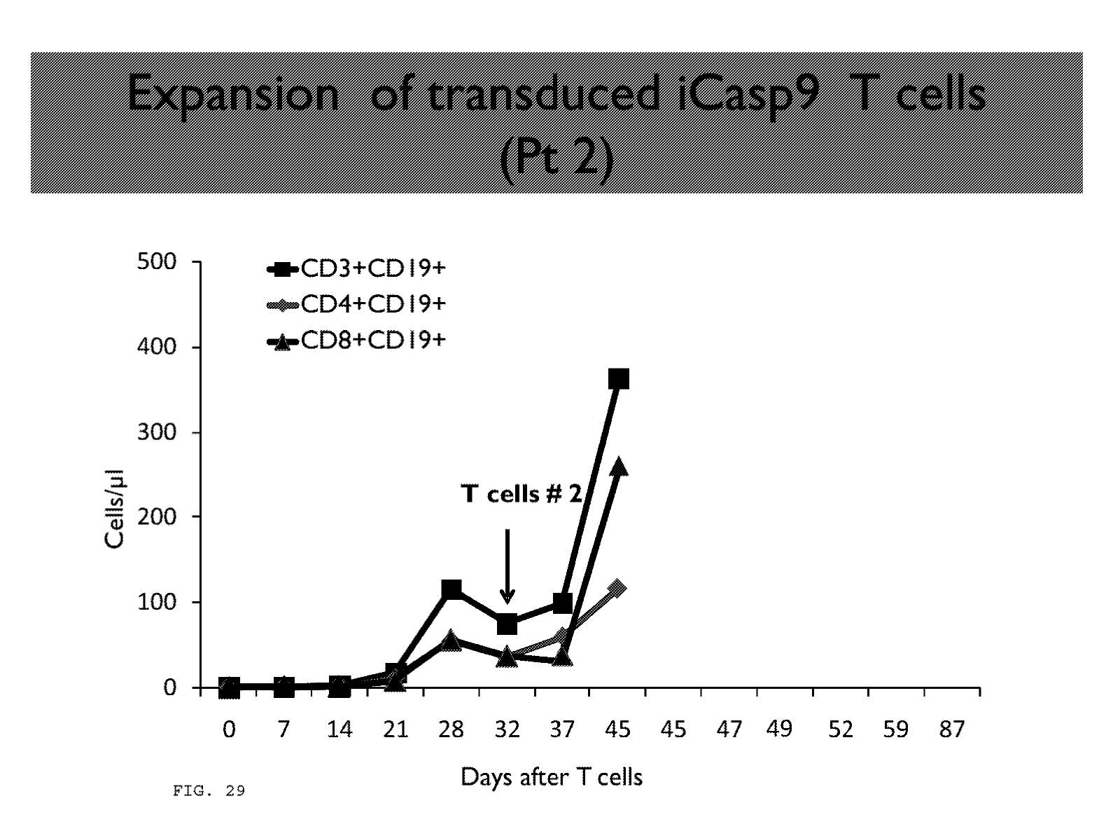

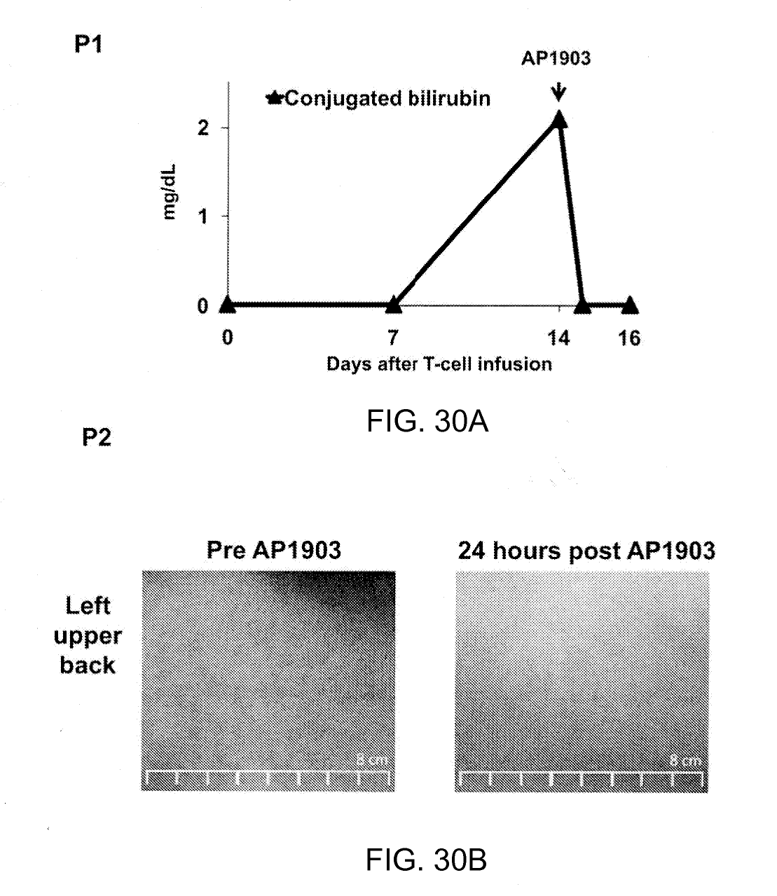

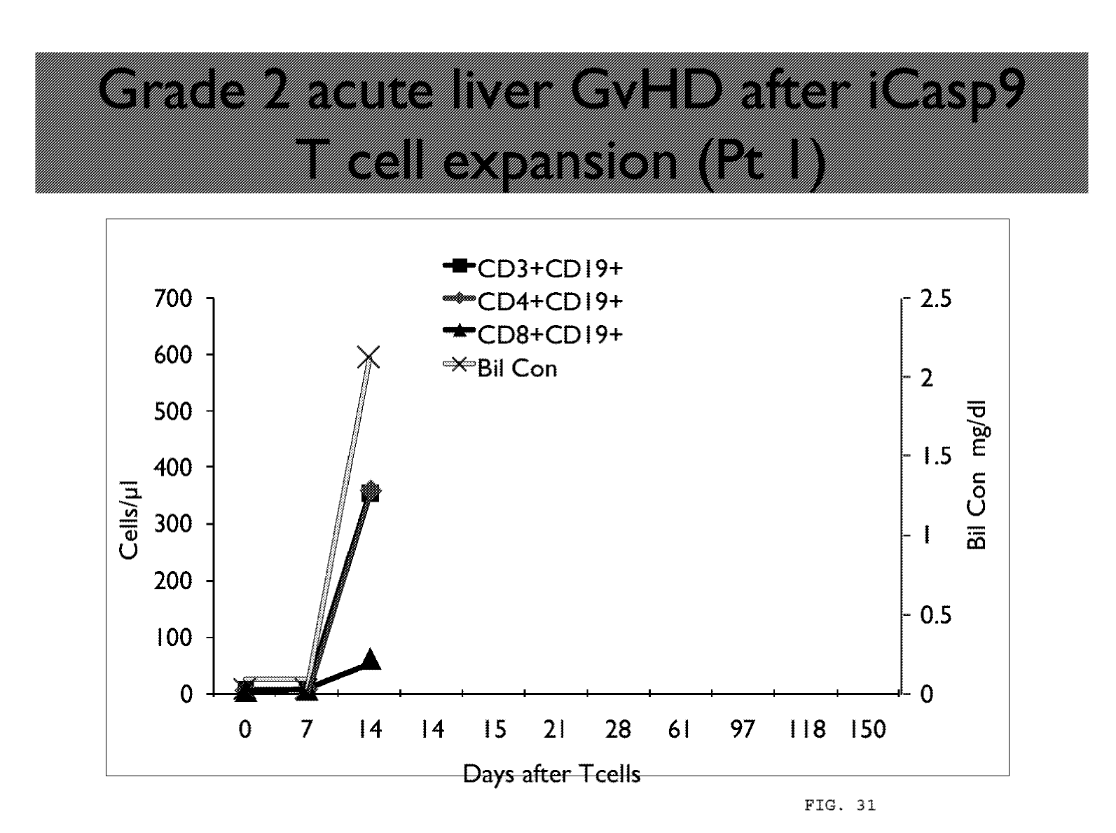

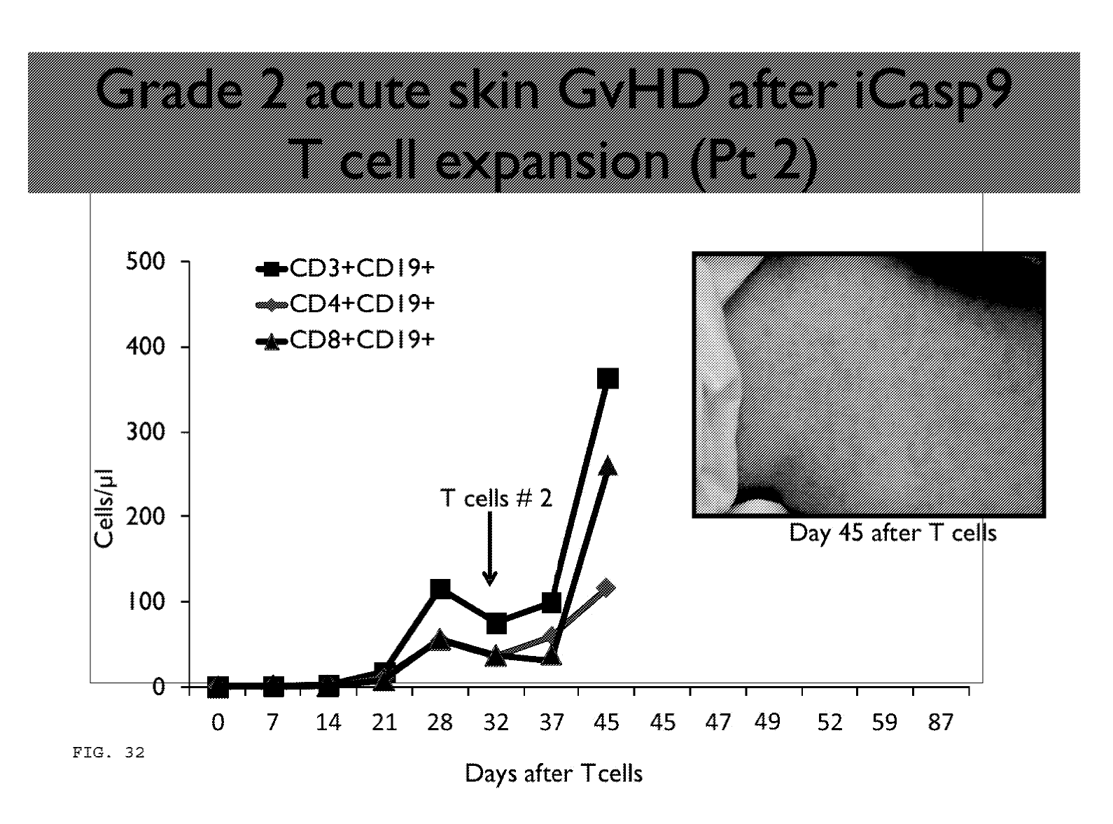

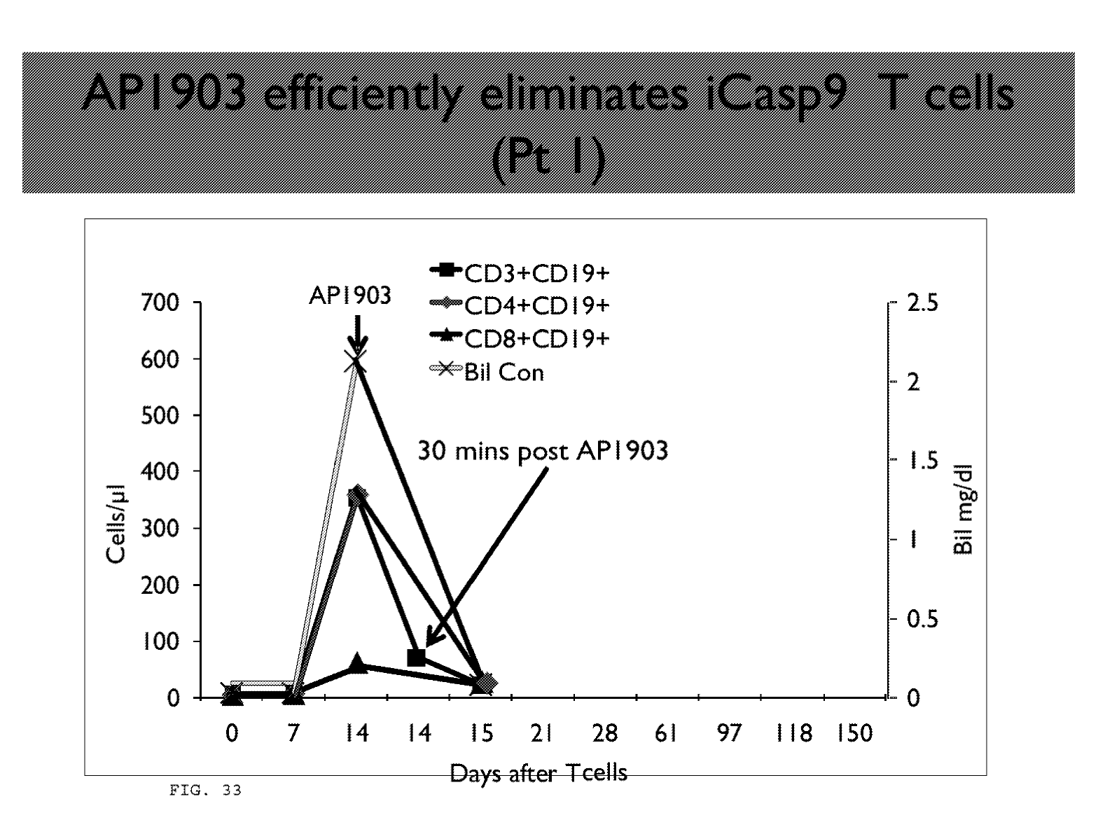

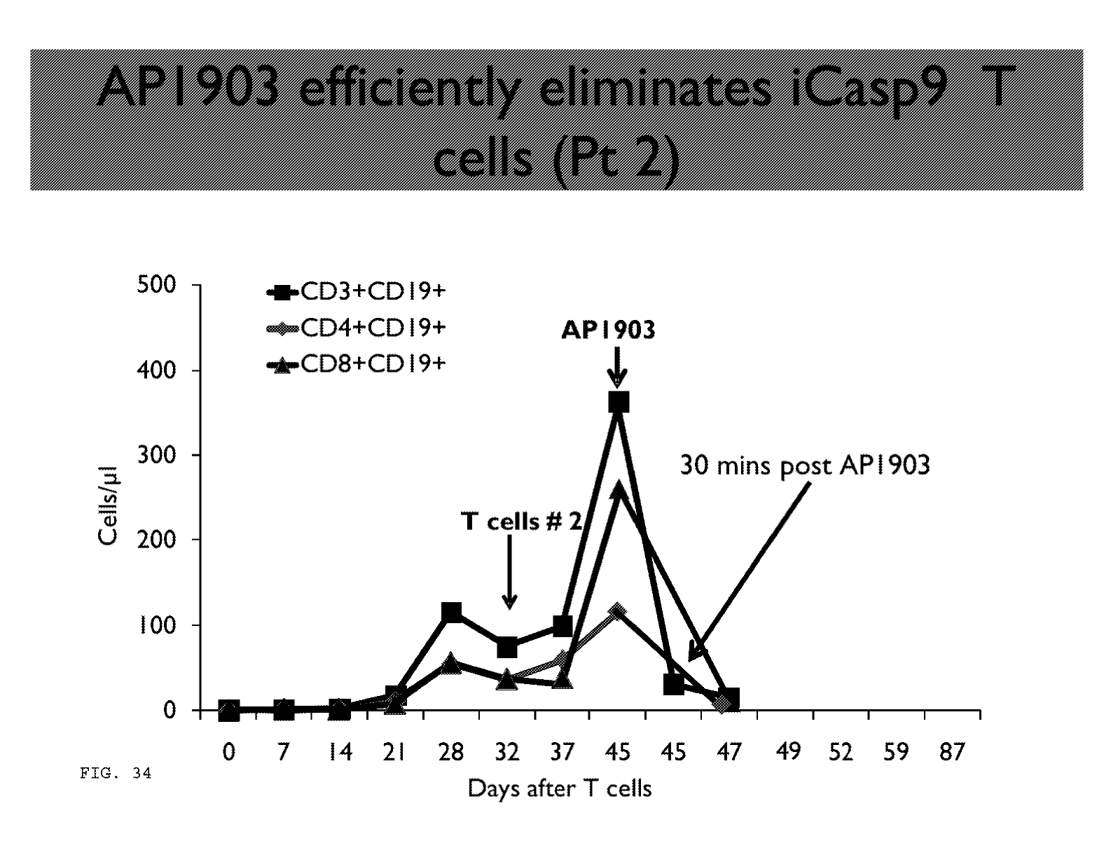

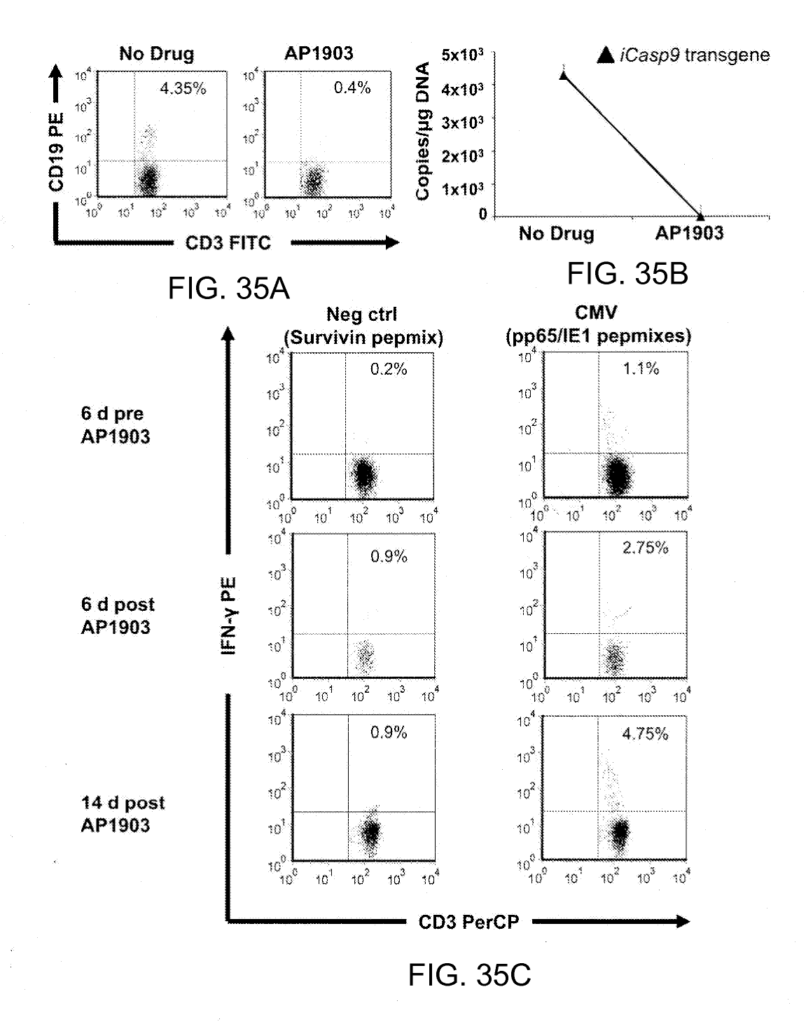

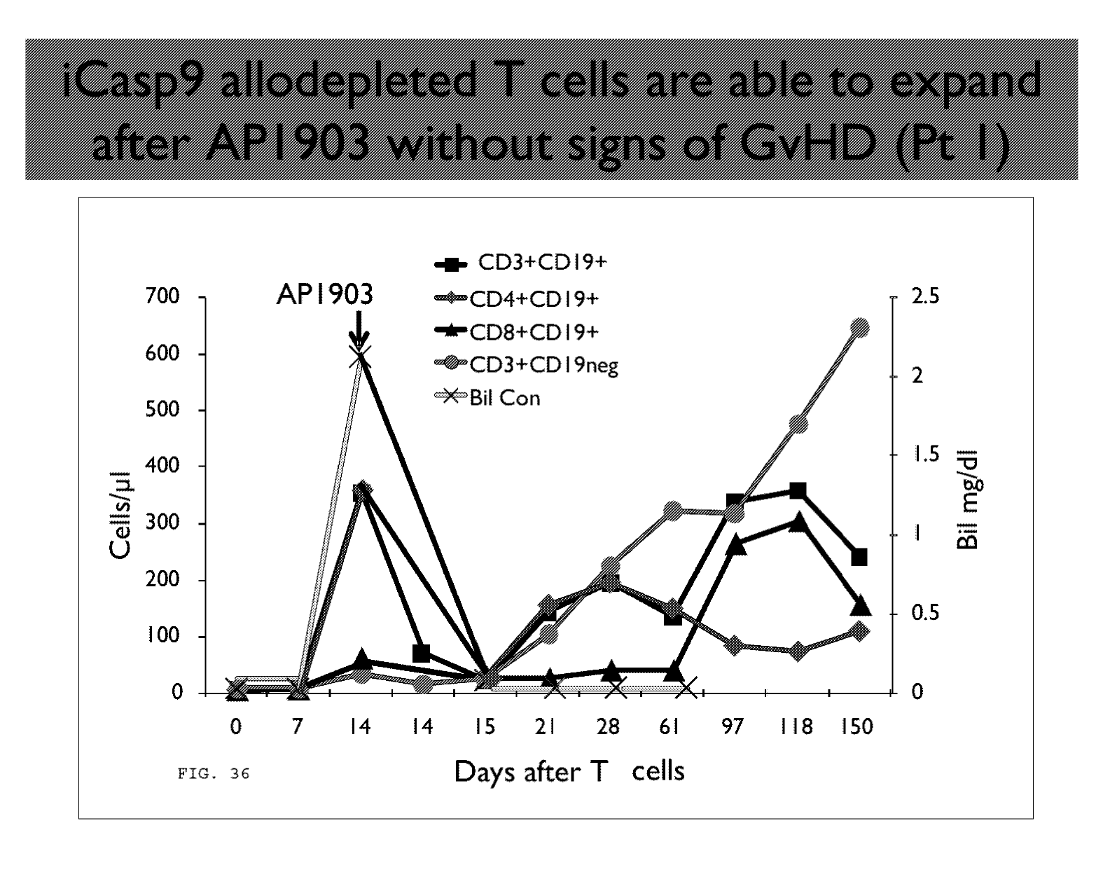

Infused T cells were detected in vivo by flow cytometry (CD3+ΔCD19+) or qPCR as early as day 7 after infusion, with a maximum fold expansion of 170±5 (day 29±9 after infusion), as illustrated in FIGS. 27, 28, and 29. Two patients developed grade I/II aGVHD (see FIGS. 31-32) and AP1903 administration caused>90% ablation of CD3+ΔCD19+ cells, within 30 minutes of infusion (see FIGS. 30, 33, and 34), with a further log reduction within 24 hours, and resolution of skin and liver aGvHD within 24 hrs (see FIG. 35), showing that iCasp9 transgene was functional in vivo.

TABLE 3
Patients with GvHD (dose level 1)

| | SCT to GvHD | T cells to GvHD | GvHD |
|---|---|---|---|
| Patient | (days) | (days) | (grade/site) |
| 1 | 77 | 14 | 2 (liver, skin) |
| 2 | 124 | 45/13 | 2 (skin) |

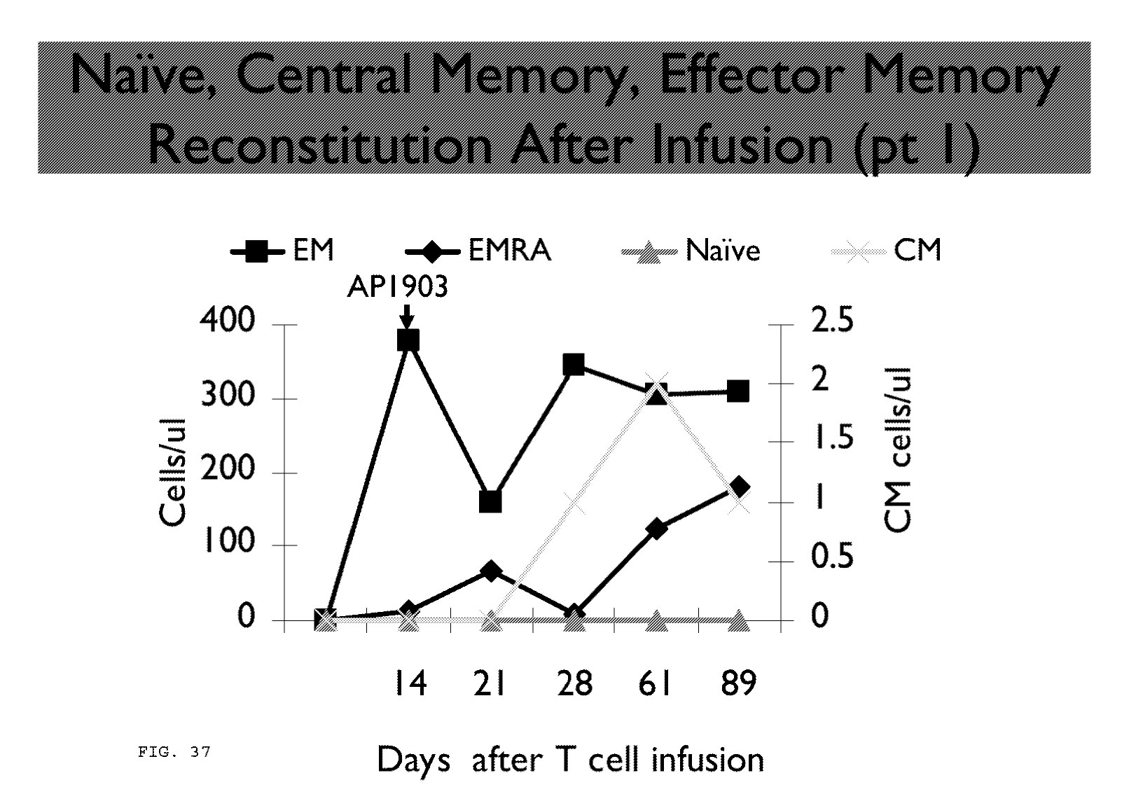

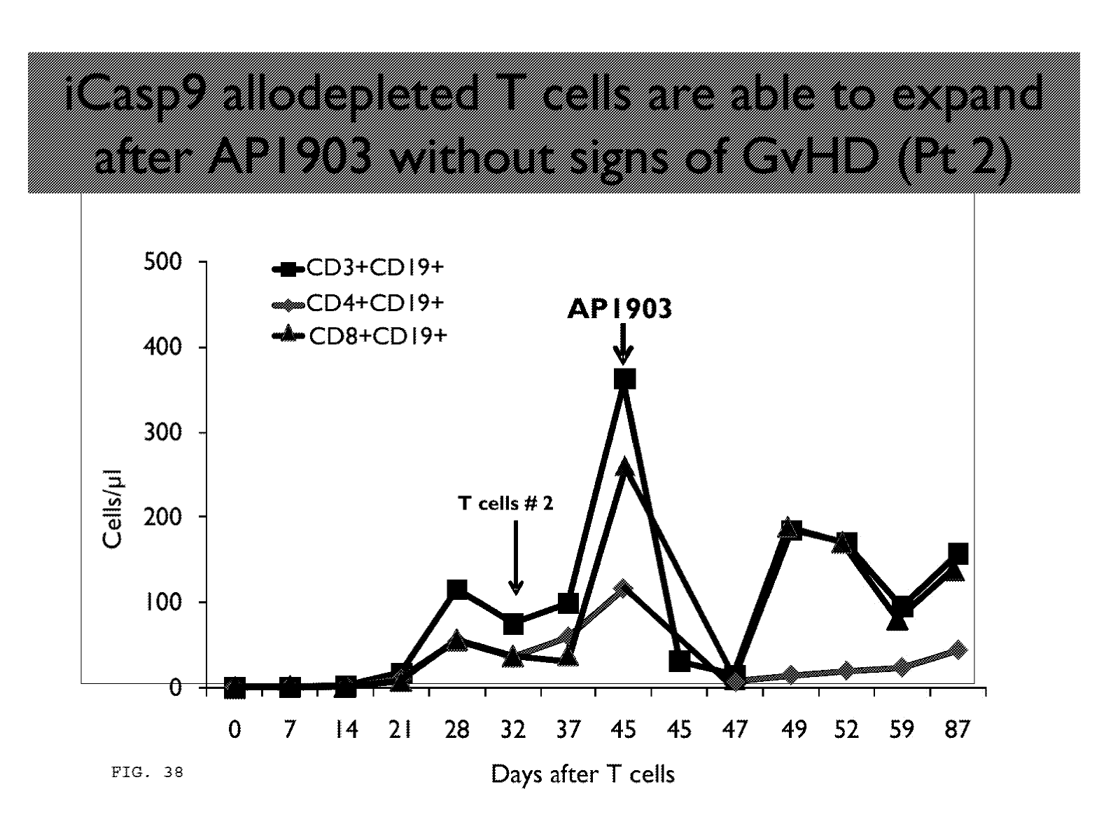

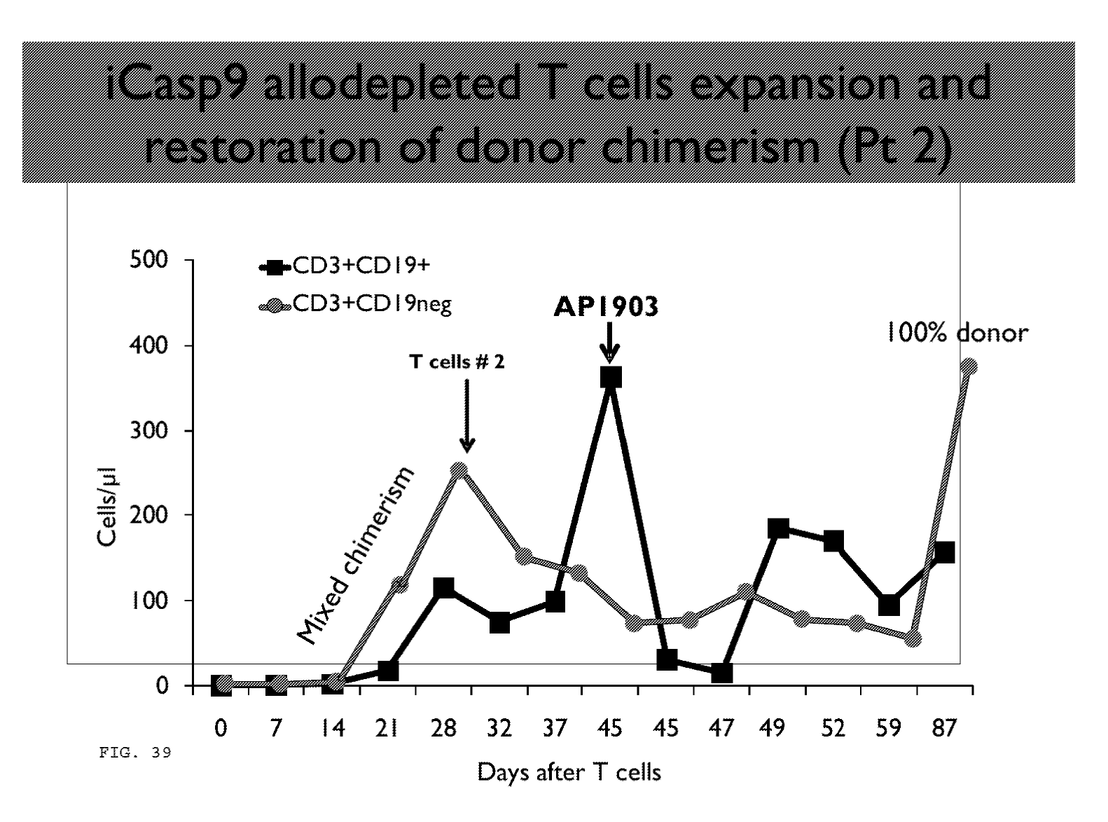

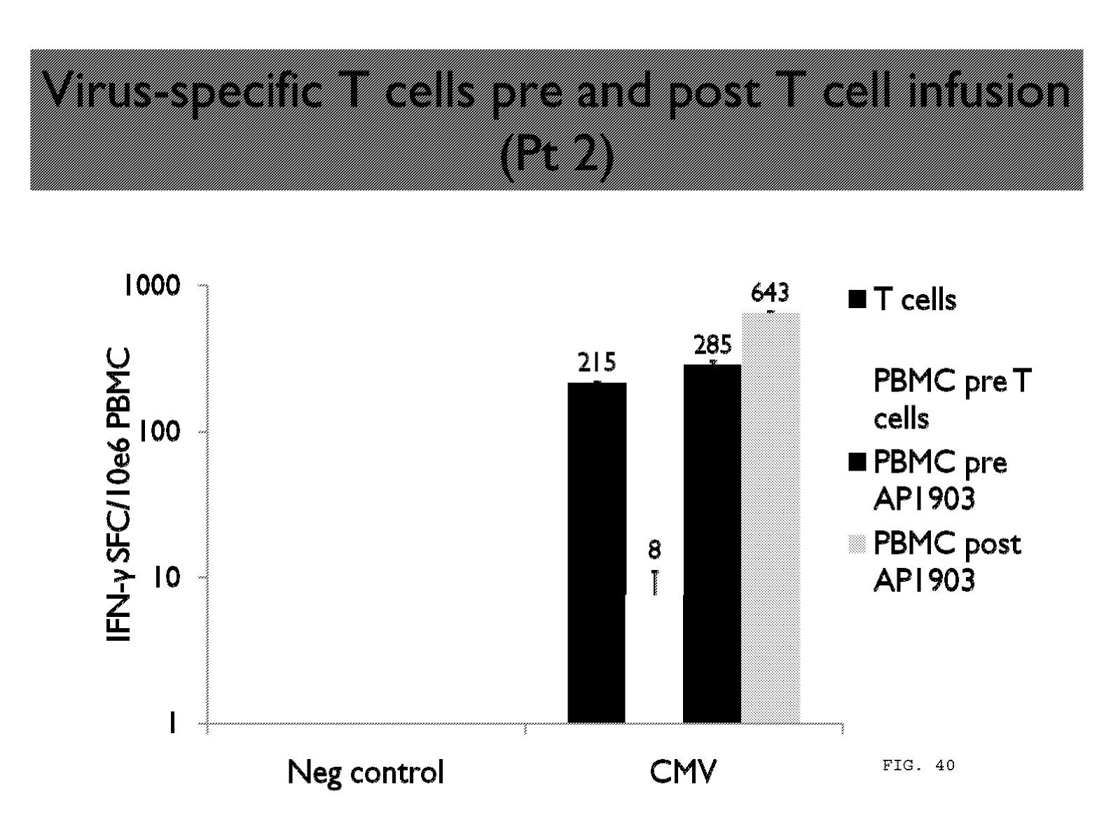

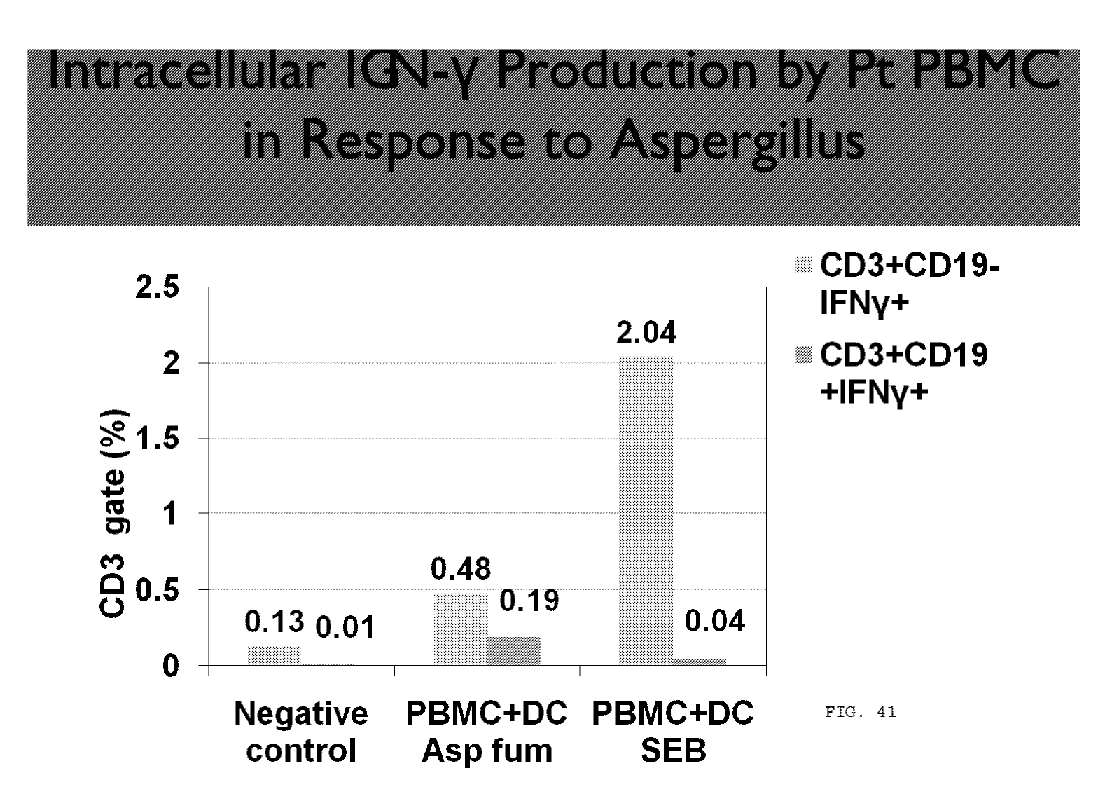

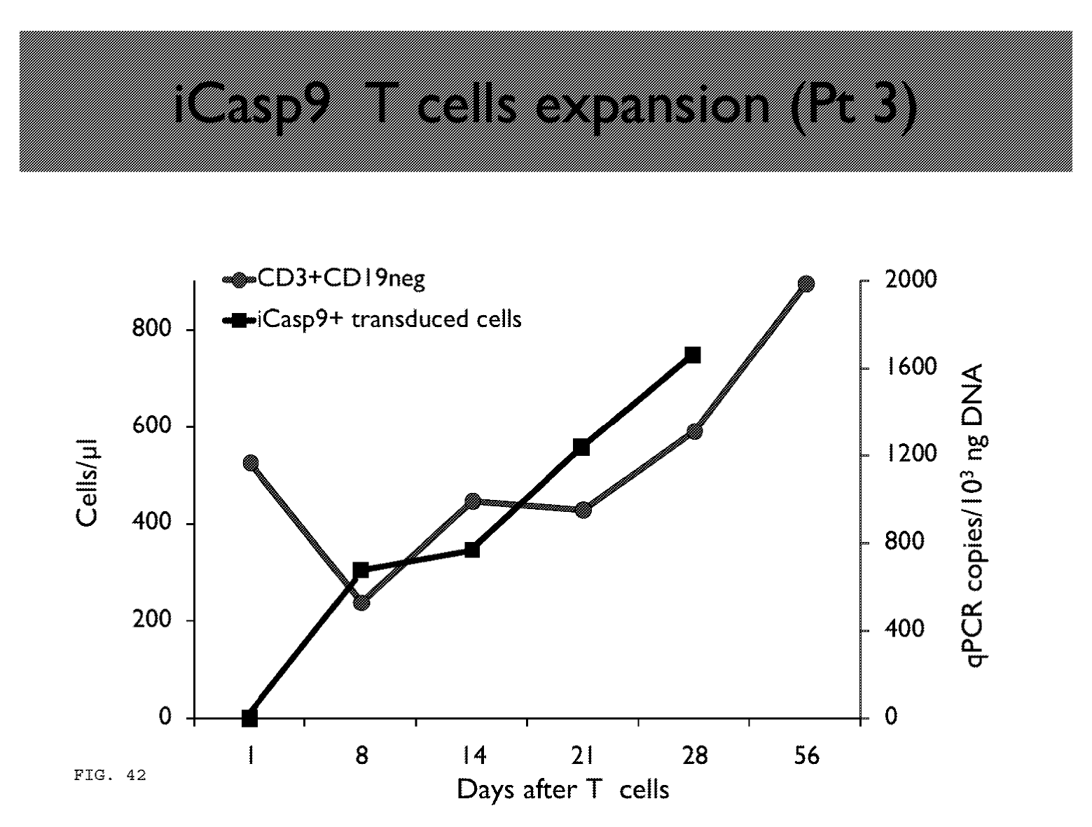

Ex vivo experiments confirmed this data. Furthermore, the residual allodepleted T cells were able to expand and were reactive to viruses (CMV) and fungi (Aspergillus fumigatus) (IFN-γ production), as shown in FIGS. 36-42. These in vivo studies found that a single dose of dimerizer drug can reduce or eliminate the subpopulation of T cells causing GvHD, but can spare virus specific CTLs, which can then re-expand.

Immune Reconstitution

Depending on availability of patient cells and reagents, immune reconstitution studies (Immunophenotyping, T and B cell function) may be obtained at serial intervals after transplant. Several parameters measuring immune reconstitution resulting from iCaspase transduced allodepleted T cells will be analyzed. The analysis includes repeated measurements of total lymphocyte counts, T and CD19 B cell numbers, and FACS analysis of T cell subsets (CD3, CD4, CD8, CD16, CD19, CD27, CD28, CD44, CD62L, CCR7, CD56, CD45RA, CD45RO, alpha/beta and gamma/delta T cell receptors). Depending on the availability of a patients T cells T regulatory cell markers such as CD41 CD251 FoxP3 also are analyzed. Approximately 10-60 ml of patient blood is taken, when possible, 4 hours after infusion, weekly for 1 month, monthly×9 months, and then at 1 and 2 years. The amount of blood taken is dependent on the size of the recipient and does not exceed 1-2 cc/kg in total (allowing for blood taken for clinical care and study evaluation) at any one blood draw.

Persistence and Safety of Transduced Allodepleted T Cells

The following analysis also was performed on the peripheral blood samples o monitor function, persistence and safety of transduced T-cells at time-points indicated in the study calendar.
Phenotype to detect the presence of transgenic cells
RCR testing by PCR.
Quantitative real-time PCR for detecting retroviral integrants.

RCR testing by PCR is performed pre study, at 3, 6, and 12 months, and then yearly for a total of 15 years. Tissue, cell, and serum samples are archived for use in future studies for RCR as required by the FDA.

Statistical Analysis and Stopping Rules.

The MTD is defined to be the dose which causes grade III/IV acute GVHD in at most 25% of eligible cases. The determination is based on a modified continual reassessment method (CRM) using a logistic model with a cohort of size 2. Three dose groups are being evaluated namely, 1×10⁶, 3×10⁶, 1×10⁷ with prior probabilities of toxicity estimated at 10%, 15%, and 30%, respectively. The proposed CRM design employs modifications to the original CRM by accruing more than one subject in each cohort, limiting dose escalation to no more than one dose level, and starting patient enrollment at the lowest dose level shown to be safe for non-transduced cells. Toxicity outcome in the lowest dose cohort is used to update the dose-toxicity curve. The next patient cohort is assigned to the dose level with an associated probability of toxicity closest to the target probability of 25%. This process continues until at least 10 patients have been accrued into this dose-escalation study. Depending on patient availability, at most 18 patients may be enrolled into the Phase I trial or until 6 patients have been treated at the current MTD. The final MTD will be the dose with probability closest to the target toxicity rate at these termination points.

Simulations were performed to determine the operating characteristics of the proposed design and compared this with a standard 3+3 dose-escalation design. The proposed design delivers better estimates of the MTD based on a higher probability of declaring the appropriate dose level as the MTD, afforded smaller number of patients accrued at lower and likely ineffective dose levels, and maintained a lower average total number of patients required for the trial. A shallow dose-toxicity curve is expected over the range of doses proposed herein and therefore accelerated dose-escalations can be conducted without comprising patient safety. The simulations performed indicate that the modified CRM design does not incur a larger average number of total toxicities when compared to the standard design (total toxicities equal to 1.9 and 2.1, respectively.).

Grade III/IV GVHD that occurs within 45 days after initial infusion of allodepleted T cells will be factored into the CRM calculations to determine the recommended dose for the subsequent cohort. Real-time monitoring of patient toxicity outcome is performed during the study in order to implement estimation of the dose-toxicity curve and determine dose level for the next patient cohort using one of the pre-specified dose levels. Treatment limiting toxicities will include

a) grade 4 reactions related to infusion,
b) graft failure (defined as a subsequent decline in the ANC to <5001 mm³ for three consecutive measurements on different days, unresponsive to growth factor therapy that persists for at least 14 days) occurring within 30 days after infusion of TC-T
c) grade 4 nonhematologic and noninfectious adverse events, occurring within 30 days after infusion
d) grades 3-4 acute GVHD by 45 days after infusion of TC-T
e) treatment-related death occurring within 30 days after infusion

GVHD rates are summarized using descriptive statistics along with other measures of safety and toxicity. Likewise, descriptive statistics will be calculated to summarize the clinical and biologic response in patients who receive AP1903 due to great than Grade 1 GVHD.

Several parameters measuring immune reconstitution resulting from iCaspase transduced allodepleted T cells will be analyzed. These include repeated measurements of total lymphocyte counts, T and CD19 B cell numbers, and FACS analysis of T cell subsets (CD3, CD4, CDS, CD16, CD19, CD27, CD44, CD62L, CCR7, CD56, CD45RA, CD45RO, alpha/beta and gamma/delta T cell receptors). If sufficient T cells remain for analysis, T regulatory cell markers such as CD4/CD25/FoxP3 will also be analyzed. Each subject will be measured pre-infusion and at multiple time points post-infusion as presented above.

Descriptive summaries of these parameters in the overall patient group and by dose group as well as by time of measurement will be presented. Growth curves representing measurements over time within a patient will be generated to visualize general patterns of immune reconstitution. The proportion of iCasp9 positive cells will also be summarized at each time point. Pairwise comparisons of changes in these endpoints over time compared to pre-infusion will be implemented using paired t-tests or Wilcoxon signed-ranks test.

Longitudinal analysis of each repeatedly-measured immune reconstitution parameter using the random coefficients model, will be performed. Longitudinal analysis allows construction of model patterns of immune reconstitution per patient while allowing for varying intercepts and slopes within a patient. Dose level as an independent variable in the model to account for the different dose levels received by the patients will also be used. Testing whether there is a significant improvement in immune function over time and estimates of the magnitude of these improvements based on estimates of slopes and its standard error will be possible using the model presented herein. Evaluation of any indication of differences in rates of immune reconstitution across different dose levels of CTLs will also be performed. The normal distribution with an identity link will be utilized in these models and implemented using SAS MIXED procedure. The normality assumption of the immune reconstitution parameters will be assessed and transformations (e.g. log, square root) can be performed, if necessary to achieve normality.

A strategy similar to the one presented above can be employed to assess kinetics of T cell survival, expansion and persistence. The ratio of the absolute T cell numbers with the number of marker gene positive cells will be determined and modeled longitudinally over time. A positive estimate of the slope will indicate increasing contribution of T cells for immune recovery. Virus-specific immunity of the iCasp9 T cells will be evaluated by analysis of the number of T cells releasing IFN gamma based on ex-vivo stimulation virus-specific CTLs using longitudinal models. Separate models will be generated for analysis of EBV, CMV and adenovirus evaluations of immunity.

Finally, overall and disease-free survival in the entire patient cohort will be summarized using the Kaplan-Meier product-limit method. The proportion of patients surviving and who are disease-free at 100 days and 1 year post transplant can be estimated from the Kaplan-Meier curves.

In conclusion, addback of iCasp9+ allodepleted T cells after haplo CD34⁺ SCT allows a significant expansion of functional donor lymphocytes in vivo and a rapid clearance of alloreactive T cells with resolution of aGvHD.

### Example 4

### In Vivo T Cell Allodepletion

The protocols provided in Examples 1-3 may also be modified to provide for in vivo T cell allodepletion. To extend the approach to a larger group of subjects who might benefit from immune reconstitution without acute GvHD, the protocol may be simplified, by providing for an in vivo method of T cell depletion. In the pre-treatment allodepletion method, as discussed herein, EBV-transformed lymphoblastoid cell lines are first prepared from the recipient, which then act as alloantigen presenting cells. This procedure can take up to 8 weeks, and may fail in extensively pre-treated subjects with malignancy, particularly if they have received rituximab as a component of their initial therapy. Subsequently, the donor T cells are co-cultured with recipient EBV-LCL, and the alloreactive T cells (which express the activation antigen CD25) are then treated with CD25-ricin conjugated monoclonal antibody. This procedure may take many additional days of laboratory work for each subject.

The process may be simplified by using an in vivo method of allodepletion, building on the observed rapid in vivo depletion of alloreactive T cells by dimerizer drug and the sparing of unstimulated but virus/fungus reactive T cells.

If there is development of Grade I or greater acute GvHD, a single dose of dimerizer drug is administered, for example at a dose of 0.4 mg/kg of AP1903 as a 2 hour intravenous infusion. Up to 3 additional doses of dimerizer drug may be administered at 48 hour intervals if acute GvHD persists. In subjects with Grade II or greater acute GvHD, these additional doses of dimerizer drug may be combined with steroids. For patients with persistent GVHD who cannot receive additional doses of the dimerizer due to a Grade III or IV reaction to the dimerizer, the patient may be treated with steroids alone, after either 0 or 1 doses of the dimerizer.

Generation of Therapeutic T Cells

Up to 240 ml (in 2 collections) of peripheral blood is obtained from the transplant donor according to the procurement consent. If necessary, a leukopheresis is used to obtain sufficient T cells; (either prior to stem cell mobilization or seven days after the last dose of G-CSF). An extra 10-30 mls of blood may also be collected to test for infectious diseases such as hepatitis and HIV.

Peripheral blood mononuclear cells are be activated using anti-human CD3 antibody (e.g. from Orthotech or Miltenyi) on day 0 and expanded in the presence of recombinant human interleukin-2 (rhIL-2) on day 2. CD3 antibody-activated T cells are transduced by the icaspase-9 retroviral vector on flasks or plates coated with recombinant Fibronectin fragment CH-296 (Retronectin™, Takara Shuzo, Otsu, Japan). Virus is attached to retronectin by incubating producer supernatant in retronectin coated plates or flasks. Cells are then transferred to virus coated tissue culture devices. After transduction T cells are expanded by feeding them with rhIL-2 twice a week to reach the sufficient number of cells as per protocol.

To ensure that the majority of infused T cells carry the suicide gene, a selectable marker, truncated human CD19 (ΔCD19) and a commercial selection device, may be used to select the transduced cells to >90% purity. Immunomagnetic selection for CD19 may be performed 4 days after transduction. Cells are labeled with paramagnetic microbeads conjugated to monoclonal mouse anti-human CD19 antibodies (Miltenyi Biotech, Auburn, Calif.) and selected on a CliniMacs Plus automated selection device. Depending upon the number of cells required for clinical infusion cells might either be cryopreserved after the CliniMacs selection or further expanded with IL-2 and cryopreserved as soon as sufficient cells have expanded (up to day 14 from product initiation).

Aliquots of cells may be removed for testing of transduction efficiency, identity, phenotype, autonomous growth and microbiological examination as required for final release testing by the FDA. The cells are be cryopreserved prior to administration.

Administration of T Cells

The transduced T cells are administered to patients from, for example, between 30 and 120 days following stem cell transplantation. The cryopreserved T cells are thawed and infused through a catheter line with normal saline. For children, premedications are dosed by weight. Doses of cells may range from, for example, from about 1×10⁴ cells/kg to 1×10⁸ cells/kg, for example from about 1×10⁵ cells/kg to 1×10⁷ cells/kg, from about 1×10⁶ cells/kg to 5×10⁶ cells/kg, from about 1×10⁴ cells/kg to 5×10⁶ cells/kg, for example, about 1×10⁴, about 1×10⁵, about 2×10⁵, about 3×10⁵, about 5×10⁵, 6×10⁵, about 7×10⁵, about 8×10⁵, about 9×10⁵, about 1×10⁶, about 2×10⁶, about 3×10⁶, about 4×10⁶, or about 5×10⁶ cells/kg.

Treatment of GvHD

Patients who develop grade≧1 acute GVHD are treated with 0.4 mg/kg AP1903 as a 2-hour infusion. AP1903 for injection may be provided, for example, as a concentrated solution of 2.33 ml in a 3 ml vial, at a concentration of 5 mg/ml, (i.e. 10.66 mg per vial). Prior to administration, the calculated dose will be diluted to 100 mL in 0.9% normal saline for infusion. AP1903 for Injection (0.4 mg/kg) in a volume of 100 ml may be administered via IV infusion over 2 hours, using a non-DEHP, non-ethylene oxide sterilized infusion set and an infusion pump.

TABLE 4
Sample treatment schedule

| Time | Donor | Recipient |
|---|---|---|
| Pre-trans- | Obtain up to 240 of blood or | |
| plant | unstimulated leukapheresis | |
| | from bone marrow | |
| | transplant donor. Prepare | |
| | T cells and donor LCLs for | |
| | later immune reconstitution | |
| | studies. | |
| Day 0 | Anti-CD3 activation of | |
| | PBMC | |
| Day 2 | IL-2 feed | |
| Day 3 | Transduction | |
| Day 4 | Expansion | |
| Day 6 | CD19 selection. | |
| | Cryopreservation (*if | |
| | required dose is met) | |
| Day 8 | Assess transduction | |
| | efficiency and iCaspase9 | |
| | transgene functionality by | |
| | phenotype. | |
| | Cryopreservation (*if not yet | |
| | performed) | |
| Day 10 or Day | Cryopreservation (if not yet | |
| 12 to Day 14 | performed) | |
| From 30 to 120 | | Thaw and infuse T |
| days post | | cells 30 to 120 |
| transplant | | days post stem |
| | | cell infusion. |

Other methods may be followed for clinical therapy and assessment as provided in, for example, Examples 1-3 herein.

### Example 5

### Using the iCasp9 Suicide Gene to Improve the Safety of Mesenchymal Stromal Cell Therapies

Mesenchymal stromal cells (MSCs) have been infused into hundreds of patients to date with minimal reported deleterious side effects. The long term side effects are not known due to limited follow-up and a relatively short time since MSCs have been used in treatment of disease. Several animal models have indicated that there exists the potential for side effects, and therefore a system allowing control over the growth and survival of MSCs used therapeutically is desirable. The inducible caspase-9 suicide switch expression vector construct presented herein was investigated as a method of eliminating MSC's in vivo and in vitro.

Materials and Methods

MSC Isolation

MSCs were isolated from healthy donors. Briefly, post-infusion discarded healthy donor bone marrow collection bags and filters were washed with RPMI 1640 (HyClone, Logan, Utah) and plated on tissue culture flasks in DMEM (Invitrogen, Carlsbad, Calif.) with 10% fetal bovine serum (FBS), 2 mM alanyl-glutamine (Glutamax, Invitrogen), 100 units/mL penicillin and 100 μg/mL streptomycin (Invitrogen). After 48 hours, the supernatant was discarded and the cells were cultured in complete culture medium (CCM): α-MEM (Invitrogen) with 16.5% FBS, 2 mM alanyl-glutamine, 100 units/mL penicillin and 100 pg/mL streptomycin. Cells were grown to less then 80% confluence and replated at lower densities as appropriate.

Immunophenotyping

Phycoerythrin (PE), fluorescein isothiocyanate (FITC), peridinin chlorophyll protein (PerCP) or allophycocyanin (APC)-conjugated CD14, CD34, CD45, CD73, CD90, CD105 and CD133 monoclonal antibodies were used to stain MSCs. All antibodies were from Becton Dickinson-Pharmingen (San Diego, Calif.), except where indicated. Control samples labeled with an appropriate isotype-matched antibody were included in each experiment. Cells were analyzed by fluorescence-activated cell sorting FACScan (Becton Dickinson) equipped with a filter set for 4 fluorescence signals.

Differentiation Studies In Vitro

Adipocytic differentiation. MSCs (7.5×10⁴ cells) were plated in wells of 6-well plates in NH AdipoDiff Medium (Miltenyi Biotech, Auburn, Calif.). Medium was changed every third day for 21 days. Cells were stained with Oil Red O solution (obtained by diluting 0.5% w/v Oil Red O in isopropanol with water at a 3:2 ratio), after fixation with 4% formaldehyde in phosphate buffered saline (PBS).

Osteogenic differentiation. MSCs (4.5×10⁴ cells) were plated in 6-well plates in NH OsteoDiff Medium (Miltenyi Biotech). Medium was changed every third day for 10 days. Cells were stained for alkaline phosphatase activity using Sigma Fast BCIP/NBT substrate (Sigma-Aldrich, St. Louis, Mo.) as per manufacturer instructions, after fixation with cold methanol.

Chondroblastic differentiation. MSC pellets containing 2.5×10⁵ to 5×10⁵ cells were obtained by centrifugation in 15 mL or 1.5 mL polypropylene conical tubes and cultured in NH ChondroDiff Medium (Miltenyi Biotech). Medium was changed every third day for a total of 24 days. Cell pellets were fixed in 4% formalin in PBS and processed for routine paraffin sectioning. Sections were stained with alcian blue or using indirect immunofluorescence for type II collagen (mouse anti-collagen type II monoclonal antibody MAB8887, Millipore, Billerica, Mass.) after antigen retrieval with pepsin (Thermo Scientific, Fremont, Calif.).

iCasp9-ΔCD19 Retrovirus Production and Transduction of MSCs

The SFG.iCasp9.2A.ΔCD19 (iCasp-ΔCD19) retrovirus consists of iCasp9 linked, via a cleavable 2A-like sequence, to truncated human CD19 (ΔCD19). As noted above, iCasp9 is a human FK506-binding protein (FKBP12) with an F36V mutation, which increases the binding affinity of the protein to a synthetic homodimerizer (AP20187 or AP1903), connected via a Ser-Gly-Gly-Gly-Ser-Gly linker to human caspase-9, whose recruitment domain (CARD) has been deleted, its function replaced by FKBP12.

The 2A-like sequence encodes a 20 amino acid peptide from Thosea Asigna insect virus, which mediates more than 99% cleavage between a glycine and terminal proline residue, to ensure separation of iCasp9 and ΔCD19 upon translation. ΔCD19 consists of human CD19 truncated at amino acid 333, which removes all conserved intracytoplasmic tyrosine residues that are potential sites for phosphorylation. A stable PG13 clone producing Gibbon ape leukemia virus (Gal-V) pseudotyped retrovirus was made by transiently transfecting Phoenix Eco cell line (ATCC product #SD3444; ATCC, Manassas, Va.) with SFG.iCasp9.2A.ΔCD19, which yielded Eco-pseudotyped retrovirus. The PG13 packaging cell line (ATCC) was transduced 3 times with Eco-pseudotyped retrovirus to generate a producer line that contained multiple SFG.iCasp9.2A.ΔCD19 proviral integrants per cell. Single-cell cloning was performed, and the PG13 clone that produced the highest titer was expanded and used for vector production. Retroviral supernatant was obtained via culture of the producer cell lines in IMDM (Invitrogen) with 10% FBS, 2 mM alanyl-glutamine, 100 units/mL penicillin and 100 μg/mL streptomycin. Supernatant containing the retrovirus was collected 48 and 72 hours after initial culture. For transduction, approximately 2×10⁴ MSCs/cm² were plated in CM in 6-well plates, T75 or T175 flasks. After 24 hours, medium was replaced by viral supernatant diluted 10-fold together with polybrene (final concentration 5 μg/mL) and the cells were incubated at 37° C. in 5% CO₂ for 48 hours, after which cells were maintained in complete medium.

Cell Enrichment

For inducible iCasp9-ΔCD19-positive MSC selection for in vitro experiments, retrovirally transduced MSC were enriched for CD19-positive cells using magnetic beads (Miltenyi Biotec) conjugated with anti-CD19 (clone 4G7), per manufacturer instructions. Cell samples were stained with PE- or APC-conjugated CD19 (clone SJ25C1) antibody to assess the purity of the cellular fractions.

Apoptosis Studies In Vitro

Undifferentiated MSCs. The chemical inducer of dimerization (CID) (AP20187; ARIAD Pharmaceuticals, Cambridge, Mass.) was added at 50 nM to iCasp9-transduced MSCs cultures in complete medium. Apoptosis was evaluated 24 hours later by FACS analysis, after cell harvest and staining with annexin V-PE and 7-AAD in annexin V binding buffer (BD Biosciences, San Diego, Calif.). Control iCasp9-transduced MSCs were maintained in culture without exposure to CID.

Differentiated MSCs. Transduced MSCs were differentiated as presented above. At the end of the differentiation period, CID was added to the differentiation media at 50 nM. Cells were stained appropriately for the tissue being studied, as presented above, and a contrast stain (methylene azur or methylene blue) was used to evaluate the nuclear and cytoplasmic morphology. In parallel, tissues were processed for terminal deoxynucleotidyl-transferase dUTP nick end labeling (TUNEL) assay as per manufacturer instructions (In Situ Cell Death Detection Kit, Roche Diagnostics, Mannheim, Germany). For each time point, four random fields were photographed at a final magnification of 40× and the images were analyzed with ImageJ software version 1.43o (NIH, Bethesda, Md.). Cell density was calculated as the number of nuclei (DAPI positivity) per unit of surface area (in mm²). The percentage of apoptotic cells was determined as the ratio of the number of nuclei with positive TUNEL signal (FITC positivity) to the total number of nuclei. Controls were maintained in culture without CID.

In Vivo Killing Studies in Murine Model

All mouse experiments were performed in accordance with the Baylor College of Medicine animal husbandry guidelines. To assess the persistence of modified MSCs in vivo, a SCID mouse model was used in conjunction with an in vivo imaging system. MSCs were transduced with retroviruses coding for the enhanced green fluorescent protein-firefly luciferase (eGFP-FFLuc) gene alone or together with the iCasp9-ΔCD19 gene. Cells were sorted for eGFP positivity by fluorescence activated cell sorting using a MoFlo flow cytometer (Beckman Coulter, Fullerton, Calif.). Doubly transduced cells were also stained with PE-conjugated anti-CD19 and sorted for PE-positivity. SCID mice (8-10 weeks old) were injected subcutaneously with 5×10⁵ MSCs with and without iCasp9-ΔCD19 in opposite flanks. Mice received two intraperitoneal injections of 50 μg of CID 24 hours apart starting a week later. For in vivo imaging of MSCs expressing eGFP-FFLuc, mice were injected intraperitoneally with D-luciferin (150 mg/kg) and analyzed using the Xenogen-IVIS Imaging System. Total luminescence (a measurement proportional to the total labeled MSCs deposited) at each time point was calculated by automatically defining regions-of-interest (ROIs) over the MSC implantation sites. These ROIs included all areas with luminescence signals at least 5% above background. Total photon counts were integrated for each ROI and an average value calculated. Results were normalized so that time zero would correspond to 100% signal.

In a second set of experiments, a mixture of 2.5×10⁶ eGFP-FFLuc-labeled MSCs and 2.5×10⁶ eGFP-FFLuc-labeled, iCasp9-ΔCD19-transduced MSCs was injected subcutaneously in the right flank, and the mice received two intraperitoneal injections of 50 μg of CID 24 h apart starting 7 days later. At several time points after CID injection, the subcutaneous pellet of MSCs was harvested using tissue luminescence to identify and collect the whole human specimen and to minimize mouse tissue contamination. Genomic DNA was then isolated using QIAmp® DNA Mini (Qiagen, Valencia, Calif.). Aliquots of 100 ng of DNA were used in a quantitative PCR (qPCR) to determine the number of copies of each transgene using specific primers and probes (for the eGFP-FFLuc construct: forward primer 5′-TCCGCCCTGAGCAAAGAC-3′ (SEQ ID NO: 240), reverse 5′-ACGAACTCCAGCAGGACCAT-3′ (SEQ ID NO: 241), probe 5′ FAM, 6-carboxyfluorescein-ACGAGAAGCGCGATC-3′ MGBNFQ (SEQ ID NO: 242), minor groove binding non-fluorescent quencher; iCasp9-ΔCD19: forward 5′-CTGGAATCTGGCGGTGGAT-3′ (SEQ ID NO: 243), reverse 5′-CAAACTCTCAAGAGCACCGACAT-3′ (SEQ ID NO: 244), probe 5′ FAM-CGGAGTCGACGGATT-3′ MGBNFQ (SEQ ID NO: 245)). Known numbers of plasmids containing single copies of each transgene were used to establish standard curves. It was determined that approximately 100 ng of DNA isolated from “pure” populations of singly eGFP-FFLuc- or doubly eGFP-FFLuc- and iCasp9-transduced MSCs had similar numbers of eGFP-FFLuc gene copies (approximately 3.0×10⁴), as well as zero and 1.7×10³ of iCasp9-ΔCD19 gene copies, respectively.

Untransduced human cells and mouse tissues had zero copies of either gene in 100 ng of genomic DNA. Because the copy number of the eGFP gene is the same on identical amounts of DNA isolated from either population of MSCs (iCasp9-negative or positive), the copy number of this gene in DNA isolated from any mixture of cells will be proportional to the total number of eGFP-FFLuc-positive cells (iCasp9-positive plus negative MSCs). Moreover, because iCasp9-negative tissues do not contribute to the iCasp9 copy number, the copy number of the iCasp9 gene in any DNA sample will be proportional to the total number of iCasp9-positive cells. Therefore, if G is the total number of GFP-positive and iCasp9-negative cells and C the total number of GFP-positive and iCasp9-positive cells, for any DNA sample then N_{eGFP}=g·(C+G) and N_{iCasp9}=k·C, where N represents gene copy number and g and k are constants relating copy number and cell number for the eGFP and iCasp9 genes, respectively. Thus N_{iCasp9}/N_{eGFP}=(k/g)·[C/(C+G)], i.e., the ratio between iCasp9 copy number and eGFP copy number is proportional to the fraction of doubly transduced (iCasp9-positive) cells among all eGFP positive cells. Although the absolute values of N_{iCasp9} and N_{eGFP} will decrease with increasing contamination by murine cells in each MSC explant, for each time point the ratio will be constant regardless of the amount of murine tissue included, since both types of human cells are physically mixed. Assuming similar rates of spontaneous apoptosis in both populations (as documented by in vitro culture) the quotient between N_{iCasp9}/N_{eGFp} at any time point and that at time zero will represent the percentage of surviving iCasp9-positive cells after exposure to CID. All copy number determinations were done in triplicate.

Statistical Analysis

Paired 2-tailed Student's t-test was used to determine the statistical significance of differences between samples. All numerical data are represented as mean±1 standard deviation.

Results

MSCs are Readily Transduced with iCasp9-ΔCD19 and Maintain their Basic Phenotype

Flow cytometric analysis of MSCs from 3 healthy donors showed they were uniformly positive for CD73, CD90 and CD105 and negative for the hematopoietic markers CD45, CD14, CD133 (FIG. 15A) and CD34. The mononuclear adherent fraction isolated from bone marrow was homogenously positive for CD73, CD90 and CD105 and negative for hematopoietic markers. The differentiation potential, of isolated MSCs, into adipocytes, osteoblasts and chondroblasts was confirmed in specific assays (see FIG. 15B), demonstrating that these cells are bona fide MSCs. FIG. 15B illustrates the results of differentiation studies, the isolated MSCs were able to differentiate into adipocytes (left, oil red and methylene blue), osteoblasts (center, alkaline phosphatase-bromochloroindolyl phosphate/nitroblue tetrazolium and methylene blue) and chondroblasts (right, anti-type II collagen antibody-Texas red and DAPI) when cultured in appropriate media.

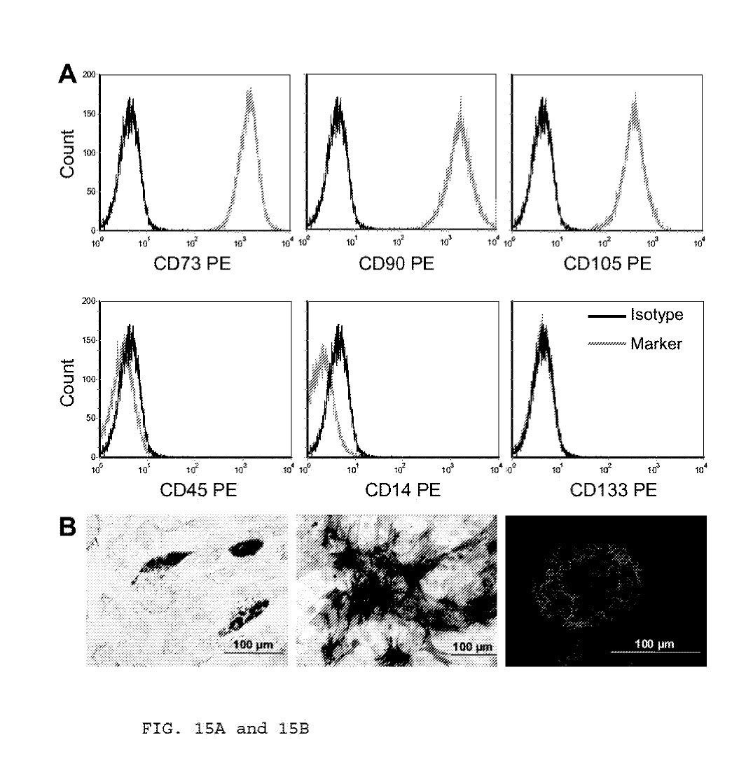

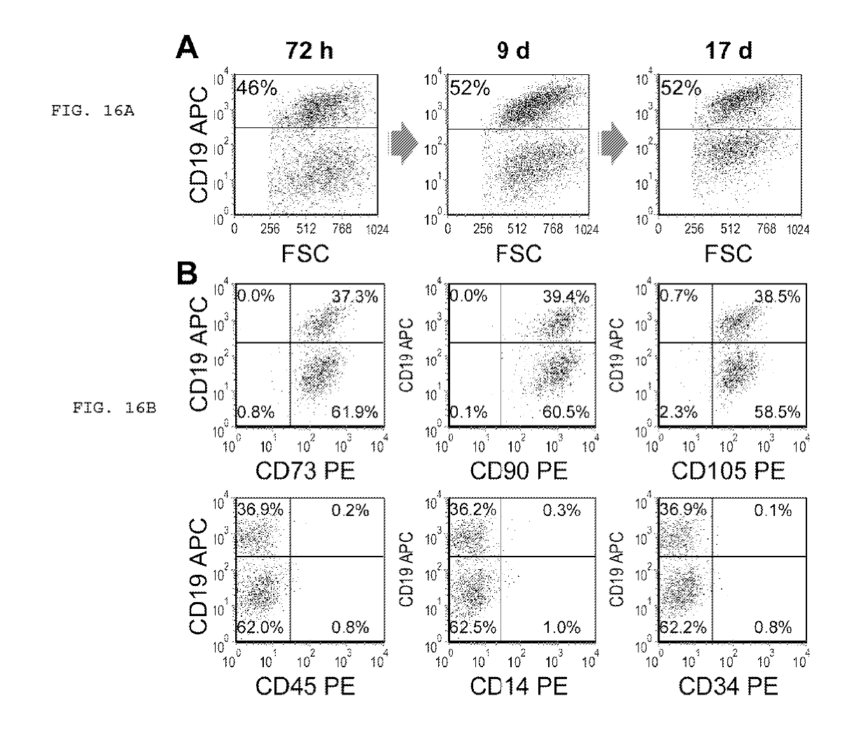

Early passage MSCs were transduced with an iCasp9-ΔCD19 retroviral vector, encoding an inducible form of caspase-9. Under optimal single transduction conditions, 47±6% of the cells expressed CD19, a truncated form of which is transcribed in cis with iCasp9, serving as a surrogate for successful transduction and allowing selection of transduced cells. The percentage of cells positive for CD19 was stable for more than two weeks in culture, suggesting no deleterious or growth advantageous effects of the construct on MSCs, as shown in FIG. 16A. FIG. 9A illustrates the results of MSCs that underwent a single round of transduction with iCasp9-ΔCD19 retrovirus. The percentage of CD19-positive cells, a surrogate for successful transduction with iCasp9, remains constant for more than 2 weeks. To further address the stability of the construct, a population of iCasp9-positive cells purified by a fluorescence activated cell sorter (FACS) was maintained in culture: no significant difference in the percentage of CD19-positive cells was observed over six weeks (96.5±1.1% at baseline versus 97.4±0.8% after 43 days, P=0.46). The phenotype of the iCasp9-CD19-positive cells was otherwise substantially identical to that of untransduced cells, with virtually all cells positive for CD73, CD90 and CD105 and negative for hematopoietic markers, as illustrated in FIG. 16B), confirming that the genetic manipulation of MSCs did not modify their basic characteristics.

iCasp9-ΔCD19 Transduced MSCs Undergo Selective Apoptosis after Exposure to CID In Vitro

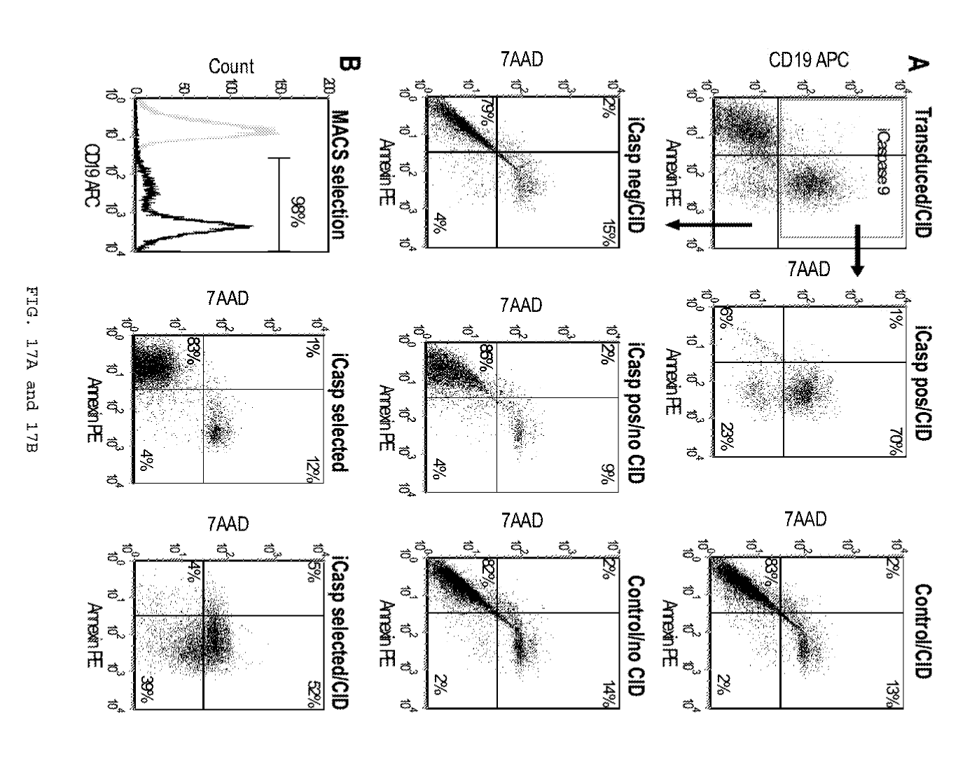

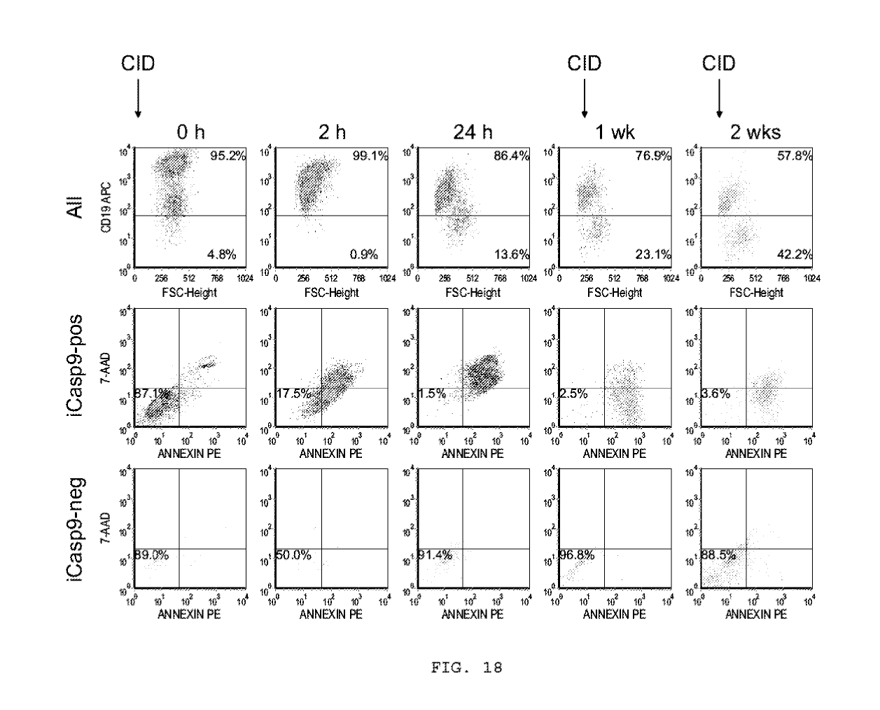

The proapoptotic gene product iCasp9 can activated by a small chemical inducer of dimerization (CID), AP20187, an analogue of tacrolimus that binds the FK506-binding domain present in the iCasp9 product. Non-transduced MSCs have a spontaneous rate of apoptosis in culture of approximately 18% (±7%) as do iCasp9-positive cells at baseline (15±6%, P=0.47). Addition of CID (50 nM) to MSC cultures after transduction with iCasp9-ΔCD19 results in the apoptotic death of more than 90% of iCasp9-positive cells within 24 hrs (93±1%, P<0.0001), while iCasp9-negative cells retain an apoptosis index similar to that of non-transduced controls (20±7%, P=0.99 and P=0.69 vs. non-transduced controls with or without CID respectively) (see FIGS. 17A and 70B). After transduction of MSCs with iCasp9, the chemical inducer of dimerization (CID) was added at 50 nM to cultures in complete medium. Apoptosis was evaluated 24 hours later by FACS analysis, after cell harvest and staining with annexin V-PE and 7-AAD. Ninety-three percent of the iCasp9-CD19-positive cells (iCasp pos/CID) became annexin positive versus only 19% of the negative population (iCasp neg/CID), a proportion comparable to non-transduced control MSC exposed to the same compound (Control/CID, 15%) and to iCasp9-CD19-positive cells unexposed to CID (iCasp pos/no CID, 13%), and similar to the baseline apoptotic rate of non-transduced MSCs (Control/no CID, 16%). Magnetic immunoselection of iCap9-CD19-positive cells can be achieved to high degree of purity. More than 95% of the selected cells become apoptotic after exposure to CID.

Analysis of a highly purified iCasp9-positive population at later time points after a single exposure to CID shows that the small fraction of iCasp9-negative cells expands and that a population of iCasp9-positive cells remains, but that the latter can be killed by re-exposure to CID. Thus, no iCasp9-positive population resistant to further killing by CID was detected (see FIG. 18). A population of iCasp9-CD19-negative MSCs emerges as early as 24 hours after CID introduction. A population of iCasp9-CD19-negative MSCs is expected since achieving a population with 100% purity is unrealistic and because the MSCs are being cultured in conditions that favor their rapid expansion in vitro. A fraction of iCasp9-CD19-positive population persists, as predicted by the fact that killing is not 100% efficient (assuming, for example, 99% killing of a 99% pure population, the resulting population would have 49.7% iCasp9-positive and 50.3% iCasp9-negative cells). The surviving cells, however, can be killed at later time points by re-exposure to CID.

iCasp9-ΔCD19 Transduced MSCs Maintain the Differentiation Potential of Unmodified MSCs and their Progeny is Killed by Exposure to CID

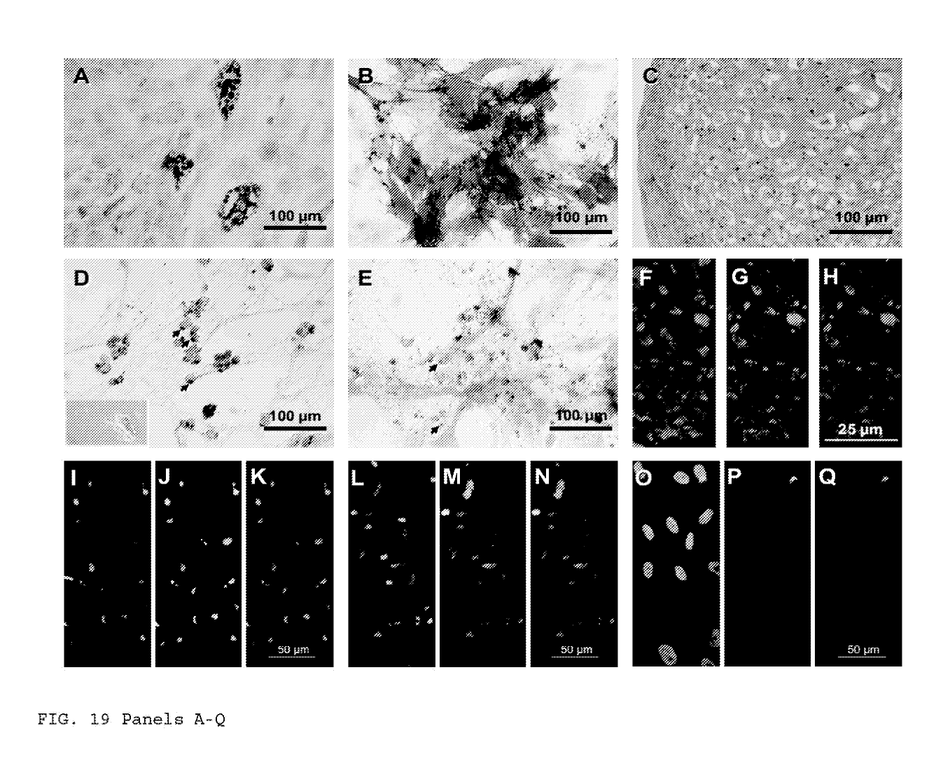

To determine if the CID can selectively kill the differentiated progeny of iCasp9-positive MSCs, immunomagnetic selection for CD19 was used to increase the purity of the modified population (>90% after one round of selection, see FIG. 16B). The iCasp9-positive cells thus selected were able to differentiate in vivo into all connective tissue lineages studied (see FIGS. 19A-19Q). Human MSCs were immunomagnetically selected for CD19 (thus iCasp9) expression, with a purity greater than 91%. After culture in specific differentiation media, iCasp9-positive cells were able to give rise to adipocytic (A, oil red and methylene azur), osteoblastic (B, alkaline phosphatase-BCIP/NBT and methylene blue) and chondroblastic lineages (C, alcian blue and nuclear red) lineages. These differentiated tissues are driven to apoptosis by exposure to 50 nM CID (D-N). Note numerous apoptotic bodies (arrows), cytoplasmic membrane blebbing (inset) and loss of cellular architecture (D and E); widespread TUNEL positivity in chondrocytic nodules (F-H), and adipogenic (I-K) and osteogenic (L-N) cultures, in contrast to that seen in untreated iCasp9-transduced controls (adipogenic condition shown, O-Q) (F, I, L, O, DAPI; G, J, M, P, TUNEL-FITC; H, K, N, Q, overlay).

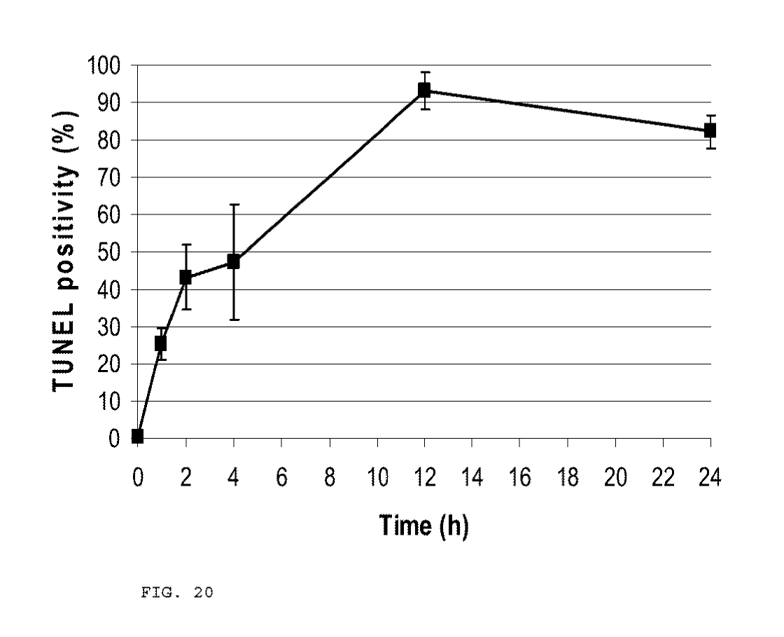

After 24 hours of exposure to 50 nM of CID, microscopic evidence of apoptosis was observed with membrane blebbing, cell shrinkage and detachment, and presence of apoptotic bodies throughout the adipogenic and osteogenic cultures. A TUNEL assay showed widespread positivity in adipogenic and osteogenic cultures and the chondrocytic nodules (see FIGS. 19A-19Q), which increased over time (see FIG. 20). After culture in adipocytic differentiation media, iCasp9-positive cells gave rise to adipocytes. After exposure to 50 nM CID, progressive apoptosis was observed as evidenced by an increasing proportion of TUNEL-positive cells. After 24 hours, there was a significant decrease in cell density (from 584 cells/mm2 to <14 cells/mm2), with almost all apoptotic cells having detached from the slides, precluding further reliable calculation of the proportion of apoptotic cells. Thus, iCasp9 remained functional even after MSC differentiation and its activation results in the death of the differentiated progeny.

iCasp9-ΔCD19 Transduced MSCs Undergo Selective Apoptosis after in Vivo Exposure to CID

Although intravenously injected MSC already appear to have a short in vivo survival time, cells injected locally may survive longer and produce correspondingly more profound adverse effects. To assess the in vivo functionality of the iCasp9 suicide system in such a setting, SCID mice were subcutaneously injected with MSCs. MSCs were doubly transduced with the eGFP-FFLuc (previously presented) and iCasp9-ΔCD19 genes. MSCs were also singly transduced with eGFP-FFLuc. The eGFP-positive (and CD19-positive, where applicable) fractions were isolated by fluorescence activated cell sorting, with a purity>95%. Each animal was injected subcutaneously with iCasp9-positive and control MSCs (both eGFP-FFLuc-positive) in opposite flanks. Localization of the MSCs was evaluated using the Xenogen-IVIS Imaging System. In another set of experiments, a 1:1 mixture of singly and doubly transduced MSCs was injected subcutaneously in the right flank and the mice received CID as above. The subcutaneous pellet of MSCs was harvested at different time points, genomic DNA was isolated and qPCR was used to determine copy numbers of the eGFP-FFLuc and iCasp9-ΔCD19 genes. Under these conditions, the ratio of the iCasp9 to eGFP gene copy numbers is proportional to the fraction of iCasp9-positive cells among total human cells (see Methods above for details). The ratios were normalized so that time zero corresponds to 100% of iCasp9-positive cells. Serial examination of animals after subcutaneous inoculation of MSCs (prior to CID injection) shows evidence of spontaneous apoptosis in both cell populations (as demonstrated by a fall in the overall luminescence signal to ˜20% of the baseline). This has been previously observed after systemic and local delivery of MSCs in xenogeneic models.

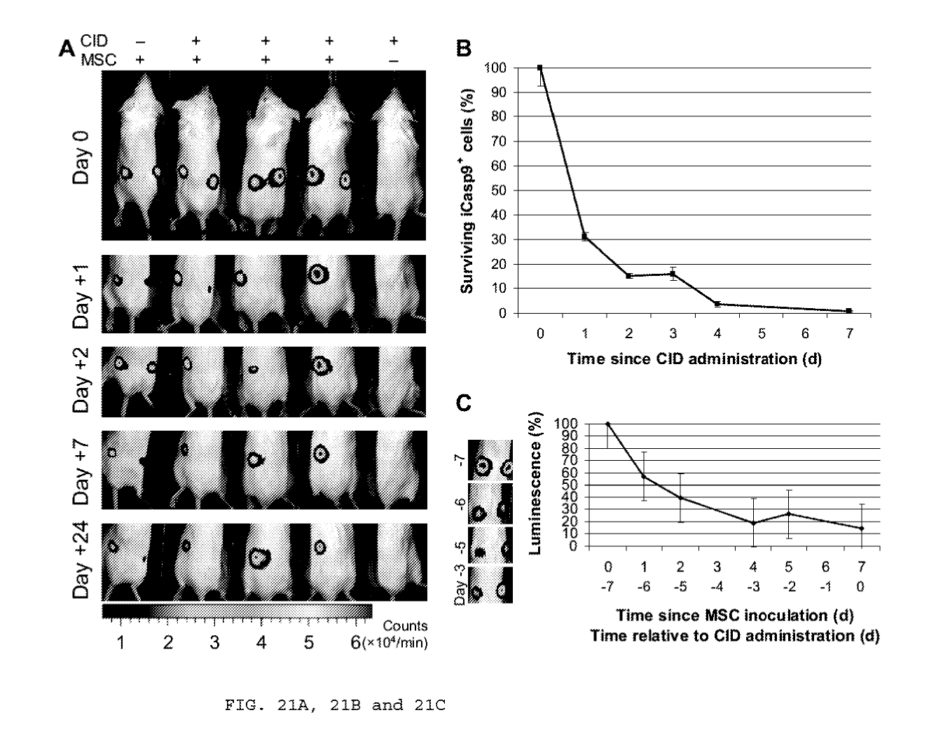

The luminescence data showed a substantial loss of human MSCs over the first 96 h (see FIG. 21C) after local delivery of MSCs, even before administration of CID, with only approximately 20% cells surviving after one week. From that time point onward, however, there were significant differences between the survival of icasp9-positive MSCs with and without dimerizer drug. Seven days after MSC implantation, animals were given two injections of 50 μg of CID, 24 hours apart. As illustrated in FIG. 21A, the MSCs transduced with iCasp9 were quickly killed by the drug, as demonstrated by the disappearance of their luminescence signal. Cells negative for iCasp9 were not affected by the drug. Animals not injected with the drug showed persistence of signal in both populations up to a month after MSC implantation. To further quantify cell killing, qPCR assays were developed to measure copy numbers of the eGFP-FFLuc and iCasp9-ΔCD19 genes. Mice were injected subcutaneously with a 1:1 mixture of doubly and singly transduced MSCs and administered CID as above, one week after MSC implantation. MSCs explants were collected at several time points, genomic DNA isolated from the samples and qPCR assays performed on substantially identical amounts of DNA. Under these conditions (see Methods), at any time point, the ratio of iCasp9-ΔCD19 to eGFP-FFLuc copy numbers is proportional to the fraction of viable iCasp9-positive cells. Progressive killing of iCasp9-positive cells was observed (>99%) so that the proportion of surviving iCasp9-positive cells was reduced to 0.7% of the original population after one week (see FIG. 21B). Therefore, MSCs transduced with iCasp9 can be selectively killed in vivo after exposure to CID, but otherwise persist.

### DISCUSSION

The feasibility of engineering human MSCs to express a safety mechanism using an inducible suicide protein is demonstrated herein. The date presented herein show that MSC can be readily transduced with the suicide gene iCasp9 coupled to the selectable surface maker CD19. Expression of the co-transduced genes is stable both in MSCs and their differentiated progeny, and does not evidently alter their phenotype or potential for differentiation. These transduced cells can be killed in vitro and in vivo when exposed to the appropriate small molecule chemical inducer of dimerization that binds to the iCasp9.

For a cell based therapy to be successful, transplanted cells must survive the period between their harvest and their ultimate in vivo clinical application. Additionally, a safe cell based therapy also should include the ability to control the unwanted growth and activity of successfully transplanted cells. Although MSCs have been administered to many patients without notable side effects, recent reports indicate additional protections, such as the safety switch presented herein, may offer additional methods of control over cell based therapies as the potential of transplanted MSC to be genetically and epigenetically modified to enhance their functionality, and to differentiate into lineages including bone and cartilage is further investigated and exploited. Subjects receiving MSCs that have been genetically modified to release biologically active proteins might particularly benefit from the added safety provided by a suicide gene.

The suicide system presented herein offers several potential advantages over other known suicide systems. Strategies involving nucleoside analogues, such as those combining Herpes Simplex Virus thymidine kinase (HSV-tk) with gancyclovir (GCV) and bacterial or yeast cytosine deaminase (CD) with 5-fluoro-cytosine (5-FC), are cell-cycle dependent and are unlikely to be effective in the post-mitotic tissues that may be formed during the application of MSCs to regenerative medicine. Moreover, even in proliferating tissues the mitotic fraction does not comprise all cells, and a significant portion of the graft may survive and remain dysfunctional. In some instance, the prodrugs required for suicide may themselves have therapeutic uses that are therefore excluded (e.g., GCV), or may be toxic (e.g., 5-FC), either as a result of their metabolism by non-target organs (e.g., many cytochrome P450 substrates), or due to diffusion to neighboring tissues after activation by target cells (e.g., CB1954, a substrate for bacterial nitroreductase).

In contrast, the small molecule chemical inducers of dimerization presented herein have shown no evidence of toxicities even at doses ten fold higher than those required to activate the iCasp9. Additionally, nonhuman enzymatic systems, such as HSV-tk and DC, carry a high risk of destructive immune responses against transduced cells. Both the iCasp9 suicide gene and the selection marker CD19, are of human origin, and thus should be less likely to induce unwanted immune responses. Although linkage of expression of the selectable marker to the suicide gene by a 2A-like cleavable peptide of nonhuman origin could pose problems, the 2A-like linker is 20 amino acids long, and is likely less immunogenic than a nonhuman protein. Finally, the effectiveness of suicide gene activation in iCasp9-positive cells compares favorably to killing of cells expressing other suicide systems, with 90% or more of iCasp9-modified T cells eliminated after a single dose of dimerizer, a level that is likely to be clinically efficacious.

The iCasp9 system presented herein also may avoid additional limitations seen with other cell based and/or suicide switch based therapies. Loss of expression due to silencing of the transduced construct is frequently observed after retroviral transduction of mammalian cells. The expression constructs presented herein showed no evidence of such an effect. No decrease in expression or induced death was evident, even after one month in culture.

Another potential problem sometimes observed in other cell based and/or suicide switch based therapies, is the development of resistance in cells that have upregulated anti-apoptotic genes. This effect has been observed in other suicide systems involving different elements of the programmed cell death pathways such as Fas. iCasp9 was chosen as the suicide gene for the expression constructs presented herein because it was less likely to have this limitation. Compared to other members of the apoptotic cascade, activation of caspase-9 occurs late in the apoptotic pathway and therefore should bypass the effects of many if not all anti-apoptotic regulators, such as c-FLIP and bcl-2 family members.

A potential limitation specific to the system presented herein may be spontaneous dimerization of iCasp9, which in turn could cause unwanted cell death and poor persistence. This effect has been observed in certain other inducible systems that utilize Fas. The observation of low spontaneous death rate in transduced cells and long term persistence of transgenic cells in vivo indicate this possibility is not a significant consideration when using iCasp9 based expression constructs.

Integration events deriving from retroviral transduction of MSCs may potentially drive deleterious mutagenesis, especially when there are multiple insertions of the retroviral vector, causing unwanted copy number effects and/or other undesirable effects. These unwanted effects could offset the benefit of a retrovirally transduced suicide system. These effects often can be minimized using clinical grade retroviral supernatant obtained from stable producer cell lines and similar culture conditions to transduce T lymphocytes. The T cells transduced and evaluated herein contain in the range of about 1 to 3 integrants (the supernatant containing in the range of about 1×10⁶ viral particles/mL). The substitution of lentiviral for retroviral vectors could further reduce the risk of genotoxicity, especially in cells with high self-renewal and differentiation potential.

While a small proportion of iCasp9-positive MSCs persists after a single exposure to CID, these surviving cells can subsequently be killed following re-exposure to CID. In vivo, there is >99% depletion with two doses, but it is likely that repeated doses of CID will be needed for maximal depletion in the clinical setting. Additional non-limiting methods of providing extra safety when using an inducible suicide switch system include additional rounds of cell sorting to further increase the purity of the cell populations administered and the use of more than one suicide gene system to enhance the efficiency of killing.

The CD19 molecule, which is physiologically expressed by B lymphocytes, was chosen as the selectable marker for transduced cells, because of its potential advantages over other available selection systems, such as neomycin phosphotransferase (neo) and truncated low affinity nerve growth factor receptor (ΔLNGFR). “neo” encodes a potentially immunogenic foreign protein and requires a 7-day culture in selection medium, increasing the complexity of the system and potentially damaging the selected cells. ΔLNGFR expression should allow for isolation strategies similar to other surface markers, but these are not widely available for clinical use and a lingering concern remains about the oncogenic potential of ΔLNGFR. In contrast, magnetic selection of iCasp9-positive cells by CD19 expression using a clinical grade device is readily available and has shown no notable effects on subsequent cell growth or differentiation.

The procedure used for preparation and administration of mesenchymal stromal cells comprising the caspase-9 safety switch may also be used for the preparation of embryonic stem cells and inducible pluripotent stem cells. Thus for the procedures outlined in the present example, either embryonic stem cells or inducible pluripotent stem cells may be substituted for the mesenchymal stromal cells provided in the example. In these cells, retroviral and lentiviral vectors may be used, with, for example, CMV promoters, or the ronin promoter.

### Example 6

### Modified Caspase-9 Polypeptides with Lower Basal Activity and Minimal Loss of Ligand IC₅₀

Basal signaling, signaling in the absence of agonist or activating agent, is prevalent in a multitude of biomolecules. For example, it has been observed in more than 60 wild-type G protein coupled receptors (GPCRs) from multiple subfamilies [1], kinases, such as ERK and abl [2], surface immunoglobulins [3], and proteases. Basal signaling has been hypothesized to contribute to a vast variety of biological events, from maintenance of embryonic stem cell pluripotency, B cell development and differentiation [4-6], T cell differentiation [2, 7], thymocyte development [8], endocytosis and drug tolerance [9], autoimmunity [10], to plant growth and development [11]. While its biological significance is not always fully understood or apparent, defective basal signaling can lead to serious consequences. Defective basal Gₛ protein signaling has led to diseases, such as retinitis pigmentosa, color blindness, nephrogenic diabetes insipidus, familial ACTH resistance, and familial hypocalciuric hypercalcemia [12, 13].

Even though homo-dimerization of wild-type initiator caspase-9 is energetically unfavorable, making them mostly monomers in solution [14-16], the low-level inherent basal activity of unprocessed caspase-9 [15, 17] is enhanced in the presence of the Apaf-1-based “apoptosome”, its natural allosteric regulator [6]. Moreover, supra-physiological expression levels and/or co-localization could lead to proximity-driven dimerization, further enhancing basal activation.

In the chimeric unmodified caspase-9 polypeptide, innate caspase-9 basal activity was significantly diminished by removal of the CAspase-Recruitment pro-Domain (CARD) [18], replacing it with the cognate high affinity AP1903-binding domain, FKBP12-F36V. Its usefulness as a pro-apoptotic “safety switch” for cell therapy has been well demonstrated in multiple studies [18-20]. While its high specific and low basal activity has made it a powerful tool in cell therapy, in contrast to G protein coupled receptors, there are currently no “inverse agonists” [21] to eliminate basal signaling, which may be desirable for manufacturing, and in some applications. Preparation of Master Cell Banks has proven challenging due to high amplification of the low-level basal activity of the chimeric polypeptide. In addition, some cells are more sensitive than others to low-level basal activity of caspase-9, leading to unintended apoptosis of transduced cells [18].

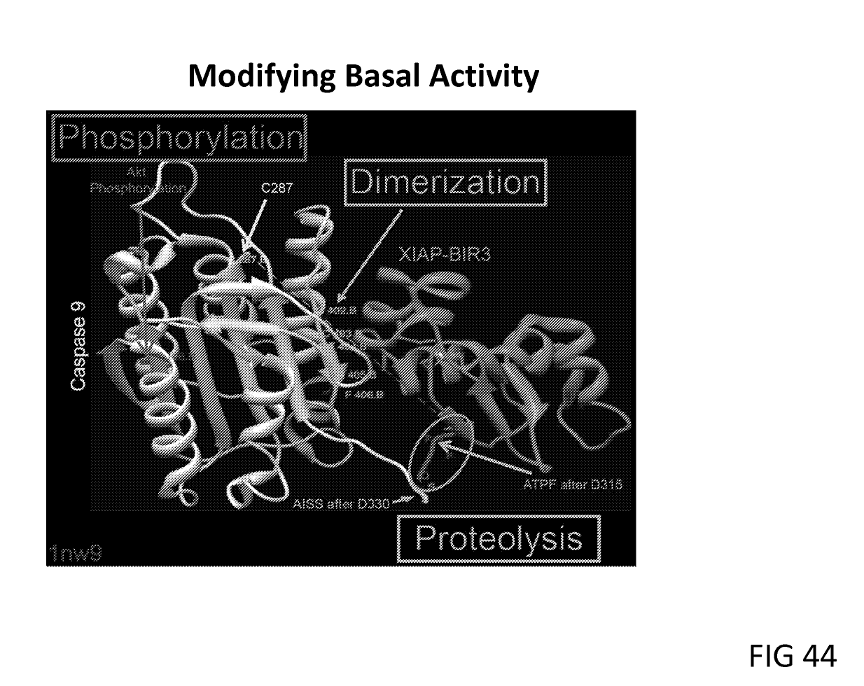

To modify the basal activity of the chimeric caspase-9 polypeptide, “rational design”-based methods were used to engineer 75i Casp9 mutants based on residues known to play crucial roles in homo-dimerization, XIAP-mediated inhibition, or phosphorylation (FIG. 44, Table below) rather than “directed evolution” [22] that use multiple cycles of screening as selective pressure on randomly generated mutants. Dimerization-driven activation of caspase-9 has been considered a dominant model of initiator Caspase activation [15, 23, 24]. To reduce spontaneous dimerization, site-directed mutagenesis was conducted of residues crucial for homo-dimerization and thus basal caspase-9 signaling. Replacement of five key residues in the β6 strand (G402-C-F-N-F406 (SEQ ID NO: 224)), the key dimerization interface of caspase-9, with those of constitutively dimeric effector Caspase-3 (C264-I-V-S-M268 (SEQ ID NO: 228)) converted it to a constitutively dimeric protein unresponsive to Apaf-1 activation without significant structural rearrangements [25]. To modify spontaneous homo-dimerization, systemic mutagenesis of the five residues was made, based on amino acid chemistry, and on corresponding residues of initiator Caspases-2, -8, -9, and -10 that exist predominately as a monomer in solution [14, 15]. After making and testing twenty-eight iCasp9 mutants by a secreted alkaline phosphatase (SEAP)-based surrogate killing assay (Table, below), the N405Q mutation was found to lower basal signaling with a moderate (<10-fold) cost of higher IC₅₀ to AP1903.

Since proteolysis, typically required for Caspase activation, is not absolutely required for caspase-9 activation [26], the thermodynamic “hurdle” was increased to inhibit auto-proteolysis. In addition, since XIAP-mediated caspase-9 binding traps caspase-9 in a monomeric state to attenuate its catalytic and basal activity [14], there was an effort to strengthen the interaction between XIAP and caspase-9 by mutagenizing the tetrapeptide critical for interaction with XIAP (A316-T-P-F319 (SEQ ID NO: 226), D330-A-I-S-S334 (SEQ ID NO: 234)). From 17 of these iCasp-9 mutants, it was determined that the D330A mutation lowered basal signaling with a minimum (<5-fold) AP1903 IC₅₀ cost.

The third approach was based on previously reported findings that caspase-9 is inhibited by kinases upon phosphorylation of S144 by PKC-ζ[27], S183 by protein kinase A [28], S196 by Akt1 [29], and activated upon phosphorylation of Y153 by c-abl [30]. These “brakes” might improve the IC₅₀, or substitutions with phosphorylation mimic (“phosphomimetic”) residues could augment these “brakes” to lower basal activity. However, none of the 15 single residue mutants based on these residues successfully lowered the IC₅₀ to AP1903.

Methods such as those discussed, for example, in Examples 1-5, and throughout the present application may be applied, with appropriate modifications, if necessary to the chimeric modified caspase-9 polypeptides, as well as to various therapeutic cells.

### Example 7

### Materials and Methods

### PCR Site-Directed Mutagenesis of Caspase-9:

To modify basal signaling of caspase-9, PCR-based site directed mutagenesis [31] was done with mutation-containing oligos and Kapa (Kapa Biosystems, Woburn, Mass.). After 18 cycles of amplification, parental plasmid was removed with methylation-dependent Dpnl restriction enzyme that leaves the PCR products intact. 2 μl of resulting reaction was used to chemically transform XL1-blue or DH5a. Positive mutants were subsequently identified via sequencing (SeqWright, Houston, Tex.).

### Cell Line Maintenance and Transfection:

Early passage HEK293T/16 cells (ATCC, Manassas, Va.) were maintained in IMDM, GlutaMAX™ (Life Technologies, Carlsbad, Calif.) supplemented with 10% FBS, 100 U/mL penicillin, and 100 U/mL streptomycin until transfection in a humidified, 37° C., 5% CO₂/95% air atmosphere. Cells in logarithmic-phase growth were transiently transfected with 800 ng to 2 μg of expression plasmid encoding iCasp9 mutants and 500 ng of an expression plasmid encoding SRα promoter driven SEAP per million cells in 15-mL conical tubes. Catalytically inactive caspase-9 (C285A) (without the FKBP domain) or “empty” expression plasmid (“pSH1-null”) were used to keep the total plasmid levels constant between transfections. GeneJammer® Transfection Reagent at a ratio of 3 μl per ug of plasmid DNA was used to transiently transfect HEK293T/16 cells in the absence of antibiotics. 100 μl or 2 mL of the transfection mixture was added to each well in 96-well or 6-well plate, respectively. For SEAP assays, log dilutions of AP1903 were added after a minimum 3-hour incubation post-transfection. For western blots, cells were incubated for 20 minutes with AP1903 (10 nM) before harvesting.

### Secreted Alkaline Phosphatase (SEAP) Assay:

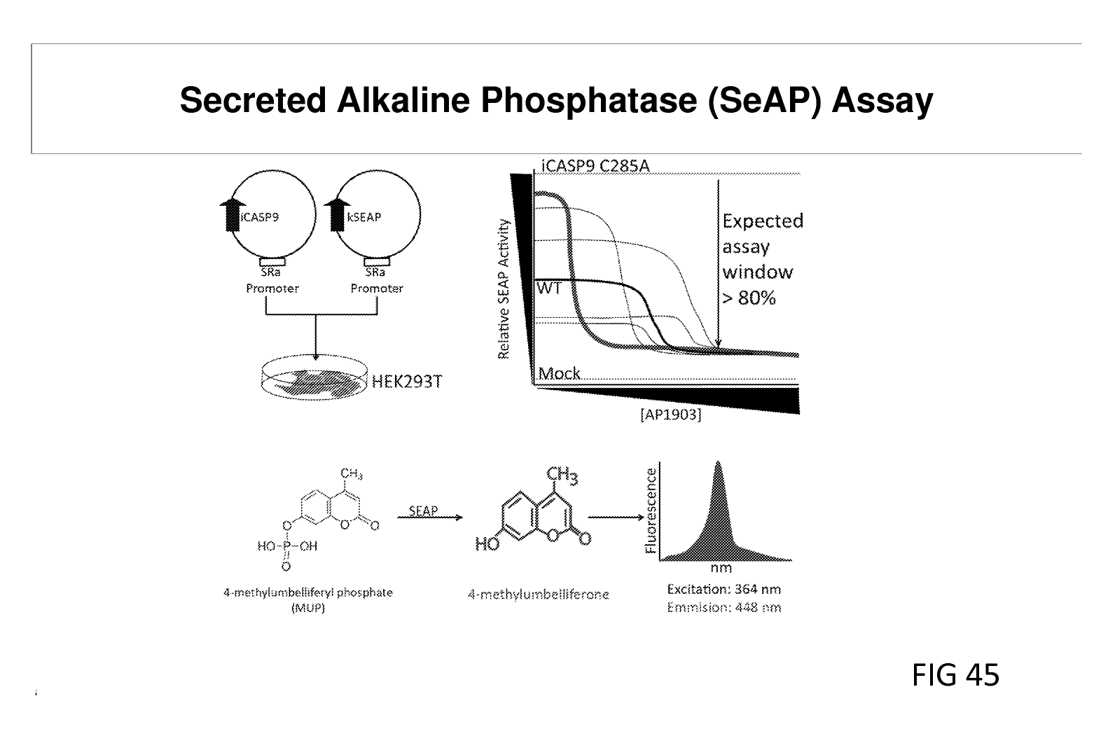

Twenty-four to forty-eight hours after AP1903 treatment, ˜100 μl of supernatants were harvested into a 96-well plate and assayed for SEAP activity as described [19, 32]. Briefly, after 65° C. heat denaturation for 45 minutes to reduce background caused by endogenous (and serum-derived) alkaline phosphatases that are sensitive to heat, 5 μl of supernatants was added to 95 μl of PBS and added to 100 μl of substrate buffer, containing 1 μl of 100 mM 4-methylumbelliferyl phosphate (4-MUP; Sigma, St. Louis, Mo.) re-suspended in 2 M diethanolamine. Hydrolysis of 4-MUP by SEAP produces a fluorescent substrate with excitation/emission (355/460 nm), which can be easily measured. Assays were performed in black opaque 96-well plates to minimize fluorescence leakage between wells (FIG. 45).

### Western Blot Analysis:

HEK293T/16 cells transiently transfected with 2 μg of plasmid for 48-72 hours were treated with AP1903 for 7.5 to 20 minutes (as indicated) at 37° C. and subsequently lysed in 500 μl of RIPA buffer (0.01 M Tris·HCl, pH 8.0/140 mM NaCl/1% Triton X-100/1 mM phenylmethylsulfonyl fluoride/1% sodium deoxycholate/0.1% SDS) with Halt™ Protease Inhibitor Cocktail. The lysates were collected and lysed on ice for 30 min. After pelleting cell debris, protein concentrations from overlying supernatants were measured in 96-well plates with BCA™ Protein Assay as recommended by the manufacturer. 30 μg of proteins were boiled in Laemmli sample buffer (Bio-Rad, Hercules, Calif.) with 2.5% 2-mercaptoethanol for 5 min at 95° C. before being separated by Criterion TGX 10% Tris/glylcine protein gel. Membranes were probed with 1/1000 rabbit anti-human caspase-9 polyclonal antibody followed by 1/10,000 HRP-conjugated goat anti-rabbit IgG F(ab′)2 secondary antibody (Bio-Rad). Protein bands were detected using Supersignal West Femto chemiluminescent substrate. To ensure equivalent sample loading, blots were stripped at 65° C. for 1 hour with Restore PLUS Western Blot Stripping Buffer before labeling with 1/10,000 rabbit anti-actin polyclonal antibody. Unless otherwise stated, all the reagents were purchased from Thermo Scientific.

Methods and constructs discussed in Examples 1-5 and throughout the present specification may also be used to assay and use the modified caspase-9 polypeptides.

### Example 8

### Evaluation and Activity of Chimeric Modified Caspase-9 Polypeptides

### Comparison of Basal Activity and AP1903 Induced Activity:

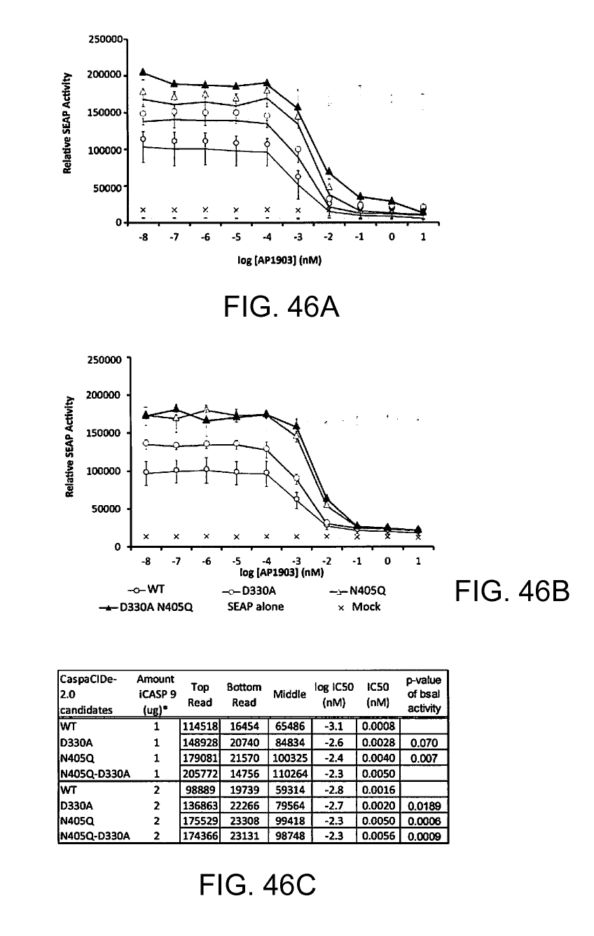

To examine both basal activity and AP1903 induced activity of the chimeric modified caspase-9 polypeptides, SEAP activities of HEK293T/16 cells co-transfected with SEAP and different amounts of iCasp9 mutants were examined. iCasp9 D330A, N405Q, and D330A-N405Q showed significantly less basal activity than unmodified iCasp9 for cells transfected with either 1 μg iCasp9 per million cells (relative SEAP activity Units of 148928, 179081, 205772 vs. 114518) or 2 μg iCasp9 per million cells (136863, 175529, 174366 vs. 98889) (FIG. 46A, 46B). The basal signaling of all three chimeric modified caspase-9 polypeptides when transfected at 2 μg per million cells was significantly higher (p value<0.05). iCasp9 D330A, N405Q, and D330A-N405Q also showed increased estimated IC₅₀s for AP1903, but they are all still less than 6 pM (based on the SEAP assay), compared to 1 pM for WT (FIG. 46C), making them potentially useful apoptosis switches.

### Evaluation of Protein Expression Levels and Proteolysis:

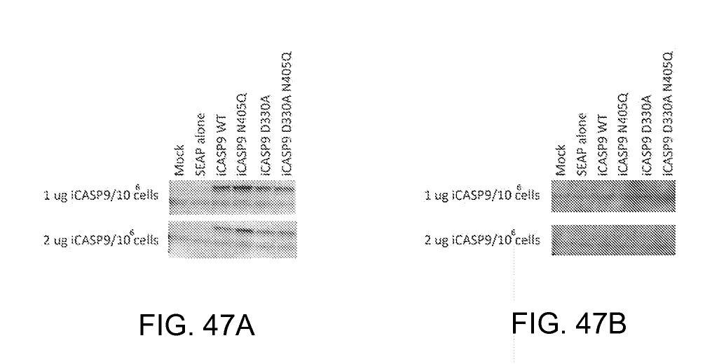

To exclude the possibility that the observed reduction in basal activity of the chimeric modified caspase-9 polypeptides was attributable to decreased protein stability or variation in transfection efficiency, and to examine auto-proteolysis of iCasp9, the protein expression levels of caspase-9 variants in transfected HEK293T/16 cells was assayed. Protein levels of chimeric unmodified caspase-9 polypeptide, iCasp9 D330A, and iCasp9 D330A-N405Q all showed similar protein levels under the transfection conditions used in this study (FIG. 47A). In contrast, the iCasp9 N405Q band appeared darker than the others, particularly when 2 μg of expression plasmids was used. Auto-proteolysis was not easily detectable at the transfection conditions used, likely because only viable cells were collected. Anti-actin protein reblotting confirmed that comparable lysate amounts were loaded into each lane (FIG. 47B). These results support the observed lower basal signaling in the iCasp9 D330A, N405Q, and D330A-N405Q mutants, observed by SEAP assays.

### DISCUSSION

Based on the SEAP screening assay, these three chimeric modified caspase-9 polypeptides showed higher AP1903-independent SEAP activity, compared to iCasp9 WT transfectants, and hence lower basal signaling. However, the double mutation (D330-N405Q) failed to further decrease either basal activity or IC₅₀ (0.05 nM) vs. the single amino acid mutants (FIG. 46A, 46B, 46C). The differences observed did not appear to be due to protein instability or differential amount of plasmids used during transfection (FIG. 47B).

### Example 9

### Evaluation and Activity of Chimeric Modified Caspase-9 Polypeptides

Inducible caspase-9 provides for rapid, cell-cycle-independent, cell autonomous killing in an AP1903-dependent fashion. Improving the characteristics of this inducible caspase-9 polypeptide would allow for even broader applicability. It is desirable to decrease the protein's ligand-independent cytotoxicity, and increase its killing at low levels of expression. Although ligand-independent cytotoxicity is not a concern at relatively low levels of expression, it can have a material impact where levels of expression can reach one or more orders of magnitude higher than in primary target cells, such as during vector production. Also, cells can be differentially sensitive to low levels of caspase expression due to the level of apoptosis inhibitors, like XIAP and Bcl-2, which cells express. Therefore, to re-engineer the caspase polypeptide to have a lower basal activity and possibly higher sensitivity to AP1903 ligand, four mutagenesis strategies were devised.

Dimerization Domain: Although caspase-9 is a monomer in solution at physiological levels, at high levels of expression, such as occurs in the pro-apoptotic, Apaf-driven “apoptosome”, caspase-9 can dimerize, leading to auto-proteolysis at D315 and a large increase in catalytic activity. Since C285 is part of the active site, mutation C285A is catalytically inactive and is used as a negative control construct. Dimerization involves very close interaction of five residues in particular, namely G402, C403, F404, N405, and F406. For each residue, a variety of amino acid substitutions, representing different classes of amino acids (e.g., hydrophobic, polar, etc.) were constructed. Interestingly, all mutants at G402 (i.e., G402A, G402I, G402Q, G402Y) and C403P led to a catalytically inactive caspase polypeptide. Additional C403 mutations (i.e., C403A, C403S, and C403T) were similar to the wild type caspase and were not pursued further. Mutations at F404 all lowered basal activity, but also reflected reduced sensitivity to IC₅₀, from ˜1 log to unmeasurable. In order of efficacy, they are: F404Y>F404T, F404W>>F404A, F4045. Mutations at N405 either had no effect, as with N405A, increased basal activity, as in N405T, or lowered basal activity concomitant with either a small (˜5-fold) or larger deleterious effect on IC₅₀, as with N405Q and N405F, respectively. Finally, like F404, mutations at F406 all lowered basal activity, and reflected reduced sensitivity to IC₅₀, from ˜1 log to unmeasurable. In order of efficacy, they are: F406A F406W, F406Y>F406T>>F406L.

Some polypeptides were constructed and tested that had compound mutations within the dimerization domain, but substituting the analogous 5 residues from other caspases, known to be monomers (e.g., Caspase-2, -8, -10) or dimers (e.g., Caspase-3) in solution. caspase-9 polypeptides, containing the 5-residue change from Caspase-2, -3, and -8, along with an AAAAA (SEQ ID NO: 229) alanine substitution were all catalytically inactive, while the equivalent residues from Caspase-10 (ISAQT (SEQ ID NO: 225)), led to reduced basal activity but higher IC₅₀.

Overall, based on the combination of consistently lower basal activity, combined with only a mild effect on IC₅₀, N405Q was selected for further experiments. To improve on efficacy, a codon-optimized version of the modified caspase-9 polypeptide, having the N405Q substitution, called N405Qco, was tested. This polypeptide appeared marginally more sensitive to AP1903 than the wild type N405Q-substituted caspase-9 polypeptide.

Cleavage site mutants: Following aggregation of caspase-9 within the apoptosome or via AP1903-enforced homodimerization, auto-proteolysis at D315 occurs. This creates a new amino-terminus at A316, at least transiently. Interestingly, the newly revealed tetrapeptide, ³¹⁶ATPF³¹⁹ (SEQ ID NO: 226), binds to the caspase-9 inhibitor, XIAP, which competes for dimerization with caspase-9 itself at the dimerization motif, GCFNF (SEQ ID NO: 224), described above. Therefore, the initial outcome of D315 cleavage is XIAP binding, attenuating further caspase-9 activation. However, a second caspase cleavage site exists at D330, which is the target of downstream effector caspase, caspase-3. As the pro-apoptotic pressure builds, D330 becomes increasingly cleaved, releasing the XIAP-binding small peptide within residue 316 to 330, and hence, removing this mitigating caspase-9 inhibitor. A D330A mutant was constructed, which lowered basal activity, but not as low as in N405Q. By SEAP assay at high copy number, it also revealed a slight increase in IC₅₀, but at low copy number in primary T cells, there was actually a slight increase in IC₅₀ with improved killing of target cells. Mutation at auto-proteolysis site, D315, also reduced basal activity, but this led to a large increase in IC₅₀, likely as D330 cleavage was then necessary for caspase activation. A double mutation at D315A and D330A, led to an inactive “locked” caspase-9 that could not be processed properly.

Other D330 mutants were created, including D330E, D330G, D330N, D330S, and D330V. Mutation at D327, also prevented cleavage at D330, as the consensus Caspase-3 cleavage site is DxxD, but several D327 mutations (i.e., D327G, D327K, and D327R) along with F326K, Q328K, Q328R, L329K, L329G, and A331K, unlike D330 mutations, did not lower basal activity and were not pursued further.

XIAP-binding mutants: As described above, autoproteolysis at D315 reveals an XIAP-binding tetrapeptide, ³¹⁶ATPF³¹⁹ (SEQ ID NO: 226), which “lures” XIAP into the caspase-9 complex. Substitution of ATPF (SEQ ID NO: 226) with the analogous XIAP-binding tetrapeptide, AVPI (SEQ ID NO: 227), from mitochondria-derived anti-XIAP inhibitor, SMAC/DIABLO, might bind more tightly to XIAP and lower basal activity. However, this 4-residue substitution had no effect. Other substitutions within the ATPF motif (SEQ ID NO: 226) ranged from no effect, (i.e., T317C, P318A, F319A) to lower basal activity with either a very mild (i.e., T317S, mild (i.e., T317A) to large (i.e., A316G, F319W) increase in IC₅₀. Overall, the effects of changing the XIAP-binding tetrapeptide were mild; nonetheless, T317S was selected for testing in double mutations (described below), since the effects on IC₅₀ were the most mild of the group.

Phosphorylation mutants: A small number of caspase-9 residues were reported to be the targets of either inhibitory (e.g., S144, S183, S195, S196, S307, T317) or activating (i.e., Y153) phosphorylations. Therefore, mutations that either mimic the phosphorylation (“phosphomimetics”) by substitution with an acidic residue (e.g., Asp) or eliminate phosphorylation were tested. In general, most mutations, regardless of whether a phosphomimetic or not was tried, lowered basal activity. Among the mutants with lower basal activity, mutations at S144 (i.e., S144A and S144D) and 51496D had no discernable effect on IC₅₀, mutants S183A, S195A, and S196A increased the IC₅₀ mildly, and mutants Y153A, Y153A, and S307A had a big deleterious effect on IC₅₀. Due to the combination of lower basal activity and minimal, if any effect on IC₅₀, S144A was chosen for double mutations (described below).

Double mutants: In order to combine the slightly improved efficacy of D330A variant with possible residues that could further lower basal activity, numerous D330A double mutants were constructed and tested. Typically, they maintained lower basal activity with only a slight increase in IC₅₀, including 2nd mutations at N405Q, S144A, S144D, S183A, and S196A. Double mutant D330A-N405T had higher basal activity and double mutants at D330A with Y153A, Y153F, and T317E were catalytically inactive. A series of double mutants with low basal activity N405Q, intended to improve efficacy or decrease the IC₅₀ was tested. These all appeared similar to N405Q in terms of low basal activity and slightly increased IC₅₀ relative to CaspaClDe-1.0, and included N405Q with S144A, S144D, S196D, and T317S.

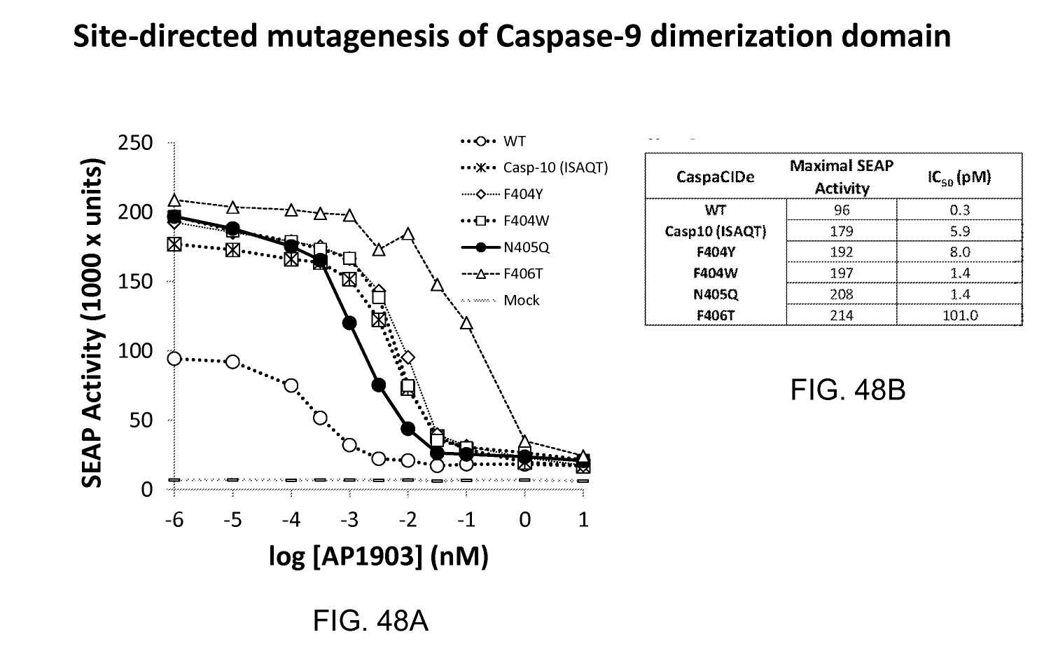

FIG. 48A represents a SEAP assay to study the basal activity and CID sensitivity of some of the dimerization domain mutants. This shows that N405Q (black circles) was the most AP1903-sensitive of the mutants tested with lower basal activity than the WT caspase-9, as determined by a shift upwards of AP1903-independent signaling. F406T was the least CID-sensitive from this group. 48B shows a table of Maximal SEAP activity (reflecting basal activity) and IC₅₀s.

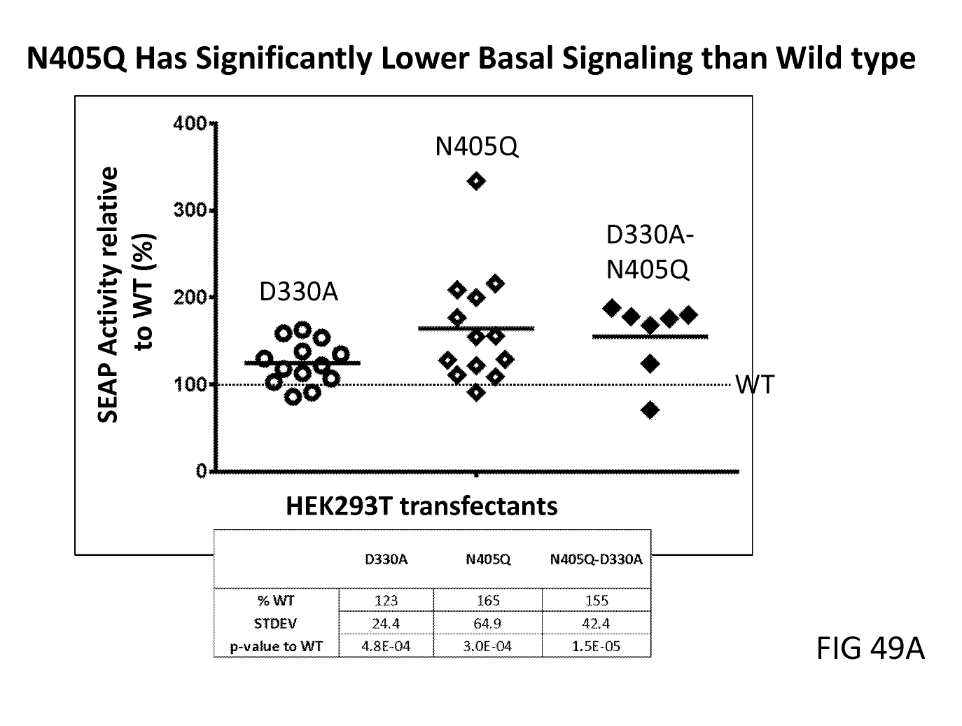

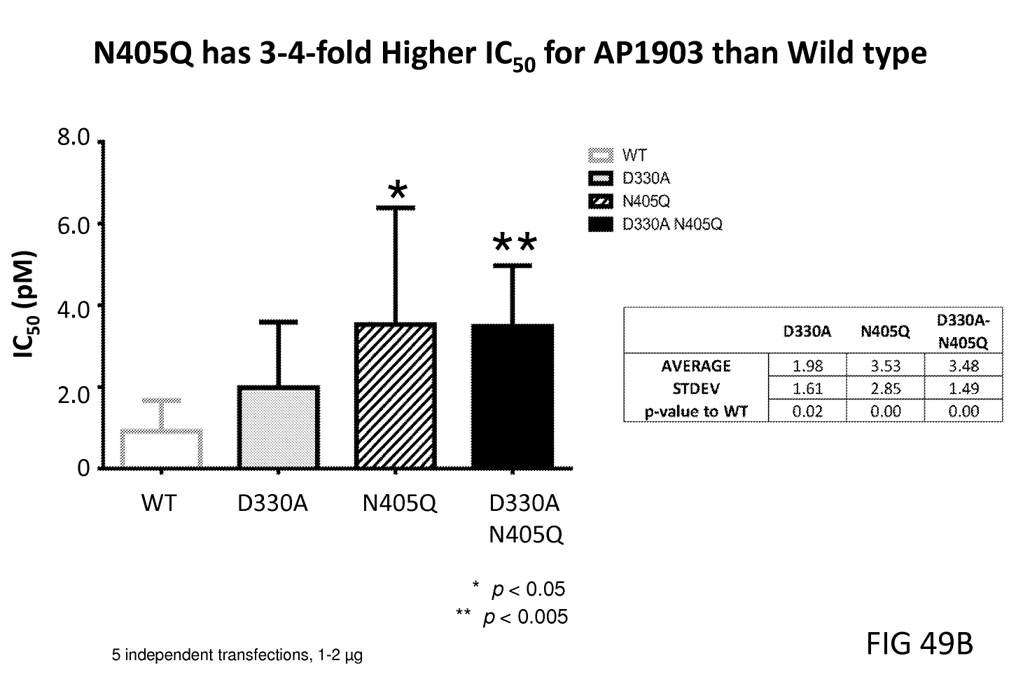

FIG. 49A represents the dimer-independent SEAP activity of mutant caspase polypeptides D330A and N405Q, along with double mutant D330A-N405Q. The results of multiple transfections (N=7 to 13) are shown, illustrating that N405Q has lower basal activity than D330A and the double mutant is intermediate.

FIG. 49B represents the average (+stdev, n=5) IC₅₀ of mutant caspase polypeptides D330A and N405Q, along with double mutant D330A-N405Q. The results show that D330A is somewhat more sensitive to AP1903 than N405Q mutants but about 2-fold less sensitive than WT caspase-9 in a transient transfection assay.

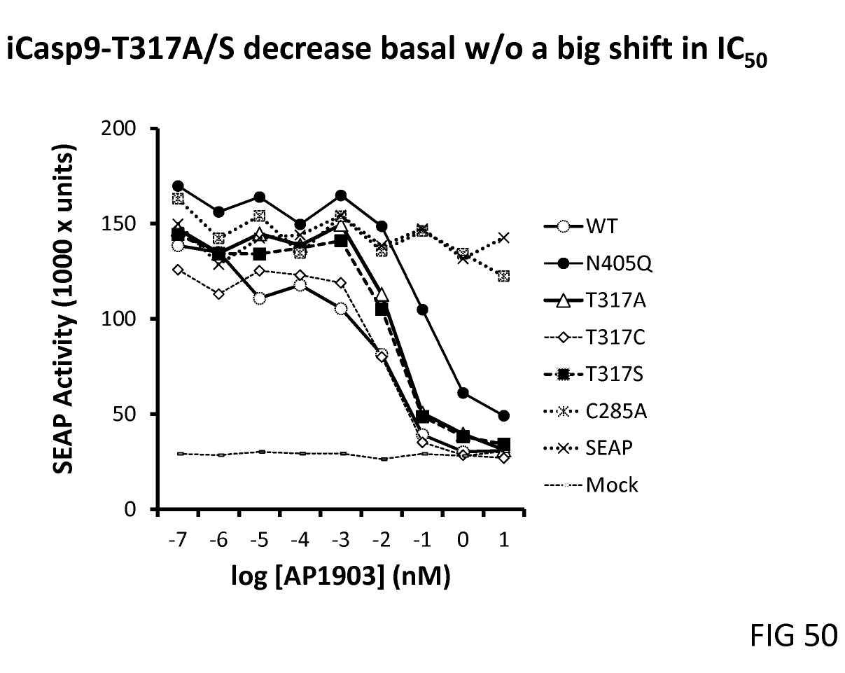

FIG. 50 represents a SEAP assay reflecting WT caspase-9, N405Q, inactive C285A, and several T317 mutants within the XIAP-binding domain. The results show that T317S and T317A can reduce basal activity without a large shift in the IC₅₀ to APf1903. Therefore, T317S was chosen to make double mutants with N405Q.

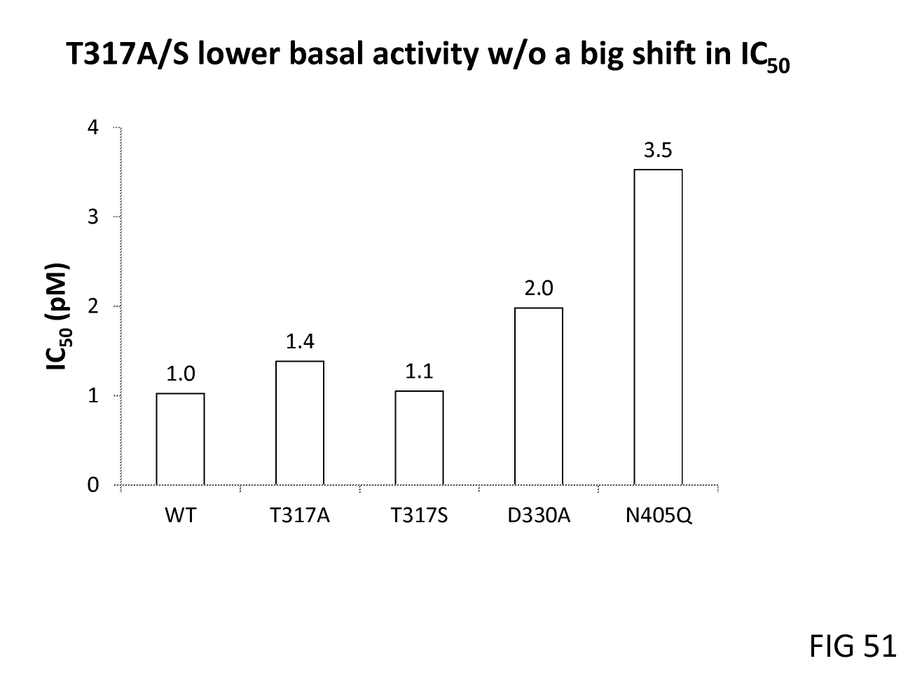

FIG. 51 represents the IC₅₀s from experiment 50B, showing that T317A and T317S have similar IC₅₀s to wild type caspase-9 polypeptide despite having lower basal activity.

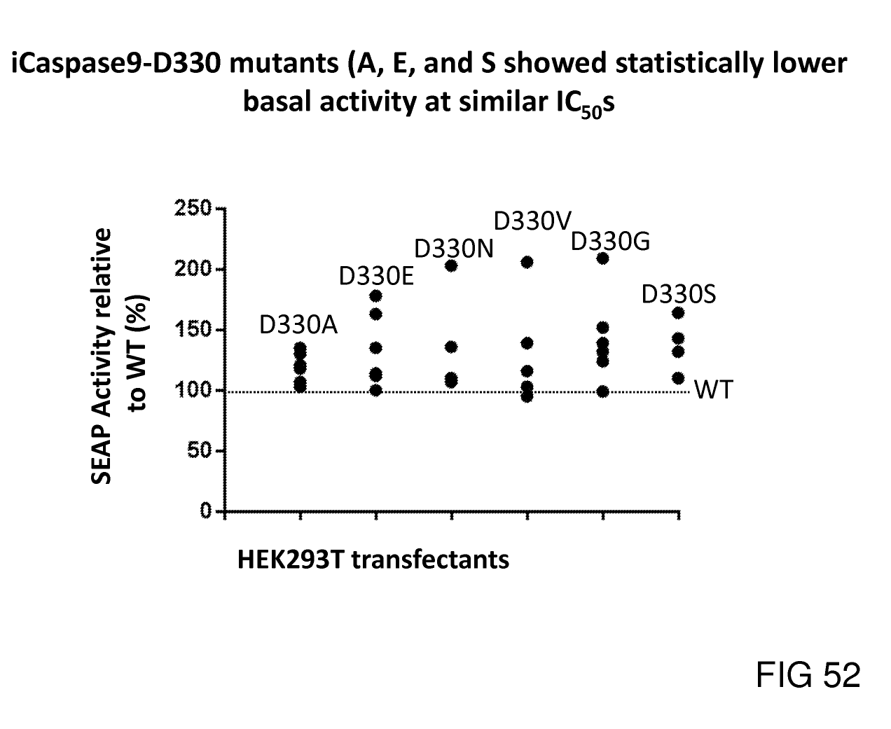

FIG. 52 represents the dimer-independent SEAP activity from several D330 mutants showing that all members of this class tested, including D330A, D330E, D330N, D330V, D330G, and D330S, have less basal activity than wild type caspase-9.

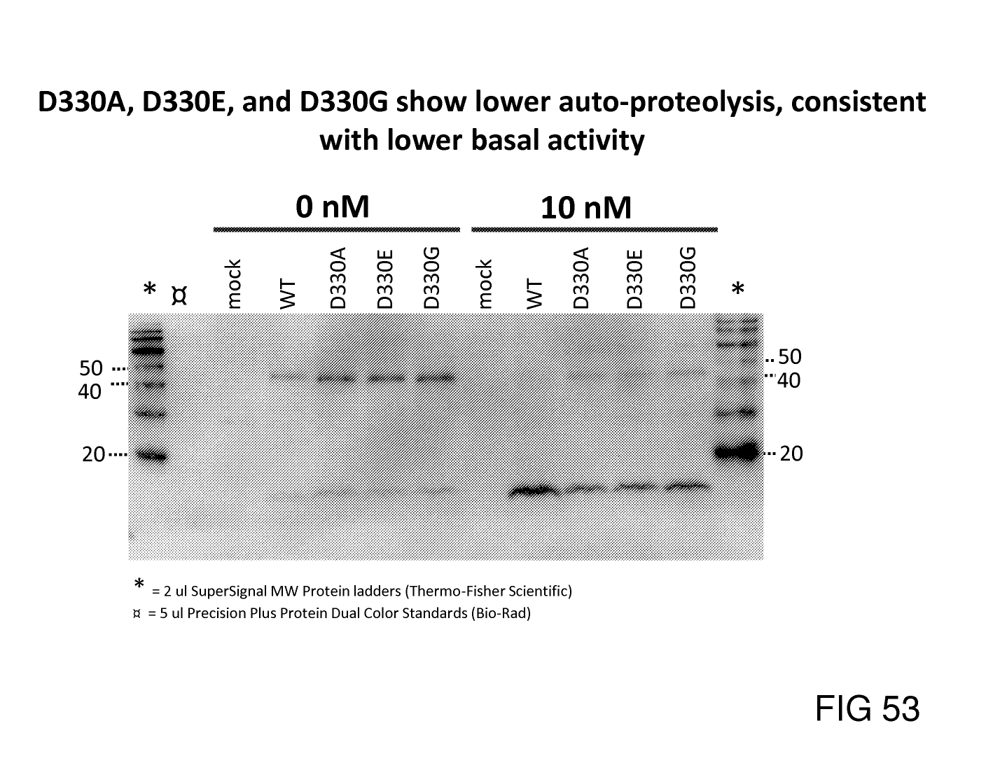

FIG. 53 shows the result of a western blot illustrating that the D330 mutations block cleavage at D330, leading to a slightly largely (slower migrating) small band (<20 kDa marker). Other blots show that D327 mutation also blocks cleavage.

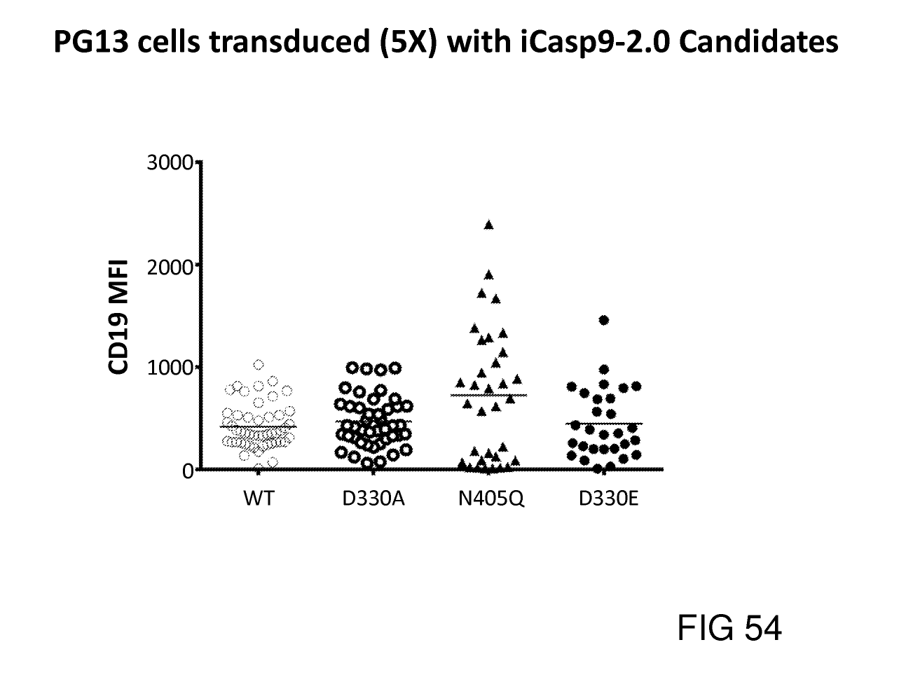

FIG. 54 shows the mean fluorescence intensities of multiple clones of PG13 transduced 5× with retroviruses encoding the indicated caspase-9 polypeptides. Lower basal activity typically translates to higher levels of expression of the caspase-9 gene along with the genetically linked reporter, CD19. The results show that on the average, clones expressing the N405Q mutant express higher levels of CD19, reflecting the lower basal activity of N405Q over D330 mutants or WT caspase-9.

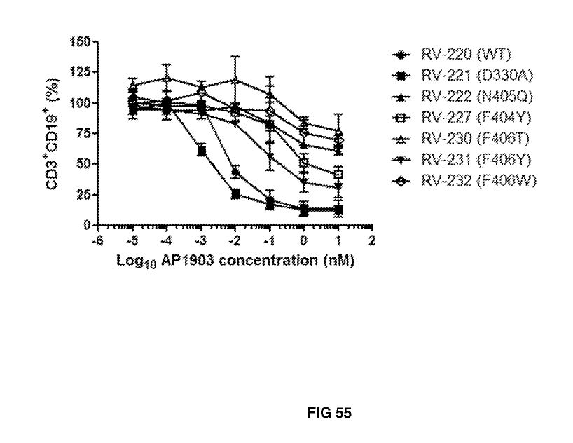

FIG. 55 shows the effects of various caspase polypeptides at mostly single copy in primary T cells. This may reflect more accurately how these suicide genes will be used therapeutically. Surprisingly, the data show that the D330A mutant is actually more sensitive to AP1903 at low titers and kills at least as well as WT caspase-9 when tested in a 24-hour assay. The N405Q mutant is less sensitive to AP1903 and cannot kill target cells as efficiently within 24 hours.

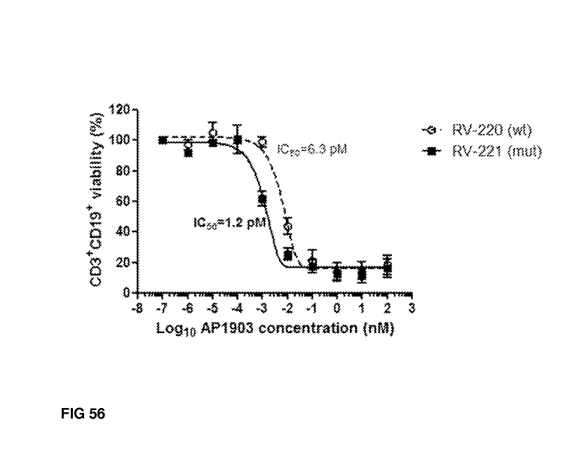

FIG. 56 shows the results of transducing 6 independent T cell samples from separate healthy donors. These results confirm that the D330A mutant (mut) is more sensitive to AP1903 than the wild type caspase-9 polypeptide.

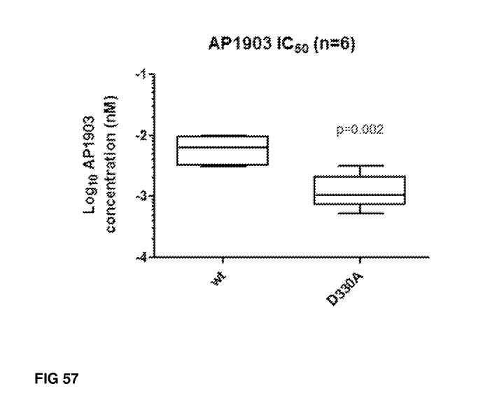

FIG. 57 shows the average IC₅₀, range and standard deviation from the 6 healthy donors shown in FIG. 56. This data shows that the improvement is statistically significant.

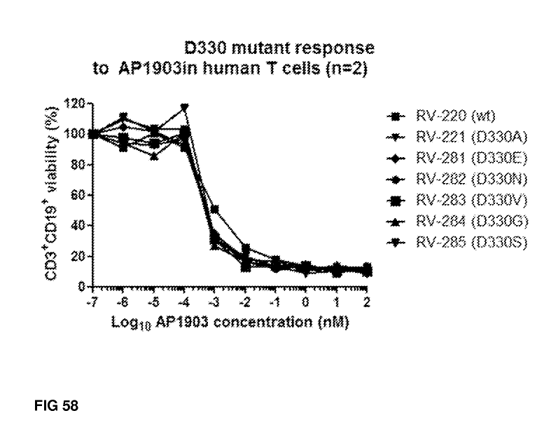

FIG. 58 shows the results of several D330 mutants, revealing that all six D330 mutants tested (D330A, E, N, V, G, and S) are more sensitive to AP1903 than wild type caspase-9 polypeptide.

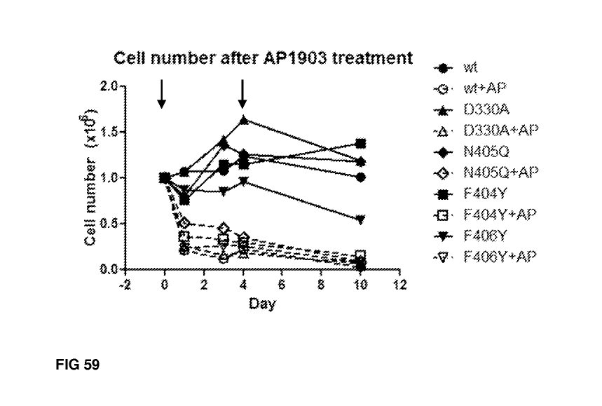

FIG. 59 shows that the N405Q mutant along with other dimerization domain mutants, including N404Y and N406Y, can kill target T cells indistinguishable from wild type caspase-9 polypeptide or D330A within 10 days. Cells that received AP1903 at Day 0 received a second dose of AP1903 at day 4. This data supports the use of reduced sensitivity caspase-9 mutants, like N405Q as part of a regulated efficacy switch.

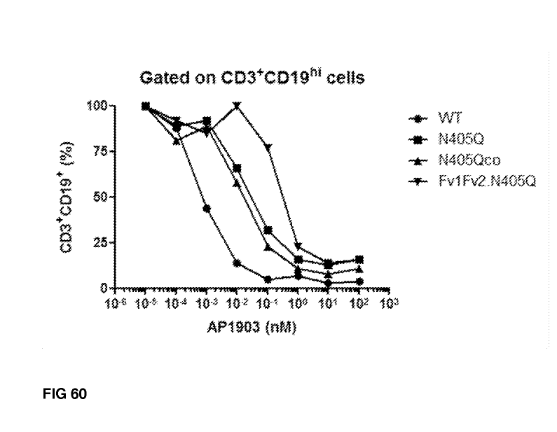

FIG. 60 shows the results of codon optimization of N405Q caspase polypeptide, called “N405Qco”, revealing that codon optimization, likely leading to an increase in expression only has a very subtle effect on inducible caspase function. This likely reflects the use of common codons in the original caspase-9 gene.

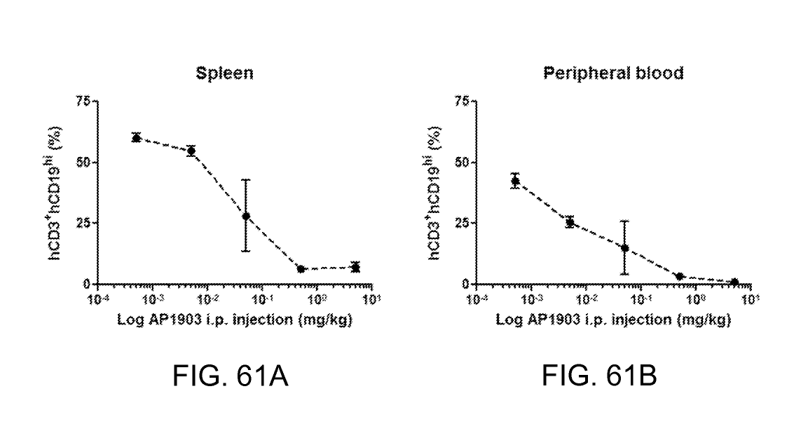

FIG. 61 shows that the caspase-9 polypeptide has a dose-response curve in vivo, which could be used to eliminate a variable fraction of T cells expressing the caspase-9 polypeptide. The data also shows that a dose of 0.5 mg/kg AP1903 is sufficient to eliminate most modified T cells in vivo.

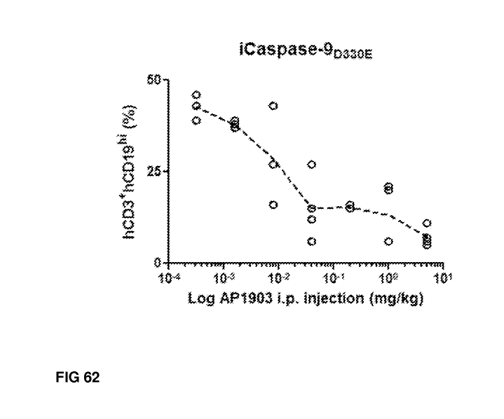

FIG. 62 shows the dose-response curve of the D330E mutant in vivo. This study also shows that elimination of T cells in titratable in vivo.

Conclusions: As described, from this analysis of 78 mutants so far, out of the single mutant mutations, the D330 mutations combine somewhat improved efficacy with slightly reduced basal activity. N405Q mutants are also attractive since they have very low basal activity with only slightly decreased efficacy, reflected by a 4-5-fold increase in IC₅₀. Experiments in primary T cells have shown that N405Q mutants can effectively kill target cells, but with somewhat slower kinetics than D330 mutants, making this potentially very useful for a graduated suicide switch that kills partially after an initial dose of AP1903, and up to full killing can be achieved upon a second dose of AP1903.

The following table provides a summary of basal activity and IC₅₀ for various chimeric modified caspase-9 polypeptides prepared and assayed according to the methods discussed herein. The results are based on a minimum of two independent SEAP assays, except for a subset (i.e., A316G, T317E, F326K, D327G, D327K, D327R, Q328K, Q328R, L329G, L329K, A331K, S196A, S196D, and the following double mutants: D330A with S144A, S144D, or S183A; and N405Q with S144A, S144D, S196D, or T317S) that were tested once. Four multi-pronged approaches were taken to generate the tested chimeric modified caspase-9 polypeptides. “Dead” modified caspase-9 polypeptides were no longer responsive to AP1903. Double mutants are indicated by a hyphen, for example, D330A-N405Q denotes a modified caspase-9 polypeptide having a substitution at position 330 and a substitution at position 405.

TABLE 5
Caspase Mutant Classes

| Basal | Homodimerization | Cleavage sites & | | Double | Total |
|---|---|---|---|---|---|
| Activity | domain | XIAP Interaction | Phosphorylation | mutants, Misc. | mutants |
| Decreased | | | S144A | | 80 |
| basal and | | | S144D | | *, predicted |
| similar IC₅₀ | | T317S | S196D | | |
| Decreased | N405Q | D330A | S183A | D330A-N405Q | Bold, |
| basal but | | | | | Tested in T cells |
| higher IC₅₀ | ⁴⁰²GCFNF⁴⁰⁶ISAQT | D330E | S195A | D330A-S144A | |
| | (Casp-10) (SEQ ID | | | | |
| | NOS 224 and 225) | | | | |
| | F404Y | D330G | S196A | D330A-S144D | |
| | F406A | D330N | | D330A-S183A | |
| | F406W | D330S | | D330A-S196A | |
| | F406Y | D330V | | N405Q-S144A | |
| | N405Qco | L329E | | N405Q-S144D | |
| | | T317A | | N405Q-S196D | |
| | | | | N405Q-T317S | |
| | | | | *N405Q-S144Aco | |
| | | | | *N405Q-T317Sco | |
| Decreased | F404T | D315A | Y153A | | |
| basal but | F404W | A316G | Y153F | | |
| much | N405F | F319W | S307A | | |
| higher IC₅₀ | F406T | | | | |
| Similar | C403A | ³¹⁶ATPF³¹⁹AVPI | | | |
| basal | | (SMAC/Diablo) | | | |
| and IC₅₀ | | (SEQ ID NOS | | | |
| | | 226 and 227) | | | |
| | C403S | T317C | | | |
| | C403T | P318A | | | |
| | N405A | F319A | | | |
| Increased | N405T | T317E | | D330A-N405T | |
| basal | | F326K | | | |
| | | D327G | | | |
| | | D327K | | | |
| | | D327R | | | |
| | | Q328K | | | |
| | | Q328R | | | |
| | | L329G | | | |
| | | L329K | | | |
| | | A331K | | | |
| Catalytically | ⁴⁰²GCFNF⁴⁰⁶AAAAA | | | C285A | |
| dead | (SEQ ID NOS | | | | |
| | 224 and 229) | | | | |
| | ⁴⁰²GCFNF⁴⁰⁶YCSTL | | | D315A-D330A | |
| | (Casp-2) | | | | |
| | (SEQ ID NOS | | | | |
| | 224 and 230) | | | | |
| | ⁴⁰²GCFNF⁴⁰⁶CIVSM | | | D330A-Y153A | |
| | (Casp-3) | | | | |
| | (SEQ ID NOS | | | | |
| | 224 and 228) | | | | |
| | ⁴⁰²GCFNF⁴⁰⁶QPTFT | | | D330A-Y153F | |
| | (Casp-8) | | | | |
| | (SEQ ID NOS | | | | |
| | 224 and 231) | | | | |
| | G402A | | | D330A-T317E | |
| | G402I | | | | |
| | G402Q | | | | |
| | G402Y | | | | |
| | C403P | | | | |
| | F404A | | | | |
| | F404S | | | | |
| | F406L | | | | |

### LITERATURE REFERENCES CITED IN EXAMPLES 6-9

1. Seifert, R. and K. Wenzel-Seifert, Constitutive activity of G-protein-coupled receptors: cause of disease and common property of wild-type receptors. Naunyn Schmiedebergs Arch Pharmacol, 2002. 366(5): p. 381-416.
2. Roose, J. P., et al., T cell receptor-independent basal signaling via Erk and Abl kinases suppresses RAG gene expression. PLoS Biol, 2003. 1(2): p. E53.
3. Tze, L. E., et al., Basal immunoglobulin signaling actively maintains developmental stage in immature B cells. PLoS Biol, 2005. 3(3): p. e82.
4. Schram, B. R., et al., B cell receptor basal signaling regulates antigen-induced Ig light chain rearrangements. J Immunol, 2008. 180(7): p. 4728-41.
5. Randall, K. L., et al., Dock8 mutations cripple B cell immunological synapses, germinal centers and long-lived antibody production. Nat Immunol, 2009. 10(12): p. 1283-91.
6. Kouskoff, V., et al., B cell receptor expression level determines the fate of developing B lymphocytes: receptor editing versus selection. Proc Natl Acad Sci USA, 2000. 97(13): p. 7435-9.
7. Hong, T., et al., A simple theoretical framework for understanding heterogeneous differentiation of CD4+ T cells. BMC Syst Biol, 2012. 6: p. 66.
8. Rudd, M. L., A. Tua-Smith, and D. B. Straus, Lck SH3 domain function is required for T-cell receptor signals regulating thymocyte development. Mol Cell Biol, 2006. 26(21): p. 7892-900.
9. Sorkin, A. and M. von Zastrow, Endocytosis and signalling: intertwining molecular networks. Nat Rev Mol Cell Biol, 2009. 10(9): p. 609-22.
10. Luning Prak, E. T., M. Monestier, and R. A. Eisenberg, B cell receptor editing in tolerance and autoimmunity. Ann N Y Acad Sci, 2011. 1217: p. 96-121.
11. Boss, W. F., et al., Basal signaling regulates plant growth and development. Plant Physiol, 2010. 154(2): p. 439-43.
12. Tao, Y. X., Constitutive activation of G protein-coupled receptors and diseases: insights into mechanisms of activation and therapeutics. Pharmacol Ther, 2008. 120(2): p. 129-48.
13. Spiegel, A. M., Defects in G protein-coupled signal transduction in human disease. Annu Rev Physiol, 1996. 58: p. 143-70.
14. Shiozaki, E. N., et al., Mechanism of XIAP-mediated inhibition of caspase-9. Mol Cell, 2003. 11(2): p. 519-27.
15. Renatus, M., et al., Dimer formation drives the activation of the cell death protease caspase-9. Proc Natl Acad Sci USA, 2001. 98(25): p. 14250-5.
16. Shi, Y., Mechanisms of Caspase activation and inhibition during apoptosis. Mol Cell, 2002. 9(3): p. 459-70.
17. Shiozaki, E. N., J. Chai, and Y. Shi, Oligomerization and activation of caspase-9, induced by Apaf-1 CARD. Proc Natl Acad Sci USA, 2002. 99(7): p. 4197-202.
18. Straathof, K. C., et al., An inducible caspase-9 safety switch for T-cell therapy. Blood, 2005. 105(11): p. 4247-54.
19. MacCorkle, R. A., K. W. Freeman, and D. M. Spencer, Synthetic activation of Caspases: artificial death switches. Proc Natl Acad Sci USA, 1998. 95(7): p. 3655-60.
20. Di Stasi, A., et al., Inducible apoptosis as a safety switch for adoptive cell therapy. N Engl J Med, 2011. 365(18): p. 1673-83.
21. Chang, W. C., et al., Modifying ligand-induced and constitutive signaling of the human 5-HT4 receptor. PLoS One, 2007. 2(12): p. e1317.
22. Bloom, J. D. and F. H. Arnold, In the light of directed evolution: pathways of adaptive protein evolution. Proc Natl Acad Sci USA, 2009. 106 Suppl 1: p. 9995-10000.
23. Boatright, K. M. and G. S. Salvesen, Mechanisms of Caspase activation. Curr Opin Cell Biol, 2003. 15(6): p. 725-31.
24. Boatright, K. M., et al., A unified model for apical Caspase activation. Mol Cell, 2003. 11(2): p. 529-41.
25. Chao, Y., et al., Engineering a dimeric caspase-9: a re-evaluation of the induced proximity model for Caspase activation. PLoS Biol, 2005. 3(6): p. e183.
26. Stennicke, H. R., et al., caspase-9 can be activated without proteolytic processing. J Biol Chem, 1999. 274(13): p. 8359-62.
27. Brady, S. C., L. A. Allan, and P. R. Clarke, Regulation of caspase-9 through phosphorylation by protein kinase C zeta in response to hyperosmotic stress. Mol Cell Biol, 2005. 25(23): p. 10543-55.
28. Martin, M. C., et al., Protein kinase A regulates caspase-9 activation by Apaf-1 downstream of cytochrome c. J Biol Chem, 2005. 280(15): p. 15449-55.
29. Cardone, M. H., et al., Regulation of cell death protease caspase-9 by phosphorylation. Science, 1998. 282(5392): p. 1318-21.
30. Raina, D., et al., c-Abl tyrosine kinase regulates caspase-9 autocleavage in the apoptotic response to DNA damage. J Biol Chem, 2005. 280(12): p. 11147-51.
31. Papworth, C., Bauer, J. C., Braman, J. and Wright, D. A., Site-directed mutagenesis in one day with >80% efficiency. Strategies, 1996. 9(3): p. 3-4.
32. Spencer, D. M., et al., Functional analysis of Fas signaling in vivo using synthetic inducers of dimerization. Curr Biol, 1996. 6(7): p. 839-47.
33. Hsiao, E. C., et al., Constitutive Gs activation using a single-construct tetracycline-inducible expression system in embryonic stem cells and mice. Stem Cell Res Ther, 2011. 2(2): p. 11.
34. Waldner, C., et al., Double conditional human embryonic kidney cell line based on FLP and PhiC31 mediated transgene integration. BMC Res Notes, 2011. 4: p. 420.

### Example 10

### Examples of Particular Nucleic Acid and Amino Acid Sequences

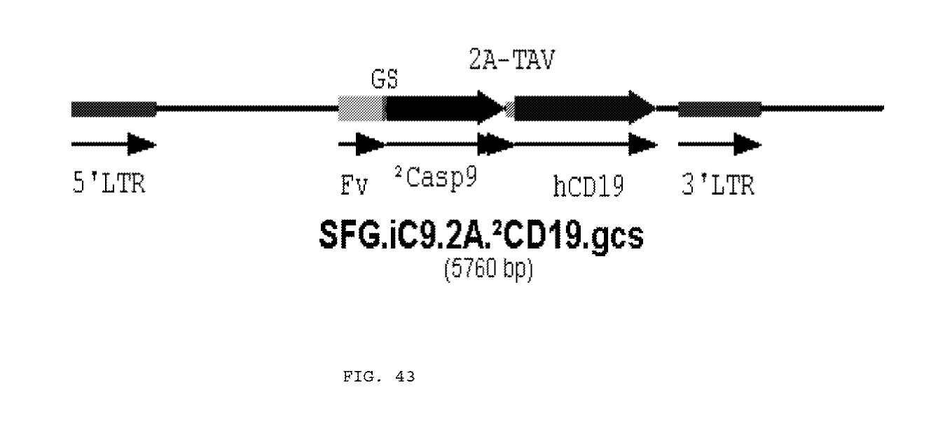

FIG. 43 presents an example of a construct that may be used for expression of the chimeric protein and CD19 marker. The figure presents the SFG.iC9.2A.ΔCD19.gcs construct

SEQ ID NO: 1, nucleotide sequence of 5′LTR sequence
TGAAAGACCCCACCTGTAGGTTTGGCAAGCTAGCTTAAGTAACGCCATTTTGCAAG
GCATGGAAAAATACATAACTGAGAATAGAAAAGTTCAGATCAAGGTCAGGAACAGA
TGGAACAGCTGAATATGGGCCAAACAGGATATCTGTGGTAAGCAGTTCCTGCCCCG
GCTCAGGGCCAAGAACAGATGGAACAGCTGAATATGGGCCAAACAGGATATCTGT
GGTAAGCAGTTCCTGCCCCGGCTCAGGGCCAAGAACAGATGGTCCCCAGATGCGG
TCCAGCCCTCAGCAGTTTCTAGAGAACCATCAGATGTTTCCAGGGTGCCCCAAGGA
CCTGAAATGACCCTGTGCCTTATTTGAACTAACCAATCAGTTCGCTTCTCGCTTCTG
TTCGCGCGCTTATGCTCCCCGAGCTCAATAAAAGAGCCCACAACCCCTCACTCGGG
GCGCCAGTCCTCCGATTGACTGAGTCGCCCGGGTACCCGTGTATCCAATAAACCCT
CTTGCAGTTGCATCCGACTTGTGGTCTCGCTGTTCCTTGGGAGGGTCTCCTCTGAG
TGATTGACTACCCGTCAGCGGGGGTCTTTCA
SEQ ID NO: 2, nucleotide sequence of Fᵥ (human FKBP12v36)
GGAGTGCAGGTGGAAACCATCTCCCCAGGAGACGGGCGCACCTTCCCCAAGCGC
GGCCAGACCTGCGTGGTGCACTACACCGGGATGCTTGAAGATGGAAAGAAAGTTG
ATTCCTCCCGGGACAGAAACAAGCCCTTTAAGTTTATGCTAGGCAAGCAGGAGGTG
ATCCGAGGCTGGGAAGAAGGGGTTGCCCAGATGAGTGTGGGTCAGAGAGCCAAAC
TGACTATATCTCCAGATTATGCCTATGGTGCCACTGGGCACCCAGGCATCATCCCA
CCACATGCCACTCTCGTCTTCGATGTGGAGCTTCTAAAACTGGAA
SEQ ID NO: 3 amino acid sequence of Fv (human FKBP12v36)
G V Q V E T I S P G D G R T F P K R G Q T C V V H Y T G M L E D
G K K V D S S R D R N K P F K F M L G K Q E V I R G W E E G V
A Q M S V G Q R A K L T I S P D Y A Y G A T G H P G I I P P H A
T L V F D V E L L K L E
SEQ ID NO: 4, GS linker (SEQ ID NO: 151) nucleotide sequence
TCTGGCGGTGGATCCGGA
SEQ ID NO: 5, GS linker (SEQ ID NO: 151) amino acid sequence
S G G G S G
SEQ ID NO: 6, linker nucleotide sequence (between GS linker (SEQ ID NO: 151) and
Casp 9)
GTCGAC
SEQ ID NO: 7, linker amino acid sequence (between GS linker (SEQ ID NO: 151) and
Casp 9)
VD
SEQ ID NO: 8, Casp 9 (truncated) nucleotide sequence
GGATTTGGTGATGTCGGTGCTCTTGAGAGTTTGAGGGGAAATGCAGATTTGGCTTA
CATCCTGAGCATGGAGCCCTGTGGCCACTGCCTCATTATCAACAATGTGAACTTCT
GCCGTGAGTCCGGGCTCCGCACCCGCACTGGCTCCAACATCGACTGTGAGAAGTT
GCGGCGTCGCTTCTCCTCGCTGCATTTCATGGTGGAGGTGAAGGGCGACCTGACT
GCCAAGAAAATGGTGCTGGCTTTGCTGGAGCTGGCGCAGCAGGACCACGGTGCTC
TGGACTGCTGCGTGGTGGTCATTCTCTCTCACGGCTGTCAGGCCAGCCACCTGCA
GTTCCCAGGGGCTGTCTACGGCACAGATGGATGCCCTGTGTCGGTCGAGAAGATT
GTGAACATCTTCAATGGGACCAGCTGCCCCAGCCTGGGAGGGAAGCCCAAGCTCT
TTTTCATCCAGGCCTGTGGTGGGGAGCAGAAAGACCATGGGTTTGAGGTGGCCTC
CACTTCCCCTGAAGACGAGTCCCCTGGCAGTAACCCCGAGCCAGATGCCACCCCG
TTCCAGGAAGGTTTGAGGACCTTCGACCAGCTGGACGCCATATCTAGTTTGCCCAC
ACCCAGTGACATCTTTGTGTCCTACTCTACTTTCCCAGGTTTTGTTTCCTGGAGGGA
CCCCAAGAGTGGCTCCTGGTACGTTGAGACCCTGGACGACATCTTTGAGCAGTGG
GCTCACTCTGAAGACCTGCAGTCCCTCCTGCTTAGGGTCGCTAATGCTGTTTCGGT
GAAAGGGATTTATAAACAGATGCCTGGTTGCTTTAATTTCCTCCGGAAAAAACTTTT
CTTTAAAACATCA
SEQ ID NO: 9, caspase-9 (truncated) amino acid sequence-CARD domain deleted
G F G D V G A L E S L R G N A D L A Y I L S M E P C G H C L I I N
N V N F C R E S G L R T R T G S N I D C E K L R R R F S S L H F
M V E V K G D L T A K K M V L A L L E L A Q Q D H G A L D C C V
V V I L S H G C Q A S H L Q F P G A V Y G T D G C P V S V E K I
V N I F N G T S C P S L G G K P K L F F I Q A C G G E Q K D H G
F E V A S T S P E D E S P G S N P E P D A T P F Q E G L R T F D
Q L D A I S S L P T P S D I F V S Y S T F P G F V S W R D P K S
G S W Y V E T L D D I F E Q W A H S E D L Q S L L L R V A N A V
S V K G I Y K Q M P G C F N F L R K K L F F K T S
SEQ ID NO: 10, linker nucleotide sequence (between caspase-9 and 2A)
GCTAGCAGA
SEQ ID NO: 11, linker amino acid sequence (between caspase-9 and 2A)
ASR
SEQ ID NO: 12, Thosea asigna virus-2A from capsid protein precursor nucleotide
sequence
GCCGAGGGCAGGGGAAGTCTTCTAACATGCGGGGACGTGGAGGAAAATCCCGGG
CCC
SEQ ID NO: 13, Thosea asigna virus-2A from capsid protein precursor amino acid
sequence
A E G R G S L L T C G D V E E N P G P
SEQ ID NO: 14, human CD19 (Δ cytoplasmic domain) nucleotide sequence
(transmembrane domain in bold)
ATGCCACCTCCTCGCCTCCTCTTCTTCCTCCTCTTCCTCACCCCCATGGAAGTCAG
GCCCGAGGAACCTCTAGTGGTGAAGGTGGAAGAGGGAGATAACGCTGTGCTGCAG
TGCCTCAAGGGGACCTCAGATGGCCCCACTCAGCAGCTGACCTGGTCTCGGGAGT
CCCCGCTTAAACCCTTCTTAAAACTCAGCCTGGGGCTGCCAGGCCTGGGAATCCAC
ATGAGGCCCCTGGCCATCTGGCTTTTCATCTTCAACGTCTCTCAACAGATGGGGGG
CTTCTACCTGTGCCAGCCGGGGCCCCCCTCTGAGAAGGCCTGGCAGCCTGGCTG
GACAGTCAATGTGGAGGGCAGCGGGGAGCTGTTCCGGTGGAATGTTTCGGACCTA
GGTGGCCTGGGCTGTGGCCTGAAGAACAGGTCCTCAGAGGGCCCCAGCTCCCCTT
CCGGGAAGCTCATGAGCCCCAAGCTGTATGTGTGGGCCAAAGACCGCCCTGAGAT
CTGGGAGGGAGAGCCTCCGTGTCTCCCACCGAGGGACAGCCTGAACCAGAGCCT
CAGCCAGGACCTCACCATGGCCCCTGGCTCCACACTCTGGCTGTCCTGTGGGGTA
CCCCCTGACTCTGTGTCCAGGGGCCCCCTCTCCTGGACCCATGTGCACCCCAAGG
GGCCTAAGTCATTGCTGAGCCTAGAGCTGAAGGACGATCGCCCGGCCAGAGATAT
GTGGGTAATGGAGACGGGTCTGTTGTTGCCCCGGGCCACAGCTCAAGACGCTGGA
AAGTATTATTGTCACCGTGGCAACCTGACCATGTCATTCCACCTGGAGATCACTGCT
CGGCCAGTACTATGGCACTGGCTGCTGAGGACTGGTGGCTGGAAGGTCTCAGCTG
TGACTTTGGCTTATCTGATCTTCTGCCTGTGTTCCCTTGTGGGCATTCTTCATCTTC
AAAGAGCCCTGGTCCTGAGGAGGAAAAGAAAGCGAATGACTGACCCCACCAGGAG
ATTC
SEQ ID NO: 15, human CD19 (Δ cytoplasmic domain) amino acid sequence
M P P P R L L F F L L F L T P M E V R P E E P L V V K V E E G D
N A V L Q C L K G T S D G P T Q Q L T W S R E S P L K P F L K L
S L G L P G L G I H M R P L A I W L F I F N V S Q Q M G G F Y L
C Q P G P P S E K A W Q P G W T V N V E G S G E L F R W N V S
D L G G L G C G L K N R S S E G P S S P S G K L M S P K L Y V
W A K D R P E I W E G E P P C L P P R D S L N Q S L S Q D L T M
A P G S T L W L S C G V P P D S V S R G P L S W T H V H P K G
P K S L L S L E L K D D R P A R D M W V M E T G L L L P R A T A
Q D A G K Y Y C H R G N L T M S F H L E I T A R P V L W H W L L
R T G G W K V S A V T L A Y L I F C L C S L V G I L H L Q R A L V
L R R K R K R M T D P T R R F
SEQ ID NO: 16, 3′LTR nucleotide sequence
TGAAAGACCCCACCTGTAGGTTTGGCAAGCTAGCTTAAGTAACGCCATTTTGCAAG
GCATGGAAAAATACATAACTGAGAATAGAGAAGTTCAGATCAAGGTCAGGAACAGA
TGGAACAGCTGAATATGGGCCAAACAGGATATCTGTGGTAAGCAGTTCCTGCCCCG
GCTCAGGGCCAAGAACAGATGGAACAGCTGAATATGGGCCAAACAGGATATCTGT
GGTAAGCAGTTCCTGCCCCGGCTCAGGGCCAAGAACAGATGGTCCCCAGATGCGG
TCCAGCCCTCAGCAGTTTCTAGAGAACCATCAGATGTTTCCAGGGTGCCCCAAGGA
CCTGAAATGACCCTGTGCCTTATTTGAACTAACCAATCAGTTCGCTTCTCGCTTCTG
TTCGCGCGCTTCTGCTCCCCGAGCTCAATAAAAGAGCCCACAACCCCTCACTCGG
GGCGCCAGTCCTCCGATTGACTGAGTCGCCCGGGTACCCGTGTATCCAATAAACC
CTCTTGCAGTTGCATCCGACTTGTGGTCTCGCTGTTCCTTGGGAGGGTCTCCTCTG
AGTGATTGACTACCCGTCAGCGGGGGTCTTTCA
SEQ ID NO: 17, Expression vector construct nucleotide sequence-nucleotide
sequence coding for the chimeric protein and 5′ and 3′ LTR sequences, and additional
vector sequence.
TGAAAGACCCCACCTGTAGGTTTGGCAAGCTAGCTTAAGTAACGCCATTTTGCAAG
GCATGGAAAAATACATAACTGAGAATAGAAAAGTTCAGATCAAGGTCAGGAACAGA
TGGAACAGCTGAATATGGGCCAAACAGGATATCTGTGGTAAGCAGTTCCTGCCCCG
GCTCAGGGCCAAGAACAGATGGAACAGCTGAATATGGGCCAAACAGGATATCTGT
GGTAAGCAGTTCCTGCCCCGGCTCAGGGCCAAGAACAGATGGTCCCCAGATGCGG
TCCAGCCCTCAGCAGTTTCTAGAGAACCATCAGATGTTTCCAGGGTGCCCCAAGGA
CCTGAAATGACCCTGTGCCTTATTTGAACTAACCAATCAGTTCGCTTCTCGCTTCTG
TTCGCGCGCTTATGCTCCCCGAGCTCAATAAAAGAGCCCACAACCCCTCACTCGGG
GCGCCAGTCCTCCGATTGACTGAGTCGCCCGGGTACCCGTGTATCCAATAAACCCT
CTTGCAGTTGCATCCGACTTGTGGTCTCGCTGTTCCTTGGGAGGGTCTCCTCTGAG
TGATTGACTACCCGTCAGCGGGGGTCTTTCATTTGGGGGCTCGTCCGGGATCGGG
AGACCCCTGCCCAGGGACCACCGACCCACCACCGGGAGGTAAGCTGGCCAGCAA
CTTATCTGTGTCTGTCCGATTGTCTAGTGTCTATGACTGATTTTATGCGCCTGCGTC
GGTACTAGTTAGCTAACTAGCTCTGTATCTGGCGGACCCGTGGTGGAACTGACGAG
TTCGGAACACCCGGCCGCAACCCTGGGAGACGTCCCAGGGACTTCGGGGGCCGT
TTTTGTGGCCCGACCTGAGTCCTAAAATCCCGATCGTTTAGGACTCTTTGGTGCAC
CCCCCTTAGAGGAGGGATATGTGGTTCTGGTAGGAGACGAGAACCTAAAACAGTTC
CCGCCTCCGTCTGAATTTTTGCTTTCGGTTTGGGACCGAAGCCGCGCCGCGCGTC
TTGTCTGCTGCAGCATCGTTCTGTGTTGTCTCTGTCTGACTGTGTTTCTGTATTTGT
CTGAAAATATGGGCCCGGGCTAGCCTGTTACCACTCCCTTAAGTTTGACCTTAGGT
CACTGGAAAGATGTCGAGCGGATCGCTCACAACCAGTCGGTAGATGTCAAGAAGA
GACGTTGGGTTACCTTCTGCTCTGCAGAATGGCCAACCTTTAACGTCGGATGGCCG
CGAGACGGCACCTTTAACCGAGACCTCATCACCCAGGTTAAGATCAAGGTCTTTTC
ACCTGGCCCGCATGGACACCCAGACCAGGTGGGGTACATCGTGACCTGGGAAGC
CTTGGCTTTTGACCCCCCTCCCTGGGTCAAGCCCTTTGTACACCCTAAGCCTCCGC
CTCCTCTTCCTCCATCCGCCCCGTCTCTCCCCCTTGAACCTCCTCGTTCGACCCCG
CCTCGATCCTCCCTTTATCCAGCCCTCACTCCTTCTCTAGGCGCCCCCATATGGCC
ATATGAGATCTTATATGGGGCACCCCCGCCCCTTGTAAACTTCCCTGACCCTGACA
TGACAAGAGTTACTAACAGCCCCTCTCTCCAAGCTCACTTACAGGCTCTCTACTTAG
TCCAGCACGAAGTCTGGAGACCTCTGGCGGCAGCCTACCAAGAACAACTGGACCG
ACCGGTGGTACCTCACCCTTACCGAGTCGGCGACACAGTGTGGGTCCGCCGACAC
CAGACTAAGAACCTAGAACCTCGCTGGAAAGGACCTTACACAGTCCTGCTGACCAC
CCCCACCGCCCTCAAAGTAGACGGCATCGCAGCTTGGATACACGCCGCCCACGTG
AAGGCTGCCGACCCCGGGGGTGGACCATCCTCTAGACTGCCATGCTCGAGGGAGT
GCAGGTGGAAACCATCTCCCCAGGAGACGGGCGCACCTTCCCCAAGCGCGGCCA
GACCTGCGTGGTGCACTACACCGGGATGCTTGAAGATGGAAAGAAAGTTGATTCCT
CCCGGGACAGAAACAAGCCCTTTAAGTTTATGCTAGGCAAGCAGGAGGTGATCCG
AGGCTGGGAAGAAGGGGTTGCCCAGATGAGTGTGGGTCAGAGAGCCAAACTGACT
ATATCTCCAGATTATGCCTATGGTGCCACTGGGCACCCAGGCATCATCCCACCACA
TGCCACTCTCGTCTTCGATGTGGAGCTTCTAAAACTGGAATCTGGCGGTGGATCCG
GAGTCGACGGATTTGGTGATGTCGGTGCTCTTGAGAGTTTGAGGGGAAATGCAGAT
TTGGCTTACATCCTGAGCATGGAGCCCTGTGGCCACTGCCTCATTATCAACAATGT
GAACTTCTGCCGTGAGTCCGGGCTCCGCACCCGCACTGGCTCCAACATCGACTGT
GAGAAGTTGCGGCGTCGCTTCTCCTCGCTGCATTTCATGGTGGAGGTGAAGGGCG
ACCTGACTGCCAAGAAAATGGTGCTGGCTTTGCTGGAGCTGGCGCAGCAGGACCA
CGGTGCTCTGGACTGCTGCGTGGTGGTCATTCTCTCTCACGGCTGTCAGGCCAGC
CACCTGCAGTTCCCAGGGGCTGTCTACGGCACAGATGGATGCCCTGTGTCGGTCG
AGAAGATTGTGAACATCTTCAATGGGACCAGCTGCCCCAGCCTGGGAGGGAAGCC
CAAGCTCTTTTTCATCCAGGCCTGTGGTGGGGAGCAGAAAGACCATGGGTTTGAG
GTGGCCTCCACTTCCCCTGAAGACGAGTCCCCTGGCAGTAACCCCGAGCCAGATG
CCACCCCGTTCCAGGAAGGTTTGAGGACCTTCGACCAGCTGGACGCCATATCTAGT
TTGCCCACACCCAGTGACATCTTTGTGTCCTACTCTACTTTCCCAGGTTTTGTTTCC
TGGAGGGACCCCAAGAGTGGCTCCTGGTACGTTGAGACCCTGGACGACATCTTTG
AGCAGTGGGCTCACTCTGAAGACCTGCAGTCCCTCCTGCTTAGGGTCGCTAATGCT
GTTTCGGTGAAAGGGATTTATAAACAGATGCCTGGTTGCTTTAATTTCCTCCGGAAA
AAACTTTTCTTTAAAACATCAGCTAGCAGAGCCGAGGGCAGGGGAAGTCTTCTAAC
ATGCGGGGACGTGGAGGAAAATCCCGGGCCCATGCCACCTCCTCGCCTCCTCTTC
TTCCTCCTCTTCCTCACCCCCATGGAAGTCAGGCCCGAGGAACCTCTAGTGGTGAA
GGTGGAAGAGGGAGATAACGCTGTGCTGCAGTGCCTCAAGGGGACCTCAGATGGC
CCCACTCAGCAGCTGACCTGGTCTCGGGAGTCCCCGCTTAAACCCTTCTTAAAACT
CAGCCTGGGGCTGCCAGGCCTGGGAATCCACATGAGGCCCCTGGCCATCTGGCTT
TTCATCTTCAACGTCTCTCAACAGATGGGGGGCTTCTACCTGTGCCAGCCGGGGCC
CCCCTCTGAGAAGGCCTGGCAGCCTGGCTGGACAGTCAATGTGGAGGGCAGCGG
GGAGCTGTTCCGGTGGAATGTTTCGGACCTAGGTGGCCTGGGCTGTGGCCTGAAG
AACAGGTCCTCAGAGGGCCCCAGCTCCCCTTCCGGGAAGCTCATGAGCCCCAAGC
TGTATGTGTGGGCCAAAGACCGCCCTGAGATCTGGGAGGGAGAGCCTCCGTGTCT
CCCACCGAGGGACAGCCTGAACCAGAGCCTCAGCCAGGACCTCACCATGGCCCCT
GGCTCCACACTCTGGCTGTCCTGTGGGGTACCCCCTGACTCTGTGTCCAGGGGCC
CCCTCTCCTGGACCCATGTGCACCCCAAGGGGCCTAAGTCATTGCTGAGCCTAGA
GCTGAAGGACGATCGCCCGGCCAGAGATATGTGGGTAATGGAGACGGGTCTGTTG
TTGCCCCGGGCCACAGCTCAAGACGCTGGAAAGTATTATTGTCACCGTGGCAACCT
GACCATGTCATTCCACCTGGAGATCACTGCTCGGCCAGTACTATGGCACTGGCTGC
TGAGGACTGGTGGCTGGAAGGTCTCAGCTGTGACTTTGGCTTATCTGATCTTCTGC
CTGTGTTCCCTTGTGGGCATTCTTCATCTTCAAAGAGCCCTGGTCCTGAGGAGGAA
AAGAAAGCGAATGACTGACCCCACCAGGAGATTCTAACGCGTCATCATCGATCCGG
ATTAGTCCAATTTGTTAAAGACAGGATATCAGTGGTCCAGGCTCTAGTTTTGACTCA
ACAATATCACCAGCTGAAGCCTATAGAGTACGAGCCATAGATAAAATAAAAGATTTT
ATTTAGTCTCCAGAAAAAGGGGGGAATGAAAGACCCCACCTGTAGGTTTGGCAAGC
TAGCTTAAGTAACGCCATTTTGCAAGGCATGGAAAAATACATAACTGAGAATAGAGA
AGTTCAGATCAAGGTCAGGAACAGATGGAACAGCTGAATATGGGCCAAACAGGATA
TCTGTGGTAAGCAGTTCCTGCCCCGGCTCAGGGCCAAGAACAGATGGAACAGCTG
AATATGGGCCAAACAGGATATCTGTGGTAAGCAGTTCCTGCCCCGGCTCAGGGCC
AAGAACAGATGGTCCCCAGATGCGGTCCAGCCCTCAGCAGTTTCTAGAGAACCATC
AGATGTTTCCAGGGTGCCCCAAGGACCTGAAATGACCCTGTGCCTTATTTGAACTA
ACCAATCAGTTCGCTTCTCGCTTCTGTTCGCGCGCTTCTGCTCCCCGAGCTCAATA
AAAGAGCCCACAACCCCTCACTCGGGGCGCCAGTCCTCCGATTGACTGAGTCGCC
CGGGTACCCGTGTATCCAATAAACCCTCTTGCAGTTGCATCCGACTTGTGGTCTCG
CTGTTCCTTGGGAGGGTCTCCTCTGAGTGATTGACTACCCGTCAGCGGGGGTCTTT
CACACATGCAGCATGTATCAAAATTAATTTGGTTTTTTTTCTTAAGTATTTACATTAAA
TGGCCATAGTACTTAAAGTTACATTGGCTTCCTTGAAATAAACATGGAGTATTCAGA
ATGTGTCATAAATATTTCTAATTTTAAGATAGTATCTCCATTGGCTTTCTACTTTTTCT
TTTATTTTTTTTTGTCCTCTGTCTTCCATTTGTTGTTGTTGTTGTTTGTTTGTTTGTTTG
GTTGGTTGGTTGGTTAATTTTTTTTTAAAGATCCTACACTATAGTTCAAGCTAGACTA
TTAGCTACTCTGTAACCCAGGGTGACCTTGAAGTCATGGGTAGCCTGCTGTTTTAG
CCTTCCCACATCTAAGATTACAGGTATGAGCTATCATTTTTGGTATATTGATTGATTG
ATTGATTGATGTGTGTGTGTGTGATTGTGTTTGTGTGTGTGACTGTGAAAATGTGTG
TATGGGTGTGTGTGAATGTGTGTATGTATGTGTGTGTGTGAGTGTGTGTGTGTGTG
TGTGCATGTGTGTGTGTGTGACTGTGTCTATGTGTATGACTGTGTGTGTGTGTGTGT
GTGTGTGTGTGTGTGTGTGTGTGTGTGTGTTGTGAAAAAATATTCTATGGTAGTGAG
AGCCAACGCTCCGGCTCAGGTGTCAGGTTGGTTTTTGAGACAGAGTCTTTCACTTA
GCTTGGAATTCACTGGCCGTCGTTTTACAACGTCGTGACTGGGAAAACCCTGGCGT
TACCCAACTTAATCGCCTTGCAGCACATCCCCCTTTCGCCAGCTGGCGTAATAGCG
AAGAGGCCCGCACCGATCGCCCTTCCCAACAGTTGCGCAGCCTGAATGGCGAATG
GCGCCTGATGCGGTATTTTCTCCTTACGCATCTGTGCGGTATTTCACACCGCATAT
GGTGCACTCTCAGTACAATCTGCTCTGATGCCGCATAGTTAAGCCAGCCCCGACAC
CCGCCAACACCCGCTGACGCGCCCTGACGGGCTTGTCTGCTCCCGGCATCCGCTT
ACAGACAAGCTGTGACCGTCTCCGGGAGCTGCATGTGTCAGAGGTTTTCACCGTCA
TCACCGAAACGCGCGATGACGAAAGGGCCTCGTGATACGCCTATTTTTATAGGTTA
ATGTCATGATAATAATGGTTTCTTAGACGTCAGGTGGCACTTTTCGGGGAAATGTGC
GCGGAACCCCTATTTGTTTATTTTTCTAAATACATTCAAATATGTATCCGCTCATGAG
ACAATAACCCTGATAAATGCTTCAATAATATTGAAAAAGGAAGAGTATGAGTATTCAA
CATTTCCGTGTCGCCCTTATTCCCTTTTTTGCGGCATTTTGCCTTCCTGTTTTTGCTC
ACCCAGAAACGCTGGTGAAAGTAAAAGATGCTGAAGATCAGTTGGGTGCACGAGT
GGGTTACATCGAACTGGATCTCAACAGCGGTAAGATCCTTGAGAGTTTTCGCCCCG
AAGAACGTTTTCCAATGATGAGCACTTTTAAAGTTCTGCTATGTGGCGCGGTATTAT
CCCGTATTGACGCCGGGCAAGAGCAACTCGGTCGCCGCATACACTATTCTCAGAAT
GACTTGGTTGAGTACTCACCAGTCACAGAAAAGCATCTTACGGATGGCATGACAGT
AAGAGAATTATGCAGTGCTGCCATAACCATGAGTGATAACACTGCGGCCAACTTAC
TTCTGACAACGATCGGAGGACCGAAGGAGCTAACCGCTTTTTTGCACAACATGGGG
GATCATGTAACTCGCCTTGATCGTTGGGAACCGGAGCTGAATGAAGCCATACCAAA
CGACGAGCGTGACACCACGATGCCTGTAGCAATGGCAACAACGTTGCGCAAACTA
TTAACTGGCGAACTACTTACTCTAGCTTCCCGGCAACAATTAATAGACTGGATGGAG
GCGGATAAAGTTGCAGGACCACTTCTGCGCTCGGCCCTTCCGGCTGGCTGGTTTA
TTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATTAA
GCATTGGTAACTGTCAGACCAAGTTTACTCATATATACTTTAGATTGATTTAAAACTT
CATTTTTAATTTAAAAGGATCTAGGTGAAGATCCTTTTTGATAATCTCATGACCAAAA
TCCCTTAACGTGAGTTTTCGTTCCACTGAGCGTCAGACCCCGTAGAAAAGATCAAA
GGATCTTCTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCTTTTTCC
GAAGGTAACTGGCTTCAGCAGAGCGCAGATACCAAATACTGTCCTTCTAGTGTAGC
CGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTT
GGACTCAAGACGATAGTTACCGGATAAGGCGCAGCGGTCGGGCTGAACGGGGGG
TTCGTGCACACAGCCCAGCTTGGAGCGAACGACCTACACCGAACTGAGATACCTAC
AGCGTGAGCATTGAGAAAGCGCCACGCTTCCCGAAGGGAGAAAGGCGGACAGGTA
TCCGGTAAGCGGCAGGGTCGGAACAGGAGAGCGCACGAGGGAGCTTCCAGGGGG
AAACGCCTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGCAACGC
GGCCTTTTTACGGTTCCTGGCCTTTTGCTGGCCTTTTGCTCACATGTTCTTTCCTGC
GTTATCCCCTGATTCTGTGGATAACCGTATTACCGCCTTTGAGTGAGCTGATACCG
CTCGCCGCAGCCGAACGACCGAGCGCAGCGAGTCAGTGAGCGAGGAAGCGGAAG
AGCGCCCAATACGCAAACCGCCTCTCCCCGCGCGTTGGCCGATTCATTAATGCAG
CTGGCACGACAGGTTTCCCGACTGGAAAGCGGGCAGTGAGCGCAACGCAATTAAT
GTGAGTTAGCTCACTCATTAGGCACCCCAGGCTTTACACTTTATGCTTCCGGCTCG
TATGTTGTGTGGAATTGTGAGCGGATAACAATTTCACACAGGAAACAGCTATGACCA
TGATTACGCCAAGCTTTGCTCTTAGGAGTTTCCTAATACATCCCAAACTCAAATATAT
AAAGCATTTGACTTGTTCTATGCCCTAGGGGGCGGGGGGAAGCTAAGCCAGCTTTT
TTTAACATTTAAAATGTTAATTCCATTTTAAATGCACAGATGTTTTTATTTCATAAGGG
TTTCAATGTGCATGAATGCTGCAATATTCCTGTTACCAAAGCTAGTATAAATAAAAAT
AGATAAACGTGGAAATTACTTAGAGTTTCTGTCATTAACGTTTCCTTCCTCAGTTGAC
AACATAAATGCGCTGCTGAGCAAGCCAGTTTGCATCTGTCAGGATCAATTTCCCATT
ATGCCAGTCATATTAATTACTAGTCAATTAGTTGATTTTTATTTTTGACATATACATGT
GAA
SEQ ID NO: 18, (nucleotide sequence of F_{v′}Fᵥₗₛ with XhoI/SalI linkers, (wobbled codons
lowercase in F_{v′}))
ctcgagGGcGTcCAaGTcGAaACcATtagtCCcGGcGAtGGcaGaACaTTtCCtAAaaGgGGaC
AaACaTGtGTcGTcCAtTAtACaGGcATGtTgGAgGAcGGcAAaAAgGTgGAcagtagtaGaGA
tcGcAAtAAaCCtTTcAAaTTcATGtTgGGaAAaCAaGAaGTcATtaGgGGaTGGGAgGAgGG
cGTgGCtCAaATGtccGTcGGcCAacGcGCtAAgCTcACcATcagcCCcGAcTAcGCaTAcGG
cGCtACcGGaCAtCCcGGaATtATtCCcCCtCAcGCtACctTgGTgTTtGAcGTcGAaCTgtTgA
AgCTcGAagtcgagggagtgcaggtggaaaccatctccccaggagacgggcgcaccttccccaagcgcggccaga
cctgcgtggtgcactacaccgggatgcttgaagatggaaagaaagttgattcctcccgggacagaaacaagccctttaagt
ttatgctaggcaagcaggaggtgatccgaggctgggaagaaggggttgcccagatgagtgtgggtcagagagccaaac
tgactatatctccagattatgcctatggtgccactgggcacccaggcatcatcccaccacatgccactctcgtcttcgatgtgg
agcttctaaaactggaatctggcggtggatccggagtcgag
SEQ ID NO: 19, (F_{V′}F_{VLS} amino acid sequence)
GlyValGlnValGluThrlIeSerProGlyAspGlyArgThrPheProLysArgGlyGlnThrCysValValHis
TyrThrGlyMetLeuGluAspGlyLysLysValAspSerSerArgAspArgAsnLysProPheLysPheMetLeu
GlyLysGlnGluValIleArgGlyTrpGluGluGlyValAlaGlnMetSerValGlyGlnArgAlaLysLeuThr
IleSerProAspTyrAlaTyrGlyAlaThrGlyHisProGlyIleIleProProHisAlaThrLeuValPheAsp
ValGluLeuLeuLysLeuGlu (ValGlu)
GlyValGlnValGluThrlIeSerProGlyAspGlyArgThrPheProLysArgGlyGlnThrCysValValHis
TyrThrGlyMetLeuGluAspGlyLysLysValAspSerSerArgAspArgAsnLysProPheLysPheMetLeu
GlyLysGlnGluValIleArgGlyTrpGluGluGlyValAlaGlnMetSerValGlyGlnArgAlaLysLeuThr
IleSerProAspTyrAlaTyrGlyAlaThrGlyHisProGlyIleIleProProHisAlaThrLeuValPheAsp
ValGluLeuLeuLysLeuGlu-SerGlyGlyGlySerGly
SEQ ID NO: 20, FKBP12v36 (res. 2-108)
SGGGSGLinker (6 aa)(SEQ ID NO: 239)
ΔCasp9 (res. 135-416)
ATGCTCGAGGGAGTGCAGGTGGAgACtATCTCCCCAGGAGACGGGCGCACCTTCC
CCAAGCGCGGCCAGACCTGCGTGGTGCACTACACCGGGATGCTTGAAGATGGAAA
GAAAGTTGATTCCTCCCGGGACAGAAACAAGCCCTTTAAGTTTATGCTAGGCAAGC
AGGAGGTGATCCGAGGCTGGGAAGAAGGGGTTGCCCAGATGAGTGTGGGTCAGA
GAGCCAAACTGACTATATCTCCAGATTATGCCTATGGTGCCACTGGGCACCCAGGC
ATCATCCCACCACATGCCACTCTCGTCTTCGATGTGGAGCTTCTAAAACTGGAATCT
GGCGGTGGATCCGGAGTCGACGGATTTGGTGATGTCGGTGCTCTTGAGAGTTTGA
GGGGAAATGCAGATTTGGCTTACATCCTGAGCATGGAGCCCTGTGGCCACTGCCT
CATTATCAACAATGTGAACTTCTGCCGTGAGTCCGGGCTCCGCACCCGCACTGGCT
CCAACATCGACTGTGAGAAGTTGCGGCGTCGCTTCTCCTCGCTGCATTTCATGGTG
GAGGTGAAGGGCGACCTGACTGCCAAGAAAATGGTGCTGGCTTTGCTGGAGCTGG
CGCgGCAGGACCACGGTGCTCTGGACTGCTGCGTGGTGGTCATTCTCTCTCACGG
CTGTCAGGCCAGCCACCTGCAGTTCCCAGGGGCTGTCTACGGCACAGATGGATGC
CCTGTGTCGGTCGAGAAGATTGTGAACATCTTCAATGGGACCAGCTGCCCCAGCCT
GGGAGGGAAGCCCAAGCTCTTTTTCATCCAGGCCTGTGGTGGGGAGCAGAAAGAC
CATGGGTTTGAGGTGGCCTCCACTTCCCCTGAAGACGAGTCCCCTGGCAGTAACC
CCGAGCCAGATGCCACCCCGTTCCAGGAAGGTTTGAGGACCTTCGACCAGCTGGA
CGCCATATCTAGTTTGCCCACACCCAGTGACATCTTTGTGTCCTACTCTACTTTCCC
AGGTTTTGTTTCCTGGAGGGACCCCAAGAGTGGCTCCTGGTACGTTGAGACCCTG
GACGACATCTTTGAGCAGTGGGCTCACTCTGAAGACCTGCAGTCCCTCCTGCTTAG
GGTCGCTAATGCTGTTTCGGTGAAAGGGATTTATAAACAGATGCCTGGTTGCTTTAA
TTCCTCCGGAAAAAACTTTTCTTTAAAACATCA
SEQ ID NO: 21, FKBP12v36 (res. 2-108)
G V Q V E T I S P G D G R T F P K R G Q T C V V H Y T G M L E D
G K K V D S S R D R N K P F K F M L G K Q E V I R G W E E G V
A Q M S V G Q R A K L T I S P D Y A Y G A T G H P G I I P P H A
T L V F D V E L L K L E
SEQ ID NO: 22, ΔCasp9 (res. 135-416)
G F G D V G A L E S L R G N A D L A Y I L S M E P C G H C L I I N
N V N F C R E S G L R T R T G S N I D C E K L R R R F S S L H F
M V E V K G D L T A K K M V L A L L E L A R Q D H G A L D C C V
V V I L S H G C Q A S H L Q F P G A V Y G T D G C P V S V E K I
V N I F N G T S C P S L G G K P K L F F I Q A C G G E Q K D H G
F E V A S T S P E D E S P G S N P E P D A T P F Q E G L R T F D
Q L D A I S S L P T P S D I F V S Y S T F P G F V S W R D P K S
G S W Y V E T L D D I F E Q W A H S E D L Q S L L L R V A N A V
S V K G I Y K Q M P G C F N F L R K K L F F K T S
SEQ ID NO: 23, ΔCasp9 (res. 135-416) D330A, nucleotide sequence
GGATTTGGTGATGTCGGTGCTCTTGAGAGTTTGAGGGGAAATGCAGATTTGGCTTA
CATCCTGAGCATGGAGCCCTGTGGCCACTGCCTCATTATCAACAATGTGAACTTCT
GCCGTGAGTCCGGGCTCCGCACCCGCACTGGCTCCAACATCGACTGTGAGAAGTT
GCGGCGTCGCTTCTCCTCGCTGCATTTCATGGTGGAGGTGAAGGGCGACCTGACT
GCCAAGAAAATGGTGCTGGCTTTGCTGGAGCTGGCGCgGCAGGACCACGGTGCTC
TGGACTGCTGCGTGGTGGTCATTCTCTCTCACGGCTGTCAGGCCAGCCACCTGCA
GTTCCCAGGGGCTGTCTACGGCACAGATGGATGCCCTGTGTCGGTCGAGAAGATT
GTGAACATCTTCAATGGGACCAGCTGCCCCAGCCTGGGAGGGAAGCCCAAGCTCT
TTTTCATCCAGGCCTGTGGTGGGGAGCAGAAAGACCATGGGTTTGAGGTGGCCTC
CACTTCCCCTGAAGACGAGTCCCCTGGCAGTAACCCCGAGCCAGATGCCACCCCG
TTCCAGGAAGGTTTGAGGACCTTCGACCAGCTGGCCGCCATATCTAGTTTGCCCAC
ACCCAGTGACATCTTTGTGTCCTACTCTACTTTCCCAGGTTTTGTTTCCTGGAGGGA
CCCCAAGAGTGGCTCCTGGTACGTTGAGACCCTGGACGACATCTTTGAGCAGTGG
GCTCACTCTGAAGACCTGCAGTCCCTCCTGCTTAGGGTCGCTAATGCTGTTTCGGT
GAAAGGGATTTATAAACAGATGCCTGGTTGCTTTAATTTCCTCCGGAAAAAACTTTT
CTTTAAAACATCA
SEQ ID NO: 24, ΔCasp9 (res. 135-416) D330A, amino acid sequence
G F G D V G A L E S L R G N A D L A Y I L S M E P C G H C L I I N
N V N F C R E S G L R T R T G S N I D C E K L R R R F S S L H F
M V E V K G D L T A K K M V L A L L E L A R Q D H G A L D C C V
V V I L S H G C Q A S H L Q F P G A V Y G T D G C P V S V E K I
V N I F N G T S C P S L G G K P K L F F I Q A C G G E Q K D H G
F E V A S T S P E D E S P G S N P E P D A T P F Q E G L R T F D
Q L A A I S S L P T P S D I F V S Y S T F P G F V S W R D P K S
G S W Y V E T L D D I F E Q W A H S E D L Q S L L L R V A N A V
S V K G I Y K Q M P G C F N F L R K K L F F K T S
SEQ ID NO: 25, ΔCasp9 (res. 135-416) N405Q nucleotide sequence
GGATTTGGTGATGTCGGTGCTCTTGAGAGTTTGAGGGGAAATGCAGATTTGGCTTA
CATCCTGAGCATGGAGCCCTGTGGCCACTGCCTCATTATCAACAATGTGAACTTCT
GCCGTGAGTCCGGGCTCCGCACCCGCACTGGCTCCAACATCGACTGTGAGAAGTT
GCGGCGTCGCTTCTCCTCGCTGCATTTCATGGTGGAGGTGAAGGGCGACCTGACT
GCCAAGAAAATGGTGCTGGCTTTGCTGGAGCTGGCGCgGCAGGACCACGGTGCTC
TGGACTGCTGCGTGGTGGTCATTCTCTCTCACGGCTGTCAGGCCAGCCACCTGCA
GTTCCCAGGGGCTGTCTACGGCACAGATGGATGCCCTGTGTCGGTCGAGAAGATT
GTGAACATCTTCAATGGGACCAGCTGCCCCAGCCTGGGAGGGAAGCCCAAGCTCT
TTTTCATCCAGGCCTGTGGTGGGGAGCAGAAAGACCATGGGTTTGAGGTGGCCTC
CACTTCCCCTGAAGACGAGTCCCCTGGCAGTAACCCCGAGCCAGATGCCACCCCG
TTCCAGGAAGGTTTGAGGACCTTCGACCAGCTGGACGCCATATCTAGTTTGCCCAC
ACCCAGTGACATCTTTGTGTCCTACTCTACTTTCCCAGGTTTTGTTTCCTGGAGGGA
CCCCAAGAGTGGCTCCTGGTACGTTGAGACCCTGGACGACATCTTTGAGCAGTGG
GCTCACTCTGAAGACCTGCAGTCCCTCCTGCTTAGGGTCGCTAATGCTGTTTCGGT
GAAAGGGATTTATAAACAGATGCCTGGTTGCTTTCAGTTCCTCCGGAAAAAACTTTT
CTTTAAAACATCA
SEQ ID NO: 26, ΔCasp9 (res. 135-416) N405Q amino acid sequence
G F G D V G A L E S L R G N A D L A Y I L S M E P C G H C L I I N
N V N F C R E S G L R T R T G S N I D C E K L R R R F S S L H F
M V E V K G D L T A K K M V L A L L E L A R Q D H G A L D C C V
V V I L S H G C Q A S H L Q F P G A V Y G T D G C P V S V E K I
V N I F N G T S C P S L G G K P K L F F I Q A C G G E Q K D H G
F E V A S T S P E D E S P G S N P E P D A T P F Q E G L R T F D
Q L D A I S S L P T P S D I F V S Y S T F P G F V S W R D P K S
G S W Y V E T L D D I F E Q W A H S E D L Q S L L L R V A N A V
S V K G I Y K Q M P G C F Q F L R K K L F F K T S
SEQ ID NO: 27, ΔCasp9 (res. 135-416) D330A N405Q nucleotide sequence
GGATTTGGTGATGTCGGTGCTCTTGAGAGTTTGAGGGGAAATGCAGATTTGGCTTA
CATCCTGAGCATGGAGCCCTGTGGCCACTGCCTCATTATCAACAATGTGAACTTCT
GCCGTGAGTCCGGGCTCCGCACCCGCACTGGCTCCAACATCGACTGTGAGAAGTT
GCGGCGTCGCTTCTCCTCGCTGCATTTCATGGTGGAGGTGAAGGGCGACCTGACT
GCCAAGAAAATGGTGCTGGCTTTGCTGGAGCTGGCGCgGCAGGACCACGGTGCTC
TGGACTGCTGCGTGGTGGTCATTCTCTCTCACGGCTGTCAGGCCAGCCACCTGCA
GTTCCCAGGGGCTGTCTACGGCACAGATGGATGCCCTGTGTCGGTCGAGAAGATT
GTGAACATCTTCAATGGGACCAGCTGCCCCAGCCTGGGAGGGAAGCCCAAGCTCT
TTTTCATCCAGGCCTGTGGTGGGGAGCAGAAAGACCATGGGTTTGAGGTGGCCTC
CACTTCCCCTGAAGACGAGTCCCCTGGCAGTAACCCCGAGCCAGATGCCACCCCG
TTCCAGGAAGGTTTGAGGACCTTCGACCAGCTGGCCGCCATATCTAGTTTGCCCAC
ACCCAGTGACATCTTTGTGTCCTACTCTACTTTCCCAGGTTTTGTTTCCTGGAGGGA
CCCCAAGAGTGGCTCCTGGTACGTTGAGACCCTGGACGACATCTTTGAGCAGTGG
GCTCACTCTGAAGACCTGCAGTCCCTCCTGCTTAGGGTCGCTAATGCTGTTTCGGT
GAAAGGGATTTATAAACAGATGCCTGGTTGCTTTCAGTTCCTCCGGAAAAAACTTTT
CTTTAAAACATCA
SEQ ID NO: 28, ΔCasp9 (res. 135-416) D330A N405Q amino acid sequence
G F G D V G A L E S L R G N A D L A Y I L S M E P C G H C L I I N
N V N F C R E S G L R T R T G S N I D C E K L R R R F S S L H F
M V E V K G D L T A K K M V L A L L E L A R Q D H G A L D C C V
V V I L S H G C Q A S H L Q F P G A V Y G T D G C P V S V E K I
V N I F N G T S C P S L G G K P K L F F I Q A C G G E Q K D H G
F E V A S T S P E D E S P G S N P E P D A T P F Q E G L R T F D
Q L A A I S S L P T P S D I F V S Y S T F P G F V S W R D P K S
G S W Y V E T L D D I F E Q W A H S E D L Q S L L L R V A N A V
S V K G I Y K Q M P G C F Q F L R K K L F F K T S
SEQ ID NO: 29, FKBPv36 (Fv1) nucleotide sequence
GGCGTTCAAGTAGAAACAATCAGCCCAGGAGACGGAAGGACTTTCCCCAAACGAG
GCCAAACATGCGTAGTTCATTATACTGGGATGCTCGAAGATGGAAAAAAAGTAGAT
AGTAGTAGAGACCGAAACAAACCATTTAAATTTATGTTGGGAAAACAAGAAGTAATA
AGGGGCTGGGAAGAAGGTGTAGCACAAATGTCTGTTGGCCAGCGCGCAAAACTCA
CAATTTCTCCTGATTATGCTTACGGAGCTACCGGCCACCCCGGCATCATACCCCCT
CATGCCACACTGGTGTTTGACGTCGAATTGCTCAAACTGGAA
SEQ ID NO: 30, FKBPv36 (Fv1) amino acid sequence
GVQVETISPGDGRTFPKRGQTCVVHYTGMLEDGKKVDSSRDRNKPFKFMLGKQEVIR
GWEEGVAQMSVGQRAKLTISPDYAYGATGHPGIIPPHATLVFDVELLKLE
SEQ ID NO: 31, FKBPv36 (Fv2) nucleotide sequence
GGaGTgCAgGTgGAgACgATtAGtCCtGGgGAtGGgAGaACcTTtCCaAAgCGcGGtCAgA
CcTGtGTtGTcCAcTAcACcGGtATGCTgGAgGAcGGgAAgAAgGTgGActcTtcacGcGAtCG
cAAtAAgCCtTTcAAgTTcATGcTcGGcAAgCAgGAgGTgATccGGGGgTGGGAgGAgGGc
GTgGCtCAgATGTCgGTcGGgCAaCGaGCgAAgCTtACcATcTCaCCcGAcTAcGCgTAtG
GgGCaACgGGgCAtCCgGGaATtATcCCtCCcCAcGCtACgCTcGTaTTcGAtGTgGAgcTctt
gAAgCTtGag
SEQ ID NO: 32, FKBPv36 (Fv2) amino acid sequence
GVQVETISPGDGRTFPKRGQTCVVHYTGMLEDGKKVDSSRDRNKPFKFMLGKQEVIR
GWEEGVAQMSVGQRAKLTISPDYAYGATGHPGIIPPHATLVFDVELLKLE
SEQ ID NO: 33, ΔCD19 nucleotide sequence
ATGCCCCCTCCTAGACTGCTGTTTTTCCTGCTCTTTCTCACCCCAATGGAAGTTAGA
CCTGAGGAACCACTGGTCGTTAAAGTGGAAGAAGGTGATAATGCTGTCCTCCAATG
CCTTAAAGGGACCAGCGACGGACCAACGCAGCAACTGACTTGGAGCCGGGAGTCC
CCTCTCAAGCCGTTTCTCAAGCTGTCACTTGGCCTGCCAGGTCTTGGTATTCACAT
GCGCCCCCTTGCCATTTGGCTCTTCATATTCAATGTGTCTCAACAAATGGGTGGATT
CTACCTTTGCCAGCCCGGCCCCCCTTCTGAGAAAGCTTGGCAGCCTGGATGGACC
GTCAATGTTGAAGGCTCCGGTGAGCTGTTTAGATGGAATGTGAGCGACCTTGGCG
GACTCGGTTGCGGACTGAAAAATAGGAGCTCTGAAGGACCCTCTTCTCCCTCCGGT
AAGTTGATGTCACCTAAGCTGTACGTGTGGGCCAAGGACCGCCCCGAAATCTGGG
AGGGCGAGCCTCCATGCCTGCCGCCTCGCGATTCACTGAACCAGTCTCTGTCCCA
GGATCTCACTATGGCGCCCGGATCTACTCTTTGGCTGTCTTGCGGCGTTCCCCCAG
ATAGCGTGTCAAGAGGACCTCTGAGCTGGACCCACGTACACCCTAAGGGCCCTAA
GAGCTTGTTGAGCCTGGAACTGAAGGACGACAGACCCGCACGCGATATGTGGGTA
ATGGAGACCGGCCTTCTGCTCCCTCGCGCTACCGCACAGGATGCAGGGAAATACT
ACTGTCATAGAGGGAATCTGACTATGAGCTTTCATCTCGAAATTACAGCACGGCCC
GTTCTTTGGCATTGGCTCCTCCGGACTGGAGGCTGGAAGGTGTCTGCCGTAACACT
CGCTTACTTGATTTTTTGCCTGTGTAGCCTGGTTGGGATCCTGCATCTTCAGCGAG
CCCTTGTATTGCGCCGAAAAAGAAAACGAATGACTGACCCTACACGACGATTCTGA
SEQ ID NO: 34, ΔCD19 amino acid sequence
MPPPRLLFFLLFLTPMEVRPEEPLVVKVEEGDNAVLQCLKGTSDGPTQQLTWSRESPL
KPFLKLSLGLPGLGIHMRPLAIWLFIFNVSQQMGGFYLCQPGPPSEKAWQPGWTVNVE
GSGELFRWNVSDLGGLGCGLKNRSSEGPSSPSGKLMSPKLYVWAKDRPEIWEGEPP
CLPPRDSLNQSLSQDLTMAPGSTLWLSCGVPPDSVSRGPLSWTHVHPKGPKSLLSLEL
KDDRPARDMVVVMETGLLLPRATAQDAGKYYCHRGNLTMSFHLEITARPVLWHWLLRT
GGWKVSAVTLAYLIFCLCSLVGILHLQRALVLRRKRKRMTDPTRRF*
Codon optimized iCasp9-N405Q-2A-ΔCD19 sequence: (the .co following the name of a
nucleotide sequence indicates that it is codon optimized (or the amino acid sequence
coded by the codon-optimized nucleotide sequence).
SEQ-ID NO: 35, FKBPv36.co (Fv3) nucleotide sequence
ATGCTGGAGGGAGTGCAGGTGGAGACTATTAGCCCCGGAGATGGCAGAACATTCC
CCAAAAGAGGACAGACTTGCGTCGTGCATTATACTGGAATGCTGGAAGACGGCAA
GAAGGTGGACAGCAGCCGGGACCGAAACAAGCCCTTCAAGTTCATGCTGGGGAAG
CAGGAAGTGATCCGGGGCTGGGAGGAAGGAGTCGCACAGATGTCAGTGGGACAG
AGGGCCAAACTGACTATTAGCCCAGACTACGCTTATGGAGCAACCGGCCACCCCG
GGATCATTCCCCCTCATGCTACACTGGTCTTCGATGTGGAGCTGCTGAAGCTGGAA
SEQ ID NO: 36, FKBPv36.co (Fv3) amino acid sequence
MLEGVQVETISPGDGRTFPKRGQTCVVHYTGMLEDGKKVDSSRDRNKPFKFMLGKQE
VIRGWEEGVAQMSVGQRAKLTISPDYAYGATGHPGIIPPHATLVFDVELLKLE
SEQ ID NO: 37, Linker.co nucleotide sequence
AGCGGAGGAGGATCCGGA
SEQ ID NO: 38, Linker.co amino acid sequence
SGGGSG
SEQ IDNO: 39, caspase-9.co nucleotide sequence
GTGGACGGGTTTGGAGATGTGGGAGCCCTGGAATCCCTGCGGGGCAATGCCGAT
CTGGCTTACATCCTGTCTATGGAGCCTTGCGGCCACTGTCTGATCATTAACAATGT
GAACTTCTGCAGAGAGAGCGGGCTGCGGACCAGAACAGGATCCAATATTGACTGT
GAAAAGCTGCGGAGAAGGTTCTCTAGTCTGCACTTTATGGTCGAGGTGAAAGGCGA
TCTGACCGCTAAGAAAATGGTGCTGGCCCTGCTGGAACTGGCTCGGCAGGACCAT
GGGGCACTGGATTGCTGCGTGGTCGTGATCCTGAGTCACGGCTGCCAGGCTTCAC
ATCTGCAGTTCCCTGGGGCAGTCTATGGAACTGACGGCTGTCCAGTCAGCGTGGA
GAAGATCGTGAACATCTTCAACGGCACCTCTTGCCCAAGTCTGGGCGGGAAGCCC
AAACTGTTCTTTATTCAGGCCTGTGGAGGCGAGCAGAAAGATCACGGCTTCGAAGT
GGCTAGCACCTCCCCCGAGGACGAATCACCTGGAAGCAACCCTGAGCCAGATGCA
ACCCCCTTCCAGGAAGGCCTGAGGACATTTGACCAGCTGGATGCCATCTCAAGCCT
GCCCACACCTTCTGACATTTTCGTCTCTTACAGTACTTTCCCTGGATTTGTGAGCTG
GCGCGATCCAAAGTCAGGCAGCTGGTACGTGGAGACACTGGACGATATCTTTGAG
CAGTGGGCCCATTCTGAAGACCTGCAGAGTCTGCTGCTGCGAGTGGCCAATGCTG
TCTCTGTGAAGGGGATCTACAAACAGATGCCAGGATGCTTCCAGTTTCTGAGAAAG
AAACTGTTCTTTAAGACCTCCGCATCTAGGGCC
SEQ ID NO: 40, caspase-9.co amino acid sequence
VDGFGDVGALESLRGNADLAYILSMEPCGHCLIINNVNFCRESGLRTRTGSNIDCEKLR
RRFSSLHFMVEVKGDLTAKKMVLALLELARQDHGALDCCVVVILSHGCQASHLQFPGA
VYGTDGCPVSVEKIVNIFNGTSCPSLGGKPKLFFIQACGGEQKDHGFEVASTSPEDESP
GSNPEPDATPFQEGLRTFDQLDAISSLPTPSDIFVSYSTFPGFVSWRDPKSGSWYVETL
DDIFEQWAHSEDLQSLLLRVANAVSVKGIYKQMPGCFQFLRKKLFFKTSASRA
SEQ ID NO: 41, Linker.co nucleotide sequence
CCGCGG
SEQ ID NO: 42, Linker.co amino acid sequence
PR
SEQ ID NO: 246: T2A.co nucleotide sequence
GAAGGCCGAGGGAGCCTGCTGACATGTGGCGATGTGGAGGAAAACCCAGGACCA
SEQ ID NO: 43: T2A.co amino acid sequence
EGRGSLLTCGDVEENPGP
SEQ ID NO: 247: ΔCD19.co nucleotide sequence
ATGCCACCACCTCGCCTGCTGTTCTTTCTGCTGTTCCTGACACCTATGGAGGTGCG
ACCTGAGGAACCACTGGTCGTGAAGGTCGAGGAAGGCGACAATGCCGTGCTGCAG
TGCCTGAAAGGCACTTCTGATGGGCCAACTCAGCAGCTGACCTGGTCCAGGGAGT
CTCCCCTGAAGCCTTTTCTGAAACTGAGCCTGGGACTGCCAGGACTGGGAATCCAC
ATGCGCCCTCTGGCTATCTGGCTGTTCATCTTCAACGTGAGCCAGCAGATGGGAG
GATTCTACCTGTGCCAGCCAGGACCACCATCCGAGAAGGCCTGGCAGCCTGGATG
GACCGTCAACGTGGAGGGGTCTGGAGAACTGTTTAGGTGGAATGTGAGTGACCTG
GGAGGACTGGGATGTGGGCTGAAGAACCGCTCCTCTGAAGGCCCAAGTTCACCCT
CAGGGAAGCTGATGAGCCCAAAACTGTACGTGTGGGCCAAAGATCGGCCCGAGAT
CTGGGAGGGAGAACCTCCATGCCTGCCACCTAGAGACAGCCTGAATCAGAGTCTG
TCACAGGATCTGACAATGGCCCCCGGGTCCACTCTGTGGCTGTCTTGTGGAGTCC
CACCCGACAGCGTGTCCAGAGGCCCTCTGTCCTGGACCCACGTGCATCCTAAGGG
GCCAAAAAGTCTGCTGTCACTGGAACTGAAGGACGATCGGCCTGCCAGAGACATG
TGGGTCATGGAGACTGGACTGCTGCTGCCACGAGCAACCGCACAGGATGCTGGAA
AATACTATTGCCACCGGGGCAATCTGACAATGTCCTTCCATCTGGAGATCACTGCA
AGGCCCGTGCTGTGGCACTGGCTGCTGCGAACCGGAGGATGGAAGGTCAGTGCT
GTGACACTGGCATATCTGATCTTTTGCCTGTGCTCCCTGGTGGGCATTCTGCATCT
GCAGAGAGCCCTGGTGCTGCGGAGAAAGAGAAAGAGAATGACTGACCCAACAAGA
AGGTTTTGA
SEQ ID NO: 248: ΔCD19.co amino acid sequence
MPPPRLLFFLLFLTPMEVRPEEPLVVKVEEGDNAVLQCLKGTSDGPTQQLTWSRESPL
KPFLKLSLGLPGLGIHMRPLAIWLFIFNVSQQMGGFYLCQPGPPSEKAWQPGWTVNVE
GSGELFRWNVSDLGGLGCGLKNRSSEGPSSPSGKLMSPKLYVWAKDRPEIWEGEPP
CLPPRDSLNQSLSQDLTMAPGSTLWLSCGVPPDSVSRGPLSWTHVHPKGPKSLLSLEL
KDDRPARDMVVVMETGLLLPRATAQDAGKYYCHRGNLTMSFHLEITARPVLWHWLLRT
GGWKVSAVTLAYLIFCLCSLVGILHLQRALVLRRKRKRMTDPTRRF*

TABLE 6
Additional Examples of caspase-9 Variants

| iCasp9 Variants | DNA sequence | Amino acid sequence |
|---|---|---|
| Fv-L-Caspase9 WT-2A | Fv disclosed as SEQ ID NO: 249, Linker | Fv disclosed as SEQ ID NO: 252, |
| | disclosed as SEQ ID NO: 250, iCasp9 | Linker disclosed as SEQ ID NO: |
| | disclose as SEQ ID NO: 44 and T2A | 253, iCasp9 disclose as SEQ ID NO: |
| | disclosed as SEQ ID NO: 251 | 45 and T2A disclosed as SEQ ID |
| | (Fv)ATGCTCGAGGGAGTGCAGGTGGAgACtA | NO: 254 |
| | TCTCCCCAGGAGACGGGCGCACCTTCCCCAA | (Fv)MLEGVQVETISPGDGRTFPKRGQ |
| | GCGCGGCCAGACCTGCGTGGTGCACTACAC | TCVVHYTGMLEDGKKVDSSRDRNKP |
| | CGGGATGCTTGAAGATGGAAAGAAAGTTGA | FKFMLGKQEVIR |
| | TTCCTCCCGGGACAGAAACAAGCCCTTTAAG | GWEEGVAQMSVGQRAKLTISPDYAY |
| | TTTATGCTAGGCAAGCAGGAGGTGATCCGA | GATGHPGIIPPHATLVFDVELLKLE- |
| | GGCTGGGAAGAAGGGGTTGCCCAGATGAG | (linker)SGGGSG-(iCasp9)VDGF |
| | TGTGGGTCAGAGAGCCAAACTGACTATATCT | GDVGALESLRGNADLAYILSMEPCGH |
| | CCAGATTATGCCTATGGTGCCACTGGGCACC | CLIINNVNFCRESGLRTRTGSNIDCEKL |
| | CAGGCATCATCCCACCACATGCCACTCTCGT | RRRFSS |
| | CTTCGATGTGGAGCTTCTAAAACTGGA- | LHFMVEVKGDLTAKKMVLALLELAR |
| | (linker)TCTGGCGGTGGATCCGGA- | QDHGALDCCVVVILSHGCQASHLQF |
| | (iCasp9)GTCGACGGATTTGGTGATGTCGGT | PGAVYGTDGC |
| | GCTCTTGAGAGTTTGAGGGGAAATGCAGAT | PVSVEKIVNIFNGTSCPSLGGKPKLFFI |
| | TTGGCTTACATCCTGAGCATGGAGCCCTGTG | QACGGEQKDHGFEVASTSPEDESPG |
| | GCCACTGCCTCATTATCAACAATGTGAACTT | SNPEPDA |
| | CTGCCGTGAGTCCGGGCTCCGCACCCGCACT | TPFQEGLRTFDQLDAISSLPTPSDIFVS |
| | GGCTCCAACATCGACTGTGAGAAGTTGCGG | YSTFPGFVSWRDPKSGSWYVETLDDI |
| | CGTCGCTTCTCCTCGCTGCATTTCATGGTGG | FEQWAH |
| | AGGTGAAGGGCGACCTGACTGCCAAGAAAA | SEDLQSLLLRVANAVSVKGIYKQMPG |
| | TGGTGCTGGCTTTGCTGGAGCTGGCGCGGC | CFNFLRKKLFFKTSASRA- |
| | AGGACCACGGTGCTCTGGACTGCTGCGTGG | EGRGSLLTCGDVEENP |
| | TGGTCATTCTCTCTCACGGCTGTCAGGCCAG | GP- |
| | CCACCTGCAGTTCCCAGGGGCTGTCTACGGC | |
| | ACAGATGGATGCCCTGTGTCGGTCGAGAAG | |
| | ATTGTGAACATCTTCAATGGGACCAGCTGCC | |
| | CCAGCCTGGGAGGGAAGCCCAAGCTCTTTTT | |
| | CATCCAGGCCTGTGGTGGGGAGCAGAAAGA | |
| | CCATGGGTTTGAGGTGGCCTCCACTTCCCCT | |
| | GAAGACGAGTCCCCTGGCAGTAACCCCGAG | |
| | CCAGATGCCACCCCGTTCCAGGAAGGTTTGA | |
| | GGACCTTCGACCAGCTGGACGCCATATCTAG | |
| | TTTGCCCACACCCAGTGACATCTTTGTGTCCT | |
| | ACTCTACTTTCCCAGGTTTTGTTTCCTGGAGG | |
| | GACCCCAAGAGTGGCTCCTGGTACGTTGAG | |
| | ACCCTGGACGACATCTTTGAGCAGTGGGCTC | |
| | ACTCTGAAGACCTGCAGTCCCTCCTGCTTAG | |
| | GGTCGCTAATGCTGTTTCGGTGAAAGGGATT | |
| | TATAAACAGATGCCTGGTTGCTTTAATTTCCT | |
| | CCGGAAAAAACTTTTCTTTAAAACATCAGCT | |
| | AGCAGAGCC- | |
| | (T2A)GAGGGCAGGGGAAGTCTTCTAACATG | |
| | CGGGGACGTGGAGGAAAATCCCGGGCCC | |
| Fv-L-iCaspase9 WT | Fv disclosed as SEQ ID NO: 255, Linker | iCaspase9 disclosed as SEQ ID NO: |
| codon optimized-T2A | disclosed as SEQ ID NO: 256, iCasp9 | 47 and T2A disclosed as SEQ ID |
| codon optimized | disclose as SEQ ID NO: 46 and T2A | NO: 258 |
| | disclosed as SEQ ID NO: 257 | (Fv-L)- |
| | (Fv)- | VDGFGDVGALESLRGNADLAYILSME |
| | GGAGTGCAGGTGGAGACTATTAGCCCCGGA | PCGHCLIINNVNFCRESGLRTRTGSNI |
| | GATGGCAGAACATTCCCCAAAAGAGGACAG | DCEKLRRRFSS |
| | ACTTGCGTCGTGCATTATACTGGAATGCTGG | LHFMVEVKGDLTAKKMVLALLELAR |
| | AAGACGGCAAGAAGGTGGACAGCAGCCGG | QDHGALDCCVVVILSHGCQASHLQF |
| | GACCGAAACAAGCCCTTCAAGTTCATGCTGG | PGAVYGTDGC |
| | GGAAGCAGGAAGTGATCCGGGGCTGGGAG | PVSVEKIVNIFNGTSCPSLGGKPKLFFI |
| | GAAGGAGTCGCACAGATGTCAGTGGGACAG | QACGGEQKDHGFEVASTSPEDESPG |
| | AGGGCCAAACTGACTATTAGCCCAGACTAC | SNPEPDA |
| | GCTTATGGAGCAACCGGCCACCCCGGGATC | TPFQEGLRTFDQLDAISSLPTPSDIFVS |
| | ATTCCCCCTCATGCTACACTGGTCTTCGATGT | YSTFPGFVSWRDPKSGSWYVETLDDI |
| | GGAGCTGCTGAAGCTGGAA-(L)- | FEQWAH |
| | AGCGGAGGAGGATCCGGA-(iCasp9)- | SEDLQSLLLRVANAVSVKGIYKQMPG |
| | GTGGACGGGTTTGGAGATGTGGGAGCCCTG | CFNFLRKKLFFKTSASRA- |
| | GAATCCCTGCGGGGCAATGCCGATCTGGCTT | EGRGSLLTCGDVEENP |
| | ACATCCTGTCTATGGAGCCTTGCGGCCACTG | GP-(T2A) |
| | TCTGATCATTAACAATGTGAACTTCTGCAGA | |
| | GAGAGCGGGCTGCGGACCAGAACAGGATC | |
| | CAATATTGACTGTGAAAAGCTGCGGAGAAG | |
| | GTTCTCTAGTCTGCACTTTATGGTCGAGGTG | |
| | AAAGGCGATCTGACCGCTAAGAAAATGGTG | |
| | CTGGCCCTGCTGGAACTGGCTCGGCAGGAC | |
| | CATGGGGCACTGGATTGCTGCGTGGTCGTG | |
| | ATCCTGAGTCACGGCTGCCAGGCTTCACATC | |
| | TGCAGTTCCCTGGGGCAGTCTATGGAACTGA | |
| | CGGCTGTCCAGTCAGCGTGGAGAAGATCGT | |
| | GAACATCTTCAACGGCACCTCTTGCCCAAGT | |
| | CTGGGCGGGAAGCCCAAACTGTTCTTTATTC | |
| | AGGCCTGTGGAGGCGAGCAGAAAGATCAC | |
| | GGCTTCGAAGTGGCTAGCACCTCCCCCGAG | |
| | GACGAATCACCTGGAAGCAACCCTGAGCCA | |
| | GATGCAACCCCCTTCCAGGAAGGCCTGAGG | |
| | ACATTTGACCAGCTGGATGCCATCTCAAGCC | |
| | TGCCCACACCTTCTGACATTTTCGTCTCTTAC | |
| | AGTACTTTCCCTGGATTTGTGAGCTGGCGCG | |
| | ATCCAAAGTCAGGCAGCTGGTACGTGGAGA | |
| | CACTGGACGATATCTTTGAGCAGTGGGCCCA | |
| | TTCTGAAGACCTGCAGAGTCTGCTGCTGCGA | |
| | GTGGCCAATGCTGTCTCTGTGAAGGGGATCT | |
| | ACAAACAGATGCCAGGATGCTTCAACTTTCT | |
| | GAGAAAGAAACTGTTCTTTAAGACCTCCGCA | |
| | TCTAGGGCC-(T2A)- | |
| | CCGCGGGAAGGCCGAGGGAGCCTGCTGAC | |
| | ATGTGGCGATGTGGAGGAAAACCCAGGACC | |
| | A | |
| Fv-iCASP9 S144A-T2A | SEQ ID NO: 48 | SEQ ID NO: 49 |
| | (Fv-L)- | (Fv-L)- |
| | GTCGACGGATTTGGTGATGTCGGTGCTCTTG | VDGFGDVGALEaLRGNADLAYILSME |
| | AGgcTTTGAGGGGAAATGCAGATTTGGCTTA | PCGHCLIINNVNFCRESGLRTRTGSNI |
| | CATCCTGAGCATGGAGCCCTGTGGCCACTGC | DCEKLRRRFSSLHFMVEVKGDLTAKK |
| | CTCATTATCAACAATGTGAACTTCTGCCGTG | MVLALLELARQDHGALDCCVVVILSH |
| | AGTCCGGGCTCCGCACCCGCACTGGCTCCAA | GCQASHLQFPGAVYGTDGCPVSVEKI |
| | CATCGACTGTGAGAAGTTGCGGCGTCGCTTC | VNIFNGTSCPSLGGKPKLFFIQACGGE |
| | TCCTCGCTGCATTTCATGGTGGAGGTGAAGG | QKDHGFEVASTSPEDESPGSNPEPDA |
| | GCGACCTGACTGCCAAGAAAATGGTGCTGG | TPFQEGLRTFDQLDAISSLPTPSDIFVS |
| | CTTTGCTGGAGCTGGCGCGGCAGGACCACG | YSTFPGFVSWRDPKSGSWYVETLDDI |
| | GTGCTCTGGACTGCTGCGTGGTGGTCATTCT | FEQWAHSEDLQSLLLRVANAVSVKGI |
| | CTCTCACGGCTGTCAGGCCAGCCACCTGCAG | YKQMPGCFNFLRKKLFFKTSASRA |
| | TTCCCAGGGGCTGTCTACGGCACAGATGGA | |
| | TGCCCTGTGTCGGTCGAGAAGATTGTGAAC | |
| | ATCTTCAATGGGACCAGCTGCCCCAGCCTGG | |
| | GAGGGAAGCCCAAGCTCTTTTTCATCCAGGC | |
| | CTGTGGTGGGGAGCAGAAAGACCATGGGTT | |
| | TGAGGTGGCCTCCACTTCCCCTGAAGACGAG | |
| | TCCCCTGGCAGTAACCCCGAGCCAGATGCCA | |
| | CCCCGTTCCAGGAAGGTTTGAGGACCTTCGA | |
| | CCAGCTGGACGCCATATCTAGTTTGCCCACA | |
| | CCCAGTGACATCTTTGTGTCCTACTCTACTTT | |
| | CCCAGGTTTTGTTTCCTGGAGGGACCCCAAG | |
| | AGTGGCTCCTGGTACGTTGAGACCCTGGAC | |
| | GACATCTTTGAGCAGTGGGCTCACTCTGAAG | |
| | ACCTGCAGTCCCTCCTGCTTAGGGTCGCTAA | |
| | TGCTGTTTCGGTGAAAGGGATTTATAAACAG | |
| | ATGCCTGGTTGCTTTAATTTCCTCCGGAAAA | |
| | AACTTTTCTTTAAAACATCAGCTAGCAGAGC | |
| | C-(T2A) | |
| Fv-iCASP9 S144D-T2A | SEQ ID NO: 50 | SEQ ID NO: 51 |
| | (Fv-L)- | (Fv-L)- |
| | GTCGACGGATTTGGTGATGTCGGTGCTCTTG | VDGFGDVGALEdLRGNADLAYILSME |
| | AGgacTTGAGGGGAAATGCAGATTTGGCTTA | PCGHCLIINNVNFCRESGLRTRTGSNI |
| | CATCCTGAGCATGGAGCCCTGTGGCCACTGC | DCEKLRRRFSSLHFMVEVKGDLTAKK |
| | CTCATTATCAACAATGTGAACTTCTGCCGTG | MVLALLELARQDHGALDCCVVVILSH |
| | AGTCCGGGCTCCGCACCCGCACTGGCTCCAA | GCQASHLQFPGAVYGTDGCPVSVEKI |
| | CATCGACTGTGAGAAGTTGCGGCGTCGCTTC | VNIFNGTSCPSLGGKPKLFFIQACGGE |
| | TCCTCGCTGCATTTCATGGTGGAGGTGAAGG | QKDHGFEVASTSPEDESPGSNPEPDA |
| | GCGACCTGACTGCCAAGAAAATGGTGCTGG | TPFQEGLRTFDQLDAISSLPTPSDIFVS |
| | CTTTGCTGGAGCTGGCGCGGCAGGACCACG | YSTFPGFVSWRDPKSGSWYVETLDDI |
| | GTGCTCTGGACTGCTGCGTGGTGGTCATTCT | FEQWAHSEDLQSLLLRVANAVSVKGI |
| | CTCTCACGGCTGTCAGGCCAGCCACCTGCAG | YKQMPGCFNFLRKKLFFKTSASRA |
| | TTCCCAGGGGCTGTCTACGGCACAGATGGA | |
| | TGCCCTGTGTCGGTCGAGAAGATTGTGAAC | |
| | ATCTTCAATGGGACCAGCTGCCCCAGCCTGG | |
| | GAGGGAAGCCCAAGCTCTTTTTCATCCAGGC | |
| | CTGTGGTGGGGAGCAGAAAGACCATGGGTT | |
| | TGAGGTGGCCTCCACTTCCCCTGAAGACGAG | |
| | TCCCCTGGCAGTAACCCCGAGCCAGATGCCA | |
| | CCCCGTTCCAGGAAGGTTTGAGGACCTTCGA | |
| | CCAGCTGGACGCCATATCTAGTTTGCCCACA | |
| | CCCAGTGACATCTTTGTGTCCTACTCTACTTT | |
| | CCCAGGTTTTGTTTCCTGGAGGGACCCCAAG | |
| | AGTGGCTCCTGGTACGTTGAGACCCTGGAC | |
| | GACATCTTTGAGCAGTGGGCTCACTCTGAAG | |
| | ACCTGCAGTCCCTCCTGCTTAGGGTCGCTAA | |
| | TGCTGTTTCGGTGAAAGGGATTTATAAACAG | |
| | ATGCCTGGTTGCTTTAATTTCCTCCGGAAAA | |
| | AACTTTTCTTTAAAACATCAGCTAGCAGAGC | |
| | C-(T2A) | |
| Fv-iCASP9 S183A-T2A | SEQ ID NO: 52 | SEQ ID NO: 53 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGaNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCgCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDQLDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 S196A-T2A | SEQ ID NO: 54 | SEQ ID NO: 55 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSaLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCgCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDQLDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 S196D-T2A | SEQ ID NO: 56 | SEQ ID NO: 57 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSdLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCgacCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDQLDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 C285A-T2A | SEQ ID NO: 58 | SEQ ID NO: 59 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQAaGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDQLDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCgcgGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 A316G-T2A | SEQ ID NO: 60 | SEQ ID NO: 61 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDg |
| | GGCGACCTGACTGCCAAGAAAATGGTGCTG | TPFQEGLRTFDQLDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGgC | |
| | ACCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 T317A-T2A | SEQ ID NO: 62 | SEQ ID NO: 63 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | aPFQEGLRTFDQLDAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CFNFLRKKLFFKTSASRA- |
| | GGAGGGAAGCCCAAGCTCTTTTTCATCCAGG | (T2A) |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | gCCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 T317C-T2A | SEQ ID NO: 64 | SEQ ID NO: 65 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKP KLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | cPFQEGLRTFDQLDAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CFNFLRKKLFFKTSASRA- |
| | GGAGGGAAGCCCAAGCTCTTTTTCATCCAGG | (T2A) |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | tgCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 T3175-T2A | SEQ ID NO: 66 | SEQ ID NO: 67 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKP KLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | sPFQEGLRTFDQLDAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CFNFLRKKLFFKTSASRA- |
| | GGAGGGAAGCCCAAGCTCTTTTTCATCCAGG | (T2A) |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | tCCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 F326K-T2A | SEQ ID NO: 68 | SEQ ID NO: 69 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTkDQLDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA |
| | GTTCCCAGGGGCTGTCTACGGCACAGATGG | |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCaagG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC | |
| Fv-iCASP9 D327K-T2A | SEQ ID NO: 70 | SEQ ID NO: 71 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFkQLDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCa | |
| | AgCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 D327R-T2A | SEQ ID NO: 72 | SEQ ID NO: 73 |
| | GTCGACGGATTTGGTGATGTCGGTGCTCTTG | (Fv-L)- |
| | AGAGTTTGAGGGGAAATGCAGATTTGGCTT | VDGFGDVGALESLRGNADLAYILSME |
| | ACATCCTGAGCATGGAGCCCTGTGGCCACTG | PCGHCLIINNVNFCRESGLRTRTGSNI |
| | CCTCATTATCAACAATGTGAACTTCTGCCGT | DCEKLRRRFSSLHFMVEVKGDLTAKK |
| | GAGTCCGGGCTCCGCACCCGCACTGGCTCCA | MVLALLELARQDHGALDCCVVVILSH |
| | ACATCGACTGTGAGAAGTTGCGGCGTCGCTT | GCQASHLQFPGAVYGTDGCPVSVEKI |
| | CTCCTCGCTGCATTTCATGGTGGAGGTGAAG | VNIFNGTSCPSLGGKPKLFFIQACGGE |
| | GGCGACCTGACTGCCAAGAAAATGGTGCTG | QKDHGFEVASTSPEDESPGSNPEPDA |
| | GCTTTGCTGGAGCTGGCGCGGCAGGACCAC | TPFQEGLRTFrQLDAISSLPTPSDIFVSY |
| | GGTGCTCTGGACTGCTGCGTGGTGGTCATTC | STFPGFVSWRDPKSGSWYVETLDDIF |
| | TCTCTCACGGCTGTCAGGCCAGCCACCTGCA | EQWAHSEDLQSLLLRVANAVSVKGIY |
| | GTTCCCAGGGGCTGTCTACGGCACAGATGG | KQMPGCFNFLRKKLFFKTSASRA- |
| | ATGCCCTGTGTCGGTCGAGAAGATTGTGAA | (T2A) |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCa | |
| | ggCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 D327G- | SEQ ID NO: 74 | SEQ ID NO: 75 |
| T2A | GTCGACGGATTTGGTGATGTCGGTGCTCTTG | (Fv-L)- |
| | AGAGTTTGAGGGGAAATGCAGATTTGGCTT | VDGFGDVGALESLRGNADLAYILSME |
| | ACATCCTGAGCATGGAGCCCTGTGGCCACTG | PCGHCLIINNVNFCRESGLRTRTGSNI |
| | CCTCATTATCAACAATGTGAACTTCTGCCGT | DCEKLRRRFSSLHFMVEVKGDLTAKK |
| | GAGTCCGGGCTCCGCACCCGCACTGGCTCCA | MVLALLELARQDHGALDCCVVVILSH |
| | ACATCGACTGTGAGAAGTTGCGGCGTCGCTT | GCQASHLQFPGAVYGTDGCPVSVEKI |
| | CTCCTCGCTGCATTTCATGGTGGAGGTGAAG | VNIFNGTSCPSLGGKPKLFFIQACGGE |
| | GGCGACCTGACTGCCAAGAAAATGGTGCTG | QKDHGFEVASTSPEDESPGSNPEPDA |
| | GCTTTGCTGGAGCTGGCGCGGCAGGACCAC | TPFQEGLRTFgQLDAISSLPTPSDIFVS |
| | GGTGCTCTGGACTGCTGCGTGGTGGTCATTC | YSTFPGFVSWRDPKSGSWYVETLDDI |
| | TCTCTCACGGCTGTCAGGCCAGCCACCTGCA | FEQWAHSEDLQSLLLRVANAVSVKGI |
| | GTTCCCAGGGGCTGTCTACGGCACAGATGG | YKQMPGCFNFLRKKLFFKTSASRA- |
| | ATGCCCTGTGTCGGTCGAGAAGATTGTGAA | (T2A) |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | gCCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 Q328K-T2A | SEQ ID NO: 76 | SEQ ID NO: 77 |
| | (Fv-L)- | VDGFGDVGALESLRGNADLAYILSME |
| | GTCGACGGATTTGGTGATGTCGGTGCTCTTG | PCGHCLIINNVNFCRESGLRTRTGSNI |
| | AGAGTTTGAGGGGAAATGCAGATTTGGCTT | DCEKLRRRFSSLHFMVEVKGDLTAKK |
| | ACATCCTGAGCATGGAGCCCTGTGGCCACTG | MVLALLELARQDHGALDCCVVVILSH |
| | CCTCATTATCAACAATGTGAACTTCTGCCGT | GCQASHLQFPGAVYGTDGCPVSVEKI |
| | GAGTCCGGGCTCCGCACCCGCACTGGCTCCA | VNIFNGTSCPSLGGKPKLFFIQACGGE |
| | ACATCGACTGTGAGAAGTTGCGGCGTCGCTT | QKDHGFEVASTSPEDESPGSNPEPDA |
| | CTCCTCGCTGCATTTCATGGTGGAGGTGAAG | TPFQEGLRTFDkLDAISSLPTPSDIFVS |
| | GGCGACCTGACTGCCAAGAAAATGGTGCTG | YSTFPGFVSWRDPKSGSWYVETLDDI |
| | GCTTTGCTGGAGCTGGCGCGGCAGGACCAC | FEQWAHSEDLQSLLLRVANAVSVKGI |
| | GGTGCTCTGGACTGCTGCGTGGTGGTCATTC | YKQMPGCFNFLRKKLFFKTSASRA- |
| | TCTCTCACGGCTGTCAGGCCAGCCACCTGCA | (T2A) |
| | GTTCCCAGGGGCTGTCTACGGCACAGATGG | |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACaAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 Q328R-T2A | SEQ ID NO: 78 | SEQ ID NO: 79 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDrLDAISSLPTPSDIFVSY |
| | GCTTTGCTGGAGCTGGCGCGGCAGGACCAC | STFPGFVSWRDPKSGSWYVETLDDIF |
| | GGTGCTCTGGACTGCTGCGTGGTGGTCATTC | EQWAHSEDLQSLLLRVANAVSVKGIY |
| | TCTCTCACGGCTGTCAGGCCAGCCACCTGCA | KQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACagGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 L329K-T2A | SEQ ID NO: 80 | SEQ ID NO: 81 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDQkDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA |
| | GTTCCCAGGGGCTGTCTACGGCACAGATGG | |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGaaGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC | |
| Fv-iCASP9 L329E-T2A | SEQ ID NO: 82 | SEQ ID NO: 83 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDQeDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGgaGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 L329G-T2A | SEQ ID NO: 84 | SEQ ID NO: 85 |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDQgDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA |
| | GTTCCCAGGGGCTGTCTACGGCACAGATGG | |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGggcGACGCCATATCTAGTTTGCCCACA | |
| | CCCAGTGACATCTTTGTGTCCTACTCTACTTT | |
| | CCCAGGTTTTGTTTCCTGGAGGGACCCCAAG | |
| | AGTGGCTCCTGGTACGTTGAGACCCTGGAC | |
| | GACATCTTTGAGCAGTGGGCTCACTCTGAAG | |
| | ACCTGCAGTCCCTCCTGCTTAGGGTCGCTAA | |
| | TGCTGTTTCGGTGAAAGGGATTTATAAACAG | |
| | ATGCCTGGTTGCTTTAATTTCCTCCGGAAAA | |
| | AACTTTTCTTTAAAACATCAGCTAGCAGAGC | |
| | C | |
| Fv-L-Caspase9 | SEQ ID NO: 86 | SEQ ID NO: 87 |
| D330A-T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | TPFQEGLRTFDQLaAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CFNFLRKKLFFKTSASRA-(T2A) |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGcCGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-L-Caspase9 D330E- | SEQ ID NO: 88 | SEQ ID NO: 89 |
| T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | TPFQEGLRTFDQLeAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CFNFLRKKLFFKTSASRA-(T2A) |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGcCGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-L-Caspase9 | SEQ ID NO: 90 | SEQ ID NO: 91 |
| D330N-T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | TPFQEGLRTFDQLnAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CFNFLRKKLFFKTSASRA-(T2A) |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGcCGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-L-Caspase9 | SEQ ID NO: 92 | SEQ ID NO: 93 |
| D330V-T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | TPFQEGLRTFDQLvAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CFNFLRKKLFFKTSASRA-(T2A) |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGcCGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-L-Caspase9 | SEQ ID NO: 94 | SEQ ID NO: 95 |
| D330G-T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | TPFQEGLRTFDQLgAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CFNFLRKKLFFKTSASRA-(T2A) |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGcCGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-L-Caspase9 D3305- | SEQ ID NO: 96 | SEQ ID NO: 97 |
| T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | TPFQEGLRTFDQLsAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CFNFLRKKLFFKTSASRA-(T2A) |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGcCGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 A331K-T2A | SEQ ID NO: 98 | SEQ ID NO: 99 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDQLDkISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGACaagATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-L-iCaspase9 | SEQ ID NO: 100 | SEQ ID NO: 101 |
| F404Y-T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | TPFQEGLRTFDQLDAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CyNFLRKKLFFKTSASRA-(T2A) |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTaTAATTTCCTCCGGAAA | |
| | AAACTTTTCTTTAAAACATCAGCTAGCAGAG | |
| | CC-(T2A) | |
| Fv-L-iCASP9 F404W- | SEQ ID NO: 102 | SEQ ID NO: 103 |
| T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | TPFQEGLRTFDQLDAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CwNFLRKKLFFKTSASRA-(T2A) |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTggAATTTCCTCCGGAAA | |
| | AAACTTTTCTTTAAAACATCAGCTAGCAGAG | |
| | CC-(T2A) | |
| Fv-L-iCaspase9 | SEQ ID NO: 104 | SEQ ID NO: 105 |
| N405Q-T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | TPFQEGLRTFDQLDAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CFqFLRKKLFFKTSASRA-(T2A) |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTcagTTCCTCCGGAAAA | |
| | AACTTTTCTTTAAAACATCAGCTAGCAGAGC | |
| | C-(T2A) | |
| Fv-L-iCaspase9 | SEQ ID NO: 106 | SEQ ID NO: 107 |
| N405Q codon | -(Fv-L)- | (Fv-L)- |
| optimized-T2A | GTGGACGGGTTTGGAGATGTGGGAGCCCTG | VDGFGDVGALESLRGNADLAYILSME |
| | GAATCCCTGCGGGGCAATGCCGATCTGGCTT | PCGHCLIINNVNFCRESGLRTRTGSNI |
| | ACATCCTGTCTATGGAGCCTTGCGGCCACTG | DCEKLRRRFSS |
| | TCTGATCATTAACAATGTGAACTTCTGCAGA | LHFMVEVKGDLTAKKMVLALLELAR |
| | GAGAGCGGGCTGCGGACCAGAACAGGATC | QDHGALDCCVVVILSHGCQASHLQF |
| | CAATATTGACTGTGAAAAGCTGCGGAGAAG | PGAVYGTDGC |
| | GTTCTCTAGTCTGCACTTTATGGTCGAGGTG | PVSVEKIVNIFNGTSCPSLGGKPKLFFI |
| | AAAGGCGATCTGACCGCTAAGAAAATGGTG | QACGGEQKDHGFEVASTSPEDESPG |
| | CTGGCCCTGCTGGAACTGGCTCGGCAGGAC | SNPEPDA |
| | CATGGGGCACTGGATTGCTGCGTGGTCGTG | TPFQEGLRTFDQLDAISSLPTPSDIFVS |
| | ATCCTGAGTCACGGCTGCCAGGCTTCACATC | YSTFPGFVSWRDPKSGSWYVETLDDI |
| | TGCAGTTCCCTGGGGCAGTCTATGGAACTGA | FEQWAH |
| | CGGCTGTCCAGTCAGCGTGGAGAAGATCGT | SEDLQSLLLRVANAVSVKGIYKQMPG |
| | GAACATCTTCAACGGCACCTCTTGCCCAAGT | CFqFLRKKLFFKTSASRA-(T2A) |
| | CTGGGCGGGAAGCCCAAACTGTTCTTTATTC | |
| | AGGCCTGTGGAGGCGAGCAGAAAGATCAC | |
| | GGCTTCGAAGTGGCTAGCACCTCCCCCGAG | |
| | GACGAATCACCTGGAAGCAACCCTGAGCCA | |
| | GATGCAACCCCCTTCCAGGAAGGCCTGAGG | |
| | ACATTTGACCAGCTGGATGCCATCTCAAGCC | |
| | TGCCCACACCTTCTGACATTTTCGTCTCTTAC | |
| | AGTACTTTCCCTGGATTTGTGAGCTGGCGCG | |
| | ATCCAAAGTCAGGCAGCTGGTACGTGGAGA | |
| | CACTGGACGATATCTTTGAGCAGTGGGCCCA | |
| | TTCTGAAGACCTGCAGAGTCTGCTGCTGCGA | |
| | GTGGCCAATGCTGTCTCTGTGAAGGGGATCT | |
| | ACAAACAGATGCCAGGATGCTTCcagTTTCT | |
| | GAGAAAGAAACTGTTCTTTAAGACCTCCGCA | |
| | TCTAGGGCC-(T2A) | |
| Fv-iCASP9 F406L-T2A | SEQ ID NO: 108 | SEQ ID NO: 109 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDQLDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNLLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATcTCCTCCGGAAA | |
| | AAACTTTTCTTTAAAACATCAGCTAGCAGAG | |
| | CC-(T2A) | |
| Fv-iCASP9 F406T-T2A | SEQ ID NO: 110 | SEQ ID NO: 111 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDQLDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNtLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAAttcCCTCCGGAAAA | |
| | AACTTTTCTTTAAAACATCAGCTAGCAGAGC | |
| | C-(T2A) | |
| Fv-L-iCaspase9 S144A | SEQ ID NO: 112 | SEQ ID NO: 113 |
| N405Q-T2A codon | (Fv-L)- | (Fv-L)- |
| optimized | GTGGACGGGTTTGGAGATGTGGGAGCCCTG | VDGFGDVGALEaLRGNADLAYILSME |
| | GAAgCCCTGCGGGGCAATGCCGATCTGGCTT | PCGHCLIINNVNFCRESGLRTRTGSNI |
| | ACATCCTGTCTATGGAGCCTTGCGGCCACTG | DCEKLRRRFSS |
| | TCTGATCATTAACAATGTGAACTTCTGCAGA | LHFMVEVKGDLTAKKMVLALLELAR |
| | GAGAGCGGGCTGCGGACCAGAACAGGATC | QDHGALDCCVVVILSHGCQASHLQF |
| | CAATATTGACTGTGAAAAGCTGCGGAGAAG | PGAVYGTDGC |
| | GTTCTCTAGTCTGCACTTTATGGTCGAGGTG | PVSVEKIVNIFNGTSCPSLGGKPKLFFI |
| | AAAGGCGATCTGACCGCTAAGAAAATGGTG | QACGGEQKDHGFEVASTSPEDESPG |
| | CTGGCCCTGCTGGAACTGGCTCGGCAGGAC | SNPEPDA |
| | CATGGGGCACTGGATTGCTGCGTGGTCGTG | TPFQEGLRTFDQLDAISSLPTPSDIFVS |
| | ATCCTGAGTCACGGCTGCCAGGCTTCACATC | YSTFPGFVSWRDPKSGSWYVETLDDI |
| | TGCAGTTCCCTGGGGCAGTCTATGGAACTGA | FEQWAH |
| | CGGCTGTCCAGTCAGCGTGGAGAAGATCGT | SEDLQSLLLRVANAVSVKGIYKQMPG |
| | GAACATCTTCAACGGCACCTCTTGCCCAAGT | CFqFLRKKLFFKTSASRA-(T2A) |
| | CTGGGCGGGAAGCCCAAACTGTTCTTTATTC | |
| | AGGCCTGTGGAGGCGAGCAGAAAGATCAC | |
| | GGCTTCGAAGTGGCTAGCACCTCCCCCGAG | |
| | GACGAATCACCTGGAAGCAACCCTGAGCCA | |
| | GATGCAACCCCCTTCCAGGAAGGCCTGAGG | |
| | ACATTTGACCAGCTGGATGCCATCTCAAGCC | |
| | TGCCCACACCTTCTGACATTTTCGTCTCTTAC | |
| | AGTACTTTCCCTGGATTTGTGAGCTGGCGCG | |
| | ATCCAAAGTCAGGCAGCTGGTACGTGGAGA | |
| | CACTGGACGATATCTTTGAGCAGTGGGCCCA | |
| | TTCTGAAGACCTGCAGAGTCTGCTGCTGCGA | |
| | GTGGCCAATGCTGTCTCTGTGAAGGGGATCT | |
| | ACAAACAGATGCCAGGATGCTTCcagTTTCT | |
| | GAGAAAGAAACTGTTCTTTAAGACCTCCGCA | |
| | TCTAGGGCC-(T2A) | |
| Fv-iCASP9 S144A | SEQ ID NO: 114 | SEQ ID NO: 115 |
| D330A-T2A | (Fv-L)- | (Fv-L)- |
| | GTCGACGGATTTGGTGATGTCGGTGCTCTTG | VDGFGDVGALEaLRGNADLAYILSME |
| | AGgcTTTGAGGGGAAATGCAGATTTGGCTTA | PCGHCLIINNVNFCRESGLRTRTGSNI |
| | CATCCTGAGCATGGAGCCCTGTGGCCACTGC | DCEKLRRRFSSLHFMVEVKGDLTAKK |
| | CTCATTATCAACAATGTGAACTTCTGCCGTG | MVLALLELARQDHGALDCCVVVILSH |
| | AGTCCGGGCTCCGCACCCGCACTGGCTCCAA | GCQASHLQFPGAVYGTDGCPVSVEKI |
| | CATCGACTGTGAGAAGTTGCGGCGTCGCTTC | VNIFNGTSCPSLGGKPKLFFIQACGGE |
| | TCCTCGCTGCATTTCATGGTGGAGGTGAAGG | QKDHGFEVASTSPEDESPGSNPEPDA |
| | GCGACCTGACTGCCAAGAAAATGGTGCTGG | TPFQEGLRTFDQLaAISSLPTPSDIFVS |
| | CTTTGCTGGAGCTGGCGCGGCAGGACCACG | YSTFPGFVSWRDPKSGSWYVETLDDI |
| | GTGCTCTGGACTGCTGCGTGGTGGTCATTCT | FEQWAHSEDLQSLLLRVANAVSVKGI |
| | CTCTCACGGCTGTCAGGCCAGCCACCTGCAG | YKQMPGCFNFLRKKLFFKTSASRA |
| | TTCCCAGGGGCTGTCTACGGCACAGATGGA | |
| | TGCCCTGTGTCGGTCGAGAAGATTGTGAAC | |
| | ATCTTCAATGGGACCAGCTGCCCCAGCCTGG | |
| | GAGGGAAGCCCAAGCTCTTTTTCATCCAGGC | |
| | CTGTGGTGGGGAGCAGAAAGACCATGGGTT | |
| | TGAGGTGGCCTCCACTTCCCCTGAAGACGAG | |
| | TCCCCTGGCAGTAACCCCGAGCCAGATGCCA | |
| | CCCCGTTCCAGGAAGGTTTGAGGACCTTCGA | |
| | CCAGCTGGcCGCCATATCTAGTTTGCCCACA | |
| | CCCAGTGACATCTTTGTGTCCTACTCTACTTT | |
| | CCCAGGTTTTGTTTCCTGGAGGGACCCCAAG | |
| | AGTGGCTCCTGGTACGTTGAGACCCTGGAC | |
| | GACATCTTTGAGCAGTGGGCTCACTCTGAAG | |
| | ACCTGCAGTCCCTCCTGCTTAGGGTCGCTAA | |
| | TGCTGTTTCGGTGAAAGGGATTTATAAACAG | |
| | ATGCCTGGTTGCTTTAATTTCCTCCGGAAAA | |
| | AACTTTTCTTTAAAACATCAGCTAGCAGAGC | |
| | C-(T2A) | |
| Fv-iCASP9 S144D | SEQ ID NO: 116 | SEQ ID NO: 117 |
| D330A-T2A | (Fv-L)- | (Fv-L)- |
| | GTCGACGGATTTGGTGATGTCGGTGCTCTTG | VDGFGDVGALEdLRGNADLAYILSME |
| | AGgacTTGAGGGGAAATGCAGATTTGGCTTA | PCGHCLIINNVNFCRESGLRTRTGSNI |
| | CATCCTGAGCATGGAGCCCTGTGGCCACTGC | DCEKLRRRFSSLHFMVEVKGDLTAKK |
| | CTCATTATCAACAATGTGAACTTCTGCCGTG | MVLALLELARQDHGALDCCVVVILSH |
| | AGTCCGGGCTCCGCACCCGCACTGGCTCCAA | GCQASHLQFPGAVYGTDGCPVSVEKI |
| | CATCGACTGTGAGAAGTTGCGGCGTCGCTTC | VNIFNGTSCPSLGGKPKLFFIQACGGE |
| | TCCTCGCTGCATTTCATGGTGGAGGTGAAGG | QKDHGFEVASTSPEDESPGSNPEPDA |
| | GCGACCTGACTGCCAAGAAAATGGTGCTGG | TPFQEGLRTFDQLaAISSLPTPSDIFVS |
| | CTTTGCTGGAGCTGGCGCGGCAGGACCACG | YSTFPGFVSWRDPKSGSWYVETLDDI |
| | GTGCTCTGGACTGCTGCGTGGTGGTCATTCT | FEQWAHSEDLQSLLLRVANAVSVKGI |
| | CTCTCACGGCTGTCAGGCCAGCCACCTGCAG | YKQMPGCFNFLRKKLFFKTSASRA |
| | TTCCCAGGGGCTGTCTACGGCACAGATGGA | |
| | TGCCCTGTGTCGGTCGAGAAGATTGTGAAC | |
| | ATCTTCAATGGGACCAGCTGCCCCAGCCTGG | |
| | GAGGGAAGCCCAAGCTCTTTTTCATCCAGGC | |
| | CTGTGGTGGGGAGCAGAAAGACCATGGGTT | |
| | TGAGGTGGCCTCCACTTCCCCTGAAGACGAG | |
| | TCCCCTGGCAGTAACCCCGAGCCAGATGCCA | |
| | CCCCGTTCCAGGAAGGTTTGAGGACCTTCGA | |
| | CCAGCTGGcCGCCATATCTAGTTTGCCCACA | |
| | CCCAGTGACATCTTTGTGTCCTACTCTACTTT | |
| | CCCAGGTTTTGTTTCCTGGAGGGACCCCAAG | |
| | AGTGGCTCCTGGTACGTTGAGACCCTGGAC | |
| | GACATCTTTGAGCAGTGGGCTCACTCTGAAG | |
| | ACCTGCAGTCCCTCCTGCTTAGGGTCGCTAA | |
| | TGCTGTTTCGGTGAAAGGGATTTATAAACAG | |
| | ATGCCTGGTTGCTTTAATTTCCTCCGGAAAA | |
| | AACTTTTCTTTAAAACATCAGCTAGCAGAGC | |
| | C-(T2A) | |
| Fv-iCASP9 S196A | SEQ ID NO: 118 | SEQ ID NO: 119 |
| D330A-T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSaLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCgCGCTGCATTTCATGGTGGAGGTGAAG | QKDHGFEVASTSPEDESPGSNPEPDA |
| | GGCGACCTGACTGCCAAGAAAATGGTGCTG | TPFQEGLRTFDQLaAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGcCGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-iCASP9 S196D | SEQ ID NO: 120 | SEQ ID NO: 121 |
| D330A-T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSdLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCgacCTGCATTTCATGGTGGAGGTGAAG | QKDHGFEVASTSPEDESPGSNPEPDA |
| | GGCGACCTGACTGCCAAGAAAATGGTGCTGT | PFQEGLRTFDQLaAISSLPTPSDIFVS |
| | GCTTTGCTGGAGCTGGCGCGGCAGGACCACY | STFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGcCGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTTAATTTCCTCCGGAAA | |
| | AAACTTTTCTTTAAAACATCAGCTAGCAGAG | |
| | CC-(T2A) | |
| Fv-L-iCaspase9 T3175 | SEQ ID NO: 122 | SEQ ID NO: 123 |
| N405Q-T2A codon | (Fv-L)- | (Fv-L)- |
| optimized | GTGGACGGGTTTGGAGATGTGGGAGCCCTG | VDGFGDVGALESLRGNADLAYILSME |
| | GAATCCCTGCGGGGCAATGCCGATCTGGCTT | PCGHCLIINNVNFCRESGLRTRTGSNI |
| | ACATCCTGTCTATGGAGCCTTGCGGCCACTG | DCEKLRRRFSS |
| | TCTGATCATTAACAATGTGAACTTCTGCAGA | LHFMVEVKGDLTAKKMVLALLELAR |
| | GAGAGCGGGCTGCGGACCAGAACAGGATC | QDHGALDCCVVVILSHGCQASHLQF |
| | CAATATTGACTGTGAAAAGCTGCGGAGAAG | PGAVYGTDGC |
| | GTTCTCTAGTCTGCACTTTATGGTCGAGGTG | PVSVEKIVNIFNGTSCPSLGGKPKLFFI |
| | AAAGGCGATCTGACCGCTAAGAAAATGGTG | QACGGEQKDHGFEVASTSPEDESPG |
| | CTGGCCCTGCTGGAACTGGCTCGGCAGGAC | SNPEPDA |
| | CATGGGGCACTGGATTGCTGCGTGGTCGTG | sPFQEGLRTFDQLDAISSLPTPSDIFVS |
| | ATCCTGAGTCACGGCTGCCAGGCTTCACATC | YSTFPGFVSWRDPKSGSWYVETLDDI |
| | TGCAGTTCCCTGGGGCAGTCTATGGAACTGA | FEQWAH |
| | CGGCTGTCCAGTCAGCGTGGAGAAGATCGT | SEDLQSLLLRVANAVSVKGIYKQMPG |
| | GAACATCTTCAACGGCACCTCTTGCCCAAGT | CFqFLRKKLFFKTSASRA-(T2A) |
| | CTGGGCGGGAAGCCCAAACTGTTCTTTATTC | |
| | AGGCCTGTGGAGGCGAGCAGAAAGATCAC | |
| | GGCTTCGAAGTGGCTAGCACCTCCCCCGAG | |
| | GACGAATCACCTGGAAGCAACCCTGAGCCA | |
| | GATGCAAgCCCCTTCCAGGAAGGCCTGAGG | |
| | ACATTTGACCAGCTGGATGCCATCTCAAGCC | |
| | TGCCCACACCTTCTGACATTTTCGTCTCTTAC | |
| | AGTACTTTCCCTGGATTTGTGAGCTGGCGCG | |
| | ATCCAAAGTCAGGCAGCTGGTACGTGGAGA | |
| | CACTGGACGATATCTTTGAGCAGTGGGCCCA | |
| | TTCTGAAGACCTGCAGAGTCTGCTGCTGCGA | |
| | GTGGCCAATGCTGTCTCTGTGAAGGGGATCT | |
| | ACAAACAGATGCCAGGATGCTTCcagTTTCT | |
| | GAGAAAGAAACTGTTCTTTAAGACCTCCGCA | |
| | TCTAGGGCC-(T2A) | |
| Fv-L-Caspase9 D330A | SEQ ID NO: 124 | SEQ ID NO: 125 |
| N405Q-T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | TPFQEGLRTFDQLaAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPG |
| | CATCTTCAATGGGACCAGCTGCCCCAGCCTG | CFqFLRKKLFFKTSASRA-(T2A) |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGcCGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCTGGTTGCTTcagTTTCCTCCGGAAAA | |
| | AACTTTTCTTTAAAACATCAGCTAGCAGAGC | |
| | C-(T2A) | |
| Fv-iCASP9 | SEQ ID NO: 126 | SEQ ID NO: 127 |
| ATPF316AVPI-T2A | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSSLHFMVEVKGDLTAKK |
| | CCTCATTATCAACAATGTGAACTTCTGCCGT | MVLALLELARQDHGALDCCVVVILSH |
| | GAGTCCGGGCTCCGCACCCGCACTGGCTCCA | GCQASHLQFPGAVYGTDGCPVSVEKI |
| | ACATCGACTGTGAGAAGTTGCGGCGTCGCTT | VNIFNGTSCPSLGGKPKLFFIQACGGE |
| | CTCCTCGCTGCATTTCATGGTGGAGGTGAAG | QKDHGFEVASTSPEDESPGSNPEPDA |
| | GGCGACCTGACTGCCAAGAAAATGGTGCTG | vPiQEGLRTFDQLDAISSLPTPSDIFVS |
| | GCTTTGCTGGAGCTGGCGCGGCAGGACCAC | YSTFPGFVSWRDPKSGSWYVETLDDI |
| | GGTGCTCTGGACTGCTGCGTGGTGGTCATTC | FEQWAHSEDLQSLLLRVANAVSVKGI |
| | TCTCTCACGGCTGTCAGGCCAGCCACCTGCA | YKQMPGCFNFLRKKLFFKTSASRA- |
| | GTTCCCAGGGGCTGTCTACGGCACAGATGG | (T2A) |
| | ATGCCCTGTGTCGGTCGAGAAGATTGTGAA | |
| | CATCTTCAATGGGACCAGCTGCCCCAGCCTG | |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | gtgCCcaTCCAGGAAGGTTTGAGGACCTTCGA | |
| | CCAGCTGGACGCCATATCTAGTTTGCCCACA | |
| | CCCAGTGACATCTTTGTGTCCTACTCTACTTT | |
| | CCCAGGTTTTGTTTCCTGGAGGGACCCCAAG | |
| | AGTGGCTCCTGGTACGTTGAGACCCTGGAC | |
| | GACATCTTTGAGCAGTGGGCTCACTCTGAAG | |
| | ACCTGCAGTCCCTCCTGCTTAGGGTCGCTAA | |
| | TGCTGTTTCGGTGAAAGGGATTTATAAACAG | |
| | ATGCCTGGTTGCTTTAATTTCCTCCGGAAAA | |
| | AACTTTTCTTTAAAACATCAGCTAGCAGAGC | |
| | C-(T2A) | |
| Fv-ICASP9 isaqt-T2A | SEQ ID NO: 128 | SEQ ID NO: 129 |
| | (Fv-L)- | (Fv-L)- |
| | GTCGACGGATTTGGTGATGTCGGTGCTCTTG | VDGFGDVGALESLRGNADLAYILSME |
| | AGAGTTTGAGGGGAAATGCAGATTTGGCTT | PCGHCLIINNVNFCRESGLRTRTGSNI |
| | ACATCCTGAGCATGGAGCCCTGTGGCCACTG | DCEKLRRRFSS |
| | CCTCATTATCAACAATGTGAACTTCTGCCGT | LHFMVEVKGDLTAKKMVLALLELAR |
| | GAGTCCGGGCTCCGCACCCGCACTGGCTCCA | QDHGALDCCVVVILSHGCQASHLQF |
| | ACATCGACTGTGAGAAGTTGCGGCGTCGCTT | PGAVYGTDGC |
| | CTCCTCGCTGCATTTCATGGTGGAGGTGAAG | PVSVEKIVNIFNGTSCPSLGGKPKLFFI |
| | GGCGACCTGACTGCCAAGAAAATGGTGCTG | QACGGEQKDHGFEVASTSPEDESPG |
| | GCTTTGCTGGAGCTGGCGCGGCAGGACCAC | SNPEPDA |
| | GGTGCTCTGGACTGCTGCGTGGTGGTCATTC | TPFQEGLRTFDQLDAISSLPTPSDIFVS |
| | TCTCTCACGGCTGTCAGGCCAGCCACCTGCA | YSTFPGFVSWRDPKSGSWYVETLDDI |
| | GTTCCCAGGGGCTGTCTACGGCACAGATGG | FEQWAH |
| | ATGCCCTGTGTCGGTCGAGAAGATTGTGAA | SEDLQSLLLRVANAVSVKGIYKQMPis |
| | CATCTTCAATGGGACCAGCTGCCCCAGCCTG | aqtLRKKLFFKTSASRA-(T2A) |
| | GGAGGGAAGCCCAAGCTCTTTTTCATCCAGG | |
| | CCTGTGGTGGGGAGCAGAAAGACCATGGGT | |
| | TTGAGGTGGCCTCCACTTCCCCTGAAGACGA | |
| | GTCCCCTGGCAGTAACCCCGAGCCAGATGCC | |
| | ACCCCGTTCCAGGAAGGTTTGAGGACCTTCG | |
| | ACCAGCTGGACGCCATATCTAGTTTGCCCAC | |
| | ACCCAGTGACATCTTTGTGTCCTACTCTACTT | |
| | TCCCAGGTTTTGTTTCCTGGAGGGACCCCAA | |
| | GAGTGGCTCCTGGTACGTTGAGACCCTGGA | |
| | CGACATCTTTGAGCAGTGGGCTCACTCTGAA | |
| | GACCTGCAGTCCCTCCTGCTTAGGGTCGCTA | |
| | ATGCTGTTTCGGTGAAAGGGATTTATAAACA | |
| | GATGCCgatatccgcacagacaCTCCGGAAAAAA | |
| | CTTTTCTTTAAAACATCAGCTAGCAGAGCC- | |
| | (T2A) | |

Partial sequence of a plasmid insert coding for a polypeptide that encodes an inducible caspase-9 polypeptide and a chimeric antigen receptor that binds to CD19, separated by a 2A linker, wherein the Δcaspase-9 polypeptide and the chimeric antigen receptor are separated during translation. The example of a chimeric antigen receptor provided herein may be further modified by including costimulatory polypeptides such as, for example, but not limited to, CD28, 4-1 BB and OX40. The inducible caspase-9 polypeptide provided herein may be substituted by an inducible modified caspase-9 polypeptide, such as, for example, those provided herein.

SEQ ID NO: 130 FKBPv36
ATGCTGGAGGGAGTGCAGGTGGAGACTATTAGCCCCGGAGATGGCAGAACATTCC
CCAAAAGAGGACAGACTTGCGTCGTGCATTATACTGGAATGCTGGAAGACGGCAA
GAAGGTGGACAGCAGCCGGGACCGAAACAAGCCCTTCAAGTTCATGCTGGGGAAG
CAGGAAGTGATCCGGGGCTGGGAGGAAGGAGTCGCACAGATGTCAGTGGGACAG
AGGGCCAAACTGACTATTAGCCCAGACTACGCTTATGGAGCAACCGGCCACCCCG
GGATCATTCCCCCTCATGCTACACTGGTCTTCGATGTGGAGCTGCTGAAGCTGGAA
SEQ ID NO: 131 FKBPv36
MLEGVQVETISPGDGRTFPKRGQTCVVHYTGMLEDGKKVDSSRDRNKPFKFMLGKQE
VIRGWEEGVAQMSVGQRAKLTISPDYAYGATGHPGIIPPHATLVFDVELLKLE
SEQ ID NO: 132 Linker
AGCGGAGGAGGATCCGGA
SEQ ID NO: 133 Linker
SGGGSG
SEQ ID NO: 134 Caspase-9
GTGGACGGGTTTGGAGATGTGGGAGCCCTGGAATCCCTGCGGGGCAATGCCGAT
CTGGCTTACATCCTGTCTATGGAGCCTTGCGGCCACTGTCTGATCATTAACAATGT
GAACTTCTGCAGAGAGAGCGGGCTGCGGACCAGAACAGGATCCAATATTGACTGT
GAAAAGCTGCGGAGAAGGTTCTCTAGTCTGCACTTTATGGTCGAGGTGAAAGGCGA
TCTGACCGCTAAGAAAATGGTGCTGGCCCTGCTGGAACTGGCTCGGCAGGACCAT
GGGGCACTGGATTGCTGCGTGGTCGTGATCCTGAGTCACGGCTGCCAGGCTTCAC
ATCTGCAGTTCCCTGGGGCAGTCTATGGAACTGACGGCTGTCCAGTCAGCGTGGA
GAAGATCGTGAACATCTTCAACGGCACCTCTTGCCCAAGTCTGGGCGGGAAGCCC
AAACTGTTCTTTATTCAGGCCTGTGGAGGCGAGCAGAAAGATCACGGCTTCGAAGT
GGCTAGCACCTCCCCCGAGGACGAATCACCTGGAAGCAACCCTGAGCCAGATGCA
ACCCCCTTCCAGGAAGGCCTGAGGACATTTGACCAGCTGGATGCCATCTCAAGCCT
GCCCACACCTTCTGACATTTTCGTCTCTTACAGTACTTTCCCTGGATTTGTGAGCTG
GCGCGATCCAAAGTCAGGCAGCTGGTACGTGGAGACACTGGACGATATCTTTGAG
CAGTGGGCCCATTCTGAAGACCTGCAGAGTCTGCTGCTGCGAGTGGCCAATGCTG
TCTCTGTGAAGGGGATCTACAAACAGATGCCAGGATGCTTCAACTTTCTGAGAAAG
AAACTGTTCTTTAAGACCTCCGCATCTAGGGCC
SEQ ID NO: 135 Caspase-9
VDGFGDVGALESLRGNADLAYILSMEPCGHCLIINNVNFCRESGLRTRTGSNIDCEKLR
RRFSSLHFMVEVKGDLTAKKMVLALLELARQDHGALDCCVVVILSHGCQASHLQFPGA
VYGTDGCPVSVEKIVNIFNGTSCPSLGGKPKLFFIQACGGEQKDHGFEVASTSPEDESP
GSNPEPDATPFQEGLRTFDQLDAISSLPTPSDIFVSYSTFPGFVSWRDPKSGSWYVETL
DDIFEQWAHSEDLQSLLLRVANAVSVKGIYKQMPGCFNFLRKKLFFKTSASRA
SEQ ID NO: 136 Linker
CCGCGG
SEQ ID NO: 137 Linker
PR
SEQ ID NO: 138 T2A
GAAGGCCGAGGGAGCCTGCTGACATGTGGCGATGTGGAGGAAAACCCAGGACCA
SEQ ID NO: 139 T2A
EGRGSLLTCGDVEENPGP
SEQ ID NO: 140 Linker
CCATGG
SEQ ID NO: 141 Linker
PW
SEQ ID NO: 142 Signal peptide
ATGGAGTTTGGACTTTCTTGGTTGTTTTTGGTGGCAATTCTGAAGGGTGTCCAGTGT
AGCAGG
SEQ ID NO: 143 Signal peptide
MEFGLSWLFLVAILKGVQCSR
SEQ ID NO: 144 FMC63 variable light chain (anti-CD19)
GACATCCAGATGACACAGACTACATCCTCCCTGTCTGCCTCTCTGGGAGACAGAGT
CACCATCAGTTGCAGGGCAAGTCAGGACATTAGTAAATATTTAAATTGGTATCAGCA
GAAACCAGATGGAACTGTTAAACTCCTGATCTACCATACATCAAGATTACACTCAGG
AGTCCCATCAAGGTTCAGTGGCAGTGGGTCTGGAACAGATTATTCTCTCACCATTA
GCAACCTGGAGCAAGAAGATATTGCCACTTACTTTTGCCAACAGGGTAATACGCTT
CCGTACACGTTCGGAGGGGGGACTAAGTTGGAAATAACA
SEQ ID NO: 145 FMC63 variable light chain (anti CD19)
DIQMTQTTSSLSASLGDRVTISCRASQDISKYLNWYQQKPDGTVKLLIYHTSRLHSGVP
SRFSGSGSGTDYSLTISNLEQEDIATYFCQQGNTLPYTFGGGTKLEIT
SEQ ID NO: 146 Flexible linker
GGCGGAGGAAGCGGAGGTGGGGGC
SEQ ID NO: 147 Flexible linker
GGGSGGGG
SEQ ID NO: 148 FMC63 variable heavy chain (anti-CD19)
GAGGTGAAACTGCAGGAGTCAGGACCTGGCCTGGTGGCGCCCTCACAGAGCCTGT
CCGTCACATGCACTGTCTCAGGGGTCTCATTACCCGACTATGGTGTAAGCTGGATT
CGCCAGCCTCCACGAAAGGGTCTGGAGTGGCTGGGAGTAATATGGGGTAGTGAAA
CCACATACTATAATTCAGCTCTCAAATCCAGACTGACCATCATCAAGGACAACTCCA
AGAGCCAAGTTTTCTTAAAAATGAACAGTCTGCAAACTGATGACACAGCCATTTACT
ACTGTGCCAAACATTATTACTACGGTGGTAGCTATGCTATGGACTACTGGGGTCAA
GGAACCTCAGTCACCGTCTCCTCA
SEQ ID NO: 149 FMC63 variable heavy chain (anti CD19)
EVKLQESGPGLVAPSQSLSVTCTVSGVSLPDYGVSWIRQPPRKGLEWLGVIWGSETTY
YNSALKSRLTIIKDNSKSQVFLKMNSLQTDDTAIYYCAKHYYYGGSYAMDYWGQGTSV
TVSS
SEQ ID NO: 150 Linker
GGATCC
SEQ ID NO: 151 Linker
GS
SEQ ID NO: 152 CD34 minimal epitope
GAACTTCCTACTCAGGGGACTTTCTCAAACGTTAGCACAAACGTAAGT
SEQ ID NO: 153 CD34 minimal epitope
ELPTQGTFSNVSTNVS
SEQ ID NO: 154 CD8 α stalk domain
CCCGCCCCAAGACCCCCCACACCTGCGCCGACCATTGCTTCTCAACCCCTGAGTTT
GAGACCCGAGGCCTGCCGGCCAGCTGCCGGCGGGGCCGTGCATACAAGAGGACT
CGATTTCGCTTGCGAC
SEQ ID NO: 155 CD8 α stalk domain
PAPRPPTPAPTIASQPLSLRPEACRPAAGGAVHTRGLDFACD
SEQ ID NO: 156 CD8 α transmembrane domain
ATCTATATCTGGGCACCTCTCGCTGGCACCTGTGGAGTCCTTCTGCTCAGCCTGGT
TATTACTCTGTACTGTAATCACCGGAATCGCCGCCGCGTTTGTAAGTGTCCCAGG
SEQ ID NO: 157 CD8 α transmembrane domain
IYIWAPLAGTCGVLLLSLVITLYCNHRNRRRVCKCPR
SEQ ID NO: 158 Linker
GTCGAC
SEQ ID NO: 159 Linker
VD
SEQ ID NO: 160 CD3 zeta
AGAGTGAAGTTCAGCAGGAGCGCAGACGCCCCCGCGTACCAGCAGGGCCAGAAC
CAGCTCTATAACGAGCTCAATCTAGGACGAAGAGAGGAGTACGATGTTTTGGACAA
GAGACGTGGCCGGGACCCTGAGATGGGGGGAAAGCCGAGAAGGAAGAACCCTCA
GGAAGGCCTGTACAATGAACTGCAGAAAGATAAGATGGCGGAGGCCTACAGTGAG
ATTGGGATGAAAGGCGAGCGCCGGAGGGGCAAGGGGCACGATGGCCTTTACCAG
GGTCTCAGTACAGCCACCAAGGACACCTACGACGCCCTTCACATGCAGGCCCTGC
CCCCTCGC
SEQ ID NO: 161 CD3 zeta
RVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
Provided below is an example of a plasmid insert coding for a
chimeric antigen receptor that binds to Her2/Neu. The chimeric
antigen receptor may be further modified by including
costimulatory polypeptides such as, for example, but not limited
to, CD28, OX40, and 4-1BB.
SEQ ID NO: 162 Signal peptide
ATGGAGTTTGGACTTTCTTGGTTGTTTTTGGTGGCAATTCTGAAGGGTGTCCAGTGT
AGCAGG
SEQ ID NO: 163 Signal peptide
MEFGLSWLFLVAILKGVQCSR
SEQ ID NO: 164 FRP5 variable light chain (anti-Her2)
GACATCCAATTGACACAATCACACAAATTTCTCTCAACTTCTGTAGGAGACAGAGTG
AGCATAACCTGCAAAGCATCCCAGGACGTGTACAATGCTGTGGCTTGGTACCAACA
GAAGCCTGGACAATCCCCAAAATTGCTGATTTATTCTGCCTCTAGTAGGTACACTGG
GGTACCTTCTCGGTTTACGGGCTCTGGGTCCGGACCAGATTTCACGTTCACAATCA
GTTCCGTTCAAGCTGAAGACCTCGCTGTTTATTTTTGCCAGCAGCACTTCCGAACC
CCTTTTACTTTTGGCTCAGGCACTAAGTTGGAAATCAAGGCTTTG
SEQ ID NO: 165 FRP5 variable light chain (anti-Her2)
DIQLTQSHKFLSTSVGDRVSITCKASQDVYNAVAVVYQQKPGQSPKLLIYSASSRYTGVP
SRFTGSGSGPDFTFTISSVQAEDLAVYFCQQHFRTPFTFGSGTKLEIKAL
SEQ ID NO: 166 Flexible linker
GGCGGAGGAAGCGGAGGTGGGGGC
SEQ ID NO: 167 Flexible linker
GGGSGGGG
SEQ ID NO: 168 FRP5 variable heavy chain (anti-Her2/Neu)
GAAGTCCAATTGCAACAGTCAGGCCCCGAATTGAAAAAGCCCGGCGAAACAGTGA
AGATATCTTGTAAAGCCTCCGGTTACCCTTTTACGAACTATGGAATGAACTGGGTCA
AACAAGCCCCTGGACAGGGATTGAAGTGGATGGGATGGATCAATACATCAACAGG
CGAGTCTACCTTCGCAGATGATTTCAAAGGTCGCTTTGACTTCTCACTGGAGACCA
GTGCAAATACCGCCTACCTTCAGATTAACAATCTTAAAAGCGAGGATATGGCAACCT
ACTTTTGCGCAAGATGGGAAGTTTATCACGGGTACGTGCCATACTGGGGACAAGGA
ACGACAGTGACAGTTAGTAGC
SEQ ID NO: 169 FRP5 variable heavy chain (anti-Her2/Neu)
EVQLQQSGPELKKPGETVKISCKASGYPFTNYGMNWVKQAPGQGLKWMGWINTSTG
ESTFADDFKGRFDFSLETSANTAYLQINNLKSEDMATYFCARWEVYHGYVPYWGQGT
TVTVSS
SEQ ID NO: 170 Linker
GGATCC
SEQ ID NO: 171 Linker
GS
SEQ ID NO: 172 CD34 minimal epitope
GAACTTCCTACTCAGGGGACTTTCTCAAACGTTAGCACAAACGTAAGT
SEQ ID NO: 173 CD34 minimal epitope
ELPTQGTFSNVSTNVS
SEQ ID NO: 174 CD8 alpha stalk
CCCGCCCCAAGACCCCCCACACCTGCGCCGACCATTGCTTCTCAACCCCTGAGTTT
GAGACCCGAGGCCTGCCGGCCAGCTGCCGGCGGGGCCGTGCATACAAGAGGACT
CGATTTCGCTTGCGAC
SEQ ID NO: 175 CD8 alpha stalk
PAPRPPTPAPTIASQPLSLRPEACRPAAGGAVHTRGLDFACD
SEQ ID NO: 176 CD8 alpha transmembrane region
ATCTATATCTGGGCACCTCTCGCTGGCACCTGTGGAGTCCTTCTGCTCAGCCTGGT
TATTACTCTGTACTGTAATCACCGGAATCGCCGCCGCGTTTGTAAGTGTCCCAGG
SEQ ID NO: 177 CD8 alpha transmembrane region
IYIWAPLAGTCGVLLLSLVITLYCNHRNRRRVCKCPR
SEQ ID NO: 178 Linker
Ctcgag
SEQ ID NO: 179 Linker
LE
SEQ ID NO: 180 CD3 zeta cytoplasmic domain
AGAGTGAAGTTCAGCAGGAGCGCAGACGCCCCCGCGTACCAGCAGGGCCAGAAC
CAGCTCTATAACGAGCTCAATCTAGGACGAAGAGAGGAGTACGATGTTTTGGACAA
GAGACGTGGCCGGGACCCTGAGATGGGGGGAAAGCCGAGAAGGAAGAACCCTCA
GGAAGGCCTGTACAATGAACTGCAGAAAGATAAGATGGCGGAGGCCTACAGTGAG
ATTGGGATGAAAGGCGAGCGCCGGAGGGGCAAGGGGCACGATGGCCTTTACCAG
GGTCTCAGTACAGCCACCAAGGACACCTACGACGCCCTTCACATGCAGGCCCTGC
CCCCTCGC
SEQ ID NO: 181 CD3 zeta cytoplasmic domain
RVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
Additional sequences
SEQ ID NO: 182, CD28 nt
TTCTGGGTACTGGTTGTAGTCGGTGGCGTACTTGCTTGTTATTCTCTTCTTGTTACC
GTAGCCTTCATTATATTCTGGGTCCGATCAAAGCGCTCAAGACTCCTCCATTCCGAT
TATATGAACATGACACCTCGCCGACCTGGTCCTACACGCAAACATTATCAACCCTAC
GCACCCCCCCGAGACTTCGCTGCTTATCGATCC
SEQ ID NO: 183, CD28 aa
FWVLVWGGVLACYSLLVTVAFIIFWVRSKRSRLLHSDYMNMTPRRPGPTRKHYQPYA
PPRDFAAYRS
SEQ ID NO: 184, OX40 nt
GTTGCCGCCATCCTGGGCCTGGGCCTGGTGCTGGGGCTGCTGGGCCCCCTGGCC
ATCCTGCTGGCCCTGTACCTGCTCCGGGACCAGAGGCTGCCCCCCGATGCCCACA
AGCCCCCTGGGGGAGGCAGTTTCCGGACCCCCATCCAAGAGGAGCAGGCCGACG
CCCACTCCACCCTGGCCAAGATC
SEQ ID NO: 185, OX40 aa
VAAILGLGLVLGLLGPLAILLALYLLRRDQRLPPDAHKPPGGGSFRTPIQEEQADAHSTL
AKI
SEQ ID NO: 186, 4-1BB nt
AGTGTAGTTAAAAGAGGAAGAAAAAAGTTGCTGTATATATTTAAACAACCATTTATGA
GACCAGTGCAAACCACCCAAGAAGAAGACGGATGTTCATGCAGATTCCCAGAAGAA
GAAGAAGGAGGATGTGAATTG
SEQ ID NO: 187, 4-1BB aa
SVVKRGRKKLLYIFKQPFMRPVQTTQEEDGCSCRFPEEEEGGCEL

Partial sequence of a plasmid insert coding for a polypeptide that encodes an inducible caspase-9 polypeptide and a chimeric antigen receptor that binds to Her2, separated by a 2A linker, wherein the Δcaspase-9 polypeptide and the chimeric antigen receptor are separated during translation. The example of a chimeric antigen receptor provided herein includes truncated MyD88 and CD40 polypeptides; the chimeric antigen receptor may be further modified by substituting the MyD88 and CD40 polypeptides with other polypeptides including costimulatory polypeptides such as, for example, but not limited to, CD28, 4-1 BB and OX40. The inducible caspase-9 polypeptide provided herein may be substituted by an inducible modified caspase-9 polypeptide, such as, for example, those provided herein.

pBP0545-pSFG-iCasp9.2A-aHer2.Q.CD8stm.MC.z

| Fragment | Nucleotide | Peptide |
|---|---|---|
| Linker | SEQ ID NO: 188 | SEQ ID NO: 189 |
| | ATG-CTCGAG | MLE |
| FKBPv36 | SEQ ID NO: 190 | SEQ ID NO: 191 |
| | GGAGTGCAGGTGGAgACtATCTCCCCAGGAGACGGGCGCACCTTCCCC | GVQVETISPGDGRTFPKRGQTCV |
| | AAGCGCGGCCAGACCTGCGTGGTGCACTACACCGGGATGCTTGAAGA | VHYTGMLEDGKKVDSSRDRNKPF |
| | TGGAAAGAAAGTTGATTCCTCCCGGGACAGAAACAAGCCCTTTAAGTT | KFMLGKQEVIRGWEEGVAQMSV |
| | TATGCTAGGCAAGCAGGAGGTGATCCGAGGCTGGGAAGAAGGGGTT | GQRAKLTISPDYAYGATGHPGIIP |
| | GCCCAGATGAGTGTGGGTCAGAGAGCCAAACTGACTATATCTCCAGAT | PHATLVFDVELLKL |
| | TATGCCTATGGTGCCACTGGGCACCCAGGCATCATCCCACCACATGCCA | |
| | CTCTCGTCTTCGATGTGGAGCTTCTAAAACTGGA | |
| Linker | SEQ ID NO: 192 | SEQ ID NO: 193 |
| | ATCTGGCGGTGGATCCGGA | SGGGSG |
| ΔCaspase9 | SEQ ID NO: 194 | SEQ ID NO: 195 |
| | GTCGACGGATTTGGTGATGTCGGTGCTCTTGAGAGTTTGAGGGGAAAT | VDGFGDVGALESLRGNADLAYILS |
| | GCAGATTTGGCTTACATCCTGAGCATGGAGCCCTGTGGCCACTGCCTCA | MEPCGHCLIINNVNFCRESGLRTR |
| | TTATCAACAATGTGAACTTCTGCCGTGAGTCCGGGCTCCGCACCCGCAC | TGSNIDCEKLRRRFSSLHFMVEVK |
| | TGGCTCCAACATCGACTGTGAGAAGTTGCGGCGTCGCTTCTCCTCGCTG | GDLTAKKMVLALLELARQDHGAL |
| | CATTTCATGGTGGAGGTGAAGGGCGACCTGACTGCCAAGAAAATGGT | DCCVVVILSHGCQASHLQFPGAV |
| | GCTGGCTTTGCTGGAGCTGGCGCgGCAGGACCACGGTGCTCTGGACTG | YGTDGCPVSVEKIVNIFNGTSCPSL |
| | CTGCGTGGTGGTCATTCTCTCTCACGGCTGTCAGGCCAGCCACCTGCAG | GGKPKLFFIQACGGEQKDHGFEV |
| | TTCCCAGGGGCTGTCTACGGCACAGATGGATGCCCTGTGTCGGTCGAG | ASTSPEDESPGSNPEPDATPFQEG |
| | AAGATTGTGAACATCTTCAATGGGACCAGCTGCCCCAGCCTGGGAGGG | LRTFDQLDAISSLPTPSDIFVSYSTF |
| | AAGCCCAAGCTCTTTTTCATCCAGGCCTGTGGTGGGGAGCAGAAAGAC | PGFVSWRDPKSGSWYVETLDDIF |
| | CATGGGTTTGAGGTGGCCTCCACTTCCCCTGAAGACGAGTCCCCTGGC | EQWAHSEDLQSLLLRVANAVSVK |
| | AGTAACCCCGAGCCAGATGCCACCCCGTTCCAGGAAGGTTTGAGGACC | GIYKQMPGCFNFLRKKLFFKTS |
| | TTCGACCAGCTGGACGCCATATCTAGTTTGCCCACACCCAGTGACATCT | |
| | TTGTGTCCTACTCTACTTTCCCAGGTTTTGTTTCCTGGAGGGACCCCAAG | |
| | AGTGGCTCCTGGTACGTTGAGACCCTGGACGACATCTTTGAGCAGTGG | |
| | GCTCACTCTGAAGACCTGCAGTCCCTCCTGCTTAGGGTCGCTAATGCTG | |
| | TTTCGGTGAAAGGGATTTATAAACAGATGCCTGGTTGCTTTAATTTCCT | |
| | CCGGAAAAAACTTTTCTTTAAAACATCAGCTAGCAGAGCCGAGGGCAG | |
| | GGGAAGTCTTCTAACATGCGGGGACGTGGAGGAAAATCCCGGGCCC- | |
| | tga | |
| Linker | SEQ ID NO: 196 | SEQ ID NO: 197 |
| | GCTAGCAGAGCCccgcgg | ASRAPR |
| T2A | SEQ ID NO: 198 | SEQ ID NO: 199 |
| | GAGGGCAGGGGAAGTCTTCTAACATGCGGGGACGTGGAGGAAAATCC | EGRGSLLTCGDVEENPGP |
| | CGGGCCC | |
| Linker | SEQ ID NO: 200 | SEQ ID NO: 201 |
| | GCATGCGCCACC | ACAT |
| Signal | SEQ ID NO: 202 | SEQ ID NO: 203 |
| Peptide | ATGGAGTTTGGGTTGTCATGGTTGTTTCTCGTCGCTATTCTCAAAGGTG | MEFGLSWLFLVAILKGVQCSR |
| | TACAATGCTCCCGC | |
| FRP5VH | SEQ ID NO: 204 | SEQ ID NO: 205 |
| | GAAGTCCAATTGCAACAGTCAGGCCCCGAATTGAAAAAGCCCGGCGAA | EVQLQQSGPELKKPGETVKISCKA |
| | ACAGTGAAGATATCTTGTAAAGCCTCCGGTTACCCTTTTACGAACTATG | SGYPFTNYGMNWVKQAPGQGL |
| | GAATGAACTGGGTCAAACAAGCCCCTGGACAGGGATTGAAGTGGATG | KWMGWINTSTGESTFADDFKGR |
| | GGATGGATCAATACATCAACAGGCGAGTCTACCTTCGCAGATGATTTC | FDFSLETSANTAYLQINNLKSEDM |
| | AAAGGTCGCTTTGACTTCTCACTGGAGACCAGTGCAAATACCGCCTACC | ATYFCARWEVYHGYVPYWGQGT |
| | TTCAGATTAACAATCTTAAAAGCGAGGATATGGCAACCTACTTTTGCGC | TVTVSS |
| | AAGATGGGAAGTTTATCACGGGTACGTGCCATACTGGGGACAAGGAA | |
| | CGACAGTGACAGTTAGTAGC | |
| Flex Linker | SEQ ID NO: 206 | SEQ ID NO: 207 |
| | GGCGGTGGAGGCTCCGGTGGAGGCGGCTCTGGAGGAGGAGGTTCA | GGGGSGGGGSGGGGS |
| FRP5VL | SEQ ID NO: 208 | SEQ ID NO: 209 |
| | GACATCCAATTGACACAATCACACAAATTTCTCTCAACTTCTGTAGGAG | DIQLTQSHKFLSTSVGDRVSITCKA |
| | ACAGAGTGAGCATAACCTGCAAAGCATCCCAGGACGTGTACAATGCTG | SQDVYNAVAWYQQKPGQSPKLLI |
| | TGGCTTGGTACCAACAGAAGCCTGGACAATCCCCAAAATTGCTGATTTA | YSASSRYTGVPSRFTGSGSGPDFT |
| | TTCTGCCTCTAGTAGGTACACTGGGGTACCTTCTCGGTTTACGGGCTCT | FTISSVQAEDLAVYFCQQHFRTPF |
| | GGGTCCGGACCAGATTTCACGTTCACAATCAGTTCCGTTCAAGCTGAA | TFGSGTKLEIKAL |
| | GACCTCGCTGTTTATTTTTGCCAGCAGCACTTCCGAACCCCTTTTACTTT | |
| | TGGCTCAGGCACTAAGTTGGAAATCAAGGCTTTG | |
| Linker | SEQ ID NO: 210 | SEQ ID NO: 211 |
| | Atgcat | MH |
| CD34- | SEQ ID NO: 212 | SEQ ID NO: 213 |
| epitope | GAACTTCCTACTCAGGGGACTTTCTCAAACGTTAGCACAAACGTAAGT | ELPTQGTFSNVSTNVS |
| CD8 stalk | SEQ ID NO: 214 | SEQ ID NO: 215 |
| | CCCGCCCCAAGACCCCCCACACCTGCGCCGACCATTGCTTCTCAACCCC | PAPRPPTPAPTIASQPLSLRPEACR |
| | TGAGTTTGAGACCCGAGGCCTGCCGGCCAGCTGCCGGCGGGGCCGTG | PAAGGAVHTRGLDFACD |
| | CATACAAGAGGACTCGATTTCGCTTGCGAC | |
| CD8 TM, | SEQ ID NO: 216 | SEQ ID NO: 217 |
| stop tf | ATCTATATCTGGGCACCTCTCGCTGGCACCTGTGGAGTCCTTCTGCTCA | IYIWAPLAGTCGVLLLSLVIT- |
| | GCCTGGTTATTACT- | LYCNHRNRRRVCKCPRVD |
| | CTGTACTGTAATCACCGGAATCGCCGCCGCGTTTGTAAGTGTCCCAGG | |
| | GTCGAC | |
| dMyD88 | SEQ ID NO: 218 | SEQ ID NO: 219 |
| | ATGGCCGCTGGGGGCCCAGGCGCCGGATCAGCTGCTCCCGTATCTTCT | MAAGGPGAGSAAPVSSTSSLPLA |
| | ACTTCTTCTTTGCCGCTGGCTGCTCTGAACATGCGCGTGAGAAGACGCC | ALNMRVRRRLSLFLNVRTQVAAD |
| | TCTCCCTGTTCCTTAACGTTCGCACACAAGTCGCTGCCGATTGGACCGC | WTALAEEMDFEYLEIRQLETQAD |
| | CCTTGCCGAAGAAATGGACTTTGAATACCTGGAAATTAGACAACTTGA | PTGRLLDAWQGRPGASVGRLLDL |
| | AACACAGGCCGACCCCACTGGCAGACTCCTGGACGCATGGCAGGGAA | LTKLGRDDVLLELGPSIEEDCQKYI |
| | GACCTGGTGCAAGCGTTGGACGGCTCCTGGATCTCCTGACAAAACTGG | LKQQQEEAEKPLQVAAVDSSVPR |
| | GACGCGACGACGTACTGCTTGAACTCGGACCTAGCATTGAAGAAGACT | TAELAGITTLDDPLGHMPERFDAF |
| | GCCAAAAATATATCCTGAAACAACAACAAGAAGAAGCCGAAAAACCTC | ICYCPSDI |
| | TCCAAGTCGCAGCAGTGGACTCATCAGTACCCCGAACAGCTGAGCTTG | |
| | CTGGGATTACTACACTCGACGACCCACTCGGACATATGCCTGAAAGATT | |
| | CGACGCTTTCATTTGCTATTGCCCCTCTGACATA | |
| dCD40 | SEQ ID NO: 220 | SEQ ID NO: 221 |
| | AAGAAAGTTGCAAAGAAACCCACAAATAAAGCCCCACACCCTAAACAG | KKVAKKPTNKAPHPKQEPQEINFP |
| | GAACCCCAAGAAATCAATTTCCCAGATGATCTCCCTGGATCTAATACTG | DDLPGSNTAAPVQETLHGCQPVT |
| | CCGCCCCGGTCCAAGAAACCCTGCATGGTTGCCAGCCTGTCACCCAAG | QEDGKESRISVQERQ |
| | AGGACGGAAAAGAATCACGGATTAGCGTACAAGAGAGACAA | |
| CD3zeta | SEQ ID NO: 222 | SEQ ID NO: 223 |
| | AGAGTGAAGTTCAGCAGGAGCGCAGACGCCCCCGCGTACCAGCAGGG | RVKFSRSADAPAYQQGQNQLYN |
| | CCAGAACCAGCTCTATAACGAGCTCAATCTAGGACGAAGAGAGGAGTA | ELNLGRREEYDVLDKRRGRDPEM |
| | CGATGTTTTGGACAAGAGACGTGGCCGGGACCCTGAGATGGGGGGAA | GGKPRRKNPQEGLYNELQKDKM |
| | AGCCGAGAAGGAAGAACCCTCAGGAAGGCCTGTACAATGAACTGCAG | AEAYSEIGMKGERRRGKGHDGLY |
| | AAAGATAAGATGGCGGAGGCCTACAGTGAGATTGGGATGAAAGGCGA | QGLSTATKDTYDALHMQALPPR* |
| | GCGCCGGAGGGGCAAGGGGCACGATGGCCTTTACCAGGGTCTCAGTA | |
| | CAGCCACCAAGGACACCTACGACGCCCTTCACATGCAAGCTCTTCCACC | |
| | TCGT-TGA | |

### Example 11

### Representative Embodiments

Provided hereafter are examples of certain embodiments of the technology.

A1. A cell, comprising a polynucleotide that encodes a chimeric protein comprising a multimerization region and a modified caspase-9 polypeptide, wherein the modified caspase-9 polypeptide comprises an amino acid sequence having at least 90% sequence identity to SEQ ID NO: 9, and comprises at least one amino acid substitution selected from the group consisting of S144A, S144D, Y153A, Y153F, S183A, S195A, S196A, S196D, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, C403A, C403S, C403T, F404T, F404W, F404Y, N405A, N405F, N405Q, N405T, F406A, F406T, F406W, F406Y, G402A, G402I, G402Q, G402Y, C403P, F404A, F4045, and F406L.

A2. The cell of embodiment A1, wherein the at least one amino acid substitution is selected from the group consisting of S144A, S144D, Y153A, Y153F, S183A, S195A, S196A, S196D, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, C403A, C403S, C403T, F404T, F404W, F404Y, N405A, N405F, N405Q, N405T, F406A, F406T, F406W, and F406Y.

A3. The cell of embodiment A1, wherein the at least one amino acid substitution is selected from the group consisting of S144A, S144D, Y153A, Y153F, S183A, S195A, S196A, S196D, S307A, D315A, A316G, T317A, T317C, T317S, P318A, F319A, F319W, L329E, D330A, D330E, D330G, D330N, D330S, D330V, C403A, C403S, C403T, F404T, F404W, F404Y, N405A, N405F, N405Q, F406A, F406T, F406W, and F406Y.

A4. The cell of embodiment A1, wherein the at least one amino acid substitution is S144A, S144D, Y153A, Y153F, S183A, S195A, S196A, S196D, S307A, D315A, A316G, T317A, T317S, F319W, L329E, D330A, D330E, D330G, D330N, D330S, D330V, F404T, F404W, F404Y, N405F, N405Q, F406A, F406T, F406W, and F406Y.

A5. The cell of embodiment A1, wherein the at least one amino acid substitution is S144A, S144D, S183A, S195A, S196A, S196D, T317A, T317S, L329E, D330A, D330E, D330G, D330N, D330S, D330V, F404Y, N405Q, F406A, F406W, and F406Y.

A6. The cell of embodiment A1, wherein the at least one amino acid substitution is selected from the group consisting of T317S, S144A, S133, and S196D.

A7. The cell of embodiment A1, wherein the at least one amino acid substitution is selected from the group consisting of S183A, S195A, S196A, S196D, T317A, L329E, D330A, D330E, D330G, D330N, D330S, D330V, F404Y, N405Q, F406A, F406W, and F406Y.

A8. The cell of embodiment A1, wherein the at least one amino acid substitution is D330A.

A9. The cell of embodiment A1, wherein the at least one amino acid substitution is D330E.

A10. The cell of embodiment A1, wherein the at least one amino acid substitution is N405Q.

A11. The cell of embodiment A1, wherein the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, ⁴⁰²GCFNF⁴⁰⁶ISAQT (CASP-10) (SEQ ID NOS 224 and 225), ³¹⁶ATPF³¹⁹AVPI (SMAC/Diablo) (SEQ ID NOS 226 and 227), D330A-N405T, D315A-D330A, D330A-Y153A, D330A-Y153F, D330A-T317E, ⁴⁰²GCFNF⁴⁰⁶CIVSM (CASP-3) (SEQ ID NOS 224 and 228), ⁴⁰²GCFNF⁴⁰⁶AAAAA (SEQ ID NOS 224 and 229), ⁴⁰²GCFNF⁴⁰⁶YCSTL (CASP-2) (SEQ ID NOS 224 and 230), and ⁴⁰²GCFNF⁴⁰⁶QPTFT (CASP-8) (SEQ ID NOS 224 and 231).

A12. The cell of embodiment A1, wherein the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, ⁴⁰²GCFNF⁴⁰⁶ISAQT (CASP-10) (SEQ ID NOS 224 and 225), ³¹⁶ATPF³¹⁹AVPI (SMAC/Diablo) (SEQ ID NOS 226 and 227), and D330A-N405T.

A13. The cell of embodiment A1, wherein the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, ⁴⁰²GCFNF⁴⁰⁶ISAQT (CASP-10) (SEQ ID NOS 224 and 225), and ³¹⁶ATPF³¹⁹AVPI (SMAC/Diablo) (SEQ ID NOS 226 and 227).

A14. The cell of embodiment A1, wherein the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, and ⁴⁰²GCFNF⁴⁰⁶ISAQT (CASP-10) (SEQ ID NOS 224 and 225).

A15. The cell of embodiment A1, wherein the modified caspase-9 polypeptide comprises at least two amino acid substitutions selected from the group consisting of N405Q-S144Aco and N405Q-T317Sco.

A16. The cell of embodiment A1, wherein the at least one amino acid substitution is selected from the group consisting of S144A, S144D, Y153A, Y153F, S183A, S195A, S196A, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, F404T, F404W, F404Y, N405F, N405Q, and F406T.

A17. The cell of embodiment A1, wherein the at least one amino acid substitution is selected from the group consisting of S144A, S144D, Y153A, Y153F, S183A, S195A, S196A, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, F404T, F404W, F404Y, N405F, N405Q, and F406T.

A18. The cell of embodiment A1, wherein the at least one amino acid substitution is selected from the group consisting of S144A, S144D, Y153A, Y153F, S183A, S195A, S196A, S307A, D315A, A316G, T317A, T317C, T317S, P318A, F319A, F319W, L329E, D330A, D330E, D330G, D330N, D330S, D330V, F404T, F404W, F404Y, N405F, N405Q, and F406T.

A19. The cell of embodiment A1, wherein the at least one amino acid substitution is selected from the group consisting of S144A, S144D, Y153A, Y153F, S183A, S195A, S196A, S307A, D315A, A316G, T317A, T317S, F319W, D330A, F404T, F404W, F404Y, N405F, N405Q, and F406T.

A20. The cell of embodiment A1, wherein the at least one amino acid substitution is selected from the group consisting of S144A, S144D, S183A, S195A, S196A, T317A, T317S, D330A, F404Y, and N405Q.

A21. The cell of embodiment A1, wherein the at least one amino acid substitution is selected from the group consisting of S196D, T317C, T317E, P318A, F319A, F326K, D327G, D327K, D327R, Q328K, Q328R, L329E, L329G, L329K, D330E, D330G, D330N, D330S, D330V, A331K, C403A, C403S, C403T, N405A, N405T, F406A, F406W, F406Y, G402A, G402I, G402Q, G402Y, C403P, F404A, F404S, and F406L.

A22. The cell of embodiment A1, wherein the at least one amino acid substitution is selected from the group consisting of S196D, T317C, T317E, P318A, F319A, F326K, D327G, D327K, D327R, Q328K, Q328R, L329E, L329G, L329K, D330E, D330G, D330N, D330S, D330V, A331K, C403A, C403S, C403T, N405A, N405T, F406A, F406W, and F406Y.

A23. The cell of embodiment A1, wherein the at least one amino acid substitution is selected from the group consisting of S196D, T317C, P318A, F319A, L329E, D330E, D330G, D330N, D330S, D330V, C403A, C403S, C403T, N405A, F406A, F406T, F406W, and F406Y.

A24. The cell of embodiment A1, wherein the at least one amino acid substitution is selected from the group consisting of S196D, L329E, D330E, D330G, D330N, D330S, D330V, F406A, F406T, F406W, and F406Y.

A25. The cell of embodiment A1, wherein the at least one amino acid substitution is selected from the group consisting of S196D, L329E, D330E, D330G, D330N, D330S, D330V, F406A, F406W, and F406Y.

A26. The cell of any one of embodiments A1-25, wherein the polynucleotide comprises optimized codons encoding the caspase-9 polypeptide.

A27. The cell of embodiment A26, wherein the modified caspase-9 polypeptide comprises an amino acid substitution of N405Q.

A28. The cell of embodiment A26, wherein the modified caspase-9 polypeptide is encoded by the nucleic acid sequence of SEQ ID NO: 39.

A29. The cell of any one of embodiments A1-28, wherein the cell is a human cell.

A30. The cell of any one of embodiments A1-29, wherein the cell is a progenitor cell.

A31. The cell of any one of embodiments A1-30, wherein the cell is a hematopoietic progenitor cell.

A32. The cell of any one of embodiments A1-29, wherein the cell is selected from the group consisting of mesenchymal stromal cells, embryonic stem cells, and inducible pluripotent stem cells.

A33. The cell of any one of embodiments A1-29, wherein the cell is a T cell.

A34. The cell of any one of embodiments A1-29, wherein the cell is obtained or prepared from bone marrow.

A35. The cell of any one of embodiments A1-29, wherein the cell is obtained or prepared from umbilical cord blood.

A36. The cell of any one of embodiments A1-29, wherein the cell is obtained or prepared from peripheral blood.

A37. The cell of any one of embodiments A1-29, wherein the cell is obtained or prepared from peripheral blood mononuclear cells.

A38. The cell of any one of embodiments A1-37, wherein the polynucleotide is operably linked to a promoter.

A39. The cell of embodiment A38, wherein the promoter is developmentally regulated and the caspase-9 polypeptide is expressed in developmentally differentiated cells.

A40. The cell of embodiments A38 or A39, wherein the promoter is tissue-specific and the caspase-9 polypeptide is expressed in the specific tissue.

A41. The cell of embodiments A38 or A39, wherein the promoter is activated in activated T cells.

A42. The cell of embodiments A38-41 wherein the promoter comprises a 5′LTR sequence.

A43. The cell of any one of embodiments A1-43, wherein the chimeric protein further comprises a marker polypeptide.

A44. The cell of embodiment A43, wherein the marker polypeptide is a ΔCD19 polypeptide.

A45. The cell of any one of embodiments A1-45, wherein the caspase-9 polypeptide is a truncated caspase-9 polypeptide.

A46. The cell of any one of embodiments A1-46 wherein the caspase-9 polypeptide lacks the Caspase recruitment domain.

A47. The cell of embodiment A1, wherein, the at least one amino acid substitution is selected from the group consisting of N405Q, F404Y, F406A, F406W, F406Y, F404T, F404W, N405F, F406T, C403A, C403S, C403T, N405A, and N405T.

A48. The cell of embodiment A1, wherein, the at least one amino acid substitution is selected from the group consisting of N405Q, F404Y, F406A, F406W, and F406Y.

A49. The cell of embodiment A1, wherein, the at least one amino acid substitution is selected from the group consisting of T317S, D330A, D330E, D330G, D330N, D330S, D330V, L329E, T317A, D315A, A316G, T317C, P318A, F319A, T317E, F326K, D327G, D327K, D327R, Q328K, Q328R, L329G, L329K, and A331K.

A50. The cell of embodiment A1, wherein, the at least one amino acid substitution is selected from the group consisting of T317S, D330A, D330E, D330G, D330N, D330S, D330V, L329E, and T317A.

A51. The cell of embodiment A1, wherein, the at least one amino acid substitution is selected from the group consisting of S144A, S144D, S196D, S183A, S195A, S196A, Y153A, Y153F, and S307A.

A51.1 The cell of any one of embodiments A1-51, wherein the cell further comprises a polynucleotide that encodes a chimeric antigen receptor.

A51.2 The cell of any one of embodiments A1-51.1, wherein the cell comprises a polynucleotide that encodes the modified caspase-9 polypeptide and further encodes a heterologous polypeptide and a linker polypeptide linking the modified caspase-9 polypeptide and the heterologous polypeptide, wherein the linker polypeptide separates the modified caspase-9 polypeptide and the heterologous polypeptide during or after translation.

A51.3. The cell of embodiment 51.2, wherein the heterologous polypeptide is a chimeric antigen receptor (CAR).

A51.4. The cell of any one of embodiments A1-51.3, wherein the cell comprises a polynucleotide encoding a chimeric antigen receptor.

A51.5. A nucleic acid comprising

a first polynucleotide encoding a chimeric antigen receptor, and a second polynucleotide encoding a chimeric caspase-9 polypeptide comprising a multimeric ligand binding region and a caspase-9 polypeptide.

A51.6. The nucleic acid of embodiment 51.5, wherein the chimeric antigen receptor comprises

(i) a transmembrane region; (ii)) a T cell activation molecule; and (iii) an antigen recognition moiety.

A51.7. The nucleic acid of any one of embodiments 51.5 or 51.6, further comprising at least one promoter.

A51.8. The nucleic acid of any one of embodiments 51.5 or 51.6, further comprising at least two promoters.

A51.9. The nucleic acid of embodiment 51.7, wherein the at least one promoter is operably linked to both the first and second polynucleotides.

A51.10. The nucleic acid of any one of embodiments 51.5-51.9, further comprising a third polynucleotide encoding a linker polypeptide between the first and second polynucleotides, wherein the linker polypeptide separates the translation products of the first and second polynucleotides during or after translation.

A51.11. The nucleic acid of embodiment 51.10, wherein the linker polypeptide is a 2A polypeptide.

A51.12. The nucleic acid of embodiment 51.10, wherein the nucleic acid encodes a polypeptide comprising a chimeric antigen receptor, a 2A polypeptide, and a caspase-9 polypeptide.

A51.13. The nucleic acid of embodiment 51.8, wherein the first polynucleotide is operably linked to a first promoter and the second polynucleotide is operably linked to a second promoter.

A51.14. The nucleic acid of embodiment 51.5, wherein two RNA transcripts are produced complementary to the two polynucleotides.

A51.15. The nucleic acid of any one of embodiments 51.5-51.14, wherein the chimeric antigen receptor comprises (i) a transmembrane region; (ii) a T cell activation molecule; and (iii) an antigen recognition moiety.

A51.16. The nucleic acid of embodiment 51.15, wherein the chimeric antigen receptor further comprises a co-stimulatory molecule.

A51.17. The nucleic acid of embodiment 51.15, wherein the co-stimulatory molecule is selected from the group consisting of CD28, OX40, and 4-1 BB.

A51.18. The nucleic acid of any one of embodiments 51.15-51.17, wherein the T cell activation molecule is an ITAM-containing, Signal 1 conferring molecule.

A51.19. The nucleic acid of any one of embodiments 51.15-51.17, wherein the T cell activation molecule is a CD3 zeta polypeptide.

A51.20. The nucleic acid of any one of embodiments 51.15-51.17, wherein the T cell activation molecule is an Fc receptor gamma polypeptide.

A51.21. The nucleic acid of any one of embodiments 51.15-51.20, wherein the antigen recognition moiety binds to an antigen on a tumor cell.

A51.22. The nucleic acid of any one of embodiments 51.15-51.20, wherein the antigen recognition moiety binds to an antigen on a cell involved in a hyperproliferative disease.

A51.23. The nucleic acid of any one of embodiments 51.15-51.20, wherein the antigen recognition moiety binds to an antigen selected from the group consisting of PSMA, PSCA, MUC1, CD19, ROR1, Mesothelin, GD2, CD123, MUC16, and Her2/Neu.

A51.23. The nucleic acid of any one of embodiments 51.15-51.20, wherein the antigen recognition moiety binds to PSCA.

A51.24. The nucleic acid of any one of embodiments 51.15-51.20, wherein the antigen recognition moiety binds to CD19.

A51.25. The nucleic acid of any one of embodiments 51.15-51.20, wherein the antigen recognition moiety binds to a viral or bacterial antigen.

A51.26. The nucleic acid of any one of embodiments 51.15-51.25, wherein the antigen recognition moiety is a single chain variable fragment.

A51.27. The nucleic acid of any one of embodiments 51.15-51.26, wherein the transmembrane region is a CD28 transmembrane region.

A51.28. The nucleic acid of any one of embodiments 51.15-51.26, wherein the transmembrane region is a CD8 transmembrane region.

A51.29. The nucleic acid of embodiment 51.28, further comprising a CD8 stalk region.

A51.30. A modified cell transfected or transformed with a nucleic acid of any one of embodiments 51.5-51.29.

A51.31. The modified cell of any one of embodiments 51.4 or 51.30, wherein the multimeric ligand binding region is an FKB12v36 region.

A51.32. The modified cell of any one of embodiments 51.4, 51.30, or 51.31, wherein the multimeric ligand is AP1903, or AP20187.

A51.33. The modified cell of any one of embodiments 51.4 or 51.30-51.32, wherein the caspase-9 polypeptide is a modified caspase-9 polypeptide comprising an amino acid substitution selected from the group consisting of the caspase variants in Table 1.

A51.34. The cell of any one of embodiments 51.2, 51.3, 51.4, or 51.30-51.33, wherein the linker polypeptide is a 2A or a 2A-like polypeptide.

A51.35. The modified cell of any one of embodiments A1-51, or 51.2, wherein the modified cell further comprises a polynucleotide encoding a T cell receptor.

A51.36. The modified cell of any one of embodiments A1-51, or 51.2, wherein the modified cell further comprises a polynucleotide encoding a T cell receptor-based CAR.

A51.37. The modified cell of any one of embodiments 51.35 or 51.36, wherein modified cell is transfected or transformed with a nucleic acid comprising a polynucleotide encoding the T cell receptor or T cell receptor-based CAR.

A51.38. The modified cell of any one of embodiments A51.35-51.37, wherein the chimeric antigen receptor or T cell receptor binds to an antigen on a tumor cell.

A51.39. The modified cell of any one of embodiments A51.35-51.37 wherein the chimeric antigen receptor or T cell receptor binds to an antigen on a cell involved in a hyperproliferative disease.

A51.40. The modified cell of any one of embodiments A51.35-51.37, wherein the chimeric antigen receptor or T cell receptor binds to an antigen selected from the group consisting of PSMA, PSCA, MUC1, CD19, ROR1, Mesothelin, GD2, CD123, MUC16, and Her2/Neu.

A51.41. The modified cell of any one of embodiments A51.35-51.37, wherein the chimeric antigen receptor or T cell receptor binds to CD19.

A51.42. The modified cell of any one of embodiments A51.35-51.37 wherein the T cell receptor binds to Her2/Neu.

A52. The cell of any one of embodiments A1-51.4, or A51.28-51.42, wherein the multimerization region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof.

A53. The cell of any one of embodiments A1-51.4, or A51.28-52, wherein the multimerization region is an FKBP12 region.

A54. The cell of embodiment A53, wherein the FKB12 region is an FKB12v₃₆ region.

A55. The cell of any one of embodiments A1-51.4, or A51.28-54, wherein the multimerization region is Fv′Fvls.

A56. The cell of any one of embodiments A1-51.4, or A51.28-55, wherein the multimerization region binds a ligand selected from the group consisting of an FK506 dimer and a dimeric FK506 analog ligand.

A57. The cell of any one of embodiments A1-51.4, or A51.28-56, wherein the ligand is AP1903 or AP20187.

A58. The cell of any one of embodiments A1-51.4, or A51.28-57, wherein the multimerization region has an amino acid sequence of SEQ ID NO: 29 or a functional fragment thereof.

A59. The cell of any one of embodiments A1-51.4, or A51.28-58, wherein the multimerization region is encoded by a nucleotide sequence in SEQ ID NO: 30, or a functional fragment thereof.

A60. The cell of embodiment A58, wherein the multimerization region further comprises a polypeptide having an amino acid sequence of SEQ ID NO: 32, or a functional fragment thereof.

A61. The cell of embodiment A59, wherein the multimerization region further comprises a polypeptide encoded by a nucleotide sequence in SEQ ID NO: 31, or a functional fragment thereof.

A62. The cell of embodiment A60, wherein the multimerization region further comprises a polypeptide having an amino acid sequence of SEQ ID NO: 32, or a functional fragment thereof.

A63. The cell of embodiment A61, wherein the multimerization region further comprises a polypeptide encoded by a nucleotide sequence in SEQ ID NO: 31, or a functional fragment thereof.

A64. The cell of any of embodiments A58, A60, or A62, wherein the multimerization region further comprises a polypeptide having an amino acid sequence of SEQ ID NO: 29 or SEQ ID NO: 32, or a functional fragment thereof.

A65. The cell of any of embodiments A59, A61, or A63, wherein the multimerization region further comprises a polypeptide encoded by a nucleotide sequence in SEQ ID NO: 30 or SEQ ID NO: 31, or a functional fragment thereof.

A66. The cell of any one of embodiments A1-51.4, or A51.28-65, wherein the cells are transduced or transfected with a retroviral vector.

A67. The cell of embodiment A66, wherein the retroviral vector is a murine leukemia virus vector.

A68. The cell of embodiment A66, wherein the retroviral vector is an SFG vector.

A69. The cell of any one of embodiments A1-51.4, or A51.28-65, wherein the cells are transduced or transfected with an adenoviral vector.

A70. The cell of any one of embodiments A1-51.4, or A51.28-65, wherein the cells are transduced or transfected with a lentiviral vector.

A71. The cell of any one of embodiments A1-51.4, or A51.28-70, wherein the cells are further transfected or transduced with a gene expression vector.

A72. The cell of embodiment A71, wherein the cells comprise a polynucleotide that encodes the modified caspase-9 polypeptide and further comprise a second polynucleotide that encodes a heterologous polypeptide.

A73. The cell of embodiment A72, wherein the heterologous polypeptide is a chimeric antigen receptor.

A74. The cell of any one of embodiments A1-51.4, or A51.28-78, wherein the cells further comprise a polynucleotide that encodes a chimeric protein comprising a multimerization region and a caspase-9 polypeptide or a second modified caspase-9 polypeptide., wherein the first and second caspase-9 polypeptides comprise different amino acid sequences and have different basal activities or different IC₅₀s.

A75. The cell of embodiment A74, wherein
a) the modified caspase-9 polypeptide and the caspase-9 polypeptide; or
b) the modified caspase-9 polypeptide and the second modified caspase-9 polypeptide
have different IC₅₀s or different elongated dose response curves, or different IC₅₀s and different elongated dose response curves to the multimeric ligand.

A76. The cell of any one of embodiments A1-51.4, or A51.28-75, which is isolated.

A77. The cell of any one of embodiments A1-51.4, or A51.28-75, which is in a human subject.

A78. The cell of embodiment A77, which is transplanted in a human subject.

A79. A method of administering donor T cells to a human patient, comprising administering cells of any one of embodiments A1-51.4, or A51.28-78 to a human patient, wherein the cells are human donor T cells.

A80. The method of embodiment A79, wherein the cells are transduced or transfected in a donor cell culture.

A81. The method of embodiments A79 or A80, further comprising
a) detecting the presence of graft versus host disease in the patient after administering the cells to the patient; and
b) administering a multimeric ligand that binds to the multimerization region to the patient for whom the presence of graft versus host disease is detected.

A82. The method of embodiment A81, wherein the effect of the graft versus host disease is reduced following administration of the multimeric ligand.

A83. A method of stem cell transplantation, comprising
a) administering a stem cell transplant to a human patient; and
b) administering cells of any one of embodiments A1-51.4, or A51.28-78, wherein the cells are donor T cells, to the patient.

A84. The method of embodiment A83, wherein the stem cell transplant is selected from the group consisting of a matched transplant, a partially-matched transplant, a haploidentical transplant, and aCD34⁺ haploidentical stem cell transplant.

A85. The method of any one of embodiments A79-84 wherein the human donor T cells are matched, partially matched, or haploidentical to the patient's T cells.

A86. The method of any one of embodiments A74-85, wherein the patient has cancer.

A87. The method of any one of embodiments A79-86, wherein the patient has a solid tumor.

A88 The method of embodiments A86 or A87, wherein the cancer is present in the blood or bone marrow of the patient.

A89 The method of any one of embodiments A79-88, wherein the patient has a blood or bone marrow disease.

A90 The method of any one of embodiments A79-89, wherein the patient has been diagnosed with any condition or disorder that can be alleviated by stem cell transplantation.

A91 The method of any one of embodiments A79-85, or A90, wherein the patient has been diagnosed with a condition selected from the group consisting of a primary immune deficiency disorder, hemophagocytosis lymphohistiocytosis (HLH) or other hemophagocytic disorder, an inherited marrow failure disorder, a hemoglobinopathy, a metabolic disorder, and an osteoclast disorder.

A92. The method of embodiment A91, wherein the condition is selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCK 8 Deficiency, IL-10 Deficiency/IL-10 Receptor Deficiency, GATA 2 deficiency, X-linked lymphoproliferative disease (XLP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis.

A93. The method of any of embodiments A79-92, further comprising a selection step, wherein cells that express a marker are selected for administration to the patient.

A94. The method of any one of embodiments A79-93, wherein the cells are transfected in a donor cell culture that is prepared from a bone marrow sample.

A95 The method of embodiment A79-93, wherein the cells are transfected in a donor cell culture that is prepared from peripheral blood.

A96. The method of embodiment A95, wherein the donor cell culture is prepared from donor peripheral blood mononuclear cells.

A97. The method of any one of embodiments A79-96, wherein the donor T cells are allodepleted from the donor cell culture before transfection or transduction.

A98. The method of any one of embodiments A79-96, wherein the donor T cells are not allodepleted from the donor cell culture before transfection or transduction.

A99. The method of any one of embodiments A97-98, wherein the transduced or transfected T cells are cultured in the presence of IL-2 before administration to the patient.

A100. The method of any one of embodiments A79-98, further comprising administering a multimeric ligand that binds to the multimerization region.

A101. The method of embodiment A100, wherein the multimeric ligand is AP1903 or AP20187.

A102. The method of embodiments A100 or A101, wherein the multimeric ligand is administered to treat graft versus host disease.

A103. The method of any one of embodiments A81, A82, or A100-102, wherein the patient exhibits graft versus host disease symptoms before the multimeric ligand is administered.

A104. The method of embodiment A103, wherein the patient exhibits one or more Stage 0 graft versus host disease symptoms.

A105. The method of embodiment A103, wherein the patient exhibits one or more Stage 1 graft versus host disease symptoms.

A106. The method of embodiment A103, wherein the patient exhibits one or more Stage 2 graft versus host disease symptoms.

A107. The method of embodiment A103, wherein the patient exhibits one or more Stage 3 graft versus host disease symptoms.

A108. The method of embodiment A103, wherein the patient exhibits one or more Stage 4 graft versus host disease symptoms.

A109. The method of any of embodiments A81, A82, or A100-108, wherein more than one dose of the multimeric ligand is administered.

A110. The method of any of embodiments A81, A81, or A100-109, wherein after administration of the multimeric ligand, the number of alloreactive T cells is reduced.

A111. The method of any one of embodiments A81, A82, or A100-110, wherein the alloreactive T cells express the marker and CD3.

A112. The method of embodiment A111, wherein the number of alloreactive T cells is reduced by about 90% or more after administration of the multimeric ligand.

A113. The method of any one of embodiments A81, A82, or A100-112, wherein after administration of the multimeric ligand, donor T cells survive in the patient that are able to expand and are reactive to viruses and fungi.

A114. The method of any one of embodiments A81, A82, or A100-113, wherein after administration of the multimeric ligand, donor T cells survive in the patient that are able to expand and are reactive to tumor cells in the patient.

A115. The method of any one of embodiments A79-114, wherein the patients have received stem cell transplants before or at the same time as administration of the donor T cells.

A116. The method of any one of embodiments A81, A82, or A100-115, further comprising determining whether to administer an additional dose or additional doses of the multimeric ligand to the patient based upon the appearance of graft versus host disease symptoms in the patient.

A117. The method of any one of embodiments A79-116, wherein at least 1×10⁶ transduced or transfected donor T cells are administered to the patient.

A118. The method of any one of embodiments A79-117, wherein at least 1×10⁷ transduced or transfected donor T cells are administered to the patient.

A119. The method of any one of embodiments A79-117, wherein at least 1×10⁸ transduced or transfected donor T cells are administered to the patient.

A120. The method of any one of embodiments A79-119, further comprising
identifying the presence, absence or stage of graft versus host disease in the patient, and
administering a multimeric ligand that binds to the multimerization region, maintaining a subsequent dosage of the multimeric ligand, or adjusting a subsequent dosage of the multimeric ligand to the patient based on the presence, absence or stage of the graft versus host disease identified in the patient.

A121. The method of any one of embodiments A79-119, further comprising
identifying the presence, absence or stage of graft versus host disease in the patient, and
determining whether a multimeric ligand that binds to the multimerization region should be administered to the patient, or the dosage of the multimeric ligand subsequently administered to the patient is adjusted based on the presence, absence or stage of the graft versus host disease identified in the patient.

A122. The method of any one of embodiments A79-121, further comprising
receiving information comprising the presence, absence or stage of graft versus host disease in the patient; and
administering a multimeric ligand that binds to the multimerization region, maintaining a subsequent dosage of the multimeric ligand, or adjusting a subsequent dosage of the multimeric ligand to the patient based on the presence, absence or stage of the graft versus host disease identified in the patient.

A123. The method of any one of embodiments A79-119, further comprising
identifying the presence, absence or stage of graft versus host disease in the patient, and
transmitting the presence, absence or stage of the graft versus host disease to a decision maker who administers a multimeric ligand that binds to the multimerization region, maintains a subsequent dosage of the multimeric ligand, or adjusts a subsequent dosage of the multimeric ligand administered to the patient based on the presence, absence or stage of the graft versus host disease identified in the subject.

A124. The method of any one of embodiments A79-119, further comprising
identifying the presence, absence or stage of graft versus host disease in the patient, and
transmitting an indication to administer a multimeric ligand that binds to the multimeric binding region, maintain a subsequent dosage of the multimeric ligand or adjust a subsequent dosage of the multimeric ligand administered to the patient based on the presence, absence or stage of the graft versus host disease identified in the subject.

A125. A method of treating graft versus host disease in a patient who has undergone cell therapy, wherein one or more of the cells introduced for the therapy is a cell of any one of embodiments A1-51.4, or A51.28-78, comprising administering a multimeric ligand that binds to the multimerization region to the patient.

A126. The method of embodiment A125, wherein after administration of the multimeric ligand that binds to the multimeric binding region, the number of alloreactive T cells is reduced.

A127. The method of embodiment A125, wherein alloreactive T cells that are not undergoing cell division are ablated.

A128. The method of embodiment A125, wherein within 2 hours of administration of the multimeric ligand, at least 90% of CD3+ΔCD19+ cells are ablated.

A129. The method of embodiment A125, wherein within 1 hour of administration of the multimeric ligand, at least 90% of CD3+ΔCD19+ cells are ablated.

A130. The method of embodiment A125, wherein within 30 minutes of administration of the multimeric ligand, at least 90% of CD3+ΔCD19+ cells are ablated.

A131. The method of any one of embodiments A125-130, wherein within 24 hours of administration of the multimeric ligand, there is a further log reduction of CD3+ΔCD19+ cells compared to the amount of CD3+ΔCD19+ cells at 30 minutes after administration of the multimeric ligand.

A132. The method of any one of embodiments A125-131, further comprising a resolution of skin and liver GvHD within 24 hours after administration of the multimeric ligand.

A133. A method of controlling the survival of transplanted therapeutic cells in a patient, comprising administering cells of any one of embodiments A1-51.4, or A51.28-78 to a human patient, and administering a multimeric ligand to the patient, wherein the multimeric ligand binds to the multimerization region, wherein the administered cells that express the caspase-9 polypeptide are killed following administration of the multimeric ligand.

A134. A method of transplanting therapeutic cells in a human patient, comprising preparing cells of embodiments A1-51.4, or A51.28-78 for transplantation, and transplanting the therapeutic cells into the human patient.

A135. The method of any one of embodiments A133-134, wherein the patient is a human patient.

A136. The method of any one of embodiments A133-135, wherein the multimeric ligand is administered to kill transplanted therapeutic cells.

A137. The method of any one of embodiments A133-136, wherein the patient has cancer.

A138. The method of any one of embodiments A133-137, wherein the patient has a solid tumor.

A139. The method of any one of embodiments A133-138, wherein the cancer is present in the blood or bone marrow of the patient.

A140. The method of any one of embodiments A133-139 wherein the patient has a blood or bone marrow disease.

A141. The method of any one of embodiments A133-140, wherein the patient has any condition or disorder that can be alleviated by stem cell transplantation.

A142. The method of any one of embodiments A133-140, wherein the patient has been diagnosed with a condition selected from the group consisting of a primary immune deficiency disorder, hemophagocytosis lymphohistiocytosis (HLH) or other hemophagocytic disorder, an inherited marrow failure disorder, a hemoglobinopathy, a metabolic disorder, and an osteoclast disorder.

A143. The method of embodiment A142, wherein the condition is selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCK 8 Deficiency, IL-10 Deficiency/IL-10 Receptor Deficiency, GATA 2 deficiency, X-linked lymphoproliferative disease (XLP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis.

A144. The method of any one of embodiments A133-142 further comprising a selection step, wherein cells that express a marker are selected for administration to the patient.

A145. The method of any one of embodiments A133-144, wherein the multimeric ligand is AP1903 or AP201867.

A146. The method of any one of embodiments A133-145, wherein more than one dose of the multimeric ligand is administered.

A147. The method of any one of embodiments A133-146, further comprising
identifying a presence or absence of a condition in the patient that requires the removal of transfected or transduced therapeutic cells from the patient; and
administering a multimeric ligand that binds to the multimerization region, maintaining a subsequent dosage of the multimeric ligand, or adjusting a subsequent dosage of the multimeric ligand to the patient based on the presence or absence of the condition identified in the patient.

A148. The method of any one of embodiments A133-146 further comprising
identifying a presence or absence of a condition in the patient that requires the removal of transfected or transduced therapeutic cells from the patient; and
determining whether a multimeric ligand that binds to the multimerization region should be administered to the patient, or the dosage of the multimeric ligand subsequently administered to the patient is adjusted based on the presence or absence of the condition identified in the patient.

A149. The method of any one of embodiments A133-146, further comprising
receiving information comprising presence or absence of a condition in the patient that requires the removal of transfected or transduced therapeutic cells from the patient; and
administering a multimeric ligand that binds to the multimerization region, maintaining a subsequent dosage of the multimeric ligand, or adjusting a subsequent dosage of the multimeric ligand to the patient based on the presence or absence of the condition identified in the patient.

A150. The method of any one of embodiments A133-146, further comprising
identifying a presence or absence of a condition in the patient that requires the removal of transfected or transduced therapeutic cells from the patient; and
transmitting the presence, absence or stage of the condition identified in the patient to a decision maker who administers a multimeric ligand that binds to the multimerization region, maintains a subsequent dosage of the multimeric ligand, or adjusts a subsequent dosage of the multimeric ligand administered to the patient based on the presence, absence or stage of the condition identified in the patient.

A151. The method of any one of embodiments A133-146, further comprising
identifying a presence or absence of a condition in the patient that requires the removal of transfected or transduced therapeutic cells from the patient; and
transmitting an indication to administer a multimeric ligand that binds to the multimerization region, maintains a subsequent dosage of the multimeric ligand, or adjusts a subsequent dosage of the multimeric ligand administered to the patient based on the presence, absence or stage of the condition identified in the patient.

A152. The method of any one of embodiments A133-151, wherein the therapeutic cells are transduced or transfected with a second nucleic acid that encodes a second heterologous protein.

A153. The method of any one of embodiments A133-151 wherein the therapeutic cells are transduced with a heterologous gene that expresses a chimeric antigen receptor.

A154. The method of any one of embodiments A133-151, wherein the therapeutic cells are transduced with a heterologous gene that expresses a modified TGF-beta receptor.

A155. The method of any of embodiments A152-154, wherein the therapeutic cells are transduced with the heterologous gene before, at the same time as, or after being transduced with the nucleic acid encoding the chimeric protein comprising a multimerization region and a caspase-9 polypeptide;

A156. A method of administering donor T cells to a human patient, comprising administering a cell of any one of embodiments A1-51.4, or A51.28-78 to a human patient, wherein the cells are non-allodepleted human donor T cells.

A157. A method of reducing the effect of graft versus host disease in a human patient following administration of a cell of any one of embodiments A1-78, wherein the cell is a donor T cell, comprising:
a) detecting the presence or absence of graft versus host disease in the patient after administration of the donor T cells; and
b) administering a multimeric ligand that binds to the multimerization region to a patient for whom the presence of graft versus host disease is detected.

A158. A method of stem cell transplantation, comprising
a) administering a stem cell transplant to a human patient; and
b) administering a cell of any one of embodiments, A1-51.4, or A51.28-78
   wherein the cells are donor T cells, to the patient.

A159. The method of any one of embodiments A156-158, wherein at least 1×10⁶ cells/Kg body weight are administered to the patient.

A160. The method of any one of embodiments A156-158, wherein at least 3×10⁶ cells/Kg body weight are administered to the patient.

A161. The method of any one of embodiments A156-158, wherein at least 5×10⁶ cells/Kg body weight are administered to the patient.

A162. The method of any one of embodiments A156-158, wherein at least 7×10⁶ cells/Kg body weight are administered to the patient.

A163. The method of any one of embodiments A156-158, wherein at least 9×10⁶ cells/Kg body weight are administered to the patient.

A164. A modified caspase-9 polypeptide, comprising an amino acid sequence having at least 90% sequence identity to SEQ ID NO: 9, and comprising at least one amino acid substitution selected from the group consisting of S144A, S144D, Y153A, Y153F, S183A, S195A, S196A, S196D, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, C403A, C403S, C403T, F404T, F404W, F404Y, N405A, N405F, N405Q, N405T, F406A, F406T, F406W, F406Y, G402A, G402I, G402Q, G402Y, C403P, F404A, F4045, and F406L.

A165. The modified caspase-9 polypeptide of embodiment A164, wherein the at least one amino acid substitution is selected from the group consisting of S144A, S144D, Y153A, Y153F, S183A, S195A, S196A, S196D, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, C403A, C403S, C403T, F404T, F404W, F404Y, N405A, N405F, N405Q, N405T, F406A, F406T, F406W, and F406Y.

A166. The modified caspase-9 polypeptide of embodiment A164, wherein the at least one amino acid substitution is selected from the group consisting of S144A, S144D, Y153A, Y153F, S183A, S195A, S196A, S196D, S307A, D315A, A316G, T317A, T317C, T317S, P318A, F319A, F319W, L329E, D330A, D330E, D330G, D330N, D330S, D330V, C403A, C403S, C403T, F404T, F404W, F404Y, N405A, N405F, N405Q, F406A, F406T, F406W, and F406Y.

A167. The modified caspase-9 polypeptide of embodiment A164, wherein the at least one amino acid substitution is selected from the group consisting of S144A, S144D, Y153A, Y153F, S183A, S195A, S196A, S196D, S307A, D315A, A316G, T317A, T317S, F319W, L329E, D330A, D330E, D330G, D330N, D330S, D330V, F404T, F404W, F404Y, N405F, N405Q, F406A, F406T, F406W, and F406Y.

A168. The modified caspase-9 polypeptide of embodiment A164, wherein the at least one amino acid substitution is selected from the group consisting of S144A, S144D, S183A, S195A, S196A, S196D, T317A, T317S, L329E, D330A, D330E, D330G, D330N, D330S, D330V, F404Y, N405Q, F406A, F406W, and F406Y.

A169. The modified caspase-9 polypeptide of embodiment A164, wherein the at least one amino acid substitution is selected from the group consisting of T317S, S144A, S133, and S196D.

A170. The modified caspase-9 polypeptide of embodiment A164, wherein the at least one amino acid substitution is selected from the group consisting of S183A, S195A, S196A, S196D, T317A, L329E, D330A, D330E, D330G, D330N, D330S, D330V, F404Y, N405Q, F406A, F406W, and F406Y.

A171. The modified caspase-9 polypeptide of embodiment A164, wherein the at least one amino acid substitution is D330A.

A172. The modified caspase-9 polypeptide of embodiment A164, wherein the at least one amino acid substitution is D330E.

A173. The modified caspase-9 polypeptide of embodiment A164, wherein the at least one amino acid substitution is N405Q.

A174. The modified caspase-9 polypeptide of embodiment A164, wherein the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, ⁴⁰²GCFNF⁴⁰⁶ISAQT (CASP-10) (SEQ ID NOS 224 and 225), ³¹⁶ATPF³¹⁹AVPI (SMAC/Diablo) (SEQ ID NOS 226 and 227), D330A-N405T, D315A-D330A, D330A-Y153A, D330A-Y153F, D330A-T317E, ⁴⁰²GCFNF⁴⁰⁶CIVSM (CASP-3) (SEQ ID NOS 224 and 228), ⁴⁰²GCFNF⁴⁰⁶AAAAA (SEQ ID NOS 224 and 229), ⁴⁰²GCFNF⁴⁰⁶YCSTL (CASP-2) (SEQ ID NOS 224 and 230), and ⁴⁰²GCFNF⁴⁰⁶QPTFT (CASP-8) (SEQ ID NOS 224 and 231).

A175. The modified caspase-9 polypeptide of embodiment A164, wherein the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, ⁴⁰²GCFNF⁴⁰⁶ISAQT (CASP-10) (SEQ ID NOS 224 and 225), ³¹⁶ATPF³¹⁹AVPI (SMAC/Diablo) (SEQ ID NOS 226 and 227), and D330A-N405T.

A176. The modified caspase-9 polypeptide of embodiment A164, wherein the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, ⁴⁰²GCFNF⁴⁰⁶ISAQT (CASP-10) (SEQ ID NOS 224 and 225), and ³¹⁶ATPF³¹⁹AVPI (SMAC/Diablo) (SEQ ID NOS 226 and 227).

A177. The modified caspase-9 polypeptide of embodiment A164, wherein the modified caspase-9 polypeptide comprises at least two amino acid substitutions selected from the group consisting of D330A-N405Q, D330A-S144A, D330A-S144D, D330A-S183A, D330A-S196A, N405Q-S144A, N405Q-S144D, N405Q-S196D, N405Q-T317S, N405Q-S144Aco, N405Q-T317Sco, and ⁴⁰²GCFNF⁴⁰⁶ISAQT (CASP-10) (SEQ ID NOS 224 and 225).

A178. The modified caspase-9 polypeptide of embodiment A164, wherein the modified caspase-9 polypeptide comprises at least two amino acid substitutions selected from the group consisting of N405Q-S144Aco and N405Q-T317Sco.

A179. The modified caspase-9 polypeptide of embodiment A164, wherein the at least one amino acid substitution is selected from the group consisting of S144A, S144D, Y153A, Y153F, S183A, S195A, S196A, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, F404T, F404W, F404YN405F, N405Q, and F406T.

A180. The modified caspase-9 polypeptide of embodiment A164, wherein the at least one amino acid substitution is selected from the group consisting of S144A, S144D, Y153A, Y153F, S183A, S195A, S196A, S307A, D315A, A316G, T317A, T317C, T317E, T317S, P318A, F319A, F319W, F326K, D327G, D327K, D327R, Q328K, Q328R, L329E, L329G, L329K, D330A, D330E, D330G, D330N, D330S, D330V, A331K, F404T, F404W, F404Y, N405F, N405Q, and F406T.

A181. The modified caspase-9 polypeptide of embodiment A164, wherein the at least one amino acid substitution is S144A, S144D, Y153A, Y153F, S183A, S195A, S196A, S307A, D315A, A316G, T317A, T317C, T317S, P318A, F319A, F319W, L329E, D330A, D330E, D330G, D330N, D330S, D330V, F404T, F404W, F404Y, N405F, N405Q, and F406T.

A182. The modified caspase-9 polypeptide of embodiment A164, wherein the at least one amino acid substitution is S144A, S144D, Y153A, Y153F, S183A, S195A, S196A, S307A, D315A, A316G, T317A, T317S, F319W, D330A, F404T, F404W, F404Y, N405F, N405Q, and F406T.

A183. The modified caspase-9 polypeptide of embodiment A164, wherein the at least one amino acid substitution is selected from the group consisting of S144A, S144D, S183A, S195A, S196A, T317A, T317S, D330A, F404Y, and N405Q.

A184. The modified caspase-9 polypeptide of embodiment A164, wherein the at least one amino acid substitution is selected from the group consisting of S196D, T317C, T317E, P318A, F319A, F326K, D327G, D327K, D327R, Q328K, Q328R, L329E, L329G, L329K, D330E, D330G, D330N, D330S, D330V, A331K, C403A, C403S, C403T, N405A, N405T, F406A, F406W, F406Y, G402A, G402I, G402Q, G402Y, C403P, F404A, F404S, and F406L.

A185. The modified caspase-9 polypeptide of embodiment A164, wherein the at least one amino acid substitution is selected from the group consisting of S196D, T317C, T317E, P318A, F319A, F326K, D327G, D327K, D327R, Q328K, Q328R, L329E, L329G, L329K, D330E, D330G, D330N, D330S, D330V, A331K, C403A, C403S, C403T, N405A, N405T, F406A, F406W, and F406Y.

A186. The modified caspase-9 polypeptide of embodiment A164, wherein the at least one amino acid substitution is selected from the group consisting of S196D, T317C, P318A, F319A, L329E, D330E, D330G, D330N, D330S, D330V, C403A, C403S, C403T, N405A, F406A, F406T, F406W, and F406Y.

A187. The modified caspase-9 polypeptide of embodiment A164, wherein the at least one amino acid substitution is selected from the group consisting of S196D, L329E, D330E, D330G, D330N, D330S, D330V, F406A, F406T, F406W, and F406Y.

A188. The modified caspase-9 polypeptide of embodiment A164, wherein the at least one amino acid substitution is selected from the group consisting of S196D, L329E, D330E, D330G, D330N, D330S, D330V, F406A, F406W, and F406Y.

A189. The modified caspase-9 polypeptide of any one of embodiments A164-188, wherein the polynucleotide comprises optimized codons encoding the caspase-9 polypeptide.

A190. The modified caspase-9 polypeptide of embodiment A144, comprising the nucleic acid sequence of SEQ ID NO: 39.

A191. The modified caspase-9 polypeptide of embodiment A164, wherein the at least one amino acid substitution is selected from the group consisting of N405Q, F404Y, F406A, F406W, and F406Y.

A192. The modified caspase-9 polypeptide of embodiment A164, wherein the at least one amino acid substitution is selected from the group consisting of T317S, D330A, D330E, D330G, D330N, D330S, D330V, L329E, and T317A.

A193. The modified caspase-9 polypeptide of embodiment A164, wherein the at least one amino acid substitution is selected from the group consisting of S144A, S144D, S196D, S183A, S195A, S196A, Y153A, Y153F, and S307A.

A194. A chimeric polypeptide comprising a multimerization region and a modified caspase-9 polypeptide of any one of embodiments A164-193.

A195. A nucleic acid comprising a polynucleotide encoding the modified caspase-9 polypeptide of any one of embodiments A164-193.

A196. A nucleic acid comprising a polynucleotide encoding the chimeric polypeptide of any one of embodiments A1-195

A197. The nucleic acid of embodiments A195 or A196, further comprising a polynucleotide that encodes a heterologous protein.

A198. The nucleic acid of embodiment A197, wherein the heterologous protein is a chimeric antigen receptor.

A199. The nucleic acid of any one of embodiments A197-198, wherein the polynucleotide encodes the modified caspase-9 polypeptide and further encodes a heterologous polypeptide and a linker polypeptide linking the modified caspase-9 polypeptide and the heterologous polypeptide, wherein the linker polypeptide separates the modified caspase-9 polypeptide and the heterologous polypeptide during or after translation.

A200. The cell of embodiment A199, wherein the linker polypeptide is a 2A or a 2A-like polypeptide.

A201. The method of embodiment A158, wherein the stem cell transplant is haploidentical.

A202. The method of embodiment A158 or A197, wherein the donor T cells are haploidentical and are not allodepleted before administration to the patient.

A203. The cell of embodiment A1, wherein the modified caspase-9 polypeptide comprises an amino acid substitution selected from the group consisting of the caspase variants in Table 5 or Table 6.

A204. The modified caspase-9 polypeptide of embodiment A164, wherein the modified caspase-9 polypeptide comprises an amino acid substitution selected from the group consisting of the caspase variants in Table 5 or Table 6.

### Example 12

### Representative Embodiments

Provided hereafter are examples of certain embodiments of the technology.

1. A method for controlling the survival of therapeutic cells in a subject, comprising administering to a subject in need thereof
a) modified cells comprising a promoter operably linked to a polynucleotide that encodes a chimeric polypeptide comprising
   i. a multimerization region and
   ii. a modified caspase-9 polypeptide comprising an amino acid sequence having at least 90% sequence identity to SEQ ID NO: 9, and comprises at least one amino acid substitution selected from the group consisting of D330E, S196D, F406A, F406W, F406Y, N405Q (codon optimized), D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K; and
b) an amount of a multimeric ligand that binds to the multimerization region effective to reduce the number of cells administered in (a).

2. The method of embodiment 1, wherein the at least one amino acid substitution is selected from the group consisting of D330E, S196D, F406A, F406W, F406Y, N405Q (codon optimized), D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, and F319A.

3. The method of embodiment 1, wherein the at least one amino acid substitution is selected from the group consisting of S196D, F406A, F406W, F406Y, N405Q (codon optimized), D330E, D330G, D330N, D330S, D330V, and L329E.

4. The method of embodiment 1, wherein the at least one amino acid substitution is D330E.

5. The method of any one of embodiments 1 to 4, wherein the modified caspase-9 polypeptide further comprises at least one amino acid substitution selected from the group consisting of S196D, F406A, F406W, F406Y, N405Q (codon optimized), D330E, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K.

6. The method of any one of embodiments 1 to 4, wherein the modified caspase-9 polypeptide further comprises at least one amino acid substitution selected from the group consisting of S196D, F406A, F406W, F406Y, N405Q (codon optimized), D330E, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, A331K, T317S, S144A, S144D, N405Q, ⁴⁰²GCFNF⁴⁰⁶ISAQT (Casp-10) (SEQ ID NOS 224 and 225), F404Y, D330A, T317A, S183A, S195A, S196A, F404T, F404W, N405F, F406T, D315A, A316G, F319W, Y153A, Y153F, and S307A.

7. The method of embodiment 1, wherein the modified caspase-9 polypeptide comprises an N405Q-S196D amino acid substitution.

8. The method of any one of embodiments 1-7, wherein the polynucleotide comprises optimized codons encoding the modified caspase-9 polypeptide.

9. The method of any one of embodiments 1 to 8, wherein the cell further comprises a polynucleotide that encodes a heterologous polypeptide.

10. The method of embodiment 9, wherein the cell comprises a nucleic acid that comprises
a) the polynucleotide that encodes the chimeric polypeptide; and
b) the polynucleotide that encodes the heterologous polypeptide.

11. The method of embodiment 10, wherein the promoter is operably linked to the polynucleotide that encodes the chimeric polypeptide and the polynucleotide that encodes the heterologous polypeptide.

12. The method of embodiment 10, wherein the promoter is operably linked to the polynucleotide that encodes the chimeric polypeptide and a second promoter is operably linked to the polynucleotide that encodes the heterologous polypeptide.

13. The method of any one of embodiments 10 or 11, wherein the nucleic acid further comprises a polynucleotide encoding a linker polypeptide between the polynucleotide that encodes the chimeric polypeptide and the polynucleotide that encodes the heterologous polypeptide, wherein the linker polypeptide separates the translation products of the first and second polynucleotides during or after translation.

14. The method of embodiment 13, wherein the linker polypeptide is a 2A polypeptide.

15. The method of any one of embodiments 9-14, wherein the heterologous polypeptide is a T cell receptor or a T cell receptor-based chimeric antigen receptor.

16. The method of any one of embodiments 9-15, wherein the heterologous polypeptide is a chimeric antigen receptor.

17. The method of embodiment 16, wherein the chimeric antigen receptor comprises
(i) a transmembrane region; (ii) a T cell activation molecule; and (iii) an antigen recognition moiety.

18. The method of embodiment 17, wherein the T cell activation molecule is an ITAM-containing, Signal 1 conferring molecule.

19. The method of embodiment 17, wherein the T cell activation molecule is a CD3 polypeptide.

20. The method of embodiment 17, wherein the T cell activation molecule is an Fc epsilon receptor gamma (FcεR1γ) subunit polypeptide.

21. The method of any one of embodiments 17-20, wherein the antigen recognition moiety binds to an antigen on a tumor cell.

22. The method of any one of embodiments 17-20, wherein the antigen recognition moiety binds to an antigen on a cell involved in a hyperproliferative disease.

23. The method of any one of embodiments 17-20, wherein the antigen recognition moiety binds to an antigen selected from the group consisting of PSMA, PSCA, MUC1, CD19, ROR1, Mesothelin, GD2, CD123, MUC16, and Her2/Neu.

24. The method of any one of embodiments 17-20, wherein the antigen recognition moiety binds to PSCA.

25. The method of any one of embodiments 1-20, wherein the multimerization region comprises an FKBP12 region.

26. The method of embodiment 25, wherein the FKBP12 region has an amino acid substitution at position 36 selected from the group consisting of valine, leucine, isoleuceine and alanine.

27. The method of embodiment 26, wherein the FKBP12 region is an FKBP12v36 region.

28. The method of any one of embodiments 1-27, wherein the multimerization region comprises Fv′Fvls.

29. The method of any one of embodiments 1-28, wherein the multimerization region comprises a polypeptide having an amino acid sequence of SEQ ID NO: 30, or a functional fragment thereof, and a polypeptide having an amino acid sequence of SEQ ID NO: 32, or a functional fragment thereof.

30. The method of embodiment 29, wherein the multimerization region further comprises a polypeptide having an amino acid sequence of SEQ ID NO: 30 or 32 or a functional fragment thereof.

31. The method of embodiment 29, wherein the multimerization region further comprises an Fv polypeptide variant wherein residue 36 is valine.

32. The method of any one of embodiments 1-31, wherein the multimeric ligand is an FK506 dimer or a dimeric FK506 analog ligand.

33. The method of any one of embodiments 1-32, wherein the ligand is AP1903.

34. The method of any one of embodiments 1-32, wherein the ligand is AP20187.

35. The method of any one of embodiments 1-34, wherein the cell is a human cell.

36. The method of any one of embodiments 1-35, wherein the cell is a progenitor cell.

37. The method of any one of embodiments 1-35, wherein the cell is a hematopoietic progenitor cell.

38. The method of any one of embodiments 1-35, wherein the cell is selected from the group consisting of mesenchymal stromal cells, embryonic stem cells, and inducible pluripotent stem cells.

39. The method of any one of embodiments 1-35, wherein the cell is a T cell.

40. The method of any one of embodiments 1-35, wherein the cell is a T cell, tumor infiltrating lymphocyte, NK-T cell, TCR-expressing cell, or NK cell.

42. The method of any one of embodiments 1-40, wherein the modified cells are autologous cells.

43. The method of any one of embodiments 1-40, wherein the modified cells are allogeneic cells.

44. The method of any one of embodiments 1-43, wherein the cell is obtained or prepared from bone marrow.

45. The method of any one of embodiments 1-43, wherein the cell is obtained or prepared from umbilical cord blood.

46. The method of any one of embodiments 1-43, wherein the cell is obtained or prepared from peripheral blood.

47. The method of any one of embodiments 1-43, wherein the cell is obtained or prepared from peripheral blood mononuclear cells.

48. The method of any one of embodiments 1-47, wherein the promoter operably linked to the polynucleotide encoding the chimeric polypeptide is developmentally regulated and the caspase-9 polypeptide is expressed in developmentally differentiated cells.

49. The method of any one of embodiments 1-48, wherein the promoter operably linked to the polynucleotide encoding the chimeric polypeptide is tissue-specific and the caspase-9 polypeptide is expressed in the specific tissue.

50. The method of any one of embodiments 1-49, wherein the promoter operably linked to the polynucleotide encoding the chimeric polypeptide is activated in activated T cells.

51. The method of any one of embodiments 1-50, wherein the chimeric polypeptide further comprises a marker polypeptide.

52. The method of embodiment 51, wherein the marker polypeptide is a ΔCD19 polypeptide.

53. The method of any one of embodiments 1-52, wherein the caspase-9 polypeptide is a truncated caspase-9 polypeptide.

54. The method of any one of embodiments 1-53 wherein the caspase-9 polypeptide lacks the Caspase recruitment domain (CARD).

55. The method of any one of embodiments 1-54, wherein the number of therapeutic cells is reduced by about 50% following administration of the multimeric ligand.

56. The method of any one of embodiments 1-54, wherein the number of therapeutic cells is reduced by about 80% following administration of the multimeric ligand.

57. The method of any one of embodiments 1-54, wherein the number of therapeutic cells is reduced by about 90% following administration of the multimeric ligand.

58. The method of any one of embodiments 1-57, wherein more than one dose of the multimeric ligand is administered.

59. The method of any one of embodiments 1-58, further comprising
identifying a presence or absence of a condition in the subject that requires the removal of transfected or transduced therapeutic cells from the subject; and
administering a multimeric ligand that binds to the multimerization region, maintaining a subsequent dosage of the multimeric ligand, or adjusting a subsequent dosage of the multimeric ligand to the subject based on the presence or absence of the condition identified in the subject.

60. The method of any one of embodiments 1-59 further comprising
identifying a presence or absence of a condition in the subject that requires the removal of transfected or transduced therapeutic cells from the subject; and
determining whether a multimeric ligand that binds to the multimerization region should be administered to the subject, or the dosage of the multimeric ligand subsequently administered to the subject is adjusted based on the presence or absence of the condition identified in the subject.

61. The method of any one of embodiments 1-59, further comprising
receiving information comprising presence or absence of a condition in the subject that requires the removal of transfected or transduced therapeutic cells from the subject; and
administering a multimeric ligand that binds to the multimerization region, maintaining a subsequent dosage of the multimeric ligand, or adjusting a subsequent dosage of the multimeric ligand to the subject based on the presence or absence of the condition identified in the subject.

62. The method of any one of embodiments 1-59, further comprising
identifying a presence or absence of a condition in the subject that requires the removal of transfected or transduced therapeutic cells from the subject; and
transmitting the presence, absence or stage of the condition identified in the subject to a decision maker who administers a multimeric ligand that binds to the multimerization region, maintains a subsequent dosage of the multimeric ligand, or adjusts a subsequent dosage of the multimeric ligand administered to the subject based on the presence, absence or stage of the condition identified in the subject.

63. The method of any one of embodiments 1-59, further comprising
identifying a presence or absence of a condition in the subject that requires the removal of transfected or transduced therapeutic cells from the subject; and
transmitting an indication to administer a multimeric ligand that binds to the multimerization region, maintains a subsequent dosage of the multimeric ligand, or adjusts a subsequent dosage of the multimeric ligand administered to the subject based on the presence, absence or stage of the condition identified in the subject.

64. The method of any one of embodiments 1-62, wherein the therapeutic cells are selected from the group consisting of T cells, tumor infiltrating lymphocytes, NK-T cells, CAR-expressing cells, TCR-expressing cells, and NK cells, further comprising detecting the presence of graft versus host disease or cytokine release syndrome in the subject after administering the therapeutic cells to the subject, and before administering the multimeric ligand.

65. The method of embodiment 64, wherein after administration of the multimeric ligand, the number of therapeutic cells causing graft versus host disease or cytokine release syndrome in the subject cells is reduced.

66. The method of any one of embodiments 1-65, wherein the therapeutic cells are allodepleted before transfection or transduction.

67. The method of any one of embodiments 1-65, wherein the therapeutic cells are not allodepleted before transfection or transduction.

68. The method of any one of embodiments 1-67, wherein the transduced or transfected therapeutic cells are cultured in the presence of IL-2 before administration to the subject.

69. The method of embodiment 65, wherein alloreactive therapeutic cells are present in the subject and the number of alloreactive therapeutic cells is reduced by at least 90% after administration of the multimeric ligand.

70. The method of any one of embodiments 64-69, wherein, after administration of the multimeric ligand, therapeutic cells survive in the subject that are able to expand and are reactive to viruses and fungi.

71. The method of any one of embodiments 64-69, wherein after administration of the multimeric ligand, therapeutic cells survive in the subject that are able to expand and are reactive to tumor cells in the subject.

72. The method of any one of embodiments 1-71, wherein the subject has received a stem cell transplant before or at the same time as administration of the therapeutic cells.

73. The method of any one of embodiments 64-71, further comprising determining whether to administer an additional dose or additional doses of the multimeric ligand to the subject based upon the appearance of graft versus host disease symptoms in the subject.

74. The method of any one of embodiments 1-73, wherein at least 1×10⁶ transduced or transfected therapeutic cells are administered to the subject.

75. The method of any one of embodiments 1-73, wherein at least 1×10⁷ transduced or transfected donor T cells are administered to the subject.

76. The method of any one of embodiments 1-73, wherein at least 1×10⁸ transduced or transfected donor T cells are administered to the subject.

77. The method of any one of embodiments 1-76, further comprising
identifying the presence, absence or stage of graft versus host disease in the subject, and
administering a multimeric ligand that binds to the multimerization region, maintaining a subsequent dosage of the multimeric ligand, or adjusting a subsequent dosage of the multimeric ligand to the subject based on the presence, absence or stage of the graft versus host disease identified in the subject.

79-99. Reserved.

100. A nucleic acid comprising a promoter operably linked to a polynucleotide that encodes a chimeric polypeptide, wherein the chimeric polypeptide comprises: a multimerization region and a modified caspase-9 polypeptide comprising
an amino acid sequence having at least 90% sequence identity to SEQ ID NO: 9, and
at least one amino acid substitution selected from the group consisting of D330E, S196D, F406A, F406W, F406Y, N405Q (codon optimized), D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K.

101. A nucleic acid comprising a promoter operably linked to
a) a first polynucleotide that encodes a chimeric polypeptide, wherein the chimeric polypeptide comprises: a multimerization region and a modified caspase-9 polypeptide comprising an amino acid sequence having at least 90% sequence identity to SEQ ID NO: 9, and
at least one amino acid substitution selected from the group consisting of D330E, S196D, F406A, F406W, F406Y, N405Q (codon optimized), D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K; and
b) a second polynucleotide that encodes a heterologous polypeptide.

102. A nucleic acid comprising a promoter operably linked to
a) a first polynucleotide that encodes a chimeric polypeptide, wherein the chimeric polypeptide comprises: a multimerization region and a modified caspase-9 polypeptide comprising
an amino acid sequence having at least 90% sequence identity to SEQ ID NO: 9, and
at least one amino acid substitution selected from the group consisting of S196D, F406A, F406W, F406Y, N405Q (codon optimized), D330E, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, A331K, T317S, S144A, S144D, N405Q, ⁴⁰²GCFNF⁴⁰⁶ISAQT (Casp-10) (SEQ ID NOS 224 and 225), F404Y, D330A, T317A, S183A, S195A, S196A, F404T, F404W, N405F, F406T, D315A, A316G, F319W, Y153A, Y153F, and S307A; and
b) a second polynucleotide that encodes a heterologous polypeptide.

103. The nucleic acid of any one of embodiments 101 or 102, wherein the at least one amino acid substitution is selected from the group consisting of S196D, F406A, F406W, F406Y, N405Q (codon optimized), D330E, D330G, D330N, D330S, D330V, L329E, C403A, C403S, C403T, N405A, 316ATPF319AVPI (SMAC/Diablo) (SEQ ID NOS 226 and 227), T317C, P318A, and F319A.

104. The nucleic acid of any one of embodiments 101 or 102, wherein the at least one amino acid substitution is selected from the group consisting of S196D, F406A, F406W, F406Y, N405Q (codon optimized), D330E, D330G, D330N, D330S, D330V, and L329E.

105. The nucleic acid of any one of embodiments 101 or 102, wherein the at least one amino acid substitution is D330E.

106. The nucleic acid of any one of embodiments 101 to 105, wherein the modified caspase-9 polypeptide further comprises at least one amino acid substitution selected from the group consisting of S196D, F406A, F406W, F406Y, N405Q (codon optimized), D330E, D330G, D330N, D330S, D330V, L329E, C403A, C403S, C403T, N405A, 316ATPF319AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K.

107. The nucleic acid of any one of embodiments 101 to 105, wherein the modified caspase-9 polypeptide further comprises at least one amino acid substitution selected from the group consisting of S196D, F406A, F406W, F406Y, N405Q (codon optimized), D330E, D330G, D330N, D330S, D330V, L329E, C403A, C403S, C403T, N405A, 316ATPF319AVPI (SMAC/Diablo) (SEQ ID NOS 226 and 227), T317C, P318A, F319A, N405T, T317E, F326K, D327G, D327K, D327R, Q328K, Q328R, L329G, L329K, A331K, T317S, S144A, S144D, N405Q, 402GCFNF406ISAQT (Casp-10) (SEQ ID NOS 224 and 225), F404Y, D330A, T317A, S183A, S195A, S196A, F404T, F404W, N405F, F406T, D315A, A316G, F319W, Y153A, Y153F, and S307A.

108. The nucleic acid of any one of embodiments 101 or 102, wherein the modified caspase-9 polypeptide comprises an N405Q-S196D amino acid substitutions.

109. The nucleic acid of any one of embodiments 101-107, wherein the polynucleotide comprises optimized codons encoding the modified caspase-9 polypeptide.

110. The nucleic acid of any one of embodiments 101-109, wherein the promoter is operably linked to the first and the second polynucleotides.

111. The nucleic acid of any one of embodiments 101-109, wherein the promoter is operably linked to the first polynucleotide and a second promoter is operably linked to the second polynucleotide.

112. The nucleic acid of any one of embodiments 101-110, wherein the nucleic acid further comprises a polynucleotide encoding a linker polypeptide between the first and second polynucleotides, wherein the linker polypeptide separates the translation products of the first and second polynucleotides during or after translation.

113. The nucleic acid of embodiment 112, wherein the linker polypeptide is a 2A polypeptide.

114. The nucleic acid of any one of embodiments 101-113, wherein the heterologous polypeptide is a T cell receptor or a T cell receptor-based chimeric antigen receptor.

115. The nucleic acid of any one of embodiments 101-113, wherein the heterologous polypeptide is a chimeric antigen receptor.

116. The nucleic acid of embodiment 115, wherein the chimeric antigen receptor comprises

(i) a transmembrane region; (ii) a T cell activation molecule; and (iii) an antigen recognition moiety.

117. The nucleic acid of embodiment 116, wherein the T cell activation molecule is an ITAM-containing, Signal 1 conferring molecule.

118. The nucleic acid of embodiment 116, wherein the T cell activation molecule is a CD3 ζ polypeptide.

119. The nucleic acid of embodiment 116, wherein the T cell activation molecule is an Fc epsilon receptor gamma (FcεR1γ) subunit polypeptide.

120. The nucleic acid of any one of embodiments 116-119, wherein the antigen recognition moiety binds to an antigen on a tumor cell.

121. The nucleic acid of any one of embodiments 116-119, wherein the antigen recognition moiety binds to an antigen on a cell involved in a hyperproliferative disease.

122. The nucleic acid of any one of embodiments 116-119 wherein the antigen recognition moiety binds to an antigen selected from the group consisting of PSMA, PSCA, MUC1, CD19, ROR1, mesothelin, GD2, CD123, MUC16, and Her2/Neu.

123. The nucleic acid of any one of embodiments 116-119, wherein the antigen recognition moiety binds to PSCA.

124. The nucleic acid of any one of embodiments 100-123, wherein the multimerization region comprises an FKBP12 region.

125. The nucleic acid of embodiment 124, wherein the FKBP12 region has an amino acid substitution at position 36 selected from the group consisting of valine, leucine, isoleuceine and alanine.

126. The nucleic acid of embodiment 124, wherein the FKBP12 region is an FKBP12v36 region. 127. The nucleic acid of any one of embodiments 100-126, wherein the multimerization region comprises Fv′Fvls.

128. The nucleic acid of any one of embodiments 100-127, wherein the multimerization region comprises a polypeptide having an amino acid sequence of SEQ ID NO: 30, or a functional fragment thereof, and a polypeptide having an amino acid sequence of SEQ ID NO: 32, or a functional fragment thereof.

129. The nucleic acid of embodiment 128, wherein the multimerization region further comprises a polypeptide having an amino acid sequence of SEQ ID NO: 30 or 32, or a functional fragment thereof.

130. The nucleic acid of embodiment 128, wherein the multimerization region further comprises an Fv polypeptide variant wherein residue 36 is valine.

131. The nucleic acid of any one of embodiments 101-130, wherein the multimerization region binds to a multimeric ligand.

132. The nucleic acid of embodiment 131, wherein the multimeric ligand is an FK506 dimer or a dimeric FK506 analog ligand.

133. The nucleic acid of any one of embodiments 131 or 132, wherein the ligand is AP1903.

134. The nucleic acid of any one of embodiments 131 or 132, wherein the ligand is AP20187.

135. The nucleic acid of any one of embodiments 100-134, wherein the promoter operably linked to the polynucleotide that encodes the chimeric polypeptide is developmentally regulated and the caspase-9 polypeptide is expressed in developmentally differentiated cells.

136. The nucleic acid of any one of embodiments 100-135, wherein the promoter operably linked to the polynucleotide encoding the chimeric polypeptide is tissue-specific and the caspase-9 polypeptide is expressed in the specific tissue.

137. The nucleic acid of any one of embodiments 101-135, wherein the promoter operably linked to the polynucleotide encoding the chimeric polypeptide is activated in activated T cells.

138. The nucleic acid of any one of embodiments 101-113, or 124-137, wherein the heterologous polypeptide comprises a marker polypeptide.

139. The nucleic acid of embodiment 138, wherein the marker polypeptide is a ΔCD19 polypeptide.

140. The nucleic acid of any one of embodiments 100-139, wherein the caspase-9 polypeptide is a truncated caspase-9 polypeptide.

141. The nucleic acid of any one of embodiments 100-140 wherein the caspase-9 polypeptide lacks the Caspase recruitment domain.

142-199. Reserved.

200. A modified cell comprising a nucleic acid of any one of embodiments 100-140.

201. The cell of embodiment 200, wherein the cell is a human cell.

202. The cell of any one of embodiments 200-201, wherein the cell is a progenitor cell.

203. The cell of any one of embodiments 200-202, wherein the cell is a hematopoietic progenitor cell.

204. The cell of any one of embodiments 200-201, wherein the cell is selected from the group consisting of mesenchymal stromal cells, embryonic stem cells, and inducible pluripotent stem cells.

205. The cell of any one of embodiments 200-201, wherein the cell is a T cell.

206. The cell of any one of embodiments 200-201, wherein the cell is a T cell, tumor infiltrating lymphocyte, NK-T cell, TCR-expressing cell, or NK cell.

207-208. Reserved.

209. The cell of any one of embodiments 200-208, wherein the cell is obtained or prepared from bone marrow.

210. The cell of any one of embodiments 200-208, wherein the cell is obtained or prepared from umbilical cord blood.

211. The cell of any one of embodiments 200-208, wherein the cell is obtained or prepared from peripheral blood.

212. The cell of any one of embodiments 200-208, wherein the cell is obtained or prepared from peripheral blood mononuclear cells.

213. The cell of any one of embodiments 200-212, wherein the nucleic acid does not comprise a second polypeptide encoding a T cell receptor, a T cell receptor-based chimeric antigen receptor, or a chimeric antigen receptor.

214. The cell of embodiment 213, further comprising a nucleic acid that encodes a heterologous polypeptide.

215. The cell of embodiment 214, wherein the heterologous polypeptide is a T cell receptor or a T cell receptor-based chimeric antigen receptor.

216. The cell of embodiment 214, wherein the heterologous polypeptide is a chimeric antigen receptor.

217. The cell of embodiment 216, wherein the chimeric antigen receptor comprises
(i) a transmembrane region; (ii) a T cell activation molecule; and (iii) an antigen recognition moiety.

218. The cell of embodiment 217, wherein the T cell activation molecule is an ITAM-containing, Signal 1 conferring molecule.

219. The cell of embodiment 217, wherein the T cell activation molecule is a CD3 ζ polypeptide.

220. The cell of embodiment 217, wherein the T cell activation molecule is an Fc epsilon receptor gamma (FcεR1γ) subunit polypeptide.

221. The cell of any one of embodiments 217-220, wherein the antigen recognition moiety binds to an antigen on a tumor cell.

222. The cell of any one of embodiments 217-220, wherein the antigen recognition moiety binds to an antigen on a cell involved in a hyperproliferative disease.

223. The cell of any one of embodiments 217-220 wherein the antigen recognition moiety binds to an antigen selected from the group consisting of PSMA, PSCA, MUC1, CD19, ROR1, Mesothelin, GD2, CD123, MUC16, and Her2/Neu.

224. The cell of any one of embodiments 217-220, wherein the antigen recognition moiety binds to PSCA.

225. The cell of embodiment 214, wherein the heterologous polypeptide comprises a marker polypeptide.

226. The cell of embodiment 225, wherein the marker polypeptide is a ΔCD19 polypeptide.

227-299. Reserved.

300. A modified caspase-9 polypeptide, comprising an amino acid sequence having at least 90% sequence identity to SEQ ID NO: 9, and comprising at least one amino acid substitution selected from the group consisting of D330E, S196D, F406A, F406W, F406Y, N405Q (codon optimized), D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K.

301. The modified caspase-9 polypeptide of embodiment 300, wherein the at least one amino acid substitution is selected from the group consisting of S196D, F406A, F406W, F406Y, N405Q (codon optimized), D330E, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, and F319A.

302. The modified caspase-9 polypeptide of embodiment 300, wherein the at least one amino acid substitution is selected from the group consisting of S196D, F406A, F406W, F406Y, N405Q (codon optimized), D330E, D330G, D330N, D330S, D330V, and L329E.

303. The modified caspase-9 polypeptide of embodiment 300, wherein the at least one amino acid substitution is D330E.

304. The modified caspase-9 polypeptide of embodiment 300 wherein the modified caspase-9 polypeptide further comprises at least one amino acid substitution selected from the group consisting of S196D, F406A, F406W, F406Y, N405Q (codon optimized), D330E, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K.

305. The modified caspase-9 polypeptide of any one of embodiments 300 to 304, wherein the modified caspase-9 polypeptide further comprises at least one amino acid substitution selected from the group consisting of S196D, F406A, F406W, F406Y, N405Q (codon optimized), D330E, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, A331K, T317S, S144A, S144D, N405Q, ⁴⁰²GCFNF⁴⁰⁶ISAQT (Casp-10) (SEQ ID NOS 224 and 225), F404Y, D330A, T317A, S183A, S195A, S196A, F404T, F404W, N405F, F406T, D315A, A316G, F319W, Y153A, Y153F, and S307A.

306. The modified caspase-9 polypeptide of embodiment 300, wherein the modified caspase-9 polypeptide comprises N405Q-S196D or N405Q-D330E amino acid substitutions.

307. A chimeric polypeptide comprising a multimerization region and a modified caspase-9 polypeptide of any one of embodiments 300-306.

308. A nucleic acid comprising a polynucleotide encoding the modified caspase-9 polypeptide of any one of embodiments 300-306.

309. The nucleic acid of embodiment 307, wherein the multimerization region comprises an FKBP12 region.

310. The nucleic acid of embodiment 309, wherein the FKBP12 region has an amino acid substitution at position 36 selected from the group consisting of valine, leucine, isoleuceine and alanine.

311. The nucleic acid of embodiment 309, wherein the FKBP12 region is an FKBP12v36 region.

312. The nucleic acid of any one of embodiments 309-311, wherein the multimerization region comprises Fv′Fvls.

313. The nucleic acid of any one of embodiments 309-312, wherein the multimerization region comprises a polypeptide having an amino acid sequence of SEQ ID NO: 30, or a functional fragment thereof, and a polypeptide having an amino acid sequence of SEQ ID NO: 32, or a functional fragment thereof.

314. The nucleic acid of embodiment 313, wherein the multimerization region further comprises a polypeptide having an amino acid sequence of SEQ ID NO: 30 or 32, or a functional fragment thereof.

315. The nucleic acid of embodiment 313, wherein the multimerization region further comprises an Fv polypeptide variant wherein residue 36 is valine.

316-399. Reserved.

400. A method of administering transduced or transfected donor T cells to a human subject in need thereof, comprising
a) administering transduced or transfected allogeneic donor T cells to the human subject, wherein:
the donor T cells have been transduced or transfected with a nucleic acid comprising
   (i) a promoter region; and
   (ii) a polynucleotide that encodes a chimeric protein comprising a multimeric ligand binding region and a modified caspase-9 polypeptide, wherein
      (1) the promoter region is operatively linked to the polynucleotide;
      (2) the multimeric ligand binding region comprises a FKBP12 polypeptide comprising an amino acid substitution at position 36 selected from the group consisting of valine, isoleucine, leucine, and alanine; and
      (3) the modified caspase-9 polypeptide comprises an amino acid sequence having at least 90% sequence identity to SEQ ID NO: 9, and comprises at least one amino acid substitution selected from the group consisting of D330E, S196D, F406A, F406W, F406Y, N405Q (codon optimized), D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K; and
b) administering a multimeric ligand that binds to the multimeric binding region to the subject.

401. The method of embodiment 400, wherein the donor T cells are not allodepleted from the donor cell culture before transfection or transduction.

402. The method of embodiment 400, wherein the donor T cells are allodepleted from the donor cell culture before transfection or transduction.

403. The method of any one of embodiments 400-402, wherein the cells are allogeneic to the subject.

404. The method of any one of embodiments 400-402, wherein the cells are autologous to the subject.

405. The method of any one of embodiments 400-404, wherein alloreactive donor T cells are present in the subject following (a).

406. The method of any one of embodiments 400-405, wherein the subject exhibits graft versus host disease symptoms following (a).

407. The method of any one of embodiments 405-406, wherein the number of alloreactive donor T cells in the subject is reduced at least 90% within 24 hours following administration of the multimeric ligand.

408. The method of any one of embodiments 400-408, wherein the FKBP12 polypeptide has an amino acid substitution at position 36 selected from the group consisting of valine, leucine, isoleucine and alanine.

409. The method of any one of embodiments 400-408, wherein the FKBP12 polypeptide comprises an amino acid substitution of valine at position 36.

410. The method of any one of embodiments 400-409, wherein the multimeric ligand is AP1903.

411. The method of any one of embodiments 400-410, wherein alloreactive T cells are present in the subject following (a) and alloreactive T cells that are not undergoing cell division are ablated.

412. The method of any one of embodiments 400-411, wherein after administration of the multimeric ligand, donor T cells survive in the subject that are able to expand and are reactive to viruses and fungi.

413. The method of any one of embodiments 400-411, wherein after administration of the multimeric ligand, donor T cells survive in the subject that are able to expand and are reactive to tumor cells in the subject.

414. A method of treating graft versus host disease, comprising administering a multimeric ligand that binds to a multimeric ligand binding region to a human subject who has undergone cell therapy using donor T cells, wherein
a) donor T cells introduced for the therapy express a chimeric protein wherein
   (i) the multimeric ligand binding region comprises a FKBP12 polypeptide comprising an amino acid substitution at position 36 selected from the group consisting of valine, isoleucine, leucine, and alanine; and
   (ii) a modified caspase-9 polypeptide comprising an amino acid sequence having at least 90% sequence identity to SEQ ID NO: 9, and comprising at least one amino acid substitution selected from the group consisting of D330E, S196D, F406A, F406W, F406Y, N405Q (codon optimized), D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K; and
b) following the cell therapy, the subject exhibits graft versus host disease symptoms wherein alloreactive donor T cells are present in the subject.

415. The method of embodiment 414, wherein the number of donor T cells is reduced at least 90% within 24 hours following administration of the multimeric ligand.

416. The method of any one of embodiments 414-415, wherein the donor T cells are autologous to the subject.

417. The method of any one of embodiments 414-415, wherein the donor T cells are allogeneic to the subject.

418. The method of any one of embodiments 414-417, wherein the donor T cells are not allodepleted before the cell therapy.

419. The method of any one of embodiments 414-417, wherein alloreactive donor T cells are present in the subject before administration of the ligand.

420. The method of any one of embodiments 414-417, wherein the subject exhibits graft versus host disease symptoms following the cell therapy.

421. The method of any one of embodiments 400-420, further comprising administering a haploidentical stem cell transplant to the subject.

422. The method of any one of embodiments 400-420, wherein the chimeric protein further comprises a marker polypeptide.

423. The method of embodiment 422, further comprising a selection step, wherein cells that express the marker are selected for administration to the subject.

424. The method of any one of embodiments 400-423, wherein the caspase-9 polypeptide is a truncated caspase-9 polypeptide.

425. The method of any one of embodiments 414-425, wherein alloreactive donor T cells are present in the subject and the number of alloreactive donor T cells in the subject is reduced at least 90% within 30 minutes following administration of the multimeric ligand.

426. The method of any one of embodiments 400-425, wherein the donor T cells are not in an active state when administered to the subject.

427. The method of any one of embodiments 400-426, wherein the donor T cells are quiescent when administered to the subject.

428. The method of any one of embodiments 400-427, wherein the composition of the administered transduced or transfected T cells is over 90% pure.

429. The method of any one of embodiments 400-428, wherein the multimeric ligand binding region comprises two FKBP12 polypeptides, each comprising a valine substitution at position 36.

430. The method of embodiment 429, wherein one of the FKBP12 polypeptides is encoded by a wobbled nucleotide sequence.

431. The method of any one of embodiments 400-430, wherein the multimeric ligand is AP1903.

432. A method of administering a multimeric ligand to a human subject who has undergone cell therapy using modified cells that express a chimeric protein, wherein the chimeric protein comprises
(i) a multimeric ligand binding region comprises a FKBP12 polypeptide comprising an amino acid substitution at position 36 selected from the group consisting of valine, isoleucine, leucine, and alanine; and
(ii) a modified caspase-9 polypeptide comprising an amino acid sequence having at least 90% sequence identity to SEQ ID NO: 9, and comprising at least one amino acid substitution selected from the group consisting of D330E, F406A, F406W, F406Y, N405Q (codon optimized), D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K
   comprising administering the multimeric ligand to the human subject, wherein the multimeric ligand binds to the multimeric ligand binding region.

433. The method of embodiment 432, wherein the number of modified cells in the subject is reduced at least 50% within 24 hours following administration of the multimeric ligand.

434. The method of any one of embodiments 432-433, wherein the modified cells are autologous to the subject.

435. The method of any one of embodiments 432-433, wherein the modified cells are allogeneic to the subject.

436. The method of any one of embodiments 432-435, wherein the multimeric ligand is AP1903.

437. The method of any one of embodiments 432-436, wherein the modified cells comprise a nucleic acid that comprises
a) a promoter;
b) a polynucleotide that encodes the chimeric protein; and
c) a polynucleotide that encodes a heterologous polypeptide selected from the group consisting of chimeric antigen receptor, T cell receptor, and T cell receptor-based chimeric antigen receptor.

438. The method of embodiment 437, wherein the promoter is operably linked to the polynucleotide that encodes the chimeric polypeptide and the polynucleotide that encodes the heterologous polypeptide.

439. The method of any one of embodiments 432-438, wherein the at least one amino acid substitution is selected from the group consisting of S196D, F406A, F406W, F406Y, N405Q (codon optimized), D330E, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, and F319A.

440. The method of any one of embodiments 432-438, wherein the at least one amino acid substitution is selected from the group consisting of S196D, F406A, F406W, F406Y, N405Q (codon optimized), D330E, D330G, D330N, D330S, D330V, and L329E.

441. The method of any one of embodiments 432-440, wherein the at least one amino acid substitution is D330E.

442. The method of any one of embodiments 432-440, wherein the modified caspase-9 polypeptide further comprises at least one amino acid substitution selected from the group consisting of S196D, F406A, F406W, F406Y, N405Q (codon optimized), D330E, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K.

443. The method of any one of embodiments 432 to 442, wherein the modified caspase-9 polypeptide further comprises at least one amino acid substitution selected from the group consisting of S196D, F406A, F406W, F406Y, N405Q (codon optimized), D330E, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, A331K, T317S, S144A, S144D, N405Q, ⁴⁰²GCFNF⁴⁰⁶ISAQT (Casp-10) (SEQ ID NOS 224 and 225), F404Y, D330A, T317A, S183A, S195A, S196A, F404T, F404W, N405F, F406T, D315A, A316G, F319W, Y153A, Y153F, and S307A.

444. The method of any one of embodiments 432-443, wherein the modified caspase-9 polypeptide comprises an N405Q-S196D or N405Q-D330E amino acid substitution.

445. The method of any one of embodiments 432-444, wherein the polynucleotide comprises optimized codons encoding the modified caspase-9 polypeptide.

446. The method of any one of embodiments 432-445, wherein the cell further comprises a polynucleotide that encodes a heterologous polypeptide.

447. The method of embodiment 446, wherein the cell comprises a nucleic acid that comprises
a) the polynucleotide that encodes the chimeric polypeptide; and
b) the polynucleotide that encodes the heterologous polypeptide.

448. The method of embodiment 447, wherein the promoter is operably linked to the polynucleotide that encodes the chimeric polypeptide and the polynucleotide that encodes the heterologous polypeptide.

449. The method of embodiment 447, wherein the promoter is operably linked to the polynucleotide that encodes the chimeric polypeptide and a second promoter is operably linked to the polynucleotide that encodes the heterologous polypeptide.

450. The method of any one of embodiments 447 or 448, wherein the nucleic acid further comprises a polynucleotide encoding a linker polypeptide between the polynucleotide that encodes the chimeric polypeptide and the polynucleotide that encodes the heterologous polypeptide, wherein the linker polypeptide separates the translation products of the first and second polynucleotides during or after translation.

451. The method of embodiment 450, wherein the linker polypeptide is a 2A polypeptide.

452. The method of any one of embodiments 446-451, wherein the heterologous polypeptide is a T cell receptor or a T cell receptor-based chimeric antigen receptor.

453. The method of any one of embodiments 446-451, wherein the heterologous polypeptide is a chimeric antigen receptor.

454. The method of embodiment 453, wherein the chimeric antigen receptor comprises
(i) a transmembrane region; (ii) a T cell activation molecule; and (iii) an antigen recognition moiety.

455. The method of embodiment 454, wherein the T cell activation molecule is an ITAM-containing, Signal 1 conferring molecule.

456. The method of embodiment 454, wherein the T cell activation molecule is a CD3 polypeptide.

457. The method of embodiment 454, wherein the T cell activation molecule is an Fc epsilon receptor gamma (FcεR1γ) subunit polypeptide.

458. The method of any one of embodiments 454-457, wherein the antigen recognition moiety binds to an antigen on a tumor cell.

459. The method of any one of embodiments 454-457, wherein the antigen recognition moiety binds to an antigen on a cell involved in a hyperproliferative disease.

460. The method of any one of embodiments 17-20, wherein the antigen recognition moiety binds to an antigen selected from the group consisting of PSMA, PSCA, MUC1, CD19, ROR1, Mesothelin, GD2, CD123, MUC16, and Her2/Neu.

461. The method of any one of embodiments 454-457, wherein the antigen recognition moiety binds to PSCA.

463. The method of any one of embodiments 432-461, wherein the multimerization region comprises an FKBP12 region.

463. The method of embodiment 463, wherein the FKBP12 region has an amino acid substitution at position 36 selected from the group consisting of valine, leucine, isoleuceine and alanine.

464. The method of embodiment 463, wherein the FKBP12 region is an FKBP12v36 region.

465. The method of any one of embodiments 432-463, wherein the multimerization region comprises Fv′Fvls.

466. The method of any one of embodiments 432-463 wherein the multimerization region comprises a polypeptide having an amino acid sequence of SEQ ID NO: 30, or a functional fragment thereof, and a polypeptide having an amino acid sequence of SEQ ID NO: 32, or a functional fragment thereof.

467. The method of embodiment 466, wherein the multimerization region further comprises a polypeptide having an amino acid sequence of SEQ ID NO: 30 or 32 or a functional fragment thereof.

468. The method of embodiment 466, wherein the multimerization region further comprises an Fv polypeptide variant wherein residue 36 is valine.

469. The method of any one of embodiments 432-468, wherein the multimeric ligand is an FK506 dimer or a dimeric FK506 analog ligand.

470. The method of any one of embodiments 432-469, wherein the ligand is AP1903.

471. The method of any one of embodiments 432-469, wherein the ligand is AP20187.

472. The method of any one of embodiments 432-471, wherein the cell is a progenitor cell.

473. The method of any one of embodiments 432-471, wherein the cell is a hematopoietic progenitor cell.

474. The method of any one of embodiments 432-471, wherein the cell is selected from the group consisting of mesenchymal stromal cells, embryonic stem cells, and inducible pluripotent stem cells.

475. The method of any one of embodiments 432-471, wherein the cell is a T cell.

476. The method of any one of embodiments 432-471, wherein the cell is a T cell, tumor infiltrating lymphocyte, NK-T cell, TCR-expressing cell, or NK cell.

477. The method of any one of embodiments 432-476, wherein the cell is obtained or prepared from bone marrow.

478. The method of any one of embodiments 432-476, wherein the cell is obtained or prepared from umbilical cord blood.

479. The method of any one of embodiments 432-476, wherein the cell is obtained or prepared from peripheral blood.

480. The method of any one of embodiments 432-476, wherein the cell is obtained or prepared from peripheral blood mononuclear cells.

481. The method of any one of embodiments 432-480, wherein the promoter operably linked to the polynucleotide encoding the chimeric polypeptide is developmentally regulated and the caspase-9 polypeptide is expressed in developmentally differentiated cells.

482. The method of any one of embodiments 432-481, wherein the promoter operably linked to the polynucleotide encoding the chimeric polypeptide is tissue-specific and the caspase-9 polypeptide is expressed in the specific tissue.

483. The method of any one of embodiments 432-482, wherein the promoter operably linked to the polynucleotide encoding the chimeric polypeptide is activated in activated T cells.

484. The method of any one of embodiments 432-483, wherein the chimeric polypeptide further comprises a marker polypeptide.

485. The method of embodiment 484, wherein the marker polypeptide is a ΔCD19 polypeptide.

486. The method of any one of embodiments 432-485, wherein the caspase-9 polypeptide is a truncated caspase-9 polypeptide.

487. The method of any one of embodiments 432-486 wherein the caspase-9 polypeptide lacks the Caspase recruitment domain (CARD).

488. The method of any one of embodiments 432-487, wherein the number of therapeutic cells is reduced by about 80% following administration of the multimeric ligand.

489. The method of any one of embodiments 432-487, wherein the number of therapeutic cells is reduced by about 90% following administration of the multimeric ligand.

490. The method of any one of embodiments 432-489, wherein more than one dose of the multimeric ligand is administered.

491. The method of any one of embodiments 432-490, further comprising
identifying a presence or absence of a condition in the subject that requires the removal of transfected or transduced therapeutic cells from the subject; and
administering a multimeric ligand that binds to the multimerization region, maintaining a subsequent dosage of the multimeric ligand, or adjusting a subsequent dosage of the multimeric ligand to the subject based on the presence or absence of the condition identified in the subject.

492. The method of any one of embodiments 432-490 further comprising
identifying a presence or absence of a condition in the subject that requires the removal of transfected or transduced therapeutic cells from the subject; and
determining whether a multimeric ligand that binds to the multimerization region should be administered to the subject, or the dosage of the multimeric ligand subsequently administered to the subject is adjusted based on the presence or absence of the condition identified in the subject.

493. The method of any one of embodiments 432-490, further comprising
receiving information comprising presence or absence of a condition in the subject that requires the removal of transfected or transduced therapeutic cells from the subject; and
administering a multimeric ligand that binds to the multimerization region, maintaining a subsequent dosage of the multimeric ligand, or adjusting a subsequent dosage of the multimeric ligand to the subject based on the presence or absence of the condition identified in the subject.

494. The method of any one of embodiments 432-490, further comprising
identifying a presence or absence of a condition in the subject that requires the removal of transfected or transduced therapeutic cells from the subject; and
transmitting the presence, absence or stage of the condition identified in the subject to a decision maker who administers a multimeric ligand that binds to the multimerization region, maintains a subsequent dosage of the multimeric ligand, or adjusts a subsequent dosage of the multimeric ligand administered to the subject based on the presence, absence or stage of the condition identified in the subject.

495. The method of any one of embodiments 432-490, further comprising
identifying a presence or absence of a condition in the subject that requires the removal of transfected or transduced therapeutic cells from the subject; and
transmitting an indication to administer a multimeric ligand that binds to the multimerization region, maintains a subsequent dosage of the multimeric ligand, or adjusts a subsequent dosage of the multimeric ligand administered to the subject based on the presence, absence or stage of the condition identified in the subject.

496. The method of any one of embodiments 432-495, wherein the therapeutic cells are selected from the group consisting of T cells, tumor infiltrating lymphocytes, NK-T cells, CAR-expressing cells, TCR-expressing cells, and NK cells, further comprising detecting the presence of graft versus host disease or cytokine release syndrome in the subject after administering the therapeutic cells to the subject, and before administering the multimeric ligand.

497. The method of embodiment 496, wherein after administration of the multimeric ligand, the number of therapeutic cells causing graft versus host disease or cytokine release syndrome in the subject cells is reduced.

498. The method of any one of embodiments 432-497, wherein the therapeutic cells are allodepleted before transfection or transduction.

499. The method of any one of embodiments 432-497, wherein the therapeutic cells are not allodepleted before transfection or transduction.

500. The method of any one of embodiments 432-499, wherein the transduced or transfected therapeutic cells are cultured in the presence of IL-2 before administration to the subject.

501. The method of embodiment 500, wherein alloreactive therapeutic cells are present in the subject and the number of alloreactive therapeutic cells in the subject is reduced by at least 90% after administration of the multimeric ligand.

502. The method of any one of embodiments 496-501, wherein, after administration of the multimeric ligand, therapeutic cells survive in the subject that are able to expand and are reactive to viruses and fungi.

503. The method of any one of embodiments 496-501, wherein after administration of the multimeric ligand, therapeutic cells survive in the subject that are able to expand and are reactive to tumor cells in the subject.

504. The method of any one of embodiments 432-502, wherein the subject has received a stem cell transplant before or at the same time as administration of the therapeutic cells.

505. The method of any one of embodiments 491-504, further comprising determining whether to administer an additional dose or additional doses of the multimeric ligand to the subject based upon the appearance of graft versus host disease symptoms in the subject.

506. The method of any one of embodiments 432-505, wherein at least 1×10⁶ transduced or transfected therapeutic cells are administered to the subject.

507. The method of any one of embodiments 432-505, wherein at least 1×10⁷ transduced or transfected donor T cells are administered to the subject.

508. The method of any one of embodiments 432-505, wherein at least 1×10⁸ transduced or transfected donor T cells are administered to the subject.

509. The method of any one of embodiments 432-505, further comprising
identifying the presence, absence or stage of graft versus host disease in the subject, and
administering a multimeric ligand that binds to the multimerization region, maintaining a subsequent dosage of the multimeric ligand, or adjusting a subsequent dosage of the multimeric ligand to the subject based on the presence, absence or stage of the graft versus host disease identified in the subject.

510. A method of stem cell transplantation, comprising
a) administering a stem cell transplant to a human subject; and
b) administering cells of any one of embodiments 200-226 to the subject.

511. The method of embodiment 510, wherein the stem cell transplant is selected from the group consisting of a matched transplant, a partially-matched transplant, a haploidentical transplant, and aCD34⁺ haploidentical stem cell transplant.

512. The method of any one of embodiments 510-511 wherein the cells are donor T cells that are matched, partially matched, or haploidentical to the subject's T cells.

513. A method of reducing the effect of graft versus host disease in a human subject following administration of a cell of any one of embodiments 200-226, wherein the cell is a donor T cell, comprising:
a) detecting the presence or absence of graft versus host disease in the subject after administration of the donor T cells; and
b) administering a multimeric ligand that binds to the multimerization region to a subject for whom the presence of graft versus host disease is detected.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1. A method for controlling the survival of therapeutic cells in a subject, comprising administering to a subject in need thereof
a) modified cells comprising a promoter operably linked to a polynucleotide that encodes a chimeric polypeptide comprising
i. a multimerization region and
ii. a modified caspase-9 polypeptide comprising an amino acid sequence having at least 90% sequence identity to SEQ ID NO: 9, and comprises at least one amino acid substitution selected from the group consisting of D330E, S196D, F406A, F406W, F406Y, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K; and
b) an amount of a multimeric ligand that binds to the multimerization region effective to reduce the number of cells administered in (a).

2. The method of claim 1, wherein the modified caspase-9 polypeptide further comprises at least one amino acid substitution selected from the group consisting of D330E, S196D, F406A, F406W, F406Y, N405Q (codon optimized), D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, A331K, T317S, S144A, S144D, N405Q, ⁴⁰²GCFNF⁴⁰⁶ISAQT (Casp-10) (SEQ ID NOS 224 and 225), F404Y, D330A, T317A, S183A, S195A, S196A, F404T, F404W, N405F, F406T, D315A, A316G, F319W, Y153A, Y153F, and S307A.

3. The method of claim 1, wherein the cell further comprises a polynucleotide that encodes a heterologous polypeptide selected from the group consisting of chimeric antigen receptor, T cell receptor, and T cell receptor-based chimeric antigen receptor.

4. The method of claim 3, wherein the promoter is operably linked to the polynucleotide that encodes the chimeric polypeptide and the polynucleotide that encodes the heterologous polypeptide.

5. The method of claim 4, wherein the nucleic acid further comprises a polynucleotide encoding a linker polypeptide between the polynucleotide that encodes the chimeric polypeptide and the polynucleotide that encodes the heterologous polypeptide, wherein the linker polypeptide separates the translation products of the first and second polynucleotides during or after translation.

6. The method of claim 1, wherein the multimerization region comprises an FKBP12 region.

7. The method of claim 6, wherein the ligand is AP1903.

8. The method of claim 1, wherein the cell is a human cell.

9. The method of claim 1, wherein the number of therapeutic cells is reduced by about 50% following administration of the multimeric ligand.

10. The method of claim 1, wherein the therapeutic cells are selected from the group consisting of T cells, tumor infiltrating lymphocytes, NK-T cells, CAR-expressing cells, TCR-expressing cells, and NK cells, further comprising detecting the presence of graft versus host disease or cytokine release syndrome in the subject after administering the therapeutic cells to the subject, and before administering the multimeric ligand.

11. The method of claim 10, wherein after administration of the multimeric ligand, the number of therapeutic cells causing graft versus host disease or cytokine release syndrome in the subject cells is reduced.

12. A nucleic acid comprising a promoter operably linked to a polynucleotide that encodes a chimeric polypeptide, wherein the chimeric polypeptide comprises: a multimerization region and a modified caspase-9 polypeptide comprising
an amino acid sequence having at least 90% sequence identity to SEQ ID NO: 9, and
at least one amino acid substitution selected from the group consisting of D330E, S196D, F406A, F406W, F406Y, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K.

13. A nucleic acid comprising a promoter operably linked to
a) a first polynucleotide that encodes a chimeric polypeptide, wherein the chimeric polypeptide comprises: a multimerization region and a modified caspase-9 polypeptide comprising an amino acid sequence having at least 90% sequence identity to SEQ ID NO: 9, and
at least one amino acid substitution selected from the group consisting of D330E, S196D, F406A, F406W, F406Y, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K; and
b) a second polynucleotide that encodes a heterologous polypeptide selected from the group consisting of chimeric antigen receptor, T cell receptor, and T cell receptor-based chimeric antigen receptor.

14. A nucleic acid comprising a promoter operably linked to
a) a first polynucleotide that encodes a chimeric polypeptide, wherein the chimeric polypeptide comprises: a multimerization region and a modified caspase-9 polypeptide comprising
an amino acid sequence having at least 90% sequence identity to SEQ ID NO: 9, and
at least one amino acid substitution selected from the group consisting of D330E, S196D, F406A, F406W, F406Y, N405Q (codon optimized), D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, A331K, T317S, S144A, S144D, N405Q, ⁴⁰²GCFNF⁴⁰⁶ISAQT (Casp-10) (SEQ ID NOS 224 and 225), F404Y, D330A, T317A, S183A, S195A, S196A, F404T, F404W, N405F, F406T, D315A, A316G, F319W, Y153A, Y153F, and S307A; and
b) a second polynucleotide that encodes a heterologous polypeptide selected from the group consisting of chimeric antigen receptor, T cell receptor, and T cell receptor-based chimeric antigen receptor.

15. The nucleic acid of claim 12, wherein the modified caspase-9 polypeptide further comprises at least one amino acid substitution selected from the group consisting of S196D, F406A, F406W, F406Y, N405Q (codon optimized), D330E, D330G, D330N, D330S, D330V, L329E, C403A, C403S, C403T, N405A, 316ATPF319AVPI (SMAC/Diablo) (SEQ ID NOS 226 and 227), T317C, P318A, F319A, N405T, T317E, F326K, D327G, D327K, D327R, Q328K, Q328R, L329G, L329K, A331K, T317S, S144A, S144D, N405Q, 402GCFNF406ISAQT (Casp-10) (SEQ ID NOS 224 and 225), F404Y, D330A, T317A, S183A, S195A, S196A, F404T, F404W, N405F, F406T, D315A, A316G, F319W, Y153A, Y153F, and S307A.

16. The nucleic acid of claim 14, wherein the nucleic acid further comprises a polynucleotide encoding a linker polypeptide between the first and second polynucleotides, wherein the linker polypeptide separates the translation products of the first and second polynucleotides during or after translation.

17. The nucleic acid claim 12, wherein the multimerization region comprises an FKBP12 region.

18. A modified cell comprising a nucleic acid of claim 12.

19. The cell of claim 18, wherein the cell is a human cell.

20. The cell of claim 18, wherein the cell is a T cell, tumor infiltrating lymphocyte, NK-T cell, TCR-expressing cell, or NK cell.

21. The cell of claim 18, wherein the modified cell is an autologous cell.

22. The cell of claim 18, wherein the modified cell is an allogeneic cell.

23. A modified cell comprising a nucleic acid of claim 14.

24. The cell of claim 18, further comprising a nucleic acid that encodes a heterologous polypeptide selected from the group consisting of chimeric antigen receptor, T cell receptor, and T cell receptor-based chimeric antigen receptor.

25. A modified caspase-9 polypeptide, comprising an amino acid sequence having at least 90% sequence identity to SEQ ID NO: 9, and comprising at least one amino acid substitution selected from the group consisting of D330E, S196D, F406A, F406W, F406Y, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K.

26. The modified caspase-9 polypeptide of claim 25 wherein the modified caspase-9 polypeptide further comprises at least one amino acid substitution selected from the group consisting of S196D, F406A, F406W, F406Y, D330E, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K.

27. The modified caspase-9 polypeptide of claim 25, wherein the modified caspase-9 polypeptide further comprises at least one amino acid substitution selected from the group consisting of S196D, F406A, F406W, F406Y, N405Q (codon optimized), D330E, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, A331K, T317S, S144A, S144D, N405Q, ⁴⁰²GCFNF⁴⁰⁶ISAQT (Casp-10) (SEQ ID NOS 224 and 225), F404Y, D330A, T317A, S183A, S195A, S196A, F404T, F404W, N405F, F406T, D315A, A316G, F319W, Y153A, Y153F, and S307A.

28. A chimeric polypeptide comprising a multimerization region and a modified caspase-9 polypeptide of claim 25.

29. A method of administering transduced or transfected donor T cells to a human subject in need thereof, comprising
a) administering transduced or transfected allogeneic donor T cells to the human subject, wherein:
the donor T cells have been transduced or transfected with a nucleic acid comprising
(i) a promoter region; and
(ii) a polynucleotide that encodes a chimeric protein comprising a multimeric ligand binding region and a modified caspase-9 polypeptide, wherein
(1) the promoter region is operatively linked to the polynucleotide,
(2) the multimeric ligand binding region comprises a FKBP12 polypeptide comprising an amino acid substitution at position 36 selected from the group consisting of valine, isoleucine, leucine, and alanine; and
(3) the modified caspase-9 polypeptide comprises an amino acid sequence having at least 90% sequence identity to SEQ ID NO: 9, and comprises at least one amino acid substitution selected from the group consisting of D330E, S196D, F406A, F406W, F406Y, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K; and
b) administering a multimeric ligand that binds to the multimeric binding region to the subject.

30. The method of claim 29, wherein the cells are allogeneic to the subject.

31. The method of claim 29, wherein the cells are autologous to the subject.

32. The method of claim 29, wherein alloreactive donor T cells are present in the subject following (a).

33. The method of claim 32, wherein the number of alloreactive donor T cells is reduced at least 90% within 24 hours following administration of the multimeric ligand.

34. A method of treating graft versus host disease, comprising administering a multimeric ligand that binds to a multimeric ligand binding region to a human subject who has undergone cell therapy using donor T cells, wherein
a) donor T cells introduced for the therapy express a chimeric protein comprising
(i) the multimeric ligand binding region comprises a FKBP12 polypeptide comprising an amino acid substitution at position 36 selected from the group consisting of valine, isoleucine, leucine, and alanine; and
(ii) a modified caspase-9 polypeptide comprising an amino acid sequence having at least 90% sequence identity to SEQ ID NO: 9, and comprising at least one amino acid substitution selected from the group consisting of D330E, S196D, F406A, F406W, F406Y, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K; and
b) following the cell therapy, the subject exhibits graft versus host disease symptoms wherein alloreactive donor T cells are present.

35. The method of claim 34, wherein the number of donor T cells is reduced at least 90% within 24 hours following administration of the multimeric ligand.

36. The method of claim 34, wherein alloreactive donor T cells are present in the subject and the number of alloreactive donor T cells is reduced at least 90% within 30 minutes following administration of the multimeric ligand.

37. A method of stem cell transplantation, comprising
a) administering a stem cell transplant to a human subject; and
b) administering a cell of claim 18 to the subject.

38. A method of administering a multimeric ligand to a human subject who has undergone cell therapy using modified cells that express a chimeric protein, wherein the chimeric protein comprises
(i) a multimeric ligand binding region comprises a FKBP12 polypeptide comprising an amino acid substitution at position 36 selected from the group consisting of valine, isoleucine, leucine, and alanine; and
(ii) a modified caspase-9 polypeptide comprising an amino acid sequence having at least 90% sequence identity to SEQ ID NO: 9, and comprising at least one amino acid substitution selected from the group consisting of D330E, S196D, F406A, F406W, F406Y, D330G, D330N, D330S, D330V, L329E, C403A, C403S, C403T, N405A, ³¹⁶ATPF³¹⁹AVPI (SMAC/Diablo) (SEQ ID NOS 226 and 227), T317C, P318A, F319A, N405T, T317E, F326K, D327G, D327K, D327R, Q328K, Q328R, L329G, L329K, and A331K
comprising administering the multimeric ligand to the human subject, wherein the multimeric ligand binds to the multimeric ligand binding region.

39. The method of claim 38, wherein the number of modified cells in the subject is reduced at least 50% within 24 hours following administration of the multimeric ligand.

40. The method of claim 38, wherein the modified cells are autologous to the subject.

41. The method of claim 38, wherein the modified cells are allogeneic to the subject.

42. The method of claim 38, wherein the multimeric ligand is AP1903.

43. The method of claim 38, wherein the modified cells comprise a nucleic acid that comprises
a) a promoter;
b) a polynucleotide that encodes the chimeric protein; and
c) a polynucleotide that encodes a heterologous polypeptide selected from the group consisting of chimeric antigen receptor, T cell receptor, and T cell receptor-based chimeric antigen receptor.

44. The method of claim 43, wherein the promoter is operably linked to the polynucleotide that encodes the chimeric polypeptide and the polynucleotide that encodes the heterologous polypeptide.


SEQUENCE LISTING (259 sequences)

SEQ ID NO: 1 - DNA, 590 bp, Artificial Sequence
  Description of Artificial Sequence Synthetic polynucleotide
tgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 2 - DNA, 321 bp, Homo sapiens
ggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 3 - protein, 107 aa, Homo sapiens
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 4 - DNA, 18 bp, Artificial Sequence
  Description of Artificial Sequence Synthetic oligonucleotide
tctggcggtggatccgga

SEQ ID NO: 5 - protein, 6 aa, Artificial Sequence
  Description of Artificial Sequence Synthetic peptide
SGGGSG

SEQ ID NO: 6 - DNA, 6 bp, Artificial Sequence
  Description of Artificial Sequence Synthetic oligonucleotide
gtcgac

SEQ ID NO: 7 - protein, 2 aa, Artificial Sequence
  Description of Artificial Sequence Synthetic peptide
VD

SEQ ID NO: 8 - DNA, 846 bp, Artificial Sequence
  Description of Artificial Sequence Synthetic polynucleotide
ggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 9 - protein, 282 aa, Artificial Sequence
  Description of Artificial Sequence Synthetic polypeptide
GFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 10 - DNA, 9 bp, Artificial Sequence
  Description of Artificial Sequence Synthetic oligonucleotide
gctagcaga

SEQ ID NO: 11 - protein, 3 aa, Artificial Sequence
  Description of Artificial Sequence Synthetic peptide
ASR

SEQ ID NO: 12 - DNA, 57 bp, Thosea asigna virus
gccgagggcaggggaagtcttctaacatgcggggacgtggaggaaaatcccgggccc

SEQ ID NO: 13 - protein, 19 aa, Thosea asigna virus
AEGRGSLLTCGDVEENPGP

SEQ ID NO: 14 - DNA, 999 bp, Homo sapiens
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 15 - protein, 333 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 16 - DNA, 590 bp, Artificial Sequence
  Description of Artificial Sequence Synthetic polynucleotide
tgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 17 - DNA, 8622 bp, Artificial Sequence
  Description of Artificial Sequence Synthetic polynucleotide
tgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagccgagggcaggggaagtcttctaacatgcggggacgtggaggaaaatcccgggcccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattctaacgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaa

SEQ ID NO: 18 - DNA, 678 bp, Artificial Sequence
  Description of Artificial Sequence Synthetic polynucleotide
ctcgagggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaagtcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgag

SEQ ID NO: 19 - protein, 222 aa, Artificial Sequence
  Description of Artificial Sequence Synthetic polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSG

SEQ ID NO: 20 - DNA, 1200 bp, Artificial Sequence
  Description of Artificial Sequence Synthetic polynucleotide
atgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 21 - protein, 107 aa, Homo sapiens; identical to SEQ ID NO: 3

SEQ ID NO: 22 - protein, 282 aa, Artificial Sequence
  Description of Artificial Sequence Synthetic polypeptide
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 23 - DNA, 846 bp, Artificial Sequence
  Description of Artificial Sequence Synthetic polynucleotide
ggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggccgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 24 - protein, 282 aa, Artificial Sequence
  Description of Artificial Sequence Synthetic polypeptide
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 25 - DNA, 846 bp, Artificial Sequence
  Description of Artificial Sequence Synthetic polynucleotide
ggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttcagttcctccggaaaaaacttttctttaaaacatca

SEQ ID NO: 26 - protein, 282 aa, Artificial Sequence
  Description of Artificial Sequence Synthetic polypeptide
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFQFLRKKLFFKTS

SEQ ID NO: 27 - DNA, 846 bp, Artificial Sequence
  Description of Artificial Sequence Synthetic polynucleotide
ggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggccgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttcagttcctccggaaaaaacttttctttaaaacatca

SEQ ID NO: 28 - protein, 282 aa, Artificial Sequence
  Description of Artificial Sequence Synthetic polypeptide
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFQFLRKKLFFKTS

SEQ ID NO: 29 - DNA, 321 bp, Artificial Sequence
  Description of Artificial Sequence Synthetic polynucleotide
ggcgttcaagtagaaacaatcagcccaggagacggaaggactttccccaaacgaggccaaacatgcgtagttcattatactgggatgctcgaagatggaaaaaaagtagatagtagtagagaccgaaacaaaccatttaaatttatgttgggaaaacaagaagtaataaggggctgggaagaaggtgtagcacaaatgtctgttggccagcgcgcaaaactcacaatttctcctgattatgcttacggagctaccggccaccccggcatcataccccctcatgccacactggtgtttgacgtcgaattgctcaaactggaa

SEQ ID NO: 30 - protein, 107 aa, Artificial Sequence; identical to SEQ ID NO: 3
  Description of Artificial Sequence Synthetic polypeptide

SEQ ID NO: 31 - DNA, 321 bp, Artificial Sequence
  Description of Artificial Sequence Synthetic polynucleotide
ggagtgcaggtggagacgattagtcctggggatgggagaacctttccaaagcgcggtcagacctgtgttgtccactacaccggtatgctggaggacgggaagaaggtggactcttcacgcgatcgcaataagcctttcaagttcatgctcggcaagcaggaggtgatccgggggtgggaggagggcgtggctcagatgtcggtcgggcaacgagcgaagcttaccatctcacccgactacgcgtatggggcaacggggcatccgggaattatccctccccacgctacgctcgtattcgatgtggagctcttgaagcttgag

SEQ ID NO: 32 - protein, 107 aa, Artificial Sequence; identical to SEQ ID NO: 3
  Description of Artificial Sequence Synthetic polypeptide

SEQ ID NO: 33 - DNA, 1002 bp, Artificial Sequence
  Description of Artificial Sequence Synthetic polynucleotide
atgccccctcctagactgctgtttttcctgctctttctcaccccaatggaagttagacctgaggaaccactggtcgttaaagtggaagaaggtgataatgctgtcctccaatgccttaaagggaccagcgacggaccaacgcagcaactgacttggagccgggagtcccctctcaagccgtttctcaagctgtcacttggcctgccaggtcttggtattcacatgcgcccccttgccatttggctcttcatattcaatgtgtctcaacaaatgggtggattctacctttgccagcccggccccccttctgagaaagcttggcagcctggatggaccgtcaatgttgaaggctccggtgagctgtttagatggaatgtgagcgaccttggcggactcggttgcggactgaaaaataggagctctgaaggaccctcttctccctccggtaagttgatgtcacctaagctgtacgtgtgggccaaggaccgccccgaaatctgggagggcgagcctccatgcctgccgcctcgcgattcactgaaccagtctctgtcccaggatctcactatggcgcccggatctactctttggctgtcttgcggcgttcccccagatagcgtgtcaagaggacctctgagctggacccacgtacaccctaagggccctaagagcttgttgagcctggaactgaaggacgacagacccgcacgcgatatgtgggtaatggagaccggccttctgctccctcgcgctaccgcacaggatgcagggaaatactactgtcatagagggaatctgactatgagctttcatctcgaaattacagcacggcccgttctttggcattggctcctccggactggaggctggaaggtgtctgccgtaacactcgcttacttgattttttgcctgtgtagcctggttgggatcctgcatcttcagcgagcccttgtattgcgccgaaaaagaaaacgaatgactgaccctacacgacgattctga

SEQ ID NO: 34 - protein, 333 aa, Artificial Sequence; identical to SEQ ID NO: 15
  Description of Artificial Sequence Synthetic polypeptide

SEQ ID NO: 35 - DNA, 330 bp, Artificial Sequence
  Description of Artificial Sequence Synthetic polynucleotide
atgctggagggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaa

SEQ ID NO: 36 - protein, 110 aa, Artificial Sequence
  Description of Artificial Sequence Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 37 - protein, 18 aa, Artificial Sequence
  Description of Artificial Sequence Synthetic peptide
AGCGGAGGAGGATCCGGA

SEQ ID NO: 38 - protein, 6 aa, Artificial Sequence
  Description of Artificial Sequence Synthetic peptide
SGGGSG

SEQ ID NO: 39 - DNA, 864 bp, Artificial Sequence
  Description of Artificial Sequence Synthetic polynucleotide
gtggacgggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttccagtttctgagaaagaaactgttctttaagacctccgcatctagggcc

SEQ ID NO: 40 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFQFLRKKLFFKTSASRA

SEQ ID NO: 41 - DNA, 6 bp, Artificial Sequence
  Description of Artificial Sequence Synthetic oligonucleotide
ccgcgg

SEQ ID NO: 42 - protein, 2 aa, Artificial Sequence
  Description of Artificial Sequence Synthetic peptide
PR

SEQ ID NO: 43 - protein, 18 aa, Artificial Sequence
  Description of Artificial Sequence Synthetic peptide
EGRGSLLTCGDVEENPGP

SEQ ID NO: 44 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 45 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 46 - DNA, 864 bp, Artificial Sequence
  Description of Artificial Sequence Synthetic polynucleotide
gtggacgggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttcaactttctgagaaagaaactgttctttaagacctccgcatctagggcc

SEQ ID NO: 47 - protein, 288 aa, Artificial Sequence; identical to SEQ ID NO: 45
  Description of Artificial Sequence Synthetic polypeptide

SEQ ID NO: 48 - DNA, 864 bp, Artificial Sequence
  Description of Artificial Sequence Synthetic polynucleotide
gtcgacggatttggtgatgtcggtgctcttgaggctttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 49 - protein, 288 aa, Artificial Sequence
  Description of Artificial Sequence Synthetic polypeptide
VDGFGDVGALEALRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 50 - DNA, 864 bp, Artificial Sequence
  Description of Artificial Sequence Synthetic polynucleotide
gtcgacggatttggtgatgtcggtgctcttgaggacttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 51 - protein, 288 aa, Artificial Sequence
  Description of Artificial Sequence Synthetic polypeptide
VDGFGDVGALEDLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 52 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggcgccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 53 - protein, 288 aa, Artificial Sequence
  Description of Artificial Sequence Synthetic polypeptide
VDGFGDVGALESLRGNADLAYILSMEPCGHCLIINNVNFCRESGLRTRTGANIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 54 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctccgcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 55 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSALHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 56 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctccgacctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 57 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSDLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 58 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggccgcgggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 59 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQAAGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 60 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatggcaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 61 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDGTPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 62 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccgccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 63 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDAAPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 64 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgcctgcccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 65 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDACPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 66 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgcctccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 67 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDASPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 68 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccaaggaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 69 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTKDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 70 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcaagcagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 71 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFKQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 72 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcaggcagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 73 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFRQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 74 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcggccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 75 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFGQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 76 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgacaagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 77 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDKLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 78 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgacaggctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 79 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDRLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 80 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagaaggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 81 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQKDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 82 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccaggaggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 83 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQEDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 84 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagggcgacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 85 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQGDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 86 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggccgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 87 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 88 - DNA, 864 bp, Artificial Sequence; identical to SEQ ID NO: 86
  Description of Artificial Sequence Synthetic polynucleotide

SEQ ID NO: 89 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLEAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 90 - DNA, 864 bp, Artificial Sequence; identical to SEQ ID NO: 86
  Description of Artificial Sequence Synthetic polynucleotide

SEQ ID NO: 91 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLNAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 92 - DNA, 864 bp, Artificial Sequence; identical to SEQ ID NO: 86
  Description of Artificial Sequence Synthetic polynucleotide

SEQ ID NO: 93 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLVAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 94 - DNA, 864 bp, Artificial Sequence; identical to SEQ ID NO: 86
  Description of Artificial Sequence Synthetic polynucleotide

SEQ ID NO: 95 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLGAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 96 - DNA, 864 bp, Artificial Sequence; identical to SEQ ID NO: 86
  Description of Artificial Sequence Synthetic polynucleotide

SEQ ID NO: 97 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLSAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 98 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacaagatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 99 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDKISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 100 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctataatttcctccggaaaaaacttttctttaaaacatcagctagcagagcc

SEQ ID NO: 101 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCYNFLRKKLFFKTSASRA

SEQ ID NO: 102 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctggaatttcctccggaaaaaacttttctttaaaacatcagctagcagagcc

SEQ ID NO: 103 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCWNFLRKKLFFKTSASRA

SEQ ID NO: 104 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttcagttcctccggaaaaaacttttctttaaaacatcagctagcagagcc

SEQ ID NO: 105 - protein, 288 aa, Artificial Sequence; identical to SEQ ID NO: 40
  Description of Artificial Sequence Synthetic polypeptide

SEQ ID NO: 106 - DNA, 864 bp, Artificial Sequence; identical to SEQ ID NO: 39
  Description of Artificial Sequence Synthetic polynucleotide

SEQ ID NO: 107 - protein, 288 aa, Artificial Sequence; identical to SEQ ID NO: 40
  Description of Artificial Sequence Synthetic polypeptide

SEQ ID NO: 108 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatctcctccggaaaaaacttttctttaaaacatcagctagcagagcc

SEQ ID NO: 109 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNLLRKKLFFKTSASRA

SEQ ID NO: 110 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaattccctccggaaaaaacttttctttaaaacatcagctagcagagcc

SEQ ID NO: 111 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNTLRKKLFFKTSASRA

SEQ ID NO: 112 - DNA, 864 bp, Artificial Sequence
  Description of Artificial Sequence Synthetic polynucleotide
gtggacgggtttggagatgtgggagccctggaagccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttccagtttctgagaaagaaactgttctttaagacctccgcatctagggcc

SEQ ID NO: 113 - protein, 288 aa, Artificial Sequence
  Description of Artificial Sequence Synthetic polypeptide
VDGFGDVGALEALRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFQFLRKKLFFKTSASRA

SEQ ID NO: 114 - DNA, 864 bp, Artificial Sequence
  Description of Artificial Sequence Synthetic polynucleotide
gtcgacggatttggtgatgtcggtgctcttgaggctttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggccgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 115 - protein, 288 aa, Artificial Sequence
  Description of Artificial Sequence Synthetic polypeptide
VDGFGDVGALEALRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 116 - DNA, 864 bp, Artificial Sequence
  Description of Artificial Sequence Synthetic polynucleotide
gtcgacggatttggtgatgtcggtgctcttgaggacttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggccgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 117 - protein, 288 aa, Artificial Sequence
  Description of Artificial Sequence Synthetic polypeptide
VDGFGDVGALEDLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 118 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctccgcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggccgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 119 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSALHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 120 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctccgacctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggccgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 121 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSDLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 122 - DNA, 864 bp, Artificial Sequence
  Description of Artificial Sequence Synthetic polynucleotide
gtggacgggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaagccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttccagtttctgagaaagaaactgttctttaagacctccgcatctagggcc

SEQ ID NO: 123 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDASPFQEGLRTFDQLDAISSLPTPSDIFVSYSTFPGFVSWRDPKSGSWYVETLDDIFEQWAHSEDLQSLLLRVANAVSVKGIYKQMPGCFQFLRKKLFFKTSASRA

SEQ ID NO: 124 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggccgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgcttcagtttcctccggaaaaaacttttctttaaaacatcagctagcagagcc

SEQ ID NO: 125 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFQFLRKKLFFKTSASRA

SEQ ID NO: 126 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccgtgcccatccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 127 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDAVPIQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 128 - DNA, 864 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgccgatatccgcacagacactccggaaaaaacttttctttaaaacatcagctagcagagcc

SEQ ID NO: 129 - protein, 288 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPISAQTLRKKLFFKTSASRA

SEQ ID NO: 130 - DNA, 330 bp, Artificial Sequence; identical to SEQ ID NO: 35
  Description of Artificial Sequence Synthetic polynucleotide

SEQ ID NO: 131 - protein, 110 aa, Artificial Sequence; identical to SEQ ID NO: 36
  Description of Artificial Sequence Synthetic polypeptide

SEQ ID NO: 132 - DNA, 18 bp, Artificial Sequence
  Description of Artificial Sequence Synthetic oligonucleotide
agcggaggaggatccgga

SEQ ID NO: 133 - protein, 6 aa, Artificial Sequence
  Description of Artificial Sequence Synthetic peptide
SGGGSG

SEQ ID NO: 134 - DNA, 864 bp, Artificial Sequence; identical to SEQ ID NO: 46
  Description of Artificial Sequence Synthetic polynucleotide

SEQ ID NO: 135 - protein, 288 aa, Artificial Sequence; identical to SEQ ID NO: 45
  Description of Artificial Sequence Synthetic polypeptide

SEQ ID NO: 136 - DNA, 6 bp, Artificial Sequence
  Description of Artificial Sequence Synthetic oligonucleotide
ccgcgg

SEQ ID NO: 137 - protein, 2 aa, Artificial Sequence
  Description of Artificial Sequence Synthetic peptide
PR

SEQ ID NO: 138 - DNA, 54 bp, Artificial Sequence
  Description of Artificial Sequence Synthetic oligonucleotide
gaaggccgagggagcctgctgacatgtggcgatgtggaggaaaacccaggacca

SEQ ID NO: 139 - protein, 18 aa, Artificial Sequence
  Description of Artificial Sequence Synthetic peptide
EGRGSLLTCGDVEENPGP

SEQ ID NO: 140 - DNA, 6 bp, Artificial Sequence
  Description of Artificial Sequence Synthetic oligonucleotide
ccatgg

SEQ ID NO: 141 - protein, 2 aa, Artificial Sequence
  Description of Artificial Sequence Synthetic peptide
PW

SEQ ID NO: 142 - DNA, 63 bp, Artificial Sequence
  Description of Artificial Sequence Synthetic oligonucleotide
atggagtttggactttcttggttgtttttggtggcaattctgaagggtgtccagtgtagcagg

SEQ ID NO: 143 - protein, 21 aa, Artificial Sequence
  Description of Artificial Sequence Synthetic peptide
MEFGLSWLFLVAILKGVQCSR

SEQ ID NO: 144 - DNA, 321 bp, Artificial Sequence
  Description of Artificial Sequence Synthetic polynucleotide
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 145 - protein, 107 aa, Artificial Sequence
  Description of Artificial Sequence Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 146 - DNA, 24 bp, Artificial Sequence
  Description of Artificial Sequence Synthetic oligonucleotide
ggcggaggaagcggaggtgggggc

SEQ ID NO: 147 - protein, 8 aa, Artificial Sequence
  Description of Artificial Sequence Synthetic peptide
GGGSGGGG

SEQ ID NO: 148 - DNA, 360 bp, Artificial Sequence
  Description of Artificial Sequence Synthetic polynucleotide
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 149 - protein, 120 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 150 - DNA, 6 bp, Artificial Sequence
  Description of Artificial Sequence Synthetic oligonucleotide
ggatcc

SEQ ID NO: 151 - protein, 2 aa, Artificial Sequence
  Description of Artificial Sequence Synthetic peptide
GS

SEQ ID NO: 152 - DNA, 48 bp, Artificial Sequence
  Description of Artificial Sequence Synthetic oligonucleotide
gaacttcctactcaggggactttctcaaacgttagcacaaacgtaagt

SEQ ID NO: 153 - protein, 16 aa, Artificial Sequence
  Description of Artificial Sequence Synthetic peptide
ELPTQGTFSNVSTNVS

SEQ ID NO: 154 - DNA, 126 bp, Artificial Sequence
  Description of Artificial Sequence Synthetic polynucleotide
cccgccccaagaccccccacacctgcgccgaccattgcttctcaacccctgagtttgagacccgaggcctgccggccagctgccggcggggccgtgcatacaagaggactcgatttcgcttgcgac

SEQ ID NO: 155 - protein, 42 aa, Artificial Sequence
  Description of Artificial Sequence Synthetic polypeptide
PAPRPPTPAPTIASQPLSLRPEACRPAAGGAVHTRGLDFACD

SEQ ID NO: 156 - DNA, 111 bp, Artificial Sequence
  Description of Artificial Sequence Synthetic polynucleotide
atctatatctgggcacctctcgctggcacctgtggagtccttctgctcagcctggttattactctgtactgtaatcaccggaatcgccgccgcgtttgtaagtgtcccagg

SEQ ID NO: 157 - protein, 37 aa, Artificial Sequence
  Description of Artificial Sequence Synthetic polypeptide
IYIWAPLAGTCGVLLLSLVITLYCNHRNRRRVCKCPR

SEQ ID NO: 158 - DNA, 6 bp, Artificial Sequence
  Description of Artificial Sequence Synthetic oligonucleotide
gtcgac

SEQ ID NO: 159 - protein, 2 aa, Artificial Sequence
  Description of Artificial Sequence Synthetic peptide
VD

SEQ ID NO: 160 - DNA, 336 bp, Artificial Sequence
  Description of Artificial Sequence Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 161 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - DNA, 63 bp, Artificial Sequence; identical to SEQ ID NO: 142
  Description of Artificial Sequence Synthetic oligonucleotide

SEQ ID NO: 163 - protein, 21 aa, Artificial Sequence
  Description of Artificial Sequence Synthetic peptide
MEFGLSWLFLVAILKGVQCSR

SEQ ID NO: 164 - DNA, 327 bp, Artificial Sequence
  Description of Artificial Sequence Synthetic polynucleotide
gacatccaattgacacaatcacacaaatttctctcaacttctgtaggagacagagtgagcataacctgcaaagcatcccaggacgtgtacaatgctgtggcttggtaccaacagaagcctggacaatccccaaaattgctgatttattctgcctctagtaggtacactggggtaccttctcggtttacgggctctgggtccggaccagatttcacgttcacaatcagttccgttcaagctgaagacctcgctgtttatttttgccagcagcacttccgaaccccttttacttttggctcaggcactaagttggaaatcaaggctttg

SEQ ID NO: 165 - protein, 109 aa, Artificial Sequence
  Description of Artificial Sequence Synthetic polypeptide
DIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKAL

SEQ ID NO: 166 - DNA, 24 bp, Artificial Sequence
  Description of Artificial Sequence Synthetic oligonucleotide
ggcggaggaagcggaggtgggggc

SEQ ID NO: 167 - protein, 8 aa, Artificial Sequence
  Description of Artificial Sequence Synthetic peptide
GGGSGGGG

SEQ ID NO: 168 - DNA, 357 bp, Artificial Sequence
  Description of Artificial Sequence Synthetic polynucleotide
gaagtccaattgcaacagtcaggccccgaattgaaaaagcccggcgaaacagtgaagatatcttgtaaagcctccggttacccttttacgaactatggaatgaactgggtcaaacaagcccctggacagggattgaagtggatgggatggatcaatacatcaacaggcgagtctaccttcgcagatgatttcaaaggtcgctttgacttctcactggagaccagtgcaaataccgcctaccttcagattaacaatcttaaaagcgaggatatggcaacctacttttgcgcaagatgggaagtttatcacgggtacgtgccatactggggacaaggaacgacagtgacagttagtagc

SEQ ID NO: 169 - protein, 119 aa, Artificial Sequence
  Description of Artificial Sequence Synthetic polypeptide
EVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSS

SEQ ID NO: 170 - DNA, 6 bp, Artificial Sequence
  Description of Artificial Sequence Synthetic oligonucleotide
ggatcc

SEQ ID NO: 171 - protein, 2 aa, Artificial Sequence
  Description of Artificial Sequence Synthetic peptide
GS

SEQ ID NO: 172 - DNA, 48 bp, Artificial Sequence; identical to SEQ ID NO: 152
  Description of Artificial Sequence Synthetic oligonucleotide

SEQ ID NO: 173 - protein, 16 aa, Artificial Sequence
  Description of Artificial Sequence Synthetic peptide
ELPTQGTFSNVSTNVS

SEQ ID NO: 174 - DNA, 126 bp, Artificial Sequence; identical to SEQ ID NO: 154
  Description of Artificial Sequence Synthetic polynucleotide

SEQ ID NO: 175 - protein, 42 aa, Artificial Sequence; identical to SEQ ID NO: 155
  Description of Artificial Sequence Synthetic polypeptide

SEQ ID NO: 176 - DNA, 111 bp, Artificial Sequence; identical to SEQ ID NO: 156
  Description of Artificial Sequence Synthetic polynucleotide

SEQ ID NO: 177 - protein, 37 aa, Artificial Sequence; identical to SEQ ID NO: 157
  Description of Artificial Sequence Synthetic polypeptide

SEQ ID NO: 178 - DNA, 6 bp, Artificial Sequence
  Description of Artificial Sequence Synthetic oligonucleotide
ctcgag

SEQ ID NO: 179 - protein, 2 aa, Artificial Sequence
  Description of Artificial Sequence Synthetic peptide
LE

SEQ ID NO: 180 - DNA, 336 bp, Artificial Sequence; identical to SEQ ID NO: 160
  Description of Artificial Sequence Synthetic polynucleotide

SEQ ID NO: 181 - protein, 112 aa, Artificial Sequence; identical to SEQ ID NO: 161
  Description of Artificial Sequence Synthetic polypeptide

SEQ ID NO: 182 - DNA, 204 bp, Artificial Sequence
  Description of Artificial Sequence Synthetic polynucleotide
ttctgggtactggttgtagtcggtggcgtacttgcttgttattctcttcttgttaccgtagccttcattatattctgggtccgatcaaagcgctcaagactcctccattccgattatatgaacatgacacctcgccgacctggtcctacacgcaaacattatcaaccctacgcacccccccgagacttcgctgcttatcgatcc

SEQ ID NO: 183 - protein, 68 aa, Artificial Sequence
  Description of Artificial Sequence Synthetic polypeptide
FWVLVVVGGVLACYSLLVTVAFIIFWVRSKRSRLLHSDYMNMTPRRPGPTRKHYQPYAPPRDFAAYRS

SEQ ID NO: 184 - DNA, 186 bp, Artificial Sequence
  Description of Artificial Sequence Synthetic polynucleotide
gttgccgccatcctgggcctgggcctggtgctggggctgctgggccccctggccatcctgctggccctgtacctgctccgggaccagaggctgccccccgatgcccacaagccccctgggggaggcagtttccggacccccatccaagaggagcaggccgacgcccactccaccctggccaagatc

SEQ ID NO: 185 - protein, 63 aa, Artificial Sequence
  Description of Artificial Sequence Synthetic polypeptide
VAAILGLGLVLGLLGPLAILLALYLLRRDQRLPPDAHKPPGGGSFRTPIQEEQADAHSTLAKI

SEQ ID NO: 186 - DNA, 135 bp, Artificial Sequence
  Description of Artificial Sequence Synthetic polynucleotide
agtgtagttaaaagaggaagaaaaaagttgctgtatatatttaaacaaccatttatgagaccagtgcaaaccacccaagaagaagacggatgttcatgcagattcccagaagaagaagaaggaggatgtgaattg

SEQ ID NO: 187 - protein, 45 aa, Artificial Sequence
  Description of Artificial Sequence Synthetic polypeptide
SVVKRGRKKLLYIFKQPFMRPVQTTQEEDGCSCRFPEEEEGGCEL

SEQ ID NO: 188 - DNA, 9 bp, Artificial Sequence
  Description of Artificial Sequence Synthetic oligonucleotide
atgctcgag

SEQ ID NO: 189 - protein, 3 aa, Artificial Sequence
  Description of Artificial Sequence Synthetic peptide
MLE

SEQ ID NO: 190 - DNA, 320 bp, Artificial Sequence
  Description of Artificial Sequence Synthetic polynucleotide
ggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactgga

SEQ ID NO: 191 - protein, 106 aa, Artificial Sequence
  Description of Artificial Sequence Synthetic polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKL

SEQ ID NO: 192 - DNA, 19 bp, Artificial Sequence
  Description of Artificial Sequence Synthetic oligonucleotide
atctggcggtggatccgga

SEQ ID NO: 193 - protein, 6 aa, Artificial Sequence
  Description of Artificial Sequence Synthetic peptide
SGGGSG

SEQ ID NO: 194 - DNA, 921 bp, Artificial Sequence
  Description of Artificial Sequence Synthetic polynucleotide
gtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagccgagggcaggggaagtcttctaacatgcggggacgtggaggaaaatcccgggccctga

SEQ ID NO: 195 - protein, 284 aa, Artificial Sequence
  Description of Artificial Sequence Synthetic polypeptide
VDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 196 - DNA, 18 bp, Artificial Sequence
  Description of Artificial Sequence Synthetic oligonucleotide
gctagcagagccccgcgg

SEQ ID NO: 197 - protein, 6 aa, Artificial Sequence
  Description of Artificial Sequence Synthetic peptide
ASRAPR

SEQ ID NO: 198 - DNA, 54 bp, Artificial Sequence
  Description of Artificial Sequence Synthetic oligonucleotide
gagggcaggggaagtcttctaacatgcggggacgtggaggaaaatcccgggccc

SEQ ID NO: 199 - protein, 18 aa, Artificial Sequence
  Description of Artificial Sequence Synthetic peptide
EGRGSLLTCGDVEENPGP

SEQ ID NO: 200 - DNA, 12 bp, Artificial Sequence
  Description of Artificial Sequence Synthetic oligonucleotide
gcatgcgccacc

SEQ ID NO: 201 - protein, 4 aa, Artificial Sequence
  Description of Artificial Sequence Synthetic peptide
ACAT

SEQ ID NO: 202 - DNA, 63 bp, Artificial Sequence
  Description of Artificial Sequence Synthetic oligonucleotide
atggagtttgggttgtcatggttgtttctcgtcgctattctcaaaggtgtacaatgctcccgc

SEQ ID NO: 203 - protein, 21 aa, Artificial Sequence
  Description of Artificial Sequence Synthetic peptide
MEFGLSWLFLVAILKGVQCSR

SEQ ID NO: 204 - DNA, 357 bp, Artificial Sequence; identical to SEQ ID NO: 168
  Description of Artificial Sequence Synthetic polynucleotide

SEQ ID NO: 205 - protein, 119 aa, Artificial Sequence; identical to SEQ ID NO: 169
  Description of Artificial Sequence Synthetic polypeptide

SEQ ID NO: 206 - DNA, 45 bp, Artificial Sequence
  Description of Artificial Sequence Synthetic oligonucleotide
ggcggtggaggctccggtggaggcggctctggaggaggaggttca

SEQ ID NO: 207 - protein, 15 aa, Artificial Sequence
  Description of Artificial Sequence Synthetic peptide
GGGGSGGGGSGGGGS

SEQ ID NO: 208 - DNA, 327 bp, Artificial Sequence; identical to SEQ ID NO: 164
  Description of Artificial Sequence Synthetic polynucleotide

SEQ ID NO: 209 - protein, 109 aa, Artificial Sequence; identical to SEQ ID NO: 165
  Description of Artificial Sequence Synthetic polypeptide

SEQ ID NO: 210 - DNA, 6 bp, Artificial Sequence
  Description of Artificial Sequence Synthetic oligonucleotide
atgcat

SEQ ID NO: 211 - protein, 2 aa, Artificial Sequence
  Description of Artificial Sequence Synthetic peptide
MH

SEQ ID NO: 212 - DNA, 48 bp, Artificial Sequence; identical to SEQ ID NO: 152
  Description of Artificial Sequence Synthetic oligonucleotide

SEQ ID NO: 213 - protein, 16 aa, Artificial Sequence
  Description of Artificial Sequence Synthetic peptide
ELPTQGTFSNVSTNVS

SEQ ID NO: 214 - DNA, 126 bp, Artificial Sequence; identical to SEQ ID NO: 154
  Description of Artificial Sequence Synthetic polynucleotide

SEQ ID NO: 215 - protein, 42 aa, Artificial Sequence; identical to SEQ ID NO: 155
  Description of Artificial Sequence Synthetic polypeptide

SEQ ID NO: 216 - DNA, 117 bp, Artificial Sequence
  Description of Artificial Sequence Synthetic polynucleotide
atctatatctgggcacctctcgctggcacctgtggagtccttctgctcagcctggttattactctgtactgtaatcaccggaatcgccgccgcgtttgtaagtgtcccagggtcgac

SEQ ID NO: 217 - protein, 39 aa, Artificial Sequence
  Description of Artificial Sequence Synthetic polypeptide
IYIWAPLAGTCGVLLLSLVITLYCNHRNRRRVCKCPRVD

SEQ ID NO: 218 - DNA, 516 bp, Artificial Sequence
  Description of Artificial Sequence Synthetic polynucleotide
atggccgctgggggcccaggcgccggatcagctgctcccgtatcttctacttcttctttgccgctggctgctctgaacatgcgcgtgagaagacgcctctccctgttccttaacgttcgcacacaagtcgctgccgattggaccgcccttgccgaagaaatggactttgaatacctggaaattagacaacttgaaacacaggccgaccccactggcagactcctggacgcatggcagggaagacctggtgcaagcgttggacggctcctggatctcctgacaaaactgggacgcgacgacgtactgcttgaactcggacctagcattgaagaagactgccaaaaatatatcctgaaacaacaacaagaagaagccgaaaaacctctccaagtcgcagcagtggactcatcagtaccccgaacagctgagcttgctgggattactacactcgacgacccactcggacatatgcctgaaagattcgacgctttcatttgctattgcccctctgacata

SEQ ID NO: 219 - protein, 172 aa, Artificial Sequence
  Description of Artificial Sequence Synthetic polypeptide
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 220 - DNA, 186 bp, Artificial Sequence
  Description of Artificial Sequence Synthetic polynucleotide
aagaaagttgcaaagaaacccacaaataaagccccacaccctaaacaggaaccccaagaaatcaatttcccagatgatctccctggatctaatactgccgccccggtccaagaaaccctgcatggttgccagcctgtcacccaagaggacggaaaagaatcacggattagcgtacaagagagacaa

SEQ ID NO: 221 - protein, 62 aa, Artificial Sequence
  Description of Artificial Sequence Synthetic polypeptide
KKVAKKPTNKAPHPKQEPQEINFPDDLPGSNTAAPVQETLHGCQPVTQEDGKESRISVQERQ

SEQ ID NO: 222 - DNA, 339 bp, Artificial Sequence
  Description of Artificial Sequence Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgttga

SEQ ID NO: 223 - protein, 112 aa, Artificial Sequence; identical to SEQ ID NO: 161
  Description of Artificial Sequence Synthetic polypeptide

SEQ ID NO: 224 - protein, 5 aa, Homo sapiens
GCFNF

SEQ ID NO: 225 - protein, 5 aa, Unknown
  Description of Unknown Caspase-10 peptide
ISAQT

SEQ ID NO: 226 - protein, 4 aa, Homo sapiens
ATPF

SEQ ID NO: 227 - protein, 4 aa, Unknown
  Description of Unknown Caspase peptide
AVPI

SEQ ID NO: 228 - protein, 5 aa, Unknown
  Description of Unknown Caspase-3 peptide
CIVSM

SEQ ID NO: 229 - protein, 5 aa, Unknown
  Description of Unknown Caspase peptide
AAAAA

SEQ ID NO: 230 - protein, 5 aa, Unknown
  Description of Unknown Caspase-2 peptide
YCSTL

SEQ ID NO: 231 - protein, 5 aa, Unknown
  Description of Unknown Caspase-8 peptide
QPTFT

SEQ ID NO: 232 - protein, 8 aa, Artificial Sequence
  Description of Artificial Sequence Synthetic peptide
RAKFKQLL

SEQ ID NO: 233 - protein, 4 aa, Unknown
  Description of Unknown Caspase-9 peptide
DAPF

SEQ ID NO: 234 - protein, 5 aa, Unknown
  Description of Unknown Caspase-9 peptide
DAISS

SEQ ID NO: 235 - protein, 6 aa, Artificial Sequence
  Description of Artificial Sequence Synthetic peptide
GSGGGS

SEQ ID NO: 236 - protein, 5 aa, Artificial Sequence
  Description of Artificial Sequence Synthetic peptide
SGGGS

SEQ ID NO: 237 - protein, 7 aa, Homo sapiens
TDPTRRF

SEQ ID NO: 238 - protein, 9 aa, Artificial Sequence
  Description of Artificial Sequence Synthetic peptide
NLVPMVATV

SEQ ID NO: 239 - protein, 6 aa, Artificial Sequence
  Description of Artificial Sequence Synthetic peptide
SGGGSG

SEQ ID NO: 240 - DNA, 18 bp, Artificial Sequence
  Description of Artificial Sequence Synthetic primer
tccgccctgagcaaagac

SEQ ID NO: 241 - DNA, 20 bp, Artificial Sequence
  Description of Artificial Sequence Synthetic primer
acgaactccagcaggaccat

SEQ ID NO: 242 - DNA, 15 bp, Artificial Sequence
  Description of Artificial Sequence Synthetic probe
acgagaagcgcgatc

SEQ ID NO: 243 - DNA, 19 bp, Artificial Sequence
  Description of Artificial Sequence Synthetic primer
ctggaatctggcggtggat

SEQ ID NO: 244 - DNA, 23 bp, Artificial Sequence
  Description of Artificial Sequence Synthetic primer
caaactctcaagagcaccgacat

SEQ ID NO: 245 - DNA, 15 bp, Artificial Sequence
  Description of Artificial Sequence Synthetic probe
cggagtcgacggatt

SEQ ID NO: 246 - DNA, 54 bp, Artificial Sequence; identical to SEQ ID NO: 138
  Description of Artificial Sequence Synthetic oligonucleotide

SEQ ID NO: 247 - DNA, 1002 bp, Artificial Sequence
  Description of Artificial Sequence Synthetic polynucleotide
atgccaccacctcgcctgctgttctttctgctgttcctgacacctatggaggtgcgacctgaggaaccactggtcgtgaaggtcgaggaaggcgacaatgccgtgctgcagtgcctgaaaggcacttctgatgggccaactcagcagctgacctggtccagggagtctcccctgaagccttttctgaaactgagcctgggactgccaggactgggaatccacatgcgccctctggctatctggctgttcatcttcaacgtgagccagcagatgggaggattctacctgtgccagccaggaccaccatccgagaaggcctggcagcctggatggaccgtcaacgtggaggggtctggagaactgtttaggtggaatgtgagtgacctgggaggactgggatgtgggctgaagaaccgctcctctgaaggcccaagttcaccctcagggaagctgatgagcccaaaactgtacgtgtgggccaaagatcggcccgagatctgggagggagaacctccatgcctgccacctagagacagcctgaatcagagtctgtcacaggatctgacaatggcccccgggtccactctgtggctgtcttgtggagtcccacccgacagcgtgtccagaggccctctgtcctggacccacgtgcatcctaaggggccaaaaagtctgctgtcactggaactgaaggacgatcggcctgccagagacatgtgggtcatggagactggactgctgctgccacgagcaaccgcacaggatgctggaaaatactattgccaccggggcaatctgacaatgtccttccatctggagatcactgcaaggcccgtgctgtggcactggctgctgcgaaccggaggatggaaggtcagtgctgtgacactggcatatctgatcttttgcctgtgctccctggtgggcattctgcatctgcagagagccctggtgctgcggagaaagagaaagagaatgactgacccaacaagaaggttttga

SEQ ID NO: 248 - protein, 333 aa, Artificial Sequence; identical to SEQ ID NO: 15
  Description of Artificial Sequence Synthetic polypeptide

SEQ ID NO: 249 - DNA, 329 bp, Artificial Sequence
  Description of Artificial Sequence Synthetic polynucleotide
atgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactgga

SEQ ID NO: 250 - DNA, 18 bp, Artificial Sequence
  Description of Artificial Sequence Synthetic oligonucleotide
tctggcggtggatccgga

SEQ ID NO: 251 - DNA, 54 bp, Artificial Sequence; identical to SEQ ID NO: 198
  Description of Artificial Sequence Synthetic oligonucleotide

SEQ ID NO: 252 - protein, 110 aa, Artificial Sequence; identical to SEQ ID NO: 36
  Description of Artificial Sequence Synthetic polypeptide

SEQ ID NO: 253 - protein, 6 aa, Artificial Sequence
  Description of Artificial Sequence Synthetic peptide
SGGGSG

SEQ ID NO: 254 - protein, 18 aa, Artificial Sequence
  Description of Artificial Sequence Synthetic peptide
EGRGSLLTCGDVEENPGP

SEQ ID NO: 255 - DNA, 321 bp, Artificial Sequence
  Description of Artificial Sequence Synthetic polynucleotide
ggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaa

SEQ ID NO: 256 - DNA, 18 bp, Artificial Sequence
  Description of Artificial Sequence Synthetic oligonucleotide
agcggaggaggatccgga

SEQ ID NO: 257 - DNA, 60 bp, Artificial Sequence
  Description of Artificial Sequence Synthetic oligonucleotide
ccgcgggaaggccgagggagcctgctgacatgtggcgatgtggaggaaaacccaggacca

SEQ ID NO: 258 - protein, 18 aa, Artificial Sequence
  Description of Artificial Sequence Synthetic peptide
EGRGSLLTCGDVEENPGP

SEQ ID NO: 259 - DNA, 6 bp, Artificial Sequence
  Description of Artificial Sequence Synthetic oligonucleotide
gccacc